SEQUENCE LISTING (51 sequences)

SEQ ID NO: 1 - DNA, 2944 bp, Homo sapiens
ctcaccctctctggtcgcccctccccggattcccccaccctccgtgcctgcaggagcccctgggctttcccggaggagctcgccctgaagggcccggacctcggcgagcccaccaccgttccctccagcgccgccgccgccaccgcagcagccggagcagcatggtccagctgaggaagctgctccgcgtcctgactttgatgaagttcccctgctgcgtgctggaggtgctcctgtgcgcgctggcggcggcggcgcgcggccaggagatgtacgccccgcactcaatccggatcgagggggacgtcaccctcggggggctgttccccgtgcacgccaagggtcccagcggagtgccctgcggcgacatcaagagggaaaacgggatccacaggctggaagcgatgctctacgccctggaccagatcaacagtgatcccaacctactgcccaacgtgacgctgggcgcgcggatcctggacacttgttccagggacacttacgcgctcgaacagtcgcttactttcgtccaggcgctcatccagaaggacacctccgacgtgcgctgcaccaacggcgaaccgccggttttcgtcaagccggagaaagtagttggagtgattggggcttcggggagttcggtctccatcatggtagccaacatcctgaggctcttccagatcccccagattagttatgcatcaacggcacccgagctaagtgatgaccggcgctatgacttcttctctcgcgtggtgccacccgattccttccaagcccaggccatggtagacattgtaaaggccctaggctggaattatgtgtctaccctcgcatcggaaggaagttatggagagaaaggtgtggagtccttcacgcagatttccaaagaggcaggtggactctgcattgcccagtccgtgagaatcccccaggaacgcaaagacaggaccattgactttgatagaattatcaaacagctcctggacacccccaactccagggccgtcgtgatttttgccaacgatgaggatataaagcagatccttgcagcagccaaaagagctgaccaagttggccattttctttgggtgggatcagacagctggggatccaaaataaacccactgcaccagcatgaagatatcgcagaaggggccatcaccattcagcccaagcgagccacggtggaagggtttgatgcctactttacgtcccgtacacttgaaaacaacagaagaaatgtatggtttgccgaatactgggaggaaaacttcaactgcaagttgacgattagtgggtcaaaaaaagaagacacagatcgcaaatgcacaggacaggagagaattggaaaagattccaactatgagcaggagggtaaagtccagttcgtgattgacgcagtctatgctatggctcacgcccttcaccacatgaacaaggatctctgtgctgactaccggggtgtctgcccagagatggagcaagctggaggcaagaagttgctgaagtatatacgcaatgttaatttcaatggtagtgctggcactccagtgatgtttaacaagaacggggatgcacctgggcgttatgacatctttcagtaccagaccacaaacaccagcaacccgggttaccgtctgatcgggcagtggacagacgaacttcagctcaatatagaagacatgcagtggggtaaaggagtccgagagatacccgcctcagtgtgcacactaccatgtaagccaggacagagaaagaagacacagaaaggaactccttgctgttggacctgtgagccttgcgatggttaccagtaccagtttgatgagatgacatgccagcattgcccctatgaccagaggcccaatgaaaatcgaaccggatgccaggatattcccatcatcaaactggagtggcactccccctgggctgtgattcctgtcttcctggcaatgttggggatcattgccaccatctttgtcatggccactttcatccgctacaatgacacgcccattgtccgggcatctgggcgggaactcagctatgttcttttgacgggcatctttctttgctacatcatcactttcctgatgattgccaaaccagatgtggcagtgtgttctttccggcgagttttcttgggcttgggtatgtgcatcagttatgcagccctcttgacgaaaacaaatcggatttatcgcatatttgagcagggcaagaaatcagtaacagctcccagactcataagcccaacatcacaactggcaatcacttccagtttaatatcagttcagcttctaggggtgttcatttggtttggtgttgatccacccaacatcatcatagactacgatgaacacaagacaatgaaccctgagcaagccagaggggttctcaagtgtgacattacagatctccaaatcatttgctccttgggatatagcattcttctcatggtcacatgtactgtgtatgccatcaagactcggggtgtacccgagaattttaacgaagccaagcccattggattcactatgtacacgacatgtatagtatggcttgccttcattccaattttttttggcaccgctcaatcagcggaaaagctctacatacaaactaccacgcttacaatctccatgaacctaagtgcatcagtggcgctggggatgctatacatgccgaaagtgtacatcatcattttccaccctgaactcaatgtccagaaacggaagcgaagcttcaaggcggtagtcacagcagccaccatgtcatcgaggctgtcacacaaacccagtgacagacccaacggtgaggcaaagaccgagctctgtgaaaacgtagacccaaacagccctgctgcaaaaaagaagtatgtcagttataataacctggttatctaacctgttccattccatggaaccatggaggaggaaga

SEQ ID NO: 2 - protein, 915 aa, Homo sapiens
MVQLRKLLRVLTLMKFPCCVLEVLLCALAAAARGQEMYAPHSIRIEGDVTLGGLFPVHAKGPSGVPCGDIKRENGIHRLEAMLYALDQINSDPNLLPNVTLGARILDTCSRDTYALEQSLTFVQALIQKDTSDVRCTNGEPPVFVKPEKVVGVIGASGSSVSIMVANILRLFQIPQISYASTAPELSDDRRYDFFSRVVPPDSFQAQAMVDIVKALGWNYVSTLASEGSYGEKGVESFTQISKEAGGLCIAQSVRIPQERKDRTIDFDRIIKQLLDTPNSRAVVIFANDEDIKQILAAAKRADQVGHFLWVGSDSWGSKINPLHQHEDIAEGAITIQPKRATVEGFDAYFTSRTLENNRRNVWFAEYWEENFNCKLTISGSKKEDTDRKCTGQERIGKDSNYEQEGKVQFVIDAVYAMAHALHHMNKDLCADYRGVCPEMEQAGGKKLLKYIRNVNFNGSAGTPVMFNKNGDAPGRYDIFQYQTTNTSNPGYRLIGQWTDELQLNIEDMQWGKGVREIPASVCTLPCKPGQRKKTQKGTPCCWTCEPCDGYQYQFDEMTCQHCPYDQRPNENRTGCQDIPIIKLEWHSPWAVIPVFLAMLGIIATIFVMATFIRYNDTPIVRASGRELSYVLLTGIFLCYIITFLMIAKPDVAVCSFRRVFLGLGMCISYAALLTKTNRIYRIFEQGKKSVTAPRLISPTSQLAITSSLISVQLLGVFIWFGVDPPNIIIDYDEHKTMNPEQARGVLKCDITDLQIICSLGYSILLMVTCTVYAIKTRGVPENFNEAKPIGFTMYTTCIVWLAFIPIFFGTAQSAEKLYIQTTTLTISMNLSASVALGMLYMPKVYIIIFHPELNVQKRKRSFKAVVTAATMSSRLSHKPSDRPNGEAKTELCENVDPNSPAAKKKYVSYNNLVI

SEQ ID NO: 3 - DNA, 2811 bp, Homo sapiens
ccagaaggtgcagcctcaggtggtgccctttcttctgtggcaagaataaactttgggtcttggattgcaataccacctgtggagaaaatggtatgcgagggaaagcgatcagcctcttgcccttgtttcttcctcttgaccgccaagttctactggatcctcacaatgatgcaaagaactcacagccaggagtatgcccattccatacgggtggatggggacattattttggggggtctcttccctgtccacgcaaagggagagagaggggtgccttgtggggagctgaagaaggaaaaggggattcacagactggaggccatgctttatgcaattgaccagattaacaaggaccctgatctcctttccaacatcactctgggtgtccgcatcctcgacacgtgctctagggacacctatgctttggagcagtctctaacattcgtgcaggcattaatagagaaagatgcttcggatgtgaagtgtgctaatggagatccacccattttcaccaagcccgacaagatttctggcgtcataggtgctgcagcaagctccgtgtccatcatggttgctaacattttaagactttttaagatacctcaaatcagctatgcatccacagccccagagctaagtgataacaccaggtatgactttttctctcgagtggttccgcctgactcctaccaagcccaagccatggtggacatcgtgacagcactgggatggaattatgtttcgacactggcttctgaggggaactatggtgagagcggtgtggaggccttcacccagatctcgagggagattggtggtgtttgcattgctcagtcacagaaaatcccacgtgaaccaagacctggagaatttgaaaaaattatcaaacgcctgctagaaacacctaatgctcgagcagtgattatgtttgccaatgaggatgacatcaggaggatattggaagcagcaaaaaaactaaaccaaagtgggcattttctctggattggctcagatagttggggatccaaaatagcacctgtctatcagcaagaggagattgcagaaggggctgtgacaattttgcccaaacgagcatcaattgatggatttgatcgatactttagaagccgaactcttgccaataatcgaagaaatgtgtggtttgcagaattctgggaggagaattttggctgcaagttaggatcacatgggaaaaggaacagtcatataaagaaatgcacagggctggagcgaattgctcgggattcatcttatgaacaggaaggaaaggtccaatttgtaattgatgctgtatattccatggcttacgccctgcacaatatgcacaaagatctctgccctggatacattggcctttgtccacgaatgagtaccattgatgggaaagagctacttggttatattcgggctgtaaattttaatggcagtgctggcactcctgtcacttttaatgaaaacggagatgctcctggacgttatgatatcttccagtatcaaataaccaacaaaagcacagagtacaaagtcatcggccactggaccaatcagcttcatctaaaagtggaagacatgcagtgggctcatagagaacatactcacccggcgtctgtctgcagcctgccgtgtaagccaggggagaggaagaaaacggtgaaaggggtcccttgctgctggcactgtgaacgctgtgaaggttacaactaccaggtggatgagctgtcctgtgaactttgccctctggatcagagacccaacatgaaccgcacaggctgccagcttatccccatcatcaaattggagtggcattctccctgggctgtggtgcctgtgtttgttgcaatattgggaatcatcgccaccacctttgtgatcgtgacctttgtccgctataatgacacacctatcgtgagggcttcaggacgcgaacttagttacgtgctcctaacggggatttttctctgttattcaatcacgtttttaatgattgcagcaccagatacaatcatatgctccttccgacgggtcttcctaggacttggcatgtgtttcagctatgcagcccttctgaccaaaacaaaccgtatccaccgaatatttgagcaggggaagaaatctgtcacagcgcccaagttcattagtccagcatctcagctggtgatcaccttcagcctcatctccgtccagctccttggagtgtttgtctggtttgttgtggatcccccccacatcatcattgactatggagagcagcggacactagatccagagaaggccaggggagtgctcaagtgtgacatttctgatctctcactcatttgttcacttggatacagtatcctcttgatggtcacttgtactgtttatgccattaaaacgagaggtgtcccagagactttcaatgaagccaaacctattggatttaccatgtataccacctgcatcatttggttagctttcatccccatcttttttggtacagcccagtcagcagaaaagatgtacatccagacaacaacacttactgtctccatgagtttaagtgcttcagtatctctgggcatgctctatatgcccaaggtttatattataatttttcatccagaacagaatgttcaaaaacgcaagaggagcttcaaggctgtggtgacagctgccaccatgcaaagcaaactgatccaaaaaggaaatgacagaccaaatggcgaggtgaaaagtgaactctgtgagagtcttgaaaccaacagtaagtcatctgtagagtttccgatggtcaagagcgggagcacttcc

SEQ ID NO: 4 - protein, 908 aa, Homo sapiens
MVCEGKRSASCPCFFLLTAKFYWILTMMQRTHSQEYAHSIRVDGDIILGGLFPVHAKGERGVPCGELKKEKGIHRLEAMLYAIDQINKDPDLLSNITLGVRILDTCSRDTYALEQSLTFVQALIEKDASDVKCANGDPPIFTKPDKISGVIGAAASSVSIMVANILRLFKIPQISYASTAPELSDNTRYDFFSRVVPPDSYQAQAMVDIVTALGWNYVSTLASEGNYGESGVEAFTQISREIGGVCIAQSQKIPREPRPGEFEKIIKRLLETPNARAVIMFANEDDIRRILEAAKKLNQSGHFLWIGSDSWGSKIAPVYQQEEIAEGAVTILPKRASIDGFDRYFRSRTLANNRRNVWFAEFWEENFGCKLGSHGKRNSHIKKCTGLERIARDSSYEQEGKVQFVIDAVYSMAYALHNMHKDLCPGYIGLCPRMSTIDGKELLGYIRAVNFNGSAGTPVTFNENGDAPGRYDIFQYQITNKSTEYKVIGHWTNQLHLKVEDMQWAHREHTHPASVCSLPCKPGERKKTVKGVPCCWHCERCEGYNYQVDELSCELCPLDQRPNMNRTGCQLIPIIKLEWHSPWAVVPVFVAILGIIATTFVIVTFVRYNDTPIVRASGRELSYVLLTGIFLCYSITFLMIAAPDTIICSFRRVFLGLGMCFSYAALLTKTNRIHRIFEQGKKSVTAPKFISPASQLVITFSLISVQLLGVFVWFVVDPPHIIIDYGEQRTLDPEKARGVLKCDISDLSLICSLGYSILLMVTCTVYAIKTRGVPETFNEAKPIGFTMYTTCIIWLAFIPIFFGTAQSAEKMYIQTTTLTVSMSLSASVSLGMLYMPKVYIIIFHPEQNVQKRKRSFKAVVTAATMQSKLIQKGNDRPNGEVKSELCESLETNSKSSVEFPMVKSGSTS

SEQ ID NO: 5 - DNA, 2823 bp, Artificial Sequence
  Description of Artificial Sequence Synthetic chimeric construct
ccagaaggtgcagcctcaggtggtgccctttcttctgtggcaagaataaactttgggtcttggattgcaataccacctgtggagaaaatggtatgcgagggaaagcgatcagcctcttgcccttgtttcttcctcttgaccgccaagttctactggatcctcacaatgatgcaaagaactcacagccaggagtatgcccattccatacggatcgagggggacgtcaccctcggggggctgttccccgtgcacgccaagggtcccagcggagtgccctgcggcgacatcaagagggaaaatgggatccacaggctggaagcgatgctctacgccctggaccagatcaacagtgatcccaacctactgcccaacgtgacgctgggcgcgcggatcctggacacttgttccagggacacttacgcgctcgaacagtcgcttactttcgtccaggcgctcatccagaaggacacctccgacgtgcgctgcaccaacggcgaaccgccggttttcgtcaagccggagaaagtagttggagtgattggggcttcggggagttcggtctccatcatggtagccaacatcctgaggctcttccagatcccccagattagttatgcatcaacggcacccgagctaagtgatgaccggcgctatgacttcttctctcgcgtggtgccacccgattccttccaagcccaggccatggtagacattgtaaaggccctaggctggaattatgtgtctaccctcgcatcggaaggaagttatggagagaaaggtgtggagtccttcacgcagatttccaaagaggcaggtggactctgcattgcccagtccgtgagaatcccccaggaacgcaaagacaggaccattgactttgatagaattatcaaacagctcctggacacccccaactccagggccgtcgtgatttttgccaacgatgaggatataaagcagatccttgcagcagccaaaagagctgaccaagttggccattttctttgggtgggatcagacagctggggatccaaaataaacccactgcaccagcatgaagatatcgcagaaggggccatcaccattcagcccaagcgagccacggtggaagggtttgatgcctactttacgtcccgtacacttgaaaacaacagaagaaatgtatggtttgccgaatactgggaggaaaacttcaactgcaagttgacgattagtgggtcaaaaaaagaagacacagatcgcaaatgcacaggacaggagagaattggaaaagattccaactatgagcaggagggtaaagtccagttcgtgattgacgcagtctatgctatggctcacgcccttcaccacatgaacaaggatctctgtgctgactaccggggtgtctgcccagagatggagcaagctggaggcaagaagttgctgaagtatatacgcaatgttaatttcaatggtagtgctggcactccagtgatgtttaacaagaacggggatgcacctgggcgttatgacatctttcagtaccagaccacaaacaccagcaacccgggttaccgtctgatcgggcagtggacagacgaacttcagctcaatatagaagacatgcagtggggtaaaggagtccgagagatacccgcctcagtgtgcacactaccatgtaagccaggacagagaaagaagacacagaaaggaactccttgctgttggacctgtgaaccttgcgatggttaccagtaccagtttgatgagatgacatgccagcattgcccctatgaccagaggcccaatgaaaatcgaaccggatgccaggatattcccatcatcaaactggagtggcactccccctgggctgtgattcctgtcttcctggcaatgttggggatcattgccaccatctttgtcatggccactttcatccgctacaatgacacgcccattgtccgggcatctgggcgggaactcagctatgttcttttgacgggcatctttctttgctacatcatcactttcctgatgattgccaaaccagatgtggcagtgtgttctttccggcgagttttcttgggcttgggtatgtgcatcagttatgcagccctcttgacgaaaacaaatcggatttatcgcatatttgagcagggcaagaaatcagtaacagctcccagactcataagcccaacatcacaactggcaatcacttccagtttaatatcagttcagcttctaggggtgttcatttggtttggtgttgatccacccaacatcatcatagactacgatgaacacaagacaatgaaccctgagcaagccagaggggttctcaagtgtgacattacagatctccaaatcatttgctccttgggatatagcattcttctcatggtcacatgtactgtgtatgccatcaagactcggggtgtacccgagaattttaacgaagccaagcccattggattcactatgtacacgacatgtatagtatggcttgccttcattccaattttttttggcaccgctcaatcagcggaaaagctctacatacaaactaccacgcttacaatctccatgaacctaagtgcatcagtggcgctggggatgctatacatgccgaaagtgtacatcatcattttccaccctgaactcaatgtccagaaacggaagcgaagcttcaaggcggtagtcacagcagccaccatgtcatcgaggctgtcacacaaacccagtgacagacccaacggtgaggcaaagaccgagctctgtgaaaacgtagacccaaacagccctgctgcaaaaaagaagtatgtcagttataataacctggttatc

SEQ ID NO: 6 - protein, 912 aa, Artificial Sequence
  Description of Artificial Sequence Synthetic chimeric construct
MVCEGKRSASCPCFFLLTAKFYWILTMMQRTHSQEYAHSIRIEGDVTLGGLFPVHAKGPSGVPCGDIKRENGIHRLEAMLYALDQINSDPNLLPNVTLGARILDTCSRDTYALEQSLTFVQALIQKDTSDVRCTNGEPPVFVKPEKVVGVIGASGSSVSIMVANILRLFQIPQISYASTAPELSDDRRYDFFSRVVPPDSFQAQAMVDIVKALGWNYVSTLASEGSYGEKGVESFTQISKEAGGLCIAQSVRIPQERKDRTIDFDRIIKQLLDTPNSRAVVIFANDEDIKQILAAAKRADQVGHFLWVGSDSWGSKINPLHQHEDIAEGAITIQPKRATVEGFDAYFTSRTLENNRRNVWFAEYWEENFNCKLTISGSKKEDTDRKCTGQERIGKDSNYEQEGKVQFVIDAVYAMAHALHHMNKDLCADYRGVCPEMEQAGGKKLLKYIRNVNFNGSAGTPVMFNKNGDAPGRYDIFQYQTTNTSNPGYRLIGQWTDELQLNIEDMQWGKGVREIPASVCTLPCKPGQRKKTQKGTPCCWTCEPCDGYQYQFDEMTCQHCPYDQRPNENRTGCQDIPIIKLEWHSPWAVIPVFLAMLGIIATIFVMATFIRYNDTPIVRASGRELSYVLLTGIFLCYIITFLMIAKPDVAVCSFRRVFLGLGMCISYAALLTKTNRIYRIFEQGKKSVTAPRLISPTSQLAITSSLISVQLLGVFIWFGVDPPNIIIDYDEHKTMNPEQARGVLKCDITDLQIICSLGYSILLMVTCTVYAIKTRGVPENFNEAKPIGFTMYTTCIVWLAFIPIFFGTAQSAEKLYIQTTTLTISMNLSASVALGMLYMPKVYIIIFHPELNVQKRKRSFKAVVTAATMSSRLSHKPSDRPNGEAKTELCENVDPNSPAAKKKYVSYNNLVI

SEQ ID NO: 7 - DNA, 2770 bp, Artificial Sequence
  Description of Artificial Sequence Synthetic chimeric construct
ctagctgtctcatcccttgccctggagagacggcagaaccatggcattttatagctgctgctgggtcctcttggcactcacctggcacacctctgcctacgggccagaccagcgagcccaacgcggccaggagatgtacgccccgcactcaatccggatcgagggggacgtcaccctcggggggctgttccccgtgcacgccaagggtcccagcggagtgccctgcggcgacatcaagagggaaaatgggatccacaggctggaagcgatgctctacgccctggaccagatcaacagtgatcccaacctactgcccaacgtgacgctgggcgcgcggatcctggacacttgttccagggacacttacgcgctcgaacagtcgcttactttcgtccaggcgctcatccagaaggacacctccgacgtgcgctgcaccaacggcgaaccgccggttttcgtcaagccggagaaagtagttggagtgattggggcttcggggagttcggtctccatcatggtagccaacatcctgaggctcttccagatcccccagattagttatgcatcaacggcacccgagctaagtgatgaccggcgctatgacttcttctctcgcgtggtgccacccgattccttccaagcccaggccatggtagacattgtaaaggccctaggctggaattatgtgtctaccctcgcatcggaaggaagttatggagagaaaggtgtggagtccttcacgcagatttccaaagaggcaggtggactctgcattgcccagtccgtgagaatcccccaggaacgcaaagacaggaccattgactttgatagaattatcaaacagctcctggacacccccaactccagggccgtcgtgatttttgccaacgatgaggatataaagcagatccttgcagcagccaaaagagctgaccaagttggccattttctttgggtgggatcagacagctggggatccaaaataaacccactgcaccagcatgaagatatcgcagaaggggccatcaccattcagcccaagcgagccacggtggaagggtttgatgcctactttacgtcccgtacacttgaaaacaacagaagaaatgtatggtttgccgaatactgggaggaaaacttcaactgcaagttgacgattagtgggtcaaaaaaagaagacacagatcgcaaatgcacaggacaggagagaattggaaaagattccaactatgagcaggagggtaaagtccagttcgtgattgacgcagtctatgctatggctcacgcccttcaccacatgaacaaggatctctgtgctgactaccggggtgtctgcccagagatggagcaagctggaggcaagaagttgctgaagtatatacgcaatgttaatttcaatggtagtgctggcactccagtgatgtttaacaagaacggggatgcacctgggcgttatgacatctttcagtaccagaccacaaacaccagcaacccgggttaccgtctgatcgggcagtggacagacgaacttcagctcaatatagaagacatgcagtggggtaaaggagtccgagagatacccgcctcagtgtgcacactaccatgtaagccaggacagagaaagaagacacagaaaggaactccttgctgttggacctgtgaaccttgcgatggttaccagtaccagtttgatgagatgacatgccagcattgcccctatgaccagaggcccaatgaaaatcgaaccggatgccaggatattcccatcatcaaactggagtggcactccccctgggctgtgattcctgtcttcctggcaatgttggggatcattgccaccatctttgtcatggccactttcatccgctacaatgacacgcccattgtccgggcatctgggcgggaactcagctatgttcttttgacgggcatctttctttgctacatcatcactttcctgatgattgccaaaccagatgtggcagtgtgttctttccggcgagttttcttgggcttgggtatgtgcatcagttatgcagccctcttgacgaaaacaaatcggatttatcgcatatttgagcagggcaagaaatcagtaacagctcccagactcataagcccaacatcacaactggcaatcacttccagtttaatatcagttcagcttctaggggtgttcatttggtttggtgttgatccacccaacatcatcatagactacgatgaacacaagacaatgaaccctgagcaagccagaggggttctcaagtgtgacattacagatctccaaatcatttgctccttgggatatagcattcttctcatggtcacatgtactgtgtatgccatcaagactcggggtgtacccgagaattttaacgaagccaagcccattggattcactatgtacacgacatgtatagtatggcttgccttcattccaattttttttggcaccgctcaatcagcggaaaagctctacatacaaactaccacgcttacaatctccatgaacctaagtgcatcagtggcgctggggatgctatacatgccgaaagtgtacatcatcattttccaccctgaactcaatgtccagaaacggaagcgaagcttcaaggcggtagtcacagcagccaccatgtcatcgaggctgtcacacaaacccagtgacagacccaacggtgaggcaaagaccgagctctgtgaaaacgtagacccaaacagccctgctgcaaaaaagaagtatgtcagttataataacctggttatc

SEQ ID NO: 8 - protein, 910 aa, Artificial Sequence
  Description of Artificial Sequence Synthetic chimeric construct
MAFYSCCWVLLALTWHTSAYGPDQRAQRGQEMYAPHSIRIEGDVTLGGLFPVHAKGPSGVPCGDIKRENGIHRLEAMLYALDQINSDPNLLPNVTLGARILDTCSRDTYALEQSLTFVQALIQKDTSDVRCTNGEPPVFVKPEKVVGVIGASGSSVSIMVANILRLFQIPQISYASTAPELSDDRRYDFFSRVVPPDSFQAQAMVDIVKALGWNYVSTLASEGSYGEKGVESFTQISKEAGGLCIAQSVRIPQERKDRTIDFDRIIKQLLDTPNSRAVVIFANDEDIKQILAAAKRADQVGHFLWVGSDSWGSKINPLHQHEDIAEGAITIQPKRATVEGFDAYFTSRTLENNRRNVWFAEYWEENFNCKLTISGSKKEDTDRKCTGQERIGKDSNYEQEGKVQFVIDAVYAMAHALHHMNKDLCADYRGVCPEMEQAGGKKLLKYIRNVNFNGSAGTPVMFNKNGDAPGRYDIFQYQTTNTSNPGYRLIGQWTDELQLNIEDMQWGKGVREIPASVCTLPCKPGQRKKTQKGTPCCWTCEPCDGYQYQFDEMTCQHCPYDQRPNENRTGCQDIPIIKLEWHSPWAVIPVFLAMLGIIATIFVMATFIRYNDTPIVRASGRELSYVLLTGIFLCYIITFLMIAKPDVAVCSFRRVFLGLGMCISYAALLTKTNRIYRIFEQGKKSVTAPRLISPTSQLAITSSLISVQLLGVFIWFGVDPPNIIIDYDEHKTMNPEQARGVLKCDITDLQIICSLGYSILLMVTCTVYAIKTRGVPENFNEAKPIGFTMYTTCIVWLAFIPIFFGTAQSAEKLYIQTTTLTISMNLSASVALGMLYMPKVYIIIFHPELNVQKRKRSFKAVVTAATMSSRLSHKPSDRPNGEAKTELCENVDPNSPAAKKKYVSYNNLVI

SEQ ID NO: 9 - DNA, 2761 bp, Artificial Sequence
  Description of Artificial Sequence Synthetic chimeric construct
ctagctgtctcatcccttgccctggagagacggcagaaccatggcattttatagctgctgctgggtcctcttggcactcacctggcacacctctgcctacgggccagaccagcgagcccaagagatgtacgccccgcactcaatccggatcgagggggacgtcaccctcggggggctgttccccgtgcacgccaagggtcccagcggagtgccctgcggcgacatcaagagggaaaatgggatccacaggctggaagcgatgctctacgccctggaccagatcaacagtgatcccaacctactgcccaacgtgacgctgggcgcgcggatcctggacacttgttccagggacacttacgcgctcgaacagtcgcttactttcgtccaggcgctcatccagaaggacacctccgacgtgcgctgcaccaacggcgaaccgccggttttcgtcaagccggagaaagtagttggagtgattggggcttcggggagttcggtctccatcatggtagccaacatcctgaggctcttccagatcccccagattagttatgcatcaacggcacccgagctaagtgatgaccggcgctatgacttcttctctcgcgtggtgccacccgattccttccaagcccaggccatggtagacattgtaaaggccctaggctggaattatgtgtctaccctcgcatcggaaggaagttatggagagaaaggtgtggagtccttcacgcagatttccaaagaggcaggtggactctgcattgcccagtccgtgagaatcccccaggaacgcaaagacaggaccattgactttgatagaattatcaaacagctcctggacacccccaactccagggccgtcgtgatttttgccaacgatgaggatataaagcagatccttgcagcagccaaaagagctgaccaagttggccattttctttgggtgggatcagacagctggggatccaaaataaacccactgcaccagcatgaagatatcgcagaaggggccatcaccattcagcccaagcgagccacggtggaagggtttgatgcctactttacgtcccgtacacttgaaaacaacagaagaaatgtatggtttgccgaatactgggaggaaaacttcaactgcaagttgacgattagtgggtcaaaaaaagaagacacagatcgcaaatgcacaggacaggagagaattggaaaagattccaactatgagcaggagggtaaagtccagttcgtgattgacgcagtctatgctatggctcacgcccttcaccacatgaacaaggatctctgtgctgactaccggggtgtctgcccagagatggagcaagctggaggcaagaagttgctgaagtatatacgcaatgttaatttcaatggtagtgctggcactccagtgatgtttaacaagaacggggatgcacctgggcgttatgacatctttcagtaccagaccacaaacaccagcaacccgggttaccgtctgatcgggcagtggacagacgaacttcagctcaatatagaagacatgcagtggggtaaaggagtccgagagatacccgcctcagtgtgcacactaccatgtaagccaggacagagaaagaagacacagaaaggaactccttgctgttggacctgtgaaccttgcgatggttaccagtaccagtttgatgagatgacatgccagcattgcccctatgaccagaggcccaatgaaaatcgaaccggatgccaggatattcccatcatcaaactggagtggcactccccctgggctgtgattcctgtcttcctggcaatgttggggatcattgccaccatctttgtcatggccactttcatccgctacaatgacacgcccattgtccgggcatctgggcgggaactcagctatgttcttttgacgggcatctttctttgctacatcatcactttcctgatgattgccaaaccagatgtggcagtgtgttctttccggcgagttttcttgggcttgggtatgtgcatcagttatgcagccctcttgacgaaaacaaatcggatttatcgcatatttgagcagggcaagaaatcagtaacagctcccagactcataagcccaacatcacaactggcaatcacttccagtttaatatcagttcagcttctaggggtgttcatttggtttggtgttgatccacccaacatcatcatagactacgatgaacacaagacaatgaaccctgagcaagccagaggggttctcaagtgtgacattacagatctccaaatcatttgctccttgggatatagcattcttctcatggtcacatgtactgtgtatgccatcaagactcggggtgtacccgagaattttaacgaagccaagcccattggattcactatgtacacgacatgtatagtatggcttgccttcattccaattttttttggcaccgctcaatcagcggaaaagctctacatacaaactaccacgcttacaatctccatgaacctaagtgcatcagtggcgctggggatgctatacatgccgaaagtgtacatcatcattttccaccctgaactcaatgtccagaaacggaagcgaagcttcaaggcggtagtcacagcagccaccatgtcatcgaggctgtcacacaaacccagtgacagacccaacggtgaggcaaagaccgagctctgtgaaaacgtagacccaaacagccctgctgcaaaaaagaagtatgtcagttataataacctggttatc

SEQ ID NO: 10 - protein, 907 aa, Artificial Sequence
  Description of Artificial Sequence Synthetic chimeric construct
MAFYSCCWVLLALTWHTSAYGPDQRAQEMYAPHSIRIEGDVTLGGLFPVHAKGPSGVPCGDIKRENGIHRLEAMLYALDQINSDPNLLPNVTLGARILDTCSRDTYALEQSLTFVQALIQKDTSDVRCTNGEPPVFVKPEKVVGVIGASGSSVSIMVANILRLFQIPQISYASTAPELSDDRRYDFFSRVVPPDSFQAQAMVDIVKALGWNYVSTLASEGSYGEKGVESFTQISKEAGGLCIAQSVRIPQERKDRTIDFDRIIKQLLDTPNSRAVVIFANDEDIKQILAAAKRADQVGHFLWVGSDSWGSKINPLHQHEDIAEGAITIQPKRATVEGFDAYFTSRTLENNRRNVWFAEYWEENFNCKLTISGSKKEDTDRKCTGQERIGKDSNYEQEGKVQFVIDAVYAMAHALHHMNKDLCADYRGVCPEMEQAGGKKLLKYIRNVNFNGSAGTPVMFNKNGDAPGRYDIFQYQTTNTSNPGYRLIGQWTDELQLNIEDMQWGKGVREIPASVCTLPCKPGQRKKTQKGTPCCWTCEPCDGYQYQFDEMTCQHCPYDQRPNENRTGCQDIPIIKLEWHSPWAVIPVFLAMLGIIATIFVMATFIRYNDTPIVRASGRELSYVLLTGIFLCYIITFLMIAKPDVAVCSFRRVFLGLGMCISYAALLTKTNRIYRIFEQGKKSVTAPRLISPTSQLAITSSLISVQLLGVFIWFGVDPPNIIIDYDEHKTMNPEQARGVLKCDITDLQIICSLGYSILLMVTCTVYAIKTRGVPENFNEAKPIGFTMYTTCIVWLAFIPIFFGTAQSAEKLYIQTTTLTISMNLSASVALGMLYMPKVYIIIFHPELNVQKRKRSFKAVVTAATMSSRLSHKPSDRPNGEAKTELCENVDPNSPAAKKKYVSYNNLVI

SEQ ID NO: 11 - DNA, 2734 bp, Artificial Sequence
  Description of Artificial Sequence Synthetic chimeric construct
ctagctgtctcatcccttgccctggagagacggcagaaccatggcattttatagctgctgctgggtcctcttggcactcacctggcacacctctgcctacgggccagaccagcgagcccaaatcgagggggacgtcaccctcggggggctgttccccgtgcacgccaagggtcccagcggagtgccctgcggcgacatcaagagggaaaatgggatccacaggctggaagcgatgctctacgccctggaccagatcaacagtgatcccaacctactgcccaacgtgacgctgggcgcgcggatcctggacacttgttccagggacacttacgcgctcgaacagtcgcttactttcgtccaggcgctcatccagaaggacacctccgacgtgcgctgcaccaacggcgaaccgccggttttcgtcaagccggagaaagtagttggagtgattggggcttcggggagttcggtctccatcatggtagccaacatcctgaggctcttccagatcccccagattagttatgcatcaacggcacccgagctaagtgatgaccggcgctatgacttcttctctcgcgtggtgccacccgattccttccaagcccaggccatggtagacattgtaaaggccctaggctggaattatgtgtctaccctcgcatcggaaggaagttatggagagaaaggtgtggagtccttcacgcagatttccaaagaggcaggtggactctgcattgcccagtccgtgagaatcccccaggaacgcaaagacaggaccattgactttgatagaattatcaaacagctcctggacacccccaactccagggccgtcgtgatttttgccaacgatgaggatataaagcagatccttgcagcagccaaaagagctgaccaagttggccattttctttgggtgggatcagacagctggggatccaaaataaacccactgcaccagcatgaagatatcgcagaaggggccatcaccattcagcccaagcgagccacggtggaagggtttgatgcctactttacgtcccgtacacttgaaaacaacagaagaaatgtatggtttgccgaatactgggaggaaaacttcaactgcaagttgacgattagtgggtcaaaaaaagaagacacagatcgcaaatgcacaggacaggagagaattggaaaagattccaactatgagcaggagggtaaagtccagttcgtgattgacgcagtctatgctatggctcacgcccttcaccacatgaacaaggatctctgtgctgactaccggggtgtctgcccagagatggagcaagctggaggcaagaagttgctgaagtatatacgcaatgttaatttcaatggtagtgctggcactccagtgatgtttaacaagaacggggatgcacctgggcgttatgacatctttcagtaccagaccacaaacaccagcaacccgggttaccgtctgatcgggcagtggacagacgaacttcagctcaatatagaagacatgcagtggggtaaaggagtccgagagatacccgcctcagtgtgcacactaccatgtaagccaggacagagaaagaagacacagaaaggaactccttgctgttggacctgtgaaccttgcgatggttaccagtaccagtttgatgagatgacatgccagcattgcccctatgaccagaggcccaatgaaaatcgaaccggatgccaggatattcccatcatcaaactggagtggcactccccctgggctgtgattcctgtcttcctggcaatgttggggatcattgccaccatctttgtcatggccactttcatccgctacaatgacacgcccattgtccgggcatctgggcgggaactcagctatgttcttttgacgggcatctttctttgctacatcatcactttcctgatgattgccaaaccagatgtggcagtgtgttctttccggcgagttttcttgggcttgggtatgtgcatcagttatgcagccctcttgacgaaaacaaatcggatttatcgcatatttgagcagggcaagaaatcagtaacagctcccagactcataagcccaacatcacaactggcaatcacttccagtttaatatcagttcagcttctaggggtgttcatttggtttggtgttgatccacccaacatcatcatagactacgatgaacacaagacaatgaaccctgagcaagccagaggggttctcaagtgtgacattacagatctccaaatcatttgctccttgggatatagcattcttctcatggtcacatgtactgtgtatgccatcaagactcggggtgtacccgagaattttaacgaagccaagcccattggattcactatgtacacgacatgtatagtatggcttgccttcattccaattttttttggcaccgctcaatcagcggaaaagctctacatacaaactaccacgcttacaatctccatgaacctaagtgcatcagtggcgctggggatgctatacatgccgaaagtgtacatcatcattttccaccctgaactcaatgtccagaaacggaagcgaagcttcaaggcggtagtcacagcagccaccatgtcatcgaggctgtcacacaaacccagtgacagacccaacggtgaggcaaagaccgagctctgtgaaaacgtagacccaaacagccctgctgcaaaaaagaagtatgtcagttataataacctggttatc

SEQ ID NO: 12 - protein, 898 aa, Artificial Sequence
  Description of Artificial Sequence Synthetic chimeric construct
MAFYSCCWVLLALTWHTSAYGPDQRAQIEGDVTLGGLFPVHAKGPSGVPCGDIKRENGIHRLEAMLYALDQINSDPNLLPNVTLGARILDTCSRDTYALEQSLTFVQALIQKDTSDVRCTNGEPPVFVKPEKVVGVIGASGSSVSIMVANILRLFQIPQISYASTAPELSDDRRYDFFSRVVPPDSFQAQAMVDIVKALGWNYVSTLASEGSYGEKGVESFTQISKEAGGLCIAQSVRIPQERKDRTIDFDRIIKQLLDTPNSRAVVIFANDEDIKQILAAAKRADQVGHFLWVGSDSWGSKINPLHQHEDIAEGAITIQPKRATVEGFDAYFTSRTLENNRRNVWFAEYWEENFNCKLTISGSKKEDTDRKCTGQERIGKDSNYEQEGKVQFVIDAVYAMAHALHHMNKDLCADYRGVCPEMEQAGGKKLLKYIRNVNFNGSAGTPVMFNKNGDAPGRYDIFQYQTTNTSNPGYRLIGQWTDELQLNIEDMQWGKGVREIPASVCTLPCKPGQRKKTQKGTPCCWTCEPCDGYQYQFDEMTCQHCPYDQRPNENRTGCQDIPIIKLEWHSPWAVIPVFLAMLGIIATIFVMATFIRYNDTPIVRASGRELSYVLLTGIFLCYIITFLMIAKPDVAVCSFRRVFLGLGMCISYAALLTKTNRIYRIFEQGKKSVTAPRLISPTSQLAITSSLISVQLLGVFIWFGVDPPNIIIDYDEHKTMNPEQARGVLKCDITDLQIICSLGYSILLMVTCTVYAIKTRGVPENFNEAKPIGFTMYTTCIVWLAFIPIFFGTAQSAEKLYIQTTTLTISMNLSASVALGMLYMPKVYIIIFHPELNVQKRKRSFKAVVTAATMSSRLSHKPSDRPNGEAKTELCENVDPNSPAAKKKYVSYNNLVI

SEQ ID NO: 13 - DNA, 2652 bp, Artificial Sequence
  Description of Artificial Sequence Synthetic chimeric construct
atggcattttatagctgctgctgggtcctcttggcactcacctggcacacctctgcctacgggccagaccagcgagcccaattaggggaccataactttctaaggagagagattaaaatagaaggtgaccttgttttagggggcctgtttcctattaacgaaaaaggcactggaactgaagaatgtgggcgaatcaatgaagaccgagggattcaacgcctggaagccatgttgtttgctattgatgaaatcaacaaagatgattacttgctaccaggagtgaagttgggtgttcacattttggatacatgttcaagggatacctatgcattggagcaatcactggagtttgtcagggcatctttgacaaaagtggatgaagctgagtatatgtgtcctgatggatcctatgccattcaagaaaacatcccacttctcattgcaggggtcattggtggctcttatagcagtgtttccatacaggtggcaaacctgctgcggctcttccagatccctcagatcagctacgcatccaccagcgccaaactcagtgataagtcgcgctatgattactttgccaggaccgtgccccccgacttctaccaggccaaagccatggctgagatcttgcgcttcttcaactggacctacgtgtccacagtagcctccgagggtgattacggggagacagggatcgaggccttcgagcaggaagcccgcctgcgcaacatctgcatcgctacggcggagaaggtgggccgctccaacatccgcaagtcctacgacagcgtgatccgagaactgttgcagaagcccaacgcgcgcgtcgtggtcctcttcatgcgcagcgacgactcgcgggagctcattgcagccgccagccgcgccaatgcctccttcacctgggtggccagcgacggctggggcgcgcaggagagcatcatcaagggcagcgagcatgtggcctacggcgccatcaccctggagctggcctcccagcctgtccgccagttcgaccgctacttccagagcctcaacccctacaacaaccaccgcaacccctggttccgggacttctgggagcaaaagtttcagtgcagcctccagaacaaacgcaaccacaggcgcgtctgcgacaagcacctggccatcgacagcagcaactacgagcaagagtccaagatcatgtttgtggtgaacgcggtgtatgccatggcccacgctttgcacaaaatgcagcgcaccctctgtcccaacactaccaagctttgtgatgctatgaagatcctggatgggaagaagttgtacaaggattacttgctgaaaatcaacttcacggctccattcaacccaaataaagatgcagatagcatagtcaagtttgacacttttggagatggaatggggcgatacaacgtgttcaatttccaaaatgtaggtggaaagtattcctacttgaaagttggtcactgggcagaaaccttatcgctagatgtcaactctatccactggtcccggaactcagtccccacttcccagtgcagcgacccctgtgcccccaatgaaatgaagaatatgcaaccaggggatgtctgctgctggatttgcatcccctgtgaaccctacgaatacctggctgatgagtttacctgtatggattgtgggtctggacagtggcccactgcagacctaactggatgctatgaccttcctgaggactacatcaggtgggaagacgcctgggccattggcccagtcaccattgcctgtctgggttttatgtgtacatgcatggttgtaactgtttttatcaagcacaacaacacacccttggtcaaagcatcgggccgagaactctgctacatcttattgtttggggttggcctgtcatactgcatgacattcttcttcattgccaagccatcaccagtcatctgtgcattgcgccgactcgggctggggagttccttcgctatctgttactcagccctgctgaccaagacaaactgcattgcccgcatcttcgatggggtcaagaatggcgctcagaggccaaaattcatcagccccagttctcaggttttcatctgcctgggtctgatcctggtgcaaattgtgatggtgtctgtgtggctcatcctggaggccccaggcaccaggaggtatacccttgcagagaagcgggaaacagtcatcctaaaatgcaatgtcaaagattccagcatgttgatctctcttacctacgatgtgatcctggtgatcttatgcactgtgtacgccttcaaaacgcggaagtgcccagaaaatttcaacgaagctaagttcataggttttaccatgtacaccacgtgcatcatctggttggccttcctccctatattttatgtgacatcaagtgactacagagtgcagacgacaaccatgtgcatctctgtcagcctgagtggctttgtggtcttgggctgtttgtttgcacccaaggttcacatcatcctgtttcaaccccagaagaatgttgtcacacacagactgcacctcaacaggttcagtgtcagtggaactgggaccacatactctcagtcctctgcaagcacgtatgtgccaacggtgtgcaatgggcgggaagtcctcgactccaccacctcatctctg

SEQ ID NO: 14 - protein, 884 aa, Artificial Sequence
  Description of Artificial Sequence Synthetic chimeric construct
MAFYSCCWVLLALTWHTSAYGPDQRAQLGDHNFLRREIKIEGDLVLGGLFPINEKGTGTEECGRINEDRGIQRLEAMLFAIDEINKDDYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRFFNWTYVSTVASEGDYGETGIEAFEQEARLRNICIATAEKVGRSNIRKSYDSVIRELLQKPNARVVVLFMRSDDSRELIAAASRANASFTWVASDGWGAQESIIKGSEHVAYGAITLELASQPVRQFDRYFQSLNPYNNHRNPWFRDFWEQKFQCSLQNKRNHRRVCDKHLAIDSSNYEQESKIMFVVNAVYAMAHALHKMQRTLCPNTTKLCDAMKILDGKKLYKDYLLKINFTAPFNPNKDADSIVKFDTFGDGMGRYNVFNFQNVGGKYSYLKVGHWAETLSLDVNSIHWSRNSVPTSQCSDPCAPNEMKNMQPGDVCCWICIPCEPYEYLADEFTCMDCGSGQWPTADLTGCYDLPEDYIRWEDAWAIGPVTIACLGFMCTCMVVTVFIKHNNTPLVKASGRELCYILLFGVGLSYCMTFFFIAKPSPVICALRRLGLGSSFAICYSALLTKTNCIARIFDGVKNGAQRPKFISPSSQVFICLGLILVQIVMVSVWLILEAPGTRRYTLAEKRETVILKCNVKDSSMLISLTYDVILVILCTVYAFKTRKCPENFNEAKFIGFTMYTTCIIWLAFLPIFYVTSSDYRVQTTTMCISVSLSGFVVLGCLFAPKVHIILFQPQKNVVTHRLHLNRFSVSGTGTTYSQSSASTYVPTVCNGREVLDSTTSSL

SEQ ID NO: 15 - DNA, 2646 bp, Artificial Sequence
  Description of Artificial Sequence Synthetic chimeric construct
atggcattttatagctgctgctgggtcctcttggcactcacctggcacacctctgcctacgggccagaccagcgagcccaagaccataactttctaaggagagagattaaaatagaaggtgaccttgttttagggggcctgtttcctattaacgaaaaaggcactggaactgaagaatgtgggcgaatcaatgaagaccgagggattcaacgcctggaagccatgttgtttgctattgatgaaatcaacaaagatgattacttgctaccaggagtgaagttgggtgttcacattttggatacatgttcaagggatacctatgcattggagcaatcactggagtttgtcagggcatctttgacaaaagtggatgaagctgagtatatgtgtcctgatggatcctatgccattcaagaaaacatcccacttctcattgcaggggtcattggtggctcttatagcagtgtttccatacaggtggcaaacctgctgcggctcttccagatccctcagatcagctacgcatccaccagcgccaaactcagtgataagtcgcgctatgattactttgccaggaccgtgccccccgacttctaccaggccaaagccatggctgagatcttgcgcttcttcaactggacctacgtgtccacagtagcctccgagggtgattacggggagacagggatcgaggccttcgagcaggaagcccgcctgcgcaacatctgcatcgctacggcggagaaggtgggccgctccaacatccgcaagtcctacgacagcgtgatccgagaactgttgcagaagcccaacgcgcgcgtcgtggtcctcttcatgcgcagcgacgactcgcgggagctcattgcagccgccagccgcgccaatgcctccttcacctgggtggccagcgacggctggggcgcgcaggagagcatcatcaagggcagcgagcatgtggcctacggcgccatcaccctggagctggcctcccagcctgtccgccagttcgaccgctacttccagagcctcaacccctacaacaaccaccgcaacccctggttccgggacttctgggagcaaaagtttcagtgcagcctccagaacaaacgcaaccacaggcgcgtctgcgacaagcacctggccatcgacagcagcaactacgagcaagagtccaagatcatgtttgtggtgaacgcggtgtatgccatggcccacgctttgcacaaaatgcagcgcaccctctgtcccaacactaccaagctttgtgatgctatgaagatcctggatgggaagaagttgtacaaggattacttgctgaaaatcaacttcacggctccattcaacccaaataaagatgcagatagcatagtcaagtttgacacttttggagatggaatggggcgatacaacgtgttcaatttccaaaatgtaggtggaaagtattcctacttgaaagttggtcactgggcagaaaccttatcgctagatgtcaactctatccactggtcccggaactcagtccccacttcccagtgcagcgacccctgtgcccccaatgaaatgaagaatatgcaaccaggggatgtctgctgctggatttgcatcccctgtgaaccctacgaatacctggctgatgagtttacctgtatggattgtgggtctggacagtggcccactgcagacctaactggatgctatgaccttcctgaggactacatcaggtgggaagacgcctgggccattggcccagtcaccattgcctgtctgggttttatgtgtacatgcatggttgtaactgtttttatcaagcacaacaacacacccttggtcaaagcatcgggccgagaactctgctacatcttattgtttggggttggcctgtcatactgcatgacattcttcttcattgccaagccatcaccagtcatctgtgcattgcgccgactcgggctggggagttccttcgctatctgttactcagccctgctgaccaagacaaactgcattgcccgcatcttcgatggggtcaagaatggcgctcagaggccaaaattcatcagccccagttctcaggttttcatctgcctgggtctgatcctggtgcaaattgtgatggtgtctgtgtggctcatcctggaggccccaggcaccaggaggtatacccttgcagagaagcgggaaacagtcatcctaaaatgcaatgtcaaagattccagcatgttgatctctcttacctacgatgtgatcctggtgatcttatgcactgtgtacgccttcaaaacgcggaagtgcccagaaaatttcaacgaagctaagttcataggttttaccatgtacaccacgtgcatcatctggttggccttcctccctatattttatgtgacatcaagtgactacagagtgcagacgacaaccatgtgcatctctgtcagcctgagtggctttgtggtcttgggctgtttgtttgcacccaaggttcacatcatcctgtttcaaccccagaagaatgttgtcacacacagactgcacctcaacaggttcagtgtcagtggaactgggaccacatactctcagtcctctgcaagcacgtatgtgccaacggtgtgcaatgggcgggaagtcctcgactccaccacctcatctctg

SEQ ID NO: 16 - protein, 882 aa, Artificial Sequence
  Description of Artificial Sequence Synthetic chimeric construct
MAFYSCCWVLLALTWHTSAYGPDQRAQDHNFLRREIKIEGDLVLGGLFPINEKGTGTEECGRINEDRGIQRLEAMLFAIDEINKDDYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRFFNWTYVSTVASEGDYGETGIEAFEQEARLRNICIATAEKVGRSNIRKSYDSVIRELLQKPNARVVVLFMRSDDSRELIAAASRANASFTWVASDGWGAQESIIKGSEHVAYGAITLELASQPVRQFDRYFQSLNPYNNHRNPWFRDFWEQKFQCSLQNKRNHRRVCDKHLAIDSSNYEQESKIMFVVNAVYAMAHALHKMQRTLCPNTTKLCDAMKILDGKKLYKDYLLKINFTAPFNPNKDADSIVKFDTFGDGMGRYNVFNFQNVGGKYSYLKVGHWAETLSLDVNSIHWSRNSVPTSQCSDPCAPNEMKNMQPGDVCCWICIPCEPYEYLADEFTCMDCGSGQWPTADLTGCYDLPEDYIRWEDAWAIGPVTIACLGFMCTCMVVTVFIKHNNTPLVKASGRELCYILLFGVGLSYCMTFFFIAKPSPVICALRRLGLGSSFAICYSALLTKTNCIARIFDGVKNGAQRPKFISPSSQVFICLGLILVQIVMVSVWLILEAPGTRRYTLAEKRETVILKCNVKDSSMLISLTYDVILVILCTVYAFKTRKCPENFNEAKFIGFTMYTTCIIWLAFLPIFYVTSSDYRVQTTTMCISVSLSGFVVLGCLFAPKVHIILFQPQKNVVTHRLHLNRFSVSGTGTTYSQSSASTYVPTVCNGREVLDSTTSSL

SEQ ID NO: 17 - DNA, 2616 bp, Artificial Sequence
  Description of Artificial Sequence Synthetic chimeric construct
atggcattttatagctgctgctgggtcctcttggcactcacctggcacacctctgcctacgggccagaccagcgagcccaaatagaaggtgaccttgttttagggggcctgtttcctattaacgaaaaaggcactggaactgaagaatgtgggcgaatcaatgaagaccgagggattcaacgcctggaagccatgttgtttgctattgatgaaatcaacaaagatgattacttgctaccaggagtgaagttgggtgttcacattttggatacatgttcaagggatacctatgcattggagcaatcactggagtttgtcagggcatctttgacaaaagtggatgaagctgagtatatgtgtcctgatggatcctatgccattcaagaaaacatcccacttctcattgcaggggtcattggtggctcttatagcagtgtttccatacaggtggcaaacctgctgcggctcttccagatccctcagatcagctacgcatccaccagcgccaaactcagtgataagtcgcgctatgattactttgccaggaccgtgccccccgacttctaccaggccaaagccatggctgagatcttgcgcttcttcaactggacctacgtgtccacagtagcctccgagggtgattacggggagacagggatcgaggccttcgagcaggaagcccgcctgcgcaacatctgcatcgctacggcggagaaggtgggccgctccaacatccgcaagtcctacgacagcgtgatccgagaactgttgcagaagcccaacgcgcgcgtcgtggtcctcttcatgcgcagcgacgactcgcgggagctcattgcagccgccagccgcgccaatgcctccttcacctgggtggccagcgacggctggggcgcgcaggagagcatcatcaagggcagcgagcatgtggcctacggcgccatcaccctggagctggcctcccagcctgtccgccagttcgaccgctacttccagagcctcaacccctacaacaaccaccgcaacccctggttccgggacttctgggagcaaaagtttcagtgcagcctccagaacaaacgcaaccacaggcgcgtctgcgacaagcacctggccatcgacagcagcaactacgagcaagagtccaagatcatgtttgtggtgaacgcggtgtatgccatggcccacgctttgcacaaaatgcagcgcaccctctgtcccaacactaccaagctttgtgatgctatgaagatcctggatgggaagaagttgtacaaggattacttgctgaaaatcaacttcacggctccattcaacccaaataaagatgcagatagcatagtcaagtttgacacttttggagatggaatggggcgatacaacgtgttcaatttccaaaatgtaggtggaaagtattcctacttgaaagttggtcactgggcagaaaccttatcgctagatgtcaactctatccactggtcccggaactcagtccccacttcccagtgcagcgacccctgtgcccccaatgaaatgaagaatatgcaaccaggggatgtctgctgctggatttgcatcccctgtgaaccctacgaatacctggctgatgagtttacctgtatggattgtgggtctggacagtggcccactgcagacctaactggatgctatgaccttcctgaggactacatcaggtgggaagacgcctgggccattggcccagtcaccattgcctgtctgggttttatgtgtacatgcatggttgtaactgtttttatcaagcacaacaacacacccttggtcaaagcatcgggccgagaactctgctacatcttattgtttggggttggcctgtcatactgcatgacattcttcttcattgccaagccatcaccagtcatctgtgcattgcgccgactcgggctggggagttccttcgctatctgttactcagccctgctgaccaagacaaactgcattgcccgcatcttcgatggggtcaagaatggcgctcagaggccaaaattcatcagccccagttctcaggttttcatctgcctgggtctgatcctggtgcaaattgtgatggtgtctgtgtggctcatcctggaggccccaggcaccaggaggtatacccttgcagagaagcgggaaacagtcatcctaaaatgcaatgtcaaagattccagcatgttgatctctcttacctacgatgtgatcctggtgatcttatgcactgtgtacgccttcaaaacgcggaagtgcccagaaaatttcaacgaagctaagttcataggttttaccatgtacaccacgtgcatcatctggttggccttcctccctatattttatgtgacatcaagtgactacagagtgcagacgacaaccatgtgcatctctgtcagcctgagtggctttgtggtcttgggctgtttgtttgcacccaaggttcacatcatcctgtttcaaccccagaagaatgttgtcacacacagactgcacctcaacaggttcagtgtcagtggaactgggaccacatactctcagtcctctgcaagcacgtatgtgccaacggtgtgcaatgggcgggaagtcctcgactccaccacctcatctctg

SEQ ID NO: 18 - protein, 872 aa, Artificial Sequence
  Description of Artificial Sequence Synthetic chimeric construct
MAFYSCCWVLLALTWHTSAYGPDQRAQIEGDLVLGGLFPINEKGTGTEECGRINEDRGIQRLEAMLFAIDEINKDDYLLPGVKLGVHILDTCSRDTYALEQSLEFVRASLTKVDEAEYMCPDGSYAIQENIPLLIAGVIGGSYSSVSIQVANLLRLFQIPQISYASTSAKLSDKSRYDYFARTVPPDFYQAKAMAEILRFFNWTYVSTVASEGDYGETGIEAFEQEARLRNICIATAEKVGRSNIRKSYDSVIRELLQKPNARVVVLFMRSDDSRELIAAASRANASFTWVASDGWGAQESIIKGSEHVAYGAITLELASQPVRQFDRYFQSLNPYNNHRNPWFRDFWEQKFQCSLQNKRNHRRVCDKHLAIDSSNYEQESKIMFVVNAVYAMAHALHKMQRTLCPNTTKLCDAMKILDGKKLYKDYLLKINFTAPFNPNKDADSIVKFDTFGDGMGRYNVFNFQNVGGKYSYLKVGHWAETLSLDVNSIHWSRNSVPTSQCSDPCAPNEMKNMQPGDVCCWICIPCEPYEYLADEFTCMDCGSGQWPTADLTGCYDLPEDYIRWEDAWAIGPVTIACLGFMCTCMVVTVFIKHNNTPLVKASGRELCYILLFGVGLSYCMTFFFIAKPSPVICALRRLGLGSSFAICYSALLTKTNCIARIFDGVKNGAQRPKFISPSSQVFICLGLILVQIVMVSVWLILEAPGTRRYTLAEKRETVILKCNVKDSSMLISLTYDVILVILCTVYAFKTRKCPENFNEAKFIGFTMYTTCIIWLAFLPIFYVTSSDYRVQTTTMCISVSLSGFVVLGCLFAPKVHIILFQPQKNVVTHRLHLNRFSVSGTGTTYSQSSASTYVPTVCNGREVLDSTTSSL

SEQ ID NO: 19 - DNA, 2643 bp, Artificial Sequence
  Description of Artificial Sequence Synthetic chimeric construct
atggcattttatagctgctgctgggtcctcttggcactcacctggcacacctctgcctacgggccagaccagcgcgcccaagagggcccagccaagaaggtgctgaccctggagggagacttggtgctgggtgggctgttcccagtgcaccagaagggcggcccagcagaggactgtggtcctgtcaatgagcaccgtggcatccagcgcctggaggccatgctttttgcactggaccgcatcaaccgtgacccgcacctgctgcctggcgtgcgcctgggtgcacacatcctcgacagttgctccaaggacacacatgcgctggagcaggcactggactttgtgcgtgcctcactcagccgtggtgctgatggctcacgccacatctgccccgacggctcttatgcgacccatggtgatgctcccactgccatcactggtgttattggcggttcctacagtgatgtctccatccaggtggccaacctcttgaggctatttcagatcccacagattagctacgcctctaccagtgccaagctgagtgacaagtcccgctatgactactttgcccgcacagtgcctcctgacttcttccaagccaaggccatggctgagattctccgcttcttcaactggacctatgtgtccactgtggcgtctgagggcgactatggcgagacaggcattgaggcctttgagctagaggctcgtgcccgcaacatctgtgtggccacctcggagaaagtgggccgtgccatgagccgcgcggcctttgagggtgtggtgcgagccctgctgcagaagcccagtgcccgcgtggctgtcctgttcacccgttctgaggatgcccgggagctgcttgctgccagccagcgcctcaatgccagcttcacctgggtggccagtgatggttggggggccctggagagtgtggtggcaggcagtgagggggctgctgagggtgctatcaccatcgagctggcctcctaccccatcagtgactttgcctcctacttccagagcctggacccttggaacaacagccggaacccctggttccgtgaattctgggagcagaggttccgctgcagcttccggcagcgagactgcgcagcccactctctccgggctgtgccctttgagcaggagtccaagatcatgtttgtggtcaatgcagtgtacgccatggcccatgcgctccacaacatgcaccgtgccctctgccccaacaccacccggctctgtgacgcgatgcggccagttaacgggcgccgcctctacaaggactttgtgctcaacgtcaagtttgatgccccctttcgcccagctgacacccacaatgaggtccgctttgaccgctttggtgatggtattggccgctacaacatcttcacctatctgcgtgcaggcagtgggcgctatcgctaccagaaggtgggctactgggcagaaggcttgactctggacaccagcctcatcccatgggcctcaccctcagccggccccctgcccgcctctcgctgcagtgagccctgcctccagaatgaggtgaagagtgtgcagccgggcgaagtctgctgctggctctgcattccgtgccagccctatgagtaccgattggacgaattcacttgcgctgattgtggcctgggctactggcccaatgccagcctgactggctgcttcgaactgccccaggagtacatccgctggggcgatgcctgggctgtgggacctgtcaccatcgcctgcctcggtgccctggccaccctctttgtgctgggtgtctttgtgcggcacaatgccacaccagtggtcaaggcctcaggtcgggagctctgctacatcctgctgggtggtgtcttcctctgctactgcatgaccttcatcttcattgccaagccatccacggcagtgtgtaccttacggcgtcttggtttgggcactgccttctctgtctgctactcagccctgctcaccaagaccaaccgcattgcacgcatcttcggtggggcccgggagggtgcccagcggccacgcttcatcagtcctgcctcacaggtggccatctgcctggcacttatctcgggccagctgctcatcgtggtcgcctggctggtggtggaggcaccgggcacaggcaaggagacagcccccgaacggcgggaggtggtgacactgcgctgcaaccaccgcgatgcaagtatgttgggctcgctggcctacaatgtgctcctcatcgcgctctgcacgctttatgccttcaagactcgcaagtgccccgaaaacttcaacgaggccaagttcattggcttcaccatgtacaccacctgcatcatctggctggcattcctgcccatcttctatgtcacctccagtgactaccgggtacagaccaccaccatgtgcgtgtcagtcagcctcagcggctccgtggtgcttggctgcctctttgcgcccaagctgcacatcatcctcttccagccgcagaagaacgtggttagccaccgggcacccaccagccgctttggcagtgctgctgccagggccagctccagccttggccaagggtctggctcccagtttgtccccactgtttgcaatggccgtgaggtggtggactcgacaacgtcatcgctt

SEQ ID NO: 20 - protein, 881 aa, Artificial Sequence
  Description of Artificial Sequence Synthetic chimeric construct
MAFYSCCWVLLALTWHTSAYGPDQRAQEGPAKKVLTLEGDLVLGGLFPVHQKGGPAEDCGPVNEHRGIQRLEAMLFALDRINRDPHLLPGVRLGAHILDSCSKDTHALEQALDFVRASLSRGADGSRHICPDGSYATHGDAPTAITGVIGGSYSDVSIQVANLLRLFQIPQISYASTSAKLSDKSRYDYFARTVPPDFFQAKAMAEILRFFNWTYVSTVASEGDYGETGIEAFELEARARNICVATSEKVGRAMSRAAFEGVVRALLQKPSARVAVLFTRSEDARELLAASQRLNASFTWVASDGWGALESVVAGSEGAAEGAITIELASYPISDFASYFQSLDPWNNSRNPWFREFWEQRFRCSFRQRDCAAHSLRAVPFEQESKIMFVVNAVYAMAHALHNMHRALCPNTTRLCDAMRPVNGRRLYKDFVLNVKFDAPFRPADTHNEVRFDRFGDGIGRYNIFTYLRAGSGRYRYQKVGYWAEGLTLDTSLIPWASPSAGPLPASRCSEPCLQNEVKSVQPGEVCCWLCIPCQPYEYRLDEFTCADCGLGYWPNASLTGCFELPQEYIRWGDAWAVGPVTIACLGALATLFVLGVFVRHNATPVVKASGRELCYILLGGVFLCYCMTFIFIAKPSTAVCTLRRLGLGTAFSVCYSALLTKTNRIARIFGGAREGAQRPRFISPASQVAICLALISGQLLIVVAWLVVEAPGTGKETAPERREVVTLRCNHRDASMLGSLAYNVLLIALCTLYAFKTRKCPENFNEAKFIGFTMYTTCIIWLAFLPIFYVTSSDYRVQTTTMCVSVSLSGSVVLGCLFAPKLHIILFQPQKNVVSHRAPTSRFGSAAARASSSLGQGSGSQFVPTVCNGREVVDSTTSSL

SEQ ID NO: 21 - DNA, 2610 bp, Artificial Sequence
  Description of Artificial Sequence Synthetic chimeric construct
atggcattttatagctgctgctgggtcctcttggcactcacctggcacacctctgcctacgggccagaccagcgagcccaactggcgggcggcctgacgctgggcggcctgttcccggtgcacgcgcggggcgcggcgggccgggcgtgcgggccgctgaagaaggagcagggcgtgcaccggctggaggccatgctgtacgcgctggaccgcgtcaacgccgaccccgagctgctgcccggcgtgcgcctgggcgcgcggctgctggacacctgctcgcgggacacctacgcgctggagcaggcgctgagcttcgtgcaggcgctgatccgtggccgcggcgacggcgacgaggtgggcgtgcgctgcccgggaggcgtccctccgctgcgccccgcgccccccgagcgcgtcgtggccgtcgtgggcgcctcggccagctccgtctccatcatggtcgccaacgtgctgcgcctgtttgcgataccccagatcagctatgcctccacagccccggagctcagcgactccacacgctatgacttcttctcccgggtggtgccacccgactcctaccaggcgcaggccatggtggacatcgtgagggcactgggatggaactatgtgtccacgctggcctccgagggcaactatggcgaaagtggggttgaggccttcgttcagatctcccgagaggctgggggggtctgtattgcccagtctatcaagattcccagggaaccaaagccaggagagttcagcaaggtgatcaggagactcatggagacgcccaacgcccggggcatcatcatctttgccaatgaggatgacatcaggcgggtcctggaggcagctcgccaggccaacctgaccggccacttcctgtgggtcggctcagacagctggggagccaagacctcacccatcttgagcctggaggacgtggccgttggggccatcaccatcctgcccaaaagggcctccatcgacggatttgaccagtacttcatgactcgatccctggagaacaaccgcaggaacatctggttcgccgagttctgggaagagaattttaactgcaaactgaccagctcaggtacccagtcagatgattccacccgcaaatgcacaggcgaggaacgcatcggccgggactccacctacgagcaggagggcaaggtgcagtttgtgattgatgcggtgtatgccattgcccacgccctccacagcatgcaccaggcgctctgccctgggcacacaggcctgtgcccggcgatggaacccaccgatgggcggatgcttctgcagtacattcgagctgtccgcttcaacggcagcgcaggaacccctgtgatgttcaacgagaacggggatgcgcccgggcggtacgacatcttccagtaccaggcgaccaatggcagtgccagcagtggcgggtaccaggcagtgggccagtgggcagagaccctcagactggatgtggaggccctgcagtggtctggcgacccccacgaggtgccctcgtctctgtgcagcctgccctgcgggccgggggagcggaagaagatggtgaagggcgtcccctgctgttggcactgcgaggcctgtgacgggtaccgcttccaggtggacgagttcacatgcgaggcctgtcctggggacatgaggcccacgcccaaccacacgggctgccgccccacacctgtggtgcgcctgagctggtcctccccctgggcagccccgccgctcctcctggccgtgctgggcatcgtggccactaccacggtggtggccaccttcgtgcggtacaacaacacgcccatcgtccgggcctcgggccgagagctcagctacgtcctcctcaccggcatcttcctcatctacgccatcaccttcctcatggtggctgagcctggggccgcggtctgtgccgcccgcaggctcttcctgggcctgggcacgaccctcagctactctgccctgctcaccaagaccaaccgtatctaccgcatctttgagcagggcaagcgctcggtcacaccccctcccttcatcagccccacctcacagctggtcatcaccttcagcctcacctccctgcaggtggtggggatgatagcatggctgggggcccggcccccacacagcgtgattgactatgaggaacagcggacggtggaccccgagcaggccagaggggtgctcaagtgcgacatgtcggatctgtctctcatcggctgcctgggctacagcctcctgctcatggtcacgtgcacagtgtacgccatcaaggcccgtggcgtgcccgagaccttcaacgaggccaagcccatcggcttcaccatgtacaccacctgcatcatctggctggcattcgtgcccatcttctttggcactgcccagtcagctgaaaagatctacatccagacaaccacgctaaccgtgtccttgagcctgagtgcctcggtgtccctcggcatgctctacgtacccaaaacctacgtcatcctcttccatccagagcagaatgtgcagaagcgaaagcggagcctcaaggccacctccacggtggcagccccacccaagggcgaggatgcagaggcccacaag

SEQ ID NO: 22 - protein, 870 aa, Artificial Sequence
  Description of Artificial Sequence Synthetic chimeric construct
MAFYSCCWVLLALTWHTSAYGPDQRAQLAGGLTLGGLFPVHARGAAGRACGPLKKEQGVHRLEAMLYALDRVNADPELLPGVRLGARLLDTCSRDTYALEQALSFVQALIRGRGDGDEVGVRCPGGVPPLRPAPPERVVAVVGASASSVSIMVANVLRLFAIPQISYASTAPELSDSTRYDFFSRVVPPDSYQAQAMVDIVRALGWNYVSTLASEGNYGESGVEAFVQISREAGGVCIAQSIKIPREPKPGEFSKVIRRLMETPNARGIIIFANEDDIRRVLEAARQANLTGHFLWVGSDSWGAKTSPILSLEDVAVGAITILPKRASIDGFDQYFMTRSLENNRRNIWFAEFWEENFNCKLTSSGTQSDDSTRKCTGEERIGRDSTYEQEGKVQFVIDAVYAIAHALHSMHQALCPGHTGLCPAMEPTDGRMLLQYIRAVRFNGSAGTPVMFNENGDAPGRYDIFQYQATNGSASSGGYQAVGQWAETLRLDVEALQWSGDPHEVPSSLCSLPCGPGERKKMVKGVPCCWHCEACDGYRFQVDEFTCEACPGDMRPTPNHTGCRPTPVVRLSWSSPWAAPPLLLAVLGIVATTTVVATFVRYNNTPIVRASGRELSYVLLTGIFLIYAITFLMVAEPGAAVCAARRLFLGLGTTLSYSALLTKTNRIYRIFEQGKRSVTPPPFISPTSQLVITFSLTSLQVVGMIAWLGARPPHSVIDYEEQRTVDPEQARGVLKCDMSDLSLIGCLGYSLLLMVTCTVYAIKARGVPETFNEAKPIGFTMYTTCIIWLAFVPIFFGTAQSAEKIYIQTTTLTVSLSLSASVSLGMLYVPKTYVILFHPEQNVQKRKRSLKATSTVAAPPKGEDAEAHK

SEQ ID NO: 23 - DNA, 3654 bp, Artificial Sequence
  Description of Artificial Sequence Synthetic chimeric construct
atggcattttatagctgctgctgggtcctcttggcactcacctggcacacctctgcctacgggccagaccagcgagcccaatccagtgagaggagggtggtggctcacatgccgggtgacatcattattggagctctcttttctgttcatcaccagcctactgtggacaaagttcatgagaggaagtgtggggcggtccgtgaacagtatggcattcagagagtggaggccatgctgcataccctggaaaggatcaattcagaccccacactcttgcccaacatcacactgggctgtgagataagggactcctgctggcattcggctgtggccctagagcagagcattgagttcataagagattccctcatttcttcagaagaggaagaaggcttggtacgctgtgtggatggctcctcctcttccttccgctccaagaagcccatagtaggggtcattgggcctggctccagttctgtagccattcaggtccagaatttgctccagcttttcaacatacctcagattgcttactcagcaaccagcatggatctgagtgacaagactctgttcaaatatttcatgagggttgtgccttcagatgctcagcaggcaagggccatggtggacatagtgaagaggtacaactggacctatgtatcagccgtgcacacagaaggcaactatggagaaagtgggatggaagccttcaaagatatgtcagcgaaggaagggatttgcatcgcccactcttacaaaatctacagtaatgcaggggagcagagctttgataagctgctgaagaagctcacaagtcacttgcccaaggcccgggtggtggcctgcttctgtgagggcatgacggtgagaggtctgctgatggccatgaggcgcctgggtctagcgggagaatttctgcttctgggcagtgatggctgggctgacaggtatgatgtgacagatggatatcagcgagaagctgttggtggcatcacaatcaagctccaatctcccgatgtcaagtggtttgatgattattatctgaagctccggccagaaacaaaccaccgaaacccttggtttcaagaattttggcagcatcgttttcagtgccgactggaagggtttccacaggagaacagcaaatacaacaagacttgcaatagttctctgactctgaaaacacatcatgttcaggattccaaaatgggatttgtgatcaacgccatctattcgatggcctatgggctccacaacatgcagatgtccctctgcccaggctatgcaggactctgtgatgccatgaagccaattgatggacggaaacttttggagtccctgatgaaaaccaattttactggggtttctggagatacgatcctattcgatgagaatggagactctccaggaaggtatgaaataatgaatttcaaggaaatgggaaaagattactttgattatatcaacgttggaagttgggacaatggagaattaaaaatggatgatgatgaagtatggtccaagaaaagcaacatcatcagatctgtgtgcagtgaaccatgtgagaaaggccagatcaaggtgatccgaaagggagaagtcagctgttgttggacctgtacaccttgtaaggagaatgagtatgtctttgatgagtacacatgcaaggcatgccaactggggtcttggcccactgatgatctcacaggttgtgacttgatcccagtacagtatcttcgatggggtgaccctgaacccattgcagctgtggtgtttgcctgccttggcctcctggccaccctgtttgttactgtagtcttcatcatttaccgtgatacaccagtagtcaagtcctcaagcagggaactctgctacattatccttgctggcatctgcctgggctacttatgtaccttctgcctcattgcgaagcccaaacagatttactgctaccttcagagaattggcattggtctctccccagccatgagctactcagcccttgtaacaaagaccaaccgtattgcaaggatcctggctggcagcaagaagaagatctgtaccaaaaagcccagattcatgagtgcctgtgcccagctagtgattgctttcattctcatatgcatccagttgggcatcatcgttgccctctttataatggagcctcctgacataatgcatgactacccaagcattcgagaagtctacctgatctgtaacaccaccaacctaggagttgtcactccacttggatacaatggattgttgattttgagctgcaccttctatgcgttcaagaccagaaatgttccagctaacttcaacgaggccaagtatatcgccttcacaatgtacacgacctgcattatatggctagcttttgtgccaatctactttggcagcaactacaaaatcatcaccatgtgtttctcggtcagcctcagtgccacagtggccctaggctgcatgtttgtgccgaaggtgtacatcatcctggccaaaccagagagaaacgtgcgcagcgccttcaccacatctaccgtggtgcgcatgcatgtaggggatggcaagtcatcctccgcagccagcagatccagcagcctagtcaacctgtggaagagaaggggctcctctggggaaaccttaaggtacaaagacaggagactggcccagcacaagtcggaaatagagtgtttcacccccaaagggagtatggggaatggtgggagagcaacaatgagcagttccaatggaaaatccgtcacgtgggcccagaatgagaagagcagccgggggcagcacctgtggcagcgcctgtccatccacatcaacaagaaagaaaaccccaaccaaacggccgtcatcaagcccttccccaagagcacggagagccgtggcctgggcgctggcgctggcgcaggcgggagcgctgggggcgtgggggccacgggcggtgcgggctgcgcaggcgccggcccaggcgggcccgagtccccagacgccggccccaaggcgctgtatgatgtggccgaggctgaggagcacttcccggcgcccgcgcggccgcgctcaccgtcgcccatcagcacgctgagccaccgcgcgggctcggccagccgcacggacgacgatgtgccgtcgctgcactcggagcctgtggcgcgcagcagctcctcgcagggctccctcatggagcagatcagcagtgtggtcacccgcttcacggccaacatcagcgagctcaactccatgatgctgtccaccgcggcccccagccccggcgtcggcgccccgctctgctcgtcctacctgatccccaaagagatccagttgcccacgaccatgacgacctttgccgaaatccagcctctgccggccatcgaagtcacgggcggcgcgcagcccgcggcaggggcgcaggcggctggggacgcggcccgggagagccccgcggccggtcccgaggctgcggccgccaagccagacctggaggagctggtggctctcaccccgccgtcccccttcagagactcggtggactcggggagcacaacccccaactcaccagtgtccgagtcggccctctgtatcccgtcgtctcccaaatatgacactcttatcataagagattacactcagagctcctcgtcgttg

SEQ ID NO: 24 - protein, 1218 aa, Artificial Sequence
  Description of Artificial Sequence Synthetic chimeric construct
MAFYSCCWVLLALTWHTSAYGPDQRAQSSERRVVAHMPGDIIIGALFSVHHQPTVDKVHERKCGAVREQYGIQRVEAMLHTLERINSDPTLLPNITLGCEIRDSCWHSAVALEQSIEFIRDSLISSEEEEGLVRCVDGSSSSFRSKKPIVGVIGPGSSSVAIQVQNLLQLFNIPQIAYSATSMDLSDKTLFKYFMRVVPSDAQQARAMVDIVKRYNWTYVSAVHTEGNYGESGMEAFKDMSAKEGICIAHSYKIYSNAGEQSFDKLLKKLTSHLPKARVVACFCEGMTVRGLLMAMRRLGLAGEFLLLGSDGWADRYDVTDGYQREAVGGITIKLQSPDVKWFDDYYLKLRPETNHRNPWFQEFWQHRFQCRLEGFPQENSKYNKTCNSSLTLKTHHVQDSKMGFVINAIYSMAYGLHNMQMSLCPGYAGLCDAMKPIDGRKLLESLMKTNFTGVSGDTILFDENGDSPGRYEIMNFKEMGKDYFDYINVGSWDNGELKMDDDEVWSKKSNIIRSVCSEPCEKGQIKVIRKGEVSCCWTCTPCKENEYVFDEYTCKACQLGSWPTDDLTGCDLIPVQYLRWGDPEPIAAVVFACLGLLATLFVTVVFIIYRDTPVVKSSSRELCYIILAGICLGYLCTFCLIAKPKQIYCYLQRIGIGLSPAMSYSALVTKTNRIARILAGSKKKICTKKPRFMSACAQLVIAFILICIQLGIIVALFIMEPPDIMHDYPSIREVYLICNTTNLGVVTPLGYNGLLILSCTFYAFKTRNVPANFNEAKYIAFTMYTTCIIWLAFVPIYFGSNYKIITMCFSVSLSATVALGCMFVPKVYIILAKPERNVRSAFTTSTVVRMHVGDGKSSSAASRSSSLVNLWKRRGSSGETLRYKDRRLAQHKSEIECFTPKGSMGNGGRATMSSSNGKSVTWAQNEKSSRGQHLWQRLSIHINKKENPNQTAVIKPFPKSTESRGLGAGAGAGGSAGGVGATGGAGCAGAGPGGPESPDAGPKALYDVAEAEEHFPAPARPRSPSPISTLSHRAGSASRTDDDVPSLHSEPVARSSSSQGSLMEQISSVVTRFTANISELNSMMLSTAAPSPGVGAPLCSSYLIPKEIQLPTTMTTFAEIQPLPAIEVTGGAQPAAGAQAAGDAARESPAAGPEAAAAKPDLEELVALTPPSPFRDSVDSGSTTPNSPVSESALCIPSSPKYDTLIIRDYTQSSSSL

SEQ ID NO: 25 - DNA, 3378 bp, Artificial Sequence
  Description of Artificial Sequence Synthetic chimeric construct
cgccacaatggtccggctcctcttgattttcttcccaatgatctttttggagatgtccattttgcccaggatgcctgacagaaaagtattgctggcaggtgcctcgtcccagcgctccgtggcgagaatggacggagatgtcatcatcggagccctcttctcagtccatcaccagcctccagccgagaaggtacccgaaaggaagtgtggggagatcagggaacagtatggtatccagagggtggaggccatgttccacacgttggataagattaacgcggacccggtgctcctgcccaacatcactctgggcagtgagatccgggactcctgctggcactcttcagtggctctcgaacagagcatcgaattcatcagagactccctgatttccatccgagatgagaaggatgggctgaaccgatgcctgcctgatggccagaccctgccccctggcaggactaagaagcctattgctggagtgatcggccctggctccagctctgtggccattcaagtccagaatcttctccagctgttcgacatcccacagatcgcctattctgccacaagcatagacctgagtgacaaaactttgtacaaatacttcctgagggtggtcccttctgacactttgcaggcaagggcgatgctcgacatagtcaagcgttacaactggacctatgtctcagcagtccacacagaagggaattacggcgagagtggaatggatgctttcaaagaactggctgcccaggaaggcctctgcatcgcacactcggacaaaatctacagcaatgctggcgagaagagctttgaccggctcctgcgtaaactccgggagcggcttcccaaggccagggttgtggtctgcttctgcgagggcatgacagtgcggggcttactgagtgccatgcgccgcctgggcgtcgtgggcgagttctcactcattggaagtgatggatgggcagacagagatgaagtcatcgaaggctatgaggtggaagccaacggagggatcacaataaagcttcagtctccagaggtcaggtcatttgatgactacttcctgaagctgaggctggacaccaacacaaggaatccttggttccctgagttctggcaacatcgcttccagtgtcgcctacctggacacctcttggaaaaccccaactttaagaaagtgtgcacaggaaatgaaagcttggaagaaaactatgtccaggacagcaaaatgggatttgtcatcaatgccatctatgccatggcacatgggctgcagaacatgcaccatgctctgtgtcccggccatgtgggcctgtgtgatgctatgaaacccattgatggcaggaagctcctggatttcctcatcaaatcctcttttgtcggagtgtctggagaggaggtgtggttcgatgagaagggggatgctcccggaaggtatgacattatgaatctgcagtacacagaagctaatcgctatgactatgtccacgtggggacctggcatgaaggagtgctgaatattgatgattacaaaatccagatgaacaaaagcggaatggtacgatctgtgtgcagtgagccttgcttaaagggtcagattaaggtcatacggaaaggagaagtgagctgctgctggatctgcacggcctgcaaagagaatgagtttgtgcaggacgagttcacctgcagagcctgtgacctggggtggtggcccaacgcagagctcacaggctgtgagcccattcctgtccgttatcttgagtggagtgacatagaagggatcgcactcaccctctttgccgtgctgggcattttcctgacagcctttgtgctgggtgtgtttatcaagttccgcaacacacccattgtcaaggccaccaaccgagagctctcctacctcctcctcttctccctgctctgctgcttctccagctccctgttcttcatcggggagccccaggactggacgtgccgcctgcgccagccggcctttggcatcagcttcgtgctctgcatctcatgcatcctggtgaaaaccaaccgtgtcctcctggtgtttgaggccaagatccccaccagcttccaccgcaagtggtgggggctcaacctgcagttcctgctggttttcctctgcaccttcatgcagattgtcatctgtgtgatctggctctacaccgcgcccccctcaagctaccgcaaccaggagctggaggatgagatcatcttcatcacgtgccacgagggctccctcatggccctgggcttcctgatcggctacacctgcctgctggctgccatctgcttcttctttgccttcaagtcccggaagctgccggagaacttcaatgaagccaagttcatcaccttcagcatgctcatcttcttcatcgtctggatctccttcattccagcctatgccagcacctatggcaagtttgtctctgccgtagaggtgattgccatcctggcagccagctttggcttgctggcgtgcatcttcttcaacaagatctacatcattctcttcaagccatcccgcaacaccatcgaggaggtgcgttgcagcaccgcagctcacgctttcaaggtggctgcccgggccacgctgcgccgcagcaacgtctcccgcaagcggtccagcagccttggaggctccacgggatccaccccctcctcctccatcagcagcaagagcaacagcgaagacccattcccacagcccgagaggcagaagcagcagcagccgctggccctaacccagcaagagcagcagcagcagcccctgaccctcccacagcagcaacgatctcagcagcagcccagatgcaagcagaaggtcatctttggcagcggcacggtcaccttctcactgagctttgatgagcctcagaagaacgccatggcccacgggaattctacgcaccagaactccctggaggcccagaaaagcagcgatacgctgacccgacaccagccattactcccgctgcagtgcggggaaacggacttagatctgaccgtccaggaaacaggtctgcaaggacctgtgggtggagaccagcggccagaggtggaggaccctgaagagttgtccccagcacttgtagtgtccagttcacagagctttgtcatcagtggtggaggcagcactgttacagaaaacgtagtgaattcataaaatggaaggagaagactgggctagggagaatgcagagaggtttcttggggtcccagggatgaggaatcgccccagactcctttcctctgaggaagggataatagacacatcaaatgccccgaatttagtcacaccatcttaaatgacagtttgacccatgttccctttaaaaaaaaaaaaaaaaaagcggccgc

SEQ ID NO: 26 - protein, 1058 aa, Artificial Sequence
  Description of Artificial Sequence Synthetic chimeric construct
MVRLLLIFFPMIFLEMSILPRMPDRKVLLAGASSQRSVARMDGDVIIGALFSVHHQPPAEKVPERKCGEIREQYGIQRVEAMFHTLDKINADPVLLPNITLGSEIRDSCWHSSVALEQSIEFIRDSLISIRDEKDGLNRCLPDGQTLPPGRTKKPIAGVIGPGSSSVAIQVQNLLQLFDIPQIAYSATSIDLSDKTLYKYFLRVVPSDTLQARAMLDIVKRYNWTYVSAVHTEGNYGESGMDAFKELAAQEGLCIAHSDKIYSNAGEKSFDRLLRKLRERLPKARVVVCFCEGMTVRGLLSAMRRLGVVGEFSLIGSDGWADRDEVIEGYEVEANGGITIKLQSPEVRSFDDYFLKLRLDTNTRNPWFPEFWQHRFQCRLPGHLLENPNFKKVCTGNESLEENYVQDSKMGFVINAIYAMAHGLQNMHHALCPGHVGLCDAMKPIDGRKLLDFLIKSSFVGVSGEEVWFDEKGDAPGRYDIMNLQYTEANRYDYVHVGTWHEGVLNIDDYKIQMNKSGMVRSVCSEPCLKGQIKVIRKGEVSCCWICTACKENEFVQDEFTCRACDLGWWPNAELTGCEPIPVRYLEWSDIEGIALTLFAVLGIFLTAFVLGVFIKFRNTPIVKATNRELSYLLLFSLLCCFSSSLFFIGEPQDWTCRLRQPAFGISFVLCISCILVKTNRVLLVFEAKIPTSFHRKWWGLNLQFLLVFLCTFMQIVICVIWLYTAPPSSYRNQELEDEIIFITCHEGSLMALGFLIGYTCLLAAICFFFAFKSRKLPENFNEAKFITFSMLIFFIVWISFIPAYASTYGKFVSAVEVIAILAASFGLLACIFFNKIYIILFKPSRNTIEEVRCSTAAHAFKVAARATLRRSNVSRKRSSSLGGSTGSTPSSSISSKSNSEDPFPQPERQKQQQPLALTQQEQQQQPLTLPQQQRSQQQPRCKQKVIFGSGTVTFSLSFDEPQKNAMAHGNSTHQNSLEAQKSSDTLTRHQPLLPLQCGETDLDLTVQETGLQGPVGGDQRPEVEDPEELSPALVVSSSQSFVISGGGSTVTENVVNS

SEQ ID NO: 27 - DNA, 3219 bp, Artificial Sequence
  Description of Artificial Sequence Synthetic chimeric construct
gcggtggaccgcgtcttcgccacaatggtccggctcctcttgattttcttcccaatgatctttttggagatgtccattttgcccaggatgcctgacagaaaagtattgctggcaggtgcctcgtcccagcgctccgtggcgagaatggacggagatgtcatcatcggagccctcttctcagtccatcaccagcctccagccgagaaggtacccgaaaggaagtgtggggagatcagggaacagtatggtatccagagggtggaggccatgttccacacgttggataagattaacgcggacccggtgctcctgcccaacatcactctgggcagtgagatccgggactcctgctggcactcttcagtggctctcgaacagagcatcgaattcatcagagactccctgatttccatccgagatgagaaggatgggctgaaccgatgcctgcctgatggccagaccctgccccctggcaggactaagaagcctattgctggagtgatcggccctggctccagctctgtggccattcaagtccagaatcttctccagctgttcgacatcccacagatcgcctattctgccacaagcatagacctgagtgacaaaactttgtacaaatacttcctgagggtggtcccttctgacactttgcaggcaagggcgatgctcgacatagtcaagcgttacaactggacctatgtctcagcagtccacacagaagggaattacggcgagagtggaatggatgctttcaaagaactggctgcccaggaaggcctctgcatcgcacactcggacaaaatctacagcaatgctggcgagaagagctttgaccggctcctgcgtaaactccgggagcggcttcccaaggccagggttgtggtctgcttctgcgagggcatgacagtgcggggcttactgagtgccatgcgccgcctgggcgtcgtgggcgagttctcactcattggaagtgatggatgggcagacagagatgaagtcatcgaaggctatgaggtggaagccaacggagggatcacaataaagcttcagtctccagaggtcaggtcatttgatgactacttcctgaagctgaggctggacaccaacacaaggaatccttggttccctgagttctggcaacatcgcttccagtgtcgcctacctggacacctcttggaaaaccccaactttaagaaagtgtgcacaggaaatgaaagcttggaagaaaactatgtccaggacagcaaaatgggatttgtcatcaatgccatctatgccatggcacatgggctgcagaacatgcaccatgctctgtgtcccggccatgtgggcctgtgtgatgctatgaaacccattgatggcaggaagctcctggatttcctcatcaaatcctcttttgtcggagtgtctggagaggaggtgtggttcgatgagaagggggatgctcccggaaggtatgacattatgaatctgcagtacacagaagctaatcgctatgactatgtccacgtggggacctggcatgaaggagtgctgaatattgatgattacaaaatccagatgaacaaaagcggaatggtacgatctgtgtgcagtgagccttgcttaaagggtcagattaaggtcatacggaaaggagaagtgagctgctgctggatctgcacggcctgcaaagagaatgagtttgtgcaggacgagttcacctgcagagcctgtgacctggggtggtggcccaacgcagagctcacaggctgtgagcccattcctgtccgttatcttgagtggagtgacatagaatctatcatagccatcgccttttcttgcctgggcatcctcgtgacgctgtttgtcaccctcatcttcgttctgtaccgggacacacccgtggtcaaatcctccagtagggagctctgctatatcattctggctggtattttcctcggctatgtgtgccctttcaccctcatcgccaaacctactaccacatcctgctacctccagcgcctcctagttggcctctcttctgccatgtgctactctgctttagtgaccaaaaccaatcgtattgcacgcatcctggctggcagcaagaagaagatctgcacccggaagcccagattcatgagcgcttgggcccaagtgatcatagcctccattctgattagtgtacagctaacactagtggtgaccttgatcatcatggagcctcccatgcccattttgtcctacccgagtatcaaggaagtctaccttatctgcaataccagcaacctgggtgtggtggcccctttgggctacaatggactcctcatcatgagctgtacctactatgccttcaagacccgcaacgtgcccgccaacttcaacgaggccaaatatatcgcgttcaccatgtacaccacctgtatcatctggctagcttttgtgcccatttactttgggagcaactacaagatcatcacaacttgctttgcagtgagtctcagtgtaacagtggctctggggtgcatgttcactcccaagatgtacatcattattgccaagcctgagaggaataccatcgaggaggtgcgttgcagcaccgcagctcacgctttcaaggtggctgcccgggccacgctgcgccgcagcaacgtctcccgcaagcggtccagcagccttggaggctccacgggatccaccccctcctcctccatcagcagcaagagcaacagcgaagacccattcccacagcccgagaggcagaagcagcagcagccgctggccctaacccagcaagagcagcagcagcagcccctgaccctcccacagcagcaacgatctcagcagcagcccagatgcaagcagaaggtcatctttggcagcggcacggtcaccttctcactgagctttgatgagcctcagaagaacgccatggcccacgggaattctacgcaccagaactccctggaggcccagaaaagcagcgatacgctgacccgacaccagccattactcccgctgcagtgcggggaaacggacttagatctgaccgtccaggaaacaggtctgcaaggacctgtgggtggagaccagcggccagaggtggaggaccctgaagagttgtccccagcacttgtagtgtccagttcacagagctttgtcatcagtggtggaggcagcactgttacagaaaacgtagtgaattcataaaatggaaggagaagactgggctag

SEQ ID NO: 28 - protein, 1056 aa, Artificial Sequence
  Description of Artificial Sequence Synthetic chimeric construct
MVRLLLIFFPMIFLEMSILPRMPDRKVLLAGASSQRSVARMDGDVIIGALFSVHHQPPAEKVPERKCGEIREQYGIQRVEAMFHTLDKINADPVLLPNITLGSEIRDSCWHSSVALEQSIEFIRDSLISIRDEKDGLNRCLPDGQTLPPGRTKKPIAGVIGPGSSSVAIQVQNLLQLFDIPQIAYSATSIDLSDKTLYKYFLRVVPSDTLQARAMLDIVKRYNWTYVSAVHTEGNYGESGMDAFKELAAQEGLCIAHSDKIYSNAGEKSFDRLLRKLRERLPKARVVVCFCEGMTVRGLLSAMRRLGVVGEFSLIGSDGWADRDEVIEGYEVEANGGITIKLQSPEVRSFDDYFLKLRLDTNTRNPWFPEFWQHRFQCRLPGHLLENPNFKKVCTGNESLEENYVQDSKMGFVINAIYAMAHGLQNMHHALCPGHVGLCDAMKPIDGRKLLDFLIKSSFVGVSGEEVWFDEKGDAPGRYDIMNLQYTEANRYDYVHVGTWHEGVLNIDDYKIQMNKSGMVRSVCSEPCLKGQIKVIRKGEVSCCWICTACKENEFVQDEFTCRACDLGWWPNAELTGCEPIPVRYLEWSDIESIIAIAFSCLGILVTLFVTLIFVLYRDTPVVKSSSRELCYIILAGIFLGYVCPFTLIAKPTTTSCYLQRLLVGLSSAMCYSALVTKTNRIARILAGSKKKICTRKPRFMSAWAQVIIASILISVQLTLVVTLIIMEPPMPILSYPSIKEVYLICNTSNLGVVAPLGYNGLLIMSCTYYAFKTRNVPANFNEAKYIAFTMYTTCIIWLAFVPIYFGSNYKIITTCFAVSLSVTVALGCMFTPKMYIIIAKPERNTIEEVRCSTAAHAFKVAARATLRRSNVSRKRSSSLGGSTGSTPSSSISSKSNSEDPFPQPERQKQQQPLALTQQEQQQQPLTLPQQQRSQQQPRCKQKVIFGSGTVTFSLSFDEPQKNAMAHGNSTHQNSLEAQKSSDTLTRHQPLLPLQCGETDLDLTVQETGLQGPVGGDQRPEVEDPEELSPALVVSSSQSFVISGGGSTVTENVVNS

SEQ ID NO: 29 - DNA, 3219 bp, Artificial Sequence
  Description of Artificial Sequence Synthetic chimeric construct
gcggtggaccgcgtcttcgccacaatggtccggctcctcttgattttcttcccaatgatctttttggagatgtccattttgcccaggatgcctgacagaaaagtattgctggcaggtgcctcgtcccagcgctccgtggcgagaatggacggagatgtcatcatcggagccctcttctcagtccatcaccagcctccagccgagaaggtacccgaaaggaagtgtggggagatcagggaacagtatggtatccagagggtggaggccatgttccacacgttggataagattaacgcggacccggtgctcctgcccaacatcactctgggcagtgagatccgggactcctgctggcactcttcagtggctctcgaacagagcatcgaattcatcagagactccctgatttccatccgagatgagaaggatgggctgaaccgatgcctgcctgatggccagaccctgccccctggcaggactaagaagcctattgctggagtgatcggccctggctccagctctgtggccattcaagtccagaatcttctccagctgttcgacatcccacagatcgcctattctgccacaagcatagacctgagtgacaaaactttgtacaaatacttcctgagggttgtcccttctgacactttgcaggcaagggccatgcttgacatagtcaaacgttacaattggacctatgtctctgcagtccacacggaagggaattatggggagagcggaatggacgctttcaaagaactggctgcccaggaaggcctctgtatcgcccattctgacaaaatctacagcaacgctggggagaagagctttgaccgactcttgcgcaaactccgagagaggcttcccaaggctagagtggtggtctgcttctgtgaaggcatgacagtgcgaggactcctgagcgccatgcggcgccttggcgtcgtgggcgagttctcactcattggaagtgatggatgggcagacagagatgaagtcattgaaggttatgaggtggaagccaacgggggaatcacgataaagctgcagtctccagaggtcaggtcatttgatgattatttcctgaaactgaggctggacactaacacgaggaatccctggttccctgagttctggcaacatcggttccagtgccgccttccaggacaccttctggaaaatcccaactttaaacgaatctgcacaggcaatgaaagcttagaagaaaactatgtccaggacagtaagatggggtttgtcatcaatgccatctatgccatggcacatgggctgcagaacatgcaccatgccctctgccctggccacgtgggcctctgcgatgccatgaagcccatcgacggcagcaagctgctggacttcctcatcaagtcctcattcattggagtatctggagaggaggtgtggtttgatgagaaaggagacgctcctggaaggtatgatatcatgaatctgcagtacactgaagctaatcgctatgactatgtgcacgttggaacctggcatgaaggagtgctgaacattgatgattacaaaatccagatgaacaagagtggagtggtgcggtctgtgtgcagtgagccttgcttaaagggccagattaaggttatacggaaaggagaagtgagctgctgctggatttgcacggcctgcaaagagaatgaatatgtgcaagatgagttcacctgcaaagcttgtgacttgggatggtggcccaatgcagatctaacaggctgtgagcccattcctgtgcgctatcttgagtggagcaacatcgaatccattatagccatcgccttttcatgcctgggaatccttgttaccttgtttgtcaccctaatctttgtactgtaccgggacacaccagtggtcaaatcctccagtcgggagctctgctacatcatcctagctggcatcttccttggttatgtgtgcccattcactctcattgccaaacctactaccacctcctgctacctccagcgcctcttggttggcctctcctctgcgatgtgctactctgctttagtgactaaaaccaatcgtattgcacgcatcctggctggcagcaagaagaagatctgcacccggaagcccaggttcatgagtgcctgggctcaggtgatcattgcctcaattctgattagtgtgcaactaaccctggtggtaaccctgatcatcatggaaccccctatgcccattctgtcctacccaagtatcaaggaagtctaccttatctgcaataccagcaacctgggtgtggtggcccctttgggctacaatggactcctcatcatgagctgtacctactatgccttcaagacccgcaacgtgcccgccaacttcaacgaggccaaatatatcgcgttcaccatgtacaccacctgtatcatctggctagcttttgtgcccatttactttgggagcaactacaagatcatcacaacttgctttgcagtgagtctcagtgtaacagtggctctggggtgcatgttcactcccaagatgtacatcattattgccaagcctgagaggaataccatcgaggaggtgcgttgcagcaccgcagctcacgctttcaaggtggctgcccgggccacgctgcgccgcagcaacgtctcccgcaagcggtccagcagccttggaggctccacgggatccaccccctcctcctccatcagcagcaagagcaacagcgaagacccattcccacagcccgagaggcagaagcagcagcagccgctggccctaacccagcaagagcagcagcagcagcccctgaccctcccacagcagcaacgatctcagcagcagcccagatgcaagcagaaggtcatctttggcagcggcacggtcaccttctcactgagctttgatgagcctcagaagaacgccatggcccacgggaattctacgcaccagaactccctggaggcccagaaaagcagcgatacgctgacccgacaccagccattactcccgctgcagtgcggggaaacggacttagatctgaccgtccaggaaacaggtctgcaaggacctgtgggtggagaccagcggccagaggtggaggaccctgaagagttgtccccagcacttgtagtgtccagttcacagagctttgtcatcagtggtggaggcagcactgttacagaaaacgtagtgaattcataaaatggaaggagaagactgggctag

SEQ ID NO: 30 - protein, 1056 aa, Artificial Sequence
  Description of Artificial Sequence Synthetic chimeric construct
MVRLLLIFFPMIFLEMSILPRMPDRKVLLAGASSQRSVARMDGDVIIGALFSVHHQPPAEKVPERKCGEIREQYGIQRVEAMFHTLDKINADPVLLPNITLGSEIRDSCWHSSVALEQSIEFIRDSLISIRDEKDGLNRCLPDGQTLPPGRTKKPIAGVIGPGSSSVAIQVQNLLQLFDIPQIAYSATSIDLSDKTLYKYFLRVVPSDTLQARAMLDIVKRYNWTYVSAVHTEGNYGESGMDAFKELAAQEGLCIAHSDKIYSNAGEKSFDRLLRKLRERLPKARVVVCFCEGMTVRGLLSAMRRLGVVGEFSLIGSDGWADRDEVIEGYEVEANGGITIKLQSPEVRSFDDYFLKLRLDTNTRNPWFPEFWQHRFQCRLPGHLLENPNFKRICTGNESLEENYVQDSKMGFVINAIYAMAHGLQNMHHALCPGHVGLCDAMKPIDGSKLLDFLIKSSFIGVSGEEVWFDEKGDAPGRYDIMNLQYTEANRYDYVHVGTWHEGVLNIDDYKIQMNKSGVVRSVCSEPCLKGQIKVIRKGEVSCCWICTACKENEYVQDEFTCKACDLGWWPNADLTGCEPIPVRYLEWSNIESIIAIAFSCLGILVTLFVTLIFVLYRDTPVVKSSSRELCYIILAGIFLGYVCPFTLIAKPTTTSCYLQRLLVGLSSAMCYSALVTKTNRIARILAGSKKKICTRKPRFMSAWAQVIIASILISVQLTLVVTLIIMEPPMPILSYPSIKEVYLICNTSNLGVVAPLGYNGLLIMSCTYYAFKTRNVPANFNEAKYIAFTMYTTCIIWLAFVPIYFGSNYKIITTCFAVSLSVTVALGCMFTPKMYIIIAKPERNTIEEVRCSTAAHAFKVAARATLRRSNVSRKRSSSLGGSTGSTPSSSISSKSNSEDPFPQPERQKQQQPLALTQQEQQQQPLTLPQQQRSQQQPRCKQKVIFGSGTVTFSLSFDEPQKNAMAHGNSTHQNSLEAQKSSDTLTRHQPLLPLQCGETDLDLTVQETGLQGPVGGDQRPEVEDPEELSPALVVSSSQSFVISGGGSTVTENVVNS

SEQ ID NO: 31 - protein, 60 aa, Artificial Sequence
  Description of Artificial Sequence Synthetic construct
MGSLLALPALLLLWGAVAEGPAKKVLTLEGDLVLGGLFPVHAKGPSGVPCGDIKRENGIH

SEQ ID NO: 32 - protein, 60 aa, Artificial Sequence
  Description of Artificial Sequence Synthetic construct
MAFYSCCWVLLALTWHTSAYGPDQRAQIEGDIQIGGLFPVHAKGPSGVPCGDIKRENGIH

SEQ ID NO: 33 - protein, 60 aa, Artificial Sequence
  Description of Artificial Sequence Synthetic construct
MARPRRAREPLLVALLPLAWLAQAGLARAAGSVRLAGGLTLGGLFPVHARGAAGRACGPL

SEQ ID NO: 34 - protein, 60 aa, Artificial Sequence
  Description of Artificial Sequence Synthetic construct
MPGKRGLGWWWARLPLCLLLSLYGPWMPSSLGKPKGHPHMNSIRIDGDITLGGLFPVHGR

SEQ ID NO: 35 - protein, 60 aa, Artificial Sequence
  Description of Artificial Sequence Synthetic construct
MLTRLQVLTLALFSKGFLLSLGDHNFLRREIKIEGDLVLGGLFPINEKGTGTEECGRINE

SEQ ID NO: 36 - protein, 60 aa, Artificial Sequence
  Description of Artificial Sequence Synthetic construct
MGSLLALLALLPLWGAVAEGPAKKVLTLEGDLVLGGLFPVHQKGGPAEDCGPVNEHRGIQ

SEQ ID NO: 37 - protein, 60 aa, Artificial Sequence
  Description of Artificial Sequence Synthetic construct
MAFYSCCWVLLALTWHTSAYGPDQRAQKKGDIILGGLFPIHFGVAAKDQDLKSRPESVEC

SEQ ID NO: 38 - protein, 60 aa, Artificial Sequence
  Description of Artificial Sequence Synthetic construct
MVLLLILSVLLLKEDVRGSAQSSERRVVAHMPGDIIIGALFSVHHQPTVDKVHERKCGAV

SEQ ID NO: 39 - protein, 60 aa, Artificial Sequence
  Description of Artificial Sequence Synthetic construct
MVGLLLFFFPAIFLEVSLLPRSPGRKVLLAGASSQRSVARMDGDVIIGALFSVHHQPPAE

SEQ ID NO: 40 - DNA, 18 bp, Artificial Sequence
  Description of Artificial Sequence Synthetic primer
ctcaccctctctggtcgc

SEQ ID NO: 41 - DNA, 21 bp, Artificial Sequence
  Description of Artificial Sequence Synthetic primer
tcttcctcctccatggtacca

SEQ ID NO: 42 - DNA, 20 bp, Artificial Sequence
  Description of Artificial Sequence Synthetic primer
taatacgactcactataggg

SEQ ID NO: 43 - DNA, 42 bp, Artificial Sequence
  Description of Artificial Sequence Synthetic primer
gagggtgacgtccccctcgatccgtatggaatgggcatactc

SEQ ID NO: 44 - DNA, 19 bp, Artificial Sequence
  Description of Artificial Sequence Synthetic primer
gttcgagcgcgtaagtgtc

SEQ ID NO: 45 - DNA, 42 bp, Artificial Sequence
  Description of Artificial Sequence Synthetic primer
ggcgtacatctcctggccgcgttgggctcgctggtctggccc

SEQ ID NO: 46 - DNA, 42 bp, Artificial Sequence
  Description of Artificial Sequence Synthetic primer
tgagtgcggggcgtacatctcttgggctcgctggtctggccc

SEQ ID NO: 47 - DNA, 36 bp, Artificial Sequence
  Description of Artificial Sequence Synthetic primer
ccagaccagcgagcccaaatcgagggggacgtcacc

SEQ ID NO: 48 - DNA, 21 bp, Artificial Sequence
  Description of Artificial Sequence Synthetic primer
atcgagggggacgtcaccctc

SEQ ID NO: 49 - DNA, 42 bp, Artificial Sequence
  Description of Artificial Sequence Synthetic primer
tagaaagttatggtcccctaattgggctcgctggtctggccc

SEQ ID NO: 50 - DNA, 42 bp, Artificial Sequence
  Description of Artificial Sequence Synthetic primer
tctccttagaaagttatggtcttgggctcgctggtctggccc

SEQ ID NO: 51 - DNA, 42 bp, Artificial Sequence
  Description of Artificial Sequence Synthetic primer
gggccagaccagcgcgcccaagagggcccagccaagaaggtg

(19) United States
(12) Patent Application Publication
(10) Pub. No.: US 2005/0186658 A1
(43) Pub. Date: Aug. 25, 2005

(54) Chimeric metabotropic glutamate receptors and uses thereof

(75) Inventors: Ashwani K. Gupta, Mississauga (CA); Pamela S. Jacobson, Salt Lake City, UT (US); Keith R. Jarvie, Mississauga (CA); Karen J. Krapcho, Park City, UT (US); Laura L. Storjohann, Salt Lake City, UT (US); Thomas M. Stormann, Salt Lake City, UT (US)

Correspondence Address: NPS PHARMACEUTICALS, INC. C/O FOLEY & LARDNER, P.O. BOX 80278, SAN DIEGO, CA, 92138-0278, US

(73) Assignee: NPS Pharmaceuticals, Inc.

(21) Appl. No.: 10/967,091

(22) Filed: Oct. 15, 2004

Related U.S. Application Data
(60) Provisional application No. 60/512,221, filed on Oct. 17, 2003.

(51) Int. Cl. C07H 21/04; C07K 14/705

(52) U.S. Cl. 435/69.1; 435/320.1; 435/325; 530/350; 536/23.5

(57) ABSTRACT

The present invention provides chimeric receptors that include an extracellular domain from a metabotropic glutamate receptor and a non-native signal peptide, e.g., a calcium receptor signal peptide. The invention also includes methods of preparing such chimeric receptors, and methods of using such receptors to identify and characterize compounds which modulate the activity of metabotropic glutamate receptors. The invention also relates to compounds and methods for modulating metabotropic glutamate receptor activity and binding to metabotropic glutamate receptors. Modulation of metabotropic glutamate receptor activity can be used for different purposes such as treating neurological disorders and diseases, inducing an analgesic effect, cognition enhancement, and inducing a muscle-relaxant effect.

### CROSS-REFERENCE TO RELATED PATENT APPLICATIONS

The application claims the benefit of U.S. Provisional Application 60/512,221, filed Oct. 17, 2003, which is incorporated herein by reference in its entirety, including drawings.

### BACKGROUND OF THE INVENTION

The present invention relates to chimeric receptors containing one or more regions homologous to a metabotropic glutamate receptor and a calcium receptor or other non-native signal peptide.

The following description provides a summary of information relevant to the present invention. It is not an admission that any of the information provided herein is prior art to the presently claimed invention, nor that any of the publications specifically or implicitly referenced are prior art to that invention.

Glutamate is the major excitatory neurotransmitter in the mammalian brain. Glutamate produces its effects on central neurons by binding to and thereby activating cell surface receptors. These receptors have been subdivided into two major classes, the ionotropic and metabotropic glutamate receptors, based on the structural features of the receptor proteins, the means by which the receptors transduce signals into the cell, and pharmacological profiles.

The ionotropic glutamate receptors (iGluRs) are ligand-gated ion channels that, upon binding glutamate, open to allow the selective influx of certain monovalent and divalent cations, thereby depolarizing the cell membrane. In addition, certain iGluRs with relatively high calcium permeability can activate a variety of calcium-dependent intracellular processes. These receptors are multisubunit protein complexes that may be homomeric or heteromeric in nature. The various iGluR subunits all share common structural motifs, including a relatively large amino-terminal extracellular domain (ECD), followed by a multiple transmembrane domain (TMD) comprising two membrane-spanning regions (TMs), a second smaller intracellular loop, and a third TM, before terminating with an intracellular carboxy-terminal domain (CT). Historically the iGluRs were first subdivided pharmacologically into three classes based on preferential activation by the agonists alpha-amino-3-hydroxy-5-methyl-isoxazole-4-propionic acid (AMPA), kainate (KA), and N-methyl-D-aspartate (NMDA). Later, molecular cloning studies coupled with additional pharmacological studies revealed a greater diversity of iGluRs, in that multiple subtypes of AMPA, KA and NMDA receptors are expressed in the mammalian CNS (Hollman and Heinemann, Ann. Rev. Neurosci. 7:31, 1994).

The metabotropic glutamate receptors (mGluRs) are G protein-coupled receptors capable of activating a variety of intracellular second messenger systems following the binding of glutamate or other potent agonists including quisqualate and 1-aminocyclopentane-1,3-dicarboxylic acid (trans-ACPD) (Schoepp et al., Trends Pharmacol. Sci. 11:508, 1990; Schoepp and Conn, Trends Pharmacol. Sci. 14:13, 1993).

Activation of different metabotropic glutamate receptor subtypes in situ elicits one or more of the following responses: activation of phospholipase C, increases in phosphoinositide (PI) hydrolysis, intracellular calcium release, activation of phospholipase D, activation or inhibition of adenylyl cyclase, increases and decreases in the formation of cyclic adenosine monophosphate (cAMP), activation of guanylyl cyclase, increases in the formation of cyclic guanosine monophosphate (cGMP), activation of phospholipase A₂, increases in arachidonic acid release, and increases or decreases in the activity of voltage- and ligand-gated ion channels (Schoepp and Conn, Trends Pharmacol. Sci. 14:13, 1993; Schoepp, Neurochem. Int. 24:439, 1994; Pin and Duvoisin, Neuropharmacology 34:1, 1995).

Thus far, eight distinct mGluR subtypes have been isolated via molecular cloning, and named mGluR1 to mGluR8 according to the order in which they were discovered (Nakanishi, Neuron 13:1031, 1994, Pin and Duvoisin, Neuropharmacology 34:1, 1995; Knopfel et al., J. Med. Chem. 38:1417, 1995). Further diversity occurs through the expression of alternatively spliced forms of certain mGluR subtypes (Pin et al., PNAS 89:10331, 1992; Minakami et al., BBRC 199:1136, 1994). All of the mGluRs are structurally similar, in that they are single subunit membrane proteins possessing a large amino-terminal extracellular domain (ECD) followed by seven putative transmembrane domain (7TMD) comprising seven putative membrane spanning helices connected by three intracellular and three extracellular loops, and an intracellular carboxy-terminal domain of variable length (cytoplasmic tail) (CT) (see, Schematic receptor A in FIG. 1).

The various mGluRs have been subdivided into three groups based on amino acid sequence identities, the second messenger systems they utilize, and pharmacological characteristics (Nakanishi, Neuron 13:1031, 1994; Pin and Duvoisin, Neuropharmacology 34:1, 1995; Knopfel et al., J. Med. Chem. 38:1417, 1995). The amino acid identity between mGluRs within a given group is approximately 70% but drops to about 40% between mGluRs in different groups. For mGluRs in the same group, this relatedness is roughly paralleled by similarities in signal transduction mechanisms and pharmacological characteristics.

The Group I mGluRs comprise mGluR1, mGluR5 and their alternatively spliced variants. The binding of agonists to these receptors results in the activation of phospholipase C and the subsequent mobilization of intracellular calcium. For example, Xenopus oocytes expressing recombinant mGluR1 receptors have been utilized to demonstrate this effect indirectly by electrophysiological means (Masu et al., Nature 349:760, 1991; Pin et al., PNAS 89:10331, 1992). Similar results were achieved with oocytes expressing recombinant mGluR5 receptors (Abe et al., J. Biol. Chem. 267:13361, 1992; Minakami et al., BBRC 199:1136, 1994). Alternatively, agonist activation of recombinant mGluR1 receptors expressed in Chinese hamster ovary (CHO) cells stimulated PI hydrolysis, cAMP formation, and arachidonic acid release as measured by standard biochemical assays (Aramori and Nakanishi, Neuron 8:757, 1992). In comparison, activation of mGluR5 receptors expressed in CHO cells stimulated PI hydrolysis and subsequent intracellular calcium transients but no stimulation of cAMP formation or arachidonic acid release was observed (Abe et al., J. Biol. Chem. 267:13361, 1992). The agonist potency profile for Group I mGluRs is quisqualate>glutamate=ibotenate>(2S, 1′S,2′S)-2-carboxycyclopropyl)glycine (L-CCG-I)>(1S,3R)-1-aminocyclopentane-1,3-dicarboxylic acid (ACPD). Quisqualate is relatively selective for Group I receptors, as compared to Group II and Group III mGluRs, but it also potently activates ionotropic AMPA receptors (Pin and Duvoisin, Neuropharmacology, 34:1, Knopfel et al., J. Med. Chem. 38:1417, 1995).

The Group II mGluRs include mGluR2 and mGluR3. Activation of these receptors as expressed in CHO cells inhibits adenylyl cyclase activity via the inhibitory G protein, Gᵢ, in a pertussis toxin-sensitive fashion (Tanabe et al., Neuron 8:169, 1992; Tanabe et al., Neurosci. 13:1372, 1993). The agonist potency profile for Group II receptors is L-CCG-I>glutamate>ACPD>ibotenate>quisqualate. Preliminary studies suggest that L-CCG-I and (2S,1′R,2′R,3′R)-2-(2,3-dicarboxycyclopropyl)glycine (DCG-IV) are both relatively selective agonists for the Group II receptors (Knopfel et al., J. Med. Chem. 38:1417, 1995).

The Group III mGluRs include mGluR4, mGluR6, mGluR7 and mGluR8. Like the Group II receptors these mGluRs are negatively coupled to adenylate cyclase to inhibit intracellular cAMP accumulation in a pertussis toxin-sensitive fashion when expressed in CHO cells (Tanabe et al., J. Neurosci. 13:1372, 1993; Nakajima et al., J. Biol. Chem. 268:11868, 1993; Okamoto et al., J. Biol. Chem. 269:1231, 1994; Duvoisin et al., J Neurosci. 15:3075, 1995). As a group, their agonist potency profile is (S)-2-amino-4-phosphonobutyric acid (L-AP4)>glutamate>ACPD>quisqualate, but mGluR8 may differ slightly with glutamate being more potent than L-AP4 (Knopfel et al., J. Med Chem. 38:1417, 1995; Duvoisin et al., J. Neurosci. 15:3075, 1995). Both L-AP4 and (S)-serine-O-phosphate (L-SOP) are relatively selective agonists for the Group III receptors. The mGluR4 and mGluR7 (including splice variants 7a and 7b) are described in U.S. Pat. No. 6,288,610 which is incorporated herein by reference in its entirety.

Finally, the various mGluR subtypes have unique patterns of expression within the mammalian CNS that in many instances are overlapping (Masu et al., Nature 349:760, 1991; Martin et al., Neuron 9:259, 1992; Ohishi et al., Neurosci. 53:1009, 1993; Tanabe et al., J. Neurosci. 13:1372; Ohishi et al., Neuron 13:55, 1994, Abe et al., J. Biol. Chem. 267:13361, 1992; Nakajima et al., J. Biol. Chem. 268:11868, 1993; Okamoto et al., J. Biol. Chem. 269:1231, 1994; Duvoisin et al., J. Neurosci. 15:3075, 1995). As a result certain neurons may express only one particular mGluR subtype, while other neurons may express multiple subtypes that may be localized to similar and/or different locations on the cell (i.e., postsynaptic dendrites and/or cell bodies versus presynaptic axon terminals). Therefore, the functional consequences of mGluR activation on a given neuron will depend on the particular mGluRs being expressed; the receptors' affinities for glutamate and the concentrations of glutamate the cell is exposed to; the signal transduction pathways activated by the receptors; and the locations of the receptors on the cell. A further level of complexity may be introduced by multiple interactions between mGluR expressing neurons in a given brain region. As a result of these complexities, and the lack of subtype-specific mGluR agonists and antagonists, the roles of particular mGluRs in physiological and pathophysiological processes affecting neuronal function are not well defined. Still, work with the available agonists and antagonists have yielded some general insights about the Group I mGluRs as compared to the Group II and Group III mGluRs.

Attempts at elucidating the physiological roles of Group I mGluRs suggest that activation of these receptors elicits neuronal excitation. Various studies have demonstrated that ACPD can produce postsynaptic excitation upon application to neurons in the hippocampus, cerebral cortex, cerebellum, and thalamus as well as other brain regions. Evidence indicates that this excitation is due to direct activation of postsynaptic mGluRs, but it has also been suggested to be mediated by activation of presynaptic mGluRs resulting in increased neurotransmitter release (Baskys, Trends Pharmacol. Sci. 15:92, 1992; Schoepp, Neurochem. Int. 24:439, 1994; Pin and Duvoisin, Neuropharmacology 34:1). Pharmacological experiments implicate Group I mGluRs as the mediators of this excitation. The effect of ACPD can be reproduced by low concentrations of quisqualate in the presence of iGluR antagonists (Hu and Storm, Brain Res. 568:339, 1991; Greene et al. Eur. J. Pharmacol. 226:279, 1992), and two phenylglycine compounds known to activate mGluR1, (S)-3-hydroxyphenylglycine ((S)-3HPG) and (S)-3,5-dihydroxyphenylglycine ((S)-DHPG), also produce the excitation (Watkins and Collingridge, Trends Pharmacol. Sci. 15:333, 1994). In addition, the excitation can be blocked by (S)-4-carboxyphenylglycine ((S)-4CPG), (S)-4-carboxy-3-hydroxyphenylglycine ((S)-4C3HPG) and (+)-alpha-methyl-4-carboxyphenylglycine ((+)-MCPG), compounds known to be mGluR1 antagonists (Eaton et al., Eur. J. Pharmacol. 244:195, 1993; Watkins and Collingridge, Trends Pharmacol. Sci. 15:333, 1994).

Other studies examining the physiological roles of mGluRs indicate that activation of presynaptic mGluRs can block both excitatory and inhibitory synaptic transmission by inhibiting neurotransmitter release (Pin and Duvoisin, Neuropharmacology 34:1). Presynaptic blockade of excitatory synaptic transmission by ACPD has been observed on neurons in the visual cortex, cerebellum, hippocampus, striatum and amygdala (Pin et al., Curr. Drugs: Neurodegenerative Disorders 1:111, 1993), while similar blockade of inhibitory synaptic transmission has been demonstrated in the striatum and olfactory bulb (Calabresi et al., Neurosci. Lett. 139:41, 1992; Hayashi et al., Nature 366:687, 1993). Multiple pieces of evidence suggest that Group II mGluRs mediate this presynaptic inhibition. Group II mGluRs are strongly coupled to inhibition of adenylyl cyclase, like alpha₂-adrenergic and 5HT_{1A}-serotonergic receptors which are known to mediate presynaptic inhibition of neurotransmitter release in other neurons. The inhibitory effects of ACPD can also be mimicked by L-CCG-I and DCG-IV, which are selective agonists at Group II mGluRs (Hayashi et al., Nature 366:687, 1993; Jane et al., Br. J. Pharmacol. 112:809, 1994). Moreover, it has been demonstrated that activation of mGluR2 can strongly inhibit presynaptic, N-type calcium channel activity when the receptor is expressed in sympathetic neurons (Ikeda et al., Neuron 14:1029, 1995), and inactivation of these channels is known to inhibit neurotransmitter release. Finally, it has been observed that L-CCG-I, at concentrations selective for Group II mGluRs, inhibits the depolarization-evoked release of ³H-aspartate from rat striatal slices (Lombardi et al., Br. J. Pharmacol. 110: 1407, 1993). Evidence for physiological effects of Group II mGluR activation at the postsynaptic level is limited. However, one study suggests that postsynaptic actions of L-CCG-I can inhibit NMDA receptor activation in cultured mesencephalic neurons (Ambrosini et al., Mol. Pharmacol. 47:1057, 1995).

Physiological studies have demonstrated that L-AP4 can also inhibit excitatory synaptic transmission on a variety of CNS neurons. Included are neurons in the cortex, hippocampus, amygdala, olfactory bulb and spinal cord (Koerner and Johnson, Excitatory Amino Acid Receptors; Design of Agonists and Antagonists p. 308, 1992; Pin et al., Curr. Drugs: Neurodegenerative Disorders 1:111, 1993). The accumulated evidence indicates that the inhibition is mediated by activation of presynaptic mGluRs. Since the effects of L-AP4 can be mimicked by L-SOP, and these two agonists are selective for Group III mGluRs, members of this mGluR group are implicated as the mediators of the presynaptic inhibition (Schoepp, Neurochem. Int. 24:439, 1994; Pin and Duvoisin, Neuropharmacology 34:1). In olfactory bulb neurons it has been demonstrated that L-AP4 activation of mGluRs inhibits presynaptic calcium currents (Trombley and Westbrook, J. Neurosci. 12:2043, 1992). It is therefore likely that the mechanism of presynaptic inhibition produced by activation of Group III mGluRs is similar to that for Group II mGluRs, i.e., blockade of N-type calcium channels and inhibition of neurotransmitter release. L-AP4 is also known to act postsynaptically to hyperpolarize ON bipolar cells in the retina. It has been suggested that this action may be due to activation of a mGluR, which is coupled to the cGMP phosphodiesterase in these cells (Schoepp, Neurochem. Int. 24:439, 1994; Pin and Duvoisin, Neuropharmacology 34:1).

Metabotropic glutamate receptor activation studies using agonists, antagonists and recombinant vertebrate cell lines expressing mGluRs have been used to evaluate the cellular effects of the stimulation and the inhibition of different metabotropic glutamate receptors. For example, agonist stimulation of mGluR1 expressed in Xenopus oocytes demonstrated coupling of receptor activation to mobilization of intracellular calcium as assessed indirectly using electrophysiology techniques (Masu et al., Nature 349:760-765, 1991). Agonist stimulation of mGluR1 expressed in CHO cells stimulated PI hydrolysis, cAMP formation and arachidonic acid release (Aramori and Nakanishi, Neuron 8:757-765, 1992). Agonist stimulation of mGluR5 expressed in CHO cells also stimulated PI hydrolysis which was shown to be associated with a transient increase in cytosolic calcium as assessed by loading cells with the fluorescent calcium chelator fura-2 (Abe et al., J. Biol. Chem. 267:13361-13368, 1992). Agonist-induced activation of mGluR1 and mGluR5 induced PI hydrolysis in CHO cells was not antagonized by AP3 and AP4, which are both antagonists of glutamate-stimulated PI hydrolysis in situ (Nicoletti et al., Proc. Natl. Acad. Sci. USA 833:1931-1935, 1986; Schoepp and Johnson, J. Neurochem. 53:273-278, 1989). Agonist stimulation of CHO cells expressing mGluR2 (Tanabe et al., Neuron 8:169-179, 1992) or mGluR7 (Okamoto et al., J. Biol. Chem. 269:1231-1236, 1994) resulted in receptor-mediated inhibition of cAMP formation and also confirmed the ligand specificity previously observed in situ. Studies using agonists were also carried out in conjunction with site-directed mutagenesis to reveal specific amino acids playing important roles in glutamate binding (O'Hara et al., Neuron 11:41-52, 1993).

Metabotropic glutamate receptors (mGluRs) have been implicated in a variety of neurological pathologies including stroke, head trauma, spinal cord injury, epilepsy, ischemia, hypoglycemia, anoxia, and neurodegenerative diseases such as Alzheimer's disease (Schoepp and Conn, Trends Pharmacol. Sci. 14:13, 1993; Cunningham et al., Life Sci. 54: 135, 1994; Pin et al., Neuropharmacology 34:1, 1995; Knopfel et al., J. Med Chem. 38:1417, 1995;). A role for metabotropic glutamate receptors in nociception and analgesia has also been demonstrated (Meller et al., Neuroreport 4:879, 1993). Metabotropic glutamate receptors have also been shown to be required for the induction of hippocampal long-term potentiation and cerebellar long-term depression (Bashir et al., Nature 363:347, 1993; Bortolotto et al., Nature 368:740, 1994; Aiba et. al. Cell 79: 365 and Cell 79: 377, 1994).

Metabotropic glutamate receptor agonists have been reported to have effects on various physiological activities. For example, trans-ACPD was reported to possess both proconvulsant and anticonvulsant effects (Zheng and Gallagher, Neurosci. Lett. 125:147, 1991; Sacaan and Schoepp, Neurosci. Lett. 139:77, 1992; Taschenberger et al., Neuroreport 3:629, 1992; Sheardown, Neuroreport 3:916, 1992), and neuroprotective effects in vitro and in vivo (Pizzi et al., J. Neurochem. 61:683, 1993; Koh et al., Proc. Natl. Acad. Sci. USA 88:9431, 1991; Birrell et al., Neuropharmacol. 32:1351, 1993; Siliprandi et al., Eur. J. Pharmacol. 219:173, 1992; Chiamulera et al., Eur. J. Pharmacol. 216:335, 1992). The metabotropic glutamate receptor antagonist L-AP3 was shown to protect against hypoxic injury in vitro (Opitz and Reymann, Neuroreport 2:455, 1991). A subsequent study reported that trans-ACPD produced neuroprotection which was antagonized by L-AP3 (Opitz and Reymann, Neuropharmacol. 32:103, 1993). (5)-4C3HPG was shown to protect against audiogenic seizures in DBA/2 mice (Thomasen et al., J. Neurochem. 62:2492, 1994). Other modulatory effects expected of metabotropic glutamate receptor modulators include synaptic transmission, neuronal death, neuronal development, synaptic plasticity, spatial learning, olfactory memory, central control of cardiac activity, waking, control of movements, and control of vestibulo ocular reflex (for reviews, see Nakanishi, Neuron 13:1031-37, 1994; Pin et al., Neuropharmacology 34:1, 1995; Knopfel et al., J. Med. Chem. 38:1417, 1995).

The structures of mGluR-active molecules currently known in the art are limited to amino acids which appear to act by binding at the glutamate binding site (Pin, et al, Neuropharmacology 34:1, 1995; Knopfel et al., J. Med. Chem. 38:1418). This limits the range of pharmacological properties and potential therapeutic utilities of such compounds. Furthermore, the range of pharmacological specificities associated with these mGluR- active molecules does not allow for complete discrimination between different subtypes of metabotropic glutamate receptors (Pin et al., Neuropharmacology 34:1, 1995 and Knopfel et al., J. Med. Chem. (1995) 38:1418). Rapid progress in the field of mGluR-active molecules cannot be made until more potent and more selective mGluR agonists, antagonists and modulators are discovered (Pin et al., Neuropharmacology 34:1, 1995; Knopfel et al., J. Med. Chem. (1995) 38:1418). Indeed, no mGluR-active molecules are presently under clinical development. High throughput functional screening of compounds and compound libraries using cell lines expressing individual mGluRs represents an important approach to identifying such novel compounds (Knopfel et al., J. Med. Chem. 38:1418).

Several laboratories have constructed cell lines expressing metabotropic glutamate receptors which appear to function appropriately (Abe et al., J. Biol. Chem. 267:13361, 1992; Tanabe et al., Neuron 8:169, 1992; Aramori and Nakanishi, Neuron 8:757, 1992, Nakanishi, Science 258:597, 1992; Thomsen et al., Brain Res. 619:22, 1992; Thomsen et al., Eur. J. Pharmacol. 227:361, 1992; O'Hara et al., Neuron 11:41, 1993; Nakjima et al., J. Biol. Chem. 268:11868, 1993; Tanabe et al., J. Neurosci. 13:1372, 1993; Saugstad et al., Mol. Pharmacol. 45:367, 1994; Okamoto et al., J. Biol. Chem. 269:1231, 1994; Gabellini et al., Neurochem. Int. 24:533, 1994; Lin et al., Soc. Neurosci. Abstr. 20:468, 1994; Flor et al., Soc. Neurosci. Abstr. 20:468, 1994; Flor et al., Neuropharmacology 34:149, 1994). Other reports have noted that expression of functional mGluR expressing cell lines is not predictable. For example, Tanabe et al., (Neuron 8:169, 1992) were unable to demonstrate functional expression of mGluR3 and mGluR4, and noted difficulty obtaining expression of native mGluR1 in CHO cells. Gabellini et al., (Neurochem. Int. 24:533, 1994) also noted difficulties with mGluR1 expression in HEK 293 cells and it is possible that some of these difficulties may be due to desensitization characteristics of these receptors. Furthermore, screening methodologies useful for identification of compounds active at Class I mGluRs are not readily amenable to identification of compounds active at class II and III mGluRs and vice versa due to the differences in second messenger coupling. Finally, mGluRs have been noted to rapidly desensitize upon agonist stimulation which may adversely affect the viability of cell lines expressing these receptors and makes the use of native mGluRs for screening difficult.

Different G-protein coupled receptors exhibit differential ligand affinities and coupling to second messengers. G-protein coupled receptors all have a similar structure: an N-terminal extracellular domain (ECD), a seven-transmembrane domain (7TMD) comprising seven membrane spanning helices and therefore defining three intracellular and three extracellular loops, and a cytoplasmic tail (CT), but differ in the exact sequences comprising each region. These sequence differences are thought to provide the specificity of receptor interactions with ligands of different chemical compositions and receptor interaction with different G-proteins. Construction of chimeric receptors in which small peptide segments from related receptors are exchanged using recombinant DNA techniques has proven a useful technique to assess the participation of different sequence regions in determining this specificity. For example, exchanging the third intracellular loops between various adrenergic, muscarinic acetylcholine and angiotensin receptors results in conversion of G-protein coupling specificity. Thus, receptors whose activation normally results in inhibition or activation of adenylate cyclase can be converted to receptors with the same or similar ligand binding properties but whose activation leads to stimulation of phospholipase-C and vice versa (Kobilka et al., Science 240:1310, 1988; Wess et al., FEBS Lett. 258:133, 1989; Cotecchia et al., Proc. Nat'l Acad. Sci. U.S.A. 87:2896, 1990; Lechleiter et al., EMBO J. 9:4381, 1990; Wess et al., Mol. Pharmacol. 38:517, 1990; Wong et al., J. Biol. Chem. 265:6219, 1990; Cotecchia et al., J. Biol. Chem. 267:1633, 1992; Wang et al., J. Biol. Chem. 270:16677, 1995). In these receptors the third intracellular loop plays an important role in determining the specificity of G-protein coupling. While such experiments indicate that the third intracellular loop plays an important role in determining the specificity of G protein coupling in these related receptors, they have failed to identify any specific amino acid sequence motif which is responsible. In addition, the third intracellular loop has been shown to be at least partly responsible for desensitization of such receptors (Okamoto et al., Cell 67:723, 1991; Liggett et al., J. Biol. Chem. 267:4740, 1992).

Metabotropic glutamate receptors are related to other G-protein coupled receptors in overall topology, but not in specific amino acid sequence. An unusual feature of mGluRs is their very large ECDs (ca. 600 amino acids). In many other G-protein coupled receptors, ligand binding takes place within the 7TMDs. However, the large ECD of each mGluR is thought to provide the ligand binding determinants (Nakanishi, Science 258:597, 1992; O'Hara et al., Neuron 11:41, 1993; Shigemoto et al., Neuron 12:1245, 1994). Chimeric mGluRs in which the ECDs of mGluRs with different ligand affinities and different G-protein coupling are exchanged have been used to demonstrate that the ECD of mGluRs defines ligand specificity but not G-protein specificity (Takahashi et al., J. Bio. Chem. 268:19341, 1993). Also unlike other G-protein coupled receptors in which the third intracellular loop is variable in size and sequence, the third intracellular loops of mGluRs are small and extremely well conserved (Brown E. M. et al., Nature 366:575, 1993). Chimeric mGluRs have been prepared in which the second intracellular loops and/or cytoplasmic tails were exchanged (Pin et al., EMBO J. 13:342). These experiments lead the investigators to conclude that unlike most other G-protein coupled receptors, “both the C-terminal end of the second intracellular loop and the segment located downstream of the seventh transmembrane domain are necessary for the specific activation of phospholipase-C by mGluR1c” and to suggest that the second intracellular loop of mGluRs plays the role of the third intracellular loop of other G-protein coupled receptors.

Naturally occurring mRNA splice variants have been noted to produce prostaglandin E3 (EP3) receptors with essentially identical ligand binding properties but which preferentially activate different second messenger pathways (differential G-protein coupling) and which exhibit different desensitization properties (Namba et al., Nature 365:166, 1993; Shigemoto et al., J. Biol. Chem. 268:2712, 1993; Negishi et al., J. Biol. Chem. 268:9517, 1993). These variant receptor isoforms differ only in their cytoplasmic tails. The isoforms with the longer tails couple efficiently to phospholipase-C while those with the shorter tails do not. However, analyses of naturally occurring mRNA splice variants of mGluR1 and mGluR5 have indicated that their long cytoplasmic tails may not be directly involved in G protein coupling (Pin et al., Proc. Nat'l. Acad. Sci. U.S.A. 89:10331, 1992; Joly et al., J. Neuroscience 15:3970, 1995). In fact, Pin et al., (supra) have stated that “The very long C-terminal domain found only in PLC-coupled mGluRs (mGluR1 and 5) is, however, probably not involved in the specific interaction with PLC-activating G proteins.”

The calcium receptor has been described (Brown E. M. et al., Nature 366:575, 1993; Riccardi D., et al., Proc. Nat'l. Acad. Sci. USA 92:131-135, 1995; Garrett J. E., et al., J. Biol. Chem. 31:12919-12925, 1995). This CaR is the only known receptor which exhibits significant sequence homology with mGluRs except for other mGluRs. The CaR exhibits about ˜25% sequence homology (amino acid identities) to any one mGluR while mGluRs are >40% homologous (amino acid identities) to one another. The CaR is structurally related to mGluRs having a large ECD which has been implicated in receptor function and probable ligand binding (Brown E. M. et al., Nature 366:575, 1993; Pollak, M. R., et al., Cell 75:1297-1303, 1993). This similarity of structure does not confer close similarity in ligand binding specificity since the native ligand for the CaR is the inorganic ion, Ca²⁺, and glutamate does not modulate CaR activity. The CaR also has a large cytoplasmic tail and is coupled to the stimulation of phospholipase-C.

Thus, the CaR is structurally and functionally more related to mGluR1 and 5 than to other mGluRs. Pin et al., (EMBO J. 13:342, 1994) have noted that certain amino acids are conserved within the intracellular loops of mGluRs which couple to phospholipase-C and different amino acids are conserved in these same positions within the intracellular loops of mGluRs which couple to the inhibition of adenylate cyclase. Intracellular loops 1 and 3 are the most highly conserved sequences between mGluRs and the CaR (Brown E. M. et al., Nature 366:575, 1993), but only about half of these particular amino acids are found in the corresponding position of the CaR and only one of these is actually the amino acid predicted for a receptor which couples to phospholipase-C. Thus, sequence conservation between CaRs and mGluRs appears to be consistent mostly with conservation of structural domains involved in ligand binding and G-protein coupling and does not provide evidence for specific sequence motifs within intracellular regions predictive of G-protein coupling specificity. Cell lines expressing CaRs have been obtained and their use to identify compounds which modulate the activity of CaRs disclosed (U.S. Pat. Nos. 6,011,068, 5,858,684, 5,688,938, 5,763,569, 6,031,003, 6,313,146, and 6,211,244, all hereby incorporated by reference herein in their entireties).

An advantageous screening procedure for identifying molecules specifically affecting the activity of different mGluRs would provide cell lines expressing each functional mGluR in such a manner that each was coupled to the same second messenger system and amenable to high throughput screening.

None of the references mentioned herein are admitted to be prior art to the claims.

### SUMMARY OF THE INVENTION

The present invention concerns chimeric receptors that are advantageous for screening for compounds active on metabotropic glutamate receptors. Thus, the invention concerns (1) chimeric receptor proteins having sequences from metabotropic glutamate receptors and a signal peptide from a calcium receptor or other non-native signal peptide, and fragments of metabotropic glutamate receptors, calcium receptors, and chimeric receptors, which can be isolated and/or can have such a non-native signal peptide; (2) nucleic acids encoding such chimeric receptor proteins and fragments; (3) uses of such receptor proteins, fragments and nucleic acids; (4) cell lines expressing such nucleic acids; (5) methods of screening for compounds that bind to or modulate the activity or cellular disposition of metabotropic glutamate receptors or calcium receptors using such chimeric receptor proteins and fragments; (6) compounds for modulating metabotropic glutamate receptors or calcium receptors identified by such methods of screening; (7) methods for modulating metabotropic glutamate receptors or calcium receptors utilizing such compounds; and (8) methods of treating disorders that arise from or can be treated by modulation of metabotropic glutamate receptor activity (e.g., neurological disorders) using such compounds.

As indicated, an advantageous use of the constructs and methods of the present invention is to screen for compounds which modulate metabotropic glutamate receptor activity and to use such compounds to aid in the treatment of neurological diseases or disorders.

As described in the Background of the Invention above, metabotropic glutamate receptors (mGluR) and calcium receptors (CaR) have similar structures. Both types of receptors have an extracellular domain (ECD), a seven transmembrane domain (7TMD) and an intracellular cytoplasmic tail (CT). The present chimeric receptors include an extracellular domain that is the same as or has a high level of sequence identity to an mGluR and a signal peptide (SP) that is from a non-native source (i.e., not naturally associated with the mGluR from which the chimeric receptor mGluR sequence is obtained or derived), e.g., is from a CaR or a different mGluR or has a high level of sequence identity to a SP from such different source. Changing the signal peptide in this manner can provide increased expression of an mGluR and/or provide an epitope that makes assaying and/or visualizing the presence of such receptors more convenient than native receptors. For example, an antibody can be used that recognizes a fragment derived from the non-native signal peptide or the junction between the non-native signal peptide and the mGluR sequence.

Thus, as used herein in connection with the present chimeric receptors and signal peptides, the term “non-native” means that that the signal peptide is from a different receptor type or sub-type other than the mGluR sequence to which the signal peptide is linked. Thus, for example, the signal peptide can be from a different mGluR subtype than the mGluR sequence to which it is linked, or the signal peptide can be from a CaR. Such signal peptides can also be from other receptor types. Such signal peptides can be selected by selecting a signal peptide from a receptor that is readily expressed at a useful level in the desired host cell, and can be tested in chimeric receptors as described herein. In general such signal peptides are identified as putative signal peptides by identifying a hydrophobic N-terminal sequence.

In combination with the changed signal peptide, the chimeric receptors can also include a combination of domains from mGluR and from CaR. Thus in certain embodiments, the extracellular domain is from an mGluR and a portion of the sequence of the receptor is from a CaR, and thus the respective sequences are the same as same as or has a high level of sequence identity to a portion of the sequence of a CaR. For example, the chimeric receptor can consist of the ECD of an mGluR and the 7TMD and CT of a CaR. Likewise, a chimeric receptor may include the ECD and 7TMD of an mGluR and the CT of a CaR. Such mGluR/CaR chimeric receptors (but without the signal peptide change) are described, for example, in U.S. Pat. No. 5,981,195, which is incorporated herein by reference in its entirety, including drawings. Chimeric receptors as described therein having mGluR ECDs can be used in the present invention along with a suitable signal peptide.

These chimeric receptors that include both mGluR and CaR domains are of interest, in part, because they allow the coupling of certain functional aspects of an mGluR with certain functional aspects of a CaR. Thus, experiments have shown that ligands known in the art which are agonists or antagonists on a native mGluR also exhibit such activities on chimeric receptors in which the extracellular domain is from the mGluR. Similarly, experiments have shown that ligands known in the art which modulate mGluRs act on chimeric receptors in which the extracellular domain and the 7TMD are from an mGluR.

In the context of the present invention, indicating that an amino acid or nucleic acid sequence is “from an mGluR” or “from a CaR” means that the sequence is closely related to a native sequence from the particular receptor. Generally this means that the level of sequence identity is at least 50, 60, 70, 80, 90, 95, 97, 98, or 99% based on a maximal alignment using either BLASTN or BLASTP with default parameters (available from NCBI) or is identical. In the absence of those tools, ALIGN (from Genstream Resource Center) can be used with default parameters. In the absence of either of these alignment programs, any commonly used sequence alignment tool can be used with default parameters for determining percent identity.

As used in the context of the present invention, the term “signal peptide” indicates a continuous stretch of amino acids located at or near the N-terminus of a protein. The signal peptide signals transport of a section of the protein into the lumen of the ER and, thus, serves to determine the eventual orientation of the protein in the cell membrane. A signal peptide used in the chimeric receptors of the present invention will typically include a region of hydrophobic amino acid residues and may be partially or completely cleaved off during protein maturation.

Signal peptides useful in chimeric receptors of the present invention can be identified or defined using techniques known in the art. For example, a suite of software, Vector NTI, which is available from Informax, Inc. of Bethesda, Md., provides an algorithm useful in determining the location and identity of signal peptides within proteins. In addition, multiple publications include predictions of signal peptides for various proteins included in Family C of the G-protein coupled receptors. One such article, entitled “A Family of Metabotropic Glutamate Receptors” by Tanab et al. (1992, Neuron, Vol. 8, pp. 169-179), describes the signal peptides predicted in mGluR1 through mGluR4.

The present chimeric receptors can further be constructed as fusion receptors in which the intracellular domain, an intracellular domain tail, or the transmembrane domain is covalently linked to a G-Protein. Fusion receptors are described, for example, in International Application PCT/US99/07333, International Publication WO 99/51641, which is incorporated herein by reference in its entirety, including drawings (also described in corresponding U.S. application Ser. No. 09/679,664, which is also incorporated herein by reference in its entirety), including exemplary G-proteins for fusing to the receptor. The G-protein can be selected such that the receptor couples to a different pathway that the receptor would normally couple. Such a change can, for example, allow a receptor to couple to a pathway that provides a more convenient signal for use in an assay, e.g., suitable for high throughput screening.

The use of mGluRs for screening for mGluR active compounds has been complicated by a number of factors including a rapid desensitization of the receptor upon ligand binding/activation and difficulties in stably expressing the receptors in recombinant vertebrate cells (see, for example, FIG. 6B). Certain of the chimeric receptors of the present invention can be utilized to overcome these technical difficulties and provide much improved screening methods by utilizing the more robust aspects of calcium receptors. For example, by coupling the 7TMD and the CT of the CaR with the extracellular domain of an mGluR, or the CT of the CaR to the ECD and 7TMD of the mGluR, the mGluR extracellular domain has the benefit of the Gq coupling property of a CaR as well as the improved property of a lack of rapid densensitization (see, for example, FIG. 6C). Thus, such a chimeric receptor has the ligand binding and activation properties similar to those of a native mGluR but having the improved second messenger coupling similar to a CaR. Therefore, the chimeric receptor simplifies and enables efficient, practical, and reproducible functional screens to identify mGluR active molecules.

It is recognized that the three domains described above are made up of sub-domains, for example, ligand binding sites and G protein coupling sites. Therefore, for some applications it is not necessary to include in a chimeric receptor a complete domain from a particular receptor in order to have the desired activity. For example, in a chimeric receptor, cytoplasmic loops between the membrane-spanning helices in one Family C GPCR (e.g.,mGluR) can be replaced with the homologous region from another Family C GPCR (e.g., CaR). Thus, in particular examples, one of the cytoplasmic loops of the 7TMD can be from a loop sequence of an mGluR and substantially the remainder of the sequence of the receptor can be from a CaR, or conversely, one of the cytoplasmic loops can be from a loop sequence of a CaR and substantially the remainder of the sequence of the receptor can be from an mGluR.

Thus, in a first aspect the invention features a composition including a chimeric receptor which has an extracellular domain, a seven transmembrane domain, and generally an intracellular cytoplasmic tail domain. The chimeric receptor has a non-native signal peptide linked at the N-terminus of the extracellular domain sequence, where the extracellular domain has the sequence of a metabotropic glutamate receptor or has a sequence that is at least 50, 60, 70, 80, 90, 95, 97, 98, or 99% or is 100% identical to at least a portion of the sequence of contiguous amino acid residues from the extracellular domain of such metabotropic glutamate receptor. Such a portion can, for example, be at least 10, 20, 30, 40, 50, 60, 70, 80, 90, 100 or more amino acid residues in length, and can be a full-length extracellular domain (not including any deleted native signal peptide). The receptor also includes a transmembrane domain, and optionally a cytoplasmic tail domain. The transmembrane domain sequence is the same as or includes a sequence that is at least 50, 60, 70, 80, 90, 95, 97, 98, or 99% identical to a sequence of at least 10, 20, 30, 40, 50, 60, 70, 80, 90, 100 or more contiguous amino acid residues from such metabotropic glutamate receptor or a calcium receptor, and can be a full-length domain. The cytoplasmic tail domain sequence, when present, includes a sequence that is the same as or includes a sequence that is at least 50, 60, 70, 80, 90, 95, 97, 98, or 99% identical to a sequence of at least 10, 20, 30, 40, 50, or more contiguous amino acid residues from such metabotropic glutamate receptor or a calcium receptor, and can be a full-length cytoplamic tail domain. Alternatively, the cytoplasmic tail domain may include a shortened tail of less than 10 amino acids in length (which may be from a CaR or an mGluR or have a level of identity to a cytoplasmic tail domain sequence from a CaR or mGluR as indicated for the ECD and TMD) or may be absent. In embodiments where the cytoplasmic tail is absent or less than 10 amino acid residues in length, the C-teminus of the receptor sequence is fused to a G-protein (such fusion can also be performed with longer or even full-length cytoplasmic tail domains).

In certain embodiments, all the domains are from a metabotropic glutamate receptor, at least one domain is from a domain of a calcium receptor; one domain is from a calcium receptor; two domains are from a calcium receptor; the transmembrane domain is from a calcium receptor; the cytoplasmic tail domain is from a calcium receptor.

In certain embodiments, the chimeric receptor has at least one cytoplasmic loop of the seven transmembrane domain which is from a cytoplasmic loop of a metabotropic glutamate receptor. Similarly, in other embodiments, the chimeric receptor has at least one cytoplasmic loop from a cytoplasmic loop of a calcium receptor.

Also in certain embodiments, the chimeric receptor has a sequence of at least 6 contiguous amino acids which is from an amino acid sequence of a calcium receptor, and the rest of the sequence of the chimeric receptor is from an amino acid sequence of a metabotropic glutamate receptor. In other embodiments, the sequence from an amino acid sequence of a calcium receptor may beneficially be longer, for example at least 12, 18, 24, 30, 36, 54, 72 or more amino acids in length.

In a related aspect, the invention provides a chimeric receptor as described for the aspect above.

In another related aspect, the invention provides a composition which includes an isolated, enriched, or purified nucleic acid molecule which codes for a chimeric receptor as described for the aspects above. In particular, this includes nucleic acid coding for a chimeric receptor having a non-native signal peptide linked to the N-terminus of an extracellular domain from an mGluR. As described the chimeric receptor can also include one or more sequences from a CaR and/or can have a G-protein linked on the C-terminus of the receptor sequence.

In another related aspect, the nucleic acid encoding a chimeric receptor, as described above, is present in a replicable expression vector. Thus, the vector can include nucleic acid sequences coding for any of the chimeric receptors described.

Also in a related aspect, the invention provides a recombinant host cell transformed with a replicable expression vector as described above.

The invention also features a process for the production or manufacture of a chimeric receptor; the process involves growing, under suitable nutrient conditions, procaryotic or eucaryotic host cells transformed or transfected with a replicable expression vector containing a nucleic acid sequence coding for a chimeric receptor as described above, in a manner allowing expression of the chimeric receptor.

By “isolated” in reference to a nucleic acid is meant the nucleic acid is present in a form (i.e., its association with other molecules) other than found in nature. For example, isolated receptor nucleic acid is separated from one or more nucleic acids which are present on the same chromosome. Preferably, the isolated nucleic acid is separated from at least 90% of the other nucleic acids present on the same chromosome. Preferably, the nucleic acid is provided as a substantially purified preparation representing at least 75%, more preferably 85%, most preferably 95% of the total nucleic acids present in the preparation.

Another example of an isolated nucleic acid is recombinant nucleic acid. Preferably, recombinant nucleic acid contains nucleic acid encoding a chimeric metabotropic glutamate receptor or metabotropic glutamate receptor fragment cloned in an expression vector. An expression vector contains the necessary elements for expressing a cloned nucleic acid sequence to produce a polypeptide. An expression vector contains a promoter region (which directs the initiation of RNA transcription) as well as the DNA sequences which, when transcribed into RNA, will signal synthesis initiation. “Expression vector” includes vectors which are capable of expressing DNA sequences contained therein, i.e., the coding sequences are operably linked to other sequences capable of effecting their expression. It is implied, although not always explicitly stated, that these expression vectors must be replicable in the host organisms either as episomes or as an integral part of the chromosomal DNA.

A useful, but not a necessary, element of an effective expression vector is a marker encoding sequence—i.e., a sequence encoding a protein which results in a phenotypic property (e.g. tetracycline resistance) of the cells containing the protein which permits those cells to be readily identified. In sum, “expression vector” is given a functional definition, and any DNA sequence which is capable of effecting expression of a specified contained DNA code is included in this term, as it is applied to the specified sequence. As at present, such vectors are frequently in the form of plasmids, thus “plasmid” and “expression vector” are often used interchangeably. However, the invention is intended to include such other forms of expression vectors, including viral vectors, which serve equivalent functions and which may, from time to time become known in the art. Recombinant nucleic acids may contain nucleic acids encoding for a chimeric metabotropic glutamate receptor, receptor fragment, or chimeric metabotropic glutamate receptor derivative, under the control of its genomic regulatory elements, or under the control of exogenous regulatory elements including an exogenous promoter. By “exogenous” is meant a promoter that is not normally coupled in vivo transcriptionally to the coding sequence for the metabotropic glutamate receptor or calcium receptor.

The invention also provides methods of screening for compounds which bind to and/or modulate the activity of a metabotropic glutamate receptor and/or a calcium receptor. These methods utilize chimeric receptors as described above or nucleic acid sequence encoding such chimeric receptors. Such chimeric receptors provide useful combinations of characteristics from the two types of receptors, such as combining the binding characteristics from a metabotropic glutamate receptor with the cellular signaling characteristics from a calcium receptor.

Thus, in another aspect the invention provides a method of screening for a compound that binds to or modulates the activity of a metabotropic glutamate receptor. The method involves preparing a chimeric receptor as described herein. The chimeric receptor and a test compound are introduced into an acceptable medium. The binding of a test compound to the chimeric receptor, or the modulation of the chimeric receptor by the compound, is monitored by physically detectable means to identify those compounds which bind to or modulate the activity of the chimeric receptor. Such binding or modulation is indicative that the test compound binds to and/or modulates the metabotropic glutamate receptor from which corresponding sequences are included in the chimeric receptor of a metabotropic glutamate receptor.

In another aspect the invention provides a method of screening for a compound which binds to or modulates the activity of a metabotropic glutamate receptor, utilizing a nucleic acid coding for a chimeric receptor. This method involves expressing a chimeric receptor in a host cell and measuring, determining, or monitoring the effect of the presence of a test compound on a characteristic of the host cell.

In certain embodiments the method can also involve preparing a nucleic acid sequence encoding the chimeric receptor, and/or inserting the nucleic acid sequence into a replicable expression vector capable of expressing the chimeric receptor in a suitable host cell with this vector, and/or introducing the transformed host cell and a test compound into an acceptable medium. Identification of binding or modulation by the test compound is performed by measuring, determining, and/or monitoring the effect of the compound on the cell, e.g. on an observable characteristic of the cell, such as intracellular calcium concentration.

In certain embodiments the host cell is a eucaryotic cell, which can be a vertebrate cell (e.g., a frog cell such as a Xenopus cell or oocyte), or a mammalian cell such as a human cell. Advantageously the cell is a transgenic cell with knock-in expression control.

In the context of the methods of this invention, “monitoring the effect” of a compound on a host cell refers to determining the effects of the compound on one or more cellular processes, or on the level of activity of one or more cellular components, or by detection of an interaction between the compound and a cellular component, or on the level of a component in the cell or in the medium the cell is in. Similarly, “determining the effect” refers to a measurement or observation of one or more physical properties or characteristics of the system or cell. In this context, the term “measuring” refers to a quantitative determination of one or more physical properties or characteristics.

The invention also provides methods of screening for compounds that bind to or modulate a metabotropic glutamate receptor using fragments of such receptors. Such fragments can, for example, be chosen to include a sequence which has been shown to be important in activation of the receptor's signal pathway.

Thus, in another aspect the invention features a method of screening for a compound that binds to a metabotropic glutamate receptor by methods corresponding to those for a full receptor as described above by monitoring, determining, or measuring the binding, if any, of receptor fragment with test compound., where the receptor fragment includes a mGluR sequence, e.g., an extracellular domain sequence linked with a non-native signal peptide. The method can also involve one or more of: preparing a nucleic acid encoding a fragment of such a receptor that is linked to a non-native signal peptide at its N-terminus, inserting the sequence into a replicable expression vector which can express that fragment in a host cell, transforming a suitable host cell with the vector, recovering the fragment from the host cell, introducing the fragment in a test compound into an acceptable medium and monitoring, determining, or measuring the binding of the compound to the fragment by physically detectable means.

In certain embodiments, the fragment is a fragment of a metabotropic glutamate receptor that includes the extracellular domain of that receptor. In other embodiments the fragment includes both the seven transmembrane domain and the cytoplasmic tail domain of a metabotropic glutamate receptor.

Certain receptor fragments are able to activate one or more cellular responses in a manner similar to the receptor from which the fragment was derived. Therefore, in a related aspect, the invention provides a method of screening for a compound that binds to or modulates a metabotropic glutamate receptor by methods as described above for full receptors, involving expressing the receptor sequence in a host cell and monitoring, determining, or measuring the effect of the presence of a test compound on a cellular process, characteristic, or other property. As before, this can also involve one or more of: preparing a nucleic acid sequence encoding a fragment of such a receptor that has a non-native signal peptide linked at the N-terminus, inserting that sequence into a replicable expression vector, transforming a host cell with that vector, introducing the host cell and a test compound into an acceptable medium, and monitoring the effect of the compound on the host cell.

Certain compounds can be identified which modulate the activity of both a metabotropic glutamate receptor and of a calcium receptor. Thus, this invention also provides a method for screening for such compounds by preparing a nucleic acid sequence encoding a chimeric receptor which includes an extracellular domain from a metabotropic glutamate receptor linked with a non-native signal peptide and a domain from a calcium receptor. The sequence is inserted in a replicable expression vector capable of expressing the receptor in a host cell; a suitable host cell is transformed with the vector and the transformed host cell and a test compound are introduced into an acceptable medium. The binding or modulation by the compound is observed by monitoring the effect of a compound on the host cell as described above.

The invention also provides kits that include one or more chimeric receptors or a nucleic acid encoding such receptor, or a host cell that includes such nucleic acid (e.g., in an expression vector) as described herein in a container. In most cases, the kit will include a host cell transformed or transfected with a vector comprising a nucleic acid encoding a chimeric receptor as described herein. The kit can also include one or more other components, such as without limitation, growth medium, written instructions for growing host cells and/or screening for modulator compounds, buffer(s), antibodies targeted to the chimeric receptor, and/or activity control compounds, which can be negative and/or positive controls for modulating the chimeric receptor or the corresponding mGluR.

Other features and advantages of the invention will be apparent from the following description of the preferred embodiments, and from the claims.

Additional aspects and embodiments will be apparent from the following Detailed Description and from the claims.

### BRIEF DESCRIPTION OF THE DRAWINGS

FIG. 1 A-G is a schematic illustration of the various mGluR chimeras described herein, illustrating the extracellular domains, 7-transmembrane domains, and intracellular cytoplasmic tail domains of the chimeras.

FIG. 2 shows an alignment of the first 60 amino acids of mGluR and CaR and SP constructs.

FIG. 3 is a schematic representation of an exemplary expression vector useful in expressing the present chimeric receptors.

FIG. 4 shows activation of CaSPhmGluR7(27-45) in the oocyte assay for Gαi-coupled receptors (method detailed in Example 1B) with application of 100 uM L-glutamate.

FIG. 5 shows activation of CaSPhmGluR3(27-33) in the oocyte assay for Gαi-coupled receptors (method detailed in Example 1B) with application of 100 uM L-glutamate.

FIG. 6 shows activation of CaSPhmGluR2(27-19) in the oocyte assay for Gαi-coupled receptors (method detailed in Example 1B) by 100 uM L-glutamate.

FIG. 7 is a graphical representation showing changes in intracellular calcium caused by activation of CaSphmGluR6(27-35) chimeric receptor by the Fura Assay.

FIG. 8 is a graphical representation showing activation of CaSPhmGluR5(27-22) in the oocyte assay for PLC-coupled receptors (method detailed in Example 1A) by 100 uM L-glutamate.

FIG. 9 is a graphical representation comparing the pmGluR1/CaR chimera to rat mGluR1 using the PLC-coupled oocyte assay showing activation by L-glutamate and quisqualate as measured by Cl— currents generated in response to the release of intracellular Ca2+ in the oocyte.

FIG. 10 A-C is a graphical representation showing that extracellular glutamate elicits oscillatory increases in Cl— current in Xenopus oocytes injected with A) ratmGluR1 RNA, B) human CaR RNA, and C) ratCH3 RNA. However, when oocytes are repeatedly supplied with agonist, the rat mGluR1 receptor desensitizes and does not activate the release of intracellular Ca2+. RatCH3, which encodes the cytoplasmic tail of the CaR does not desensitize like the native rat mGluR1 and is thus amenable to repeated challenges with compounds.

FIG. 11 is a graphical representation showing increases in intracellular calcium induced by extracellular calcium in fura-2 loaded stably-transfected HEK293 cells expressing pCEPCaR/R1.

### DETAILED DESCRIPTION OF THE PREFERRED EMBODIMENTS

The present invention concerns the use of non-native signal peptides with mGluR receptors or chimeric receptors that include extracellular domains from an mGluR. This invention is particularly useful for mGluRs that are difficult to express (or to obtain good levels of functional receptor) in suitable host cells using native signal peptides and/or can provide a convenient epitope for detection (e.g., in cell staining and Western blots) and/or purification.

Definitions

In addition to definitions provided in the Summary, the following is a list of some further definitions of terms used in the present disclosure. These definitions are to be understood in light of the entire disclosure provided herein.

By “adjunct in general anesthesia” is meant a compound used in conjunction with an anesthetic agent which decreases the ability to perceive pain associated with the loss of consciousness produced by the anesthetic agent.

By “allodynia” is meant pain due to a stimulus that does not normally provoke pain.

By “analgesic” is meant a compound capable of relieving pain by altering perception of nociceptive stimuli without producing anesthesia resulting in the loss of consciousness.

By “analgesic activity” is meant the ability to reduce pain in response to a stimulus that would normally be painful.

By “anticonvulsant activity” is meant efficacy in reducing convulsions such as those produced by simple partial seizures, complex partial seizures, status epilepticus, and trauma-induced seizures such as those occurring following head injury, including head surgery.

By “binds to or modulates” is meant that the agent may both bind and modulate the activity of a receptor, or the agent may either bind to or modulate the activity of a receptor.

By “causalgia” is meant a painful disorder associated with injury of peripheral nerves.

By “central pain” is meant pain associated with a lesion of the central nervous system.

By “cognition-enhancement activity” is meant the ability to improve the acquisition of memory or the performance of a learned task. Also by “cognition-enhancement activity” is meant the ability to improve compromised rational thought processes and reasoning.

By “cognition enhancer” is meant a compound capable of improving learning and memory.

By “efficacy” is meant that a statistically significant level of the desired activity is detectable with a chosen compound; by “significant” is meant a statistical significance at the p<0.05 level.

By “homologous” is meant a functional equivalent to the domain, the amino acid sequence, or the nucleic acid sequence, having similar nucleic acid and/or amino acid sequence and retaining, to some extent, one or more activities of the related receptor. Homologous domains or sequences of receptors have at least 50% sequence similarity, and can have at least 60%, 70%, 80%, 90%, 95%, 97%, 98%, or 99% sequence similarity or sequence identity to the related receptor. “Sequence similarity” refers to “homology” observed between amino acid sequences in two different polypeptides, irrespective of polypeptide origin. Thus, homologous includes situations in which the nucleic acid and/or amino acid sequences are the same. Such homologous domains or sequences can be used in the present invention.

In related phrases, as indicated in the Summary, reference to a sequence, sub-domain, or domain being “from a metabotropic glutamate receptor” or “of a metabotropic glutamate receptor” means that the portion is the same as or has the specified level of sequence identity to a portion of a metabotropic glutamate receptor; like references to portions being “from a calcium receptor” or “of a calcium receptor” also indicate the portions are the same as or have the specified level of sequence identify to portions of a calcium receptor. These phrases can be used in reference to amino acid sequences and/or nucleic sequences. If specifically indicated, these phrases can mean having at least 50%, 60%, 70%, 80%, 90%, 95%, 97%, 98%, or 99% sequence similarity determined using an alignment tool as specified for determining percent sequence identity with default parameters.

The ability of the homologous domain or sequence to retain some activity can be measured using techniques described herein. Such homologous domains may also be derivatives. Derivatives include modification occurring during or after translation, for example, by phosphorylation, glycosylation, crosslinking, acylation, proteolytic cleavage, linkage to an antibody molecule, membrane molecule or other ligand (see Ferguson et al., 1988, Ann. Rev. Biochem. 57:285-320).

Specific types of derivatives also include amino acid alterations such as deletions, substitutions, additions, and amino acid modifications. A “deletion” refers to the absence of one or more amino acid residue(s) in the related polypeptide. An “addition” refers to the presence of one or more amino acid residue(s) in the related polypeptide. Additions and deletions to a polypeptide may be at the amino terminus, the carboxy terminus, and/or internal. Amino acid “modification” refers to the alteration of a naturally occurring amino acid to produce a non-naturally occurring amino acid. A “substitution” refers to the replacement of one or more amino acid residue(s) by another amino acid residue(s) in the polypeptide. Derivatives can contain different combinations of alterations including more than one alteration and different types of alterations.

While the effect of an amino acid change varies depending upon factors such as phosphorylation, glycosylation, intra-chain linkages, tertiary structure, and the role of the amino acid in the orthosteric site or a possible allosteric site, it is generally preferred that the substituted amino acid is from the same group as the amino acid being replaced. To some extent the following groups contain amino acids which are interchangeable: the basic amino acids lysine, arginine, and histidine; the acidic amino acids aspartic and glutamic acids; the neutral polar amino acids serine, threonine, cysteine, glutamate, asparagine and, to a lesser extent, methionine; the nonpolar aliphatic amino acids glycine, alanine, valine, isoleucine, and leucine (however, because of size, glycine and alanine are more closely related and valine, isoleucine and leucine are more closely related); and the aromatic amino acids phenylalanine, tryptophan, and tyrosine. In addition, although classified in different categories, alanine, glycine, and serine seem to be interchangeable to some extent, and cysteine additionally fits into this group, or may be classified with the polar neutral amino acids.

While proline is a nonpolar neutral amino acid, its replacement represents difficulties because of its effects on conformation. Thus, substitutions by or for proline are not preferred, except when the same or similar conformational results can be obtained. The conformation conferring properties of proline residues may be obtained if one or more of these is substituted by hydroxyproline (Hyp).

Examples of modified amino acids include but are not limited to the following: altered neutral nonpolar amino acids such as amino acids of the formula H₂N(CH₂)ₙCOOH where n is 2-6, sarcosine (Sar), t-butylalanine (t-BuAla), t-butylglycine (t-BuGly), N-methyl isoleucine (N-MeIle), and norleucine (Nleu); altered neutral aromatic amino acids such as phenylglycine; altered polar, but neutral amino acids such as citrulline (Cit) and methionine sulfoxide (MSO); altered neutral and nonpolar amino acids such as cyclohexyl alanine (Cha); altered acidic amino acids such as cysteic acid (Cya); and altered basic amino acids such as omithine (Orn).

Preferred derivatives have one or more amino acid alteration(s) which do not significantly affect the receptor activity of the related receptor protein. In regions of the receptor protein not necessary for receptor activity amino acids may be deleted, added or substituted with less risk of affecting activity. In regions required for receptor activity, amino acid alterations are less preferred as there is a greater risk of affecting receptor activity. Such alterations should be conservative alterations. For example, one or more amino acid residues within the sequence can be substituted by another amino acid of a similar polarity which acts as a functional equivalent.

Conserved regions tend to be more important for protein activity than non-conserved regions. Standard procedures can be used to determine the conserved and non-conserved regions important of receptor activity using in vitro mutagenesis techniques or deletion analyses and measuring receptor activity as described by the present disclosure.

Derivatives can be produced using standard chemical techniques and recombinant nucleic acid techniques. Modifications to a specific polypeptide may be deliberate, as through site-directed mutagenesis and amino acid substitution during solid-phase synthesis, or may be accidental such as through mutations in hosts which produce the polypeptide. Polypeptides including derivatives can be obtained using standard techniques such as those described in Sambrook et al., Molecular Cloning, Cold Spring Harbor Laboratory Press (1989). For example, Chapter 15 in that manual describes procedures for site-directed mutagenesis of cloned DNA.

By “hyperalgesia” is meant an increased response to a stimulus that is normally painful.

By “minimal” is meant that any side effect of the drug is tolerated by an average individual, and thus that the drug can be used for therapy of the target disease or disorders. Such side effects are well known in the art. Preferably, minimal side effects are those which would be regarded by the FDA as tolerable for drug approval for a target disease or disorder.

By “modulate” is meant to cause an increase or decrease in an activity of a cellular receptor.

By “modulator” is meant a compound which modulates a receptor, including agonists, antagonists, allosteric modulators, and the like. Preferably, the modulator binds to the receptor.

By “muscle relaxant” is meant a compound that reduces muscular tension.

By “neuralgia” is meant pain in the distribution of a nerve or nerves.

By “neurodegenerative disease” is meant a neurological disease affecting cells of the central nervous system resulting in the progressive decrease in the ability of cells of the nervous system to function properly. Examples of neurodegenerative diseases include Alzheimer's disease, Huntington's disease, and Parkinson's disease.

By “neurological disorder or disease” is meant a disorder or disease of the nervous system. Examples of neurological disorders and diseases include global and focal ischemic and hemorrhagic stroke, head trauma, spinal cord injury, hypoxia-induced nerve cell damage as in cardiac arrest or neonatal distress, and epilepsy.

By “neuroprotectant activity” is meant efficacy in treatment of the neurological disorders or diseases.

By “physically detectable means” is meant any means known to those of ordinary skill in the art to detect binding to or modulation of mGluR or CaR receptors, including the binding and screening methods described herein. Thus, for example, such means can include spectroscopic methods, chromatographic methods, competitive binding assays, and assays of a particular cellular function, as well as other techniques.

By “potent” is meant that the compound has an EC₅₀ value (concentration which produces a half-maximal activation), or IC₅₀ (concentration which produces half-maximal inhibition), or K_{d} (concentration which produces half-maximal binding) at a metabotropic glutamate receptor, with regard to one or more receptor activities, of less than 100 μM, more preferably less than 10 μM, and even more preferably less than 1 μM.

By “selective” is meant that the compound activates, inhibits activation and/or binds to a metabotropic glutamate receptor at a lower concentration than that at which the compound activates, inhibits activation and/or binds to an ionotropic glutamate receptor. Preferably, the concentration difference is a 10-fold, more preferably 50-fold, and even more preferably 100-fold.

By “therapeutically effective amount” is meant an amount of a compound which produces a desired therapeutic effect in a patient. For example, in reference to a disease or disorder, it is the amount which reduces to some extent one or more symptoms of the disease or disorder, and ameliorates, either partially or completely, physiological or biochemical parameters associated or causative of the disease or disorder. When used to therapeutically treat a patient it is an amount expected to be between 0.1 mg/kg to 100 mg/kg, preferably less than 50 mg/kg, more preferably less than 10 mg/kg, more preferably less than 1 mg/kg. Preferably, the amount provides an effective concentration at a metabotropic glutamate receptor of about 1 nM to 10 μM of the compound. The amount of compound depend on its EC₅₀ (IC₅₀ in the case of an antagonist) and on the age, size, and disease associated with the patient.

Techniques

A. Chimeric Receptors and General Approach to Uses

As indicated above, this invention concerns chimeric receptors, which include at least a portion of a metabotropic glutamate receptor linked with a non-native signal peptide, advantageously a CaR signal peptide, and can also include a portion(s) of calcium receptor proteins. It also is concerned with fragments of metabotropic glutamate receptors and calcium receptors. Related aspects include nucleic acids encoding such chimeric receptors and fragments, uses of such receptors, fragments and nucleic acids, and cell lines expressing such nucleic acids. The uses disclosed include methods of screening for compounds that bind to or modulate the activity of metabotropic glutamate receptors using such chimeric receptors and fragments. The invention also includes compounds for modulating metabotropic glutamate receptors identified by such methods of screening, and methods for treating certain disorders or for modulating metabotropic glutamate receptors utilizing such compounds.

Signal peptides are typically identified as initial hydrophobic amino acid sequences at the N-terminus of a protein, especially membrane associated proteins such as membrane associated receptors. Such receptors include the mGluRs. Such signal peptides can be identified or predicted, for example, using software algorithms known in the art. As shown in Example 1, the precise length of a native signal peptide that should be deleted and/or the length of the non-native signal peptide to to engineered on the chimeric receptor can be varied. If desired, a number of different boundaries can be tested empirically, similar to the variation shown in Example 1. Such variation can be carried out using standard techniques, e.g., standard cloning techniques. In particular embodiments, the signal peptide is from a CaR; the signal peptide includes 20-40, 22-38, 24-32, 26-30, 27-27, or 27 amino acid residues from the N-terminus of a CaR.

Experiments carried out using exemplary chimeric receptors that include human mGluR7 (a difficult to express mGluR) with an mGluR8 signal peptide or a CaR signal peptide demonstrates that the present chimeric receptors can provide advantageous properties for receptor expression, detection, and/or purification.

Likewise, experiments carried out on several distinct G-protein coupled receptors have suggested the general principle that G-protein coupling specificity and receptor desensitization are determined primarily by amino acid sequences which are intracellular (i.e., sequences within one or more of the three cytoplasmic loops and/or the intracellular cytoplasmic tail). Recent experiments in which chimeric receptors were formed by combining distinct protein segments from different metabotropic glutamate receptors (mGluRs), suggest that, in these receptors, ligand binding specificity is determined by the extracellular domain.

Thus, embodiments of the present invention include chimeric receptors that include only mGluR sequence with the non-native signal peptide; receptors consisting of the extracellular domain (ECD) of an mGluR and the seven-transmembrane domain (7TMD) and the intracellular cytoplasmic tail (CT) of a calcium receptor (CaR) that responds to mGluR-active molecules by signal transduction analogous to that observed when CaR-active molecules act on a CaR. Similarly, in certain embodiments, the invention includes chimeric receptors in which the intracellular cytoplasmic C-terminal tail domain of a chosen mGluR is replaced by the C-terminal tail (or a portion) of a calcium receptor. The C-terminal tail encompasses the cytoplasmic region which follows the seventh transmembrane region.

Embodiments of the invention also include chimeric receptors in which the peptide sequences encompassing all or some of the cytoplasmic loop domains (between the first and second, the third and fourth, and the fifth and sixth transmembrane regions) of an mGluR have been replaced similarly with corresponding peptide sequences from a CaRs. In particular such embodiments include chimeric receptors having the ECD of an mGluR, the 7TMD of an mGluR, and the C-terminal tail of a calcium receptor, except that one or more sub-domains of the 7-TM] are substituted with sequences from a CaR. This specifically includes receptors in which one or more of the cytoplasmic loops of the 7TMD are replaced with sequences from a CaR. Such substitution of cytoplasmic loops may be done singly or in any combination. In general, using techniques known to those skilled in the art, such target “domains” and “sub-domains” may be “swapped” individually or in combination.

Experiments have shown that ligands known in the art which are agonists or antagonists on the native mGluRs also exhibit such activities on the chimeric receptors in which the extracellular domain is from an mGluR. Other ligands which bind to the ECD and modulate the activity of mGluRs, for example, agonists, antagonists, allosteric modulators and the like, are also predicted to act on such chimeric receptors. Experiments have also shown that ligands known in the art which modulate mGluRs act on the chimeric receptors in which the ECD and 7TMD are from an mGluR. Other ligands which modulate mGluR activity are also predicted to act on this type of chimeric receptors regardless of whether they bind the ECD or 7TMD of mGluRs.

The chimeric receptors can be linked to intracellular or second messenger functions in a similar fashion to the linkage known for non-modified calcium receptors. For example, as is the case for the CaR, the chimeric receptors can also couple through a G-protein(s) to the activation of phospholipase C, to the generation of inositol phosphates and/or to the release of calcium ions from intracellular stores. Although the mGluRs rapidly desensitize upon ligand binding/activation, the CaRs do not, allowing for more efficient high-throughput screening of compounds active at the CaR and stable receptor expression in recombinant cell lines. Importantly, the chimeric mGluR/CaR receptors can be efficiently used for high throughput screening. In addition, the chimeric receptors can be functionally expressed in stable cell lines.

Cells expressing such chimeric receptors can be prepared and used in functional assays to identify compounds which modulate activities of selected mGluRs. For example, increases in intracellular calcium levels resulting from receptor activation can be monitored by use of fluorescent calcium chelating dyes. Functional assays have been described for identifying molecules active at calcium receptors (see for example, published PCT patent application “Calcium Receptor-Active Molecules,” PCT No. US93/01642 (W094/18959), published September 1994 hereby incorporated herein by reference in its entirety).

An increasingly common practice in modern drug discovery is the use of various target-site-specific assays to identify specific molecules with activities of interest. These assays select drug lead molecules from large collections or libraries of molecules (e.g., combinatorial libraries, proprietary compound libraries held by large drug companies, etc.). Drug lead molecules are “selected” when they bind to pharmacological targets of interest and thus potentially modify the activities of these targets. The assays can be of many types including direct binding displacement assays or indirect functional assays. In order to successfully develop and use an assay to isolate lead therapeutic compounds, the target molecule (e.g., receptor) must first be identified and isolated. Many functional assays have been described in the literature for identifying molecules active at various receptors and these provide unique advantages over binding assays. It is not necessary to know, a priori, which ligands modulate the activity of the receptor in vivo, nor is it necessary to know the exact physiological function of the receptor. Compounds identified in functional assays and in subsequent medicinal chemistry efforts can be used as experimental test compounds to obtain such knowledge.

While eight distinct mGluRs are currently known, their discrete functions have not been fully determined. Nevertheless, molecules active at mGluRs are sought by pharmaceutical companies because these receptors are found inboth the central and peripheral nervous system and are known to be involved in the regulation of processes related to memory, motor functions, pain sensation, neurodegeneration and the like. Thus, compounds which modulate mGluRs can be useful in the treatment of disorders or diseases affecting memory, cognition, and motor function (e.g., in seizures) as well as in the treatment of pain and neurodegenerative disorders (e.g., stroke, Alzheimers disease and the like).

Screens to identify molecules active at mGluRs can be constructed using cloned mGluRs themselves. However, functional screens using native mGluRs are problematic. First, certain mGluRs are coupled through Gᵢ proteins and this limits their use in functional assays because Gᵢ proteins are linked to inhibition of adenylate cyclase and changes in adenylate cyclase are not easily measured in high throughput functional screens designed to select drug lead molecules from large compound libraries.

Receptors which couple through other G-proteins to activation of phospholipase C (e.g., G_{q}-coupled receptors) do not suffer this drawback, so it was initially thought that mGluR1 and mGluR5 could find utility in functional assays because these two mGluRs are coupled through Gq-protein(s) to measurable intracellular functions (e.g., activation of phospholipase C, generation of inositol phosphates and the release of calcium ions from intracellular stores).

A second limitation is presented here, however, because these particular mGluRs rapidly desensitize upon agonist binding. That is, the functional response disappears rapidly and cannot quickly be recovered. Furthermore, it has not always been possible to obtain fully functional stable cell lines expressing mGluRs regardless of the G-protein to which they couple (Tanabe et al., 1992, Neuron 8:169-179; Gabellini et al., 1994, Neurochem Int. 24:533-539). One such difficult-to-express mGluR is mGluR7. Thus, nontrivial technical difficulties must be overcome in order to use native mGluRs in an optimal manner in high throughput functional screening assays.

The invention described herein overcomes certain of these technical difficulties and provides a much improved screening method by utilizing signal peptides that improve the level of functional receptor in cellular expression.

Further, as previously described, such chimera can also incorporate the more robust aspects of the calcium receptors which do not rapidly desensitize upon ligand binding/activation and can be expressed stably in recombinant vertebrate cells (see published PCT patent application “Calcium Receptor-Active Molecules,” PCT No. US93/01642 (WO94/18959), published September 1994, hereby incorporated herein by reference). Thus, for example, by coupling the 7TMD and the CT of the CaR to the extracellular domain of mGluR, or the CT of the CaR to the ECD and 7TMD of the mGluR, the mGluR extracellular domain has the benefit of the Gq coupling property of a CaR, as well as the improved property of a lack of rapid desensitization. Such receptors have ligand binding and activation properties similar to those of the native mGluRs, but with improved second messenger coupling similar to CaRs.

Thus, since the chimeric receptors simplify and enable, efficient, practical and reproducible functional screens to identify mGluR-active molecules, compositions and methods of the present invention are useful for the identification of molecules which modulate mGluR activity. These can, for example, include agonists, antagonists, allosteric modulators, and the like.

Further, chimeric receptors constructed to screen compounds active at metabotropic glutamate receptors may employ the signaling properties of certain domains of a calcium receptor. Such a chimeric receptor would take advantage of certain unique properties associated with the agonist-induced coupling of the calcium receptor to G-proteins which activate phospholipase C and mobilize intracellular calcium. These properties include, for example, the lack of ligand induced down-regulation/desensitization which is associated with ligand activation of metabotropic glutamate receptors. Thus the superior signaling properties of the calcium receptor can be transferred to metabotropic glutamate receptors which normally do not couple to G-proteins that activate phospholipase C and mobilize intracellular calcium such as those which couple to Gᵢ.

In certain embodiments, recombinant cells expressing such chimeric receptors are used in screening methods. The cells will obtain properties, such as those indicated above, which facilitate their use in high-throughput functional assays, and thus provide a more efficient method of screening for compounds which bind to or modulate metabotropic glutamate receptor activity.

In connection with chimeric receptors that include portions of mGluRs and CaRs, such portions can confer a desired binding, signal coupling, or other functional characteristic to the chimeric receptor. The length of a sequence from a particular receptor can be of different sizes in different applications. In addition, the sequence of a portion from a particular receptor may be identical to the corresponding sequence in the mGluR or CaR, or it may be a homologous sequence, which retains the relevant function of the mGluR or CaR sequence.

In certain embodiments, the portion from a CaR is a subdomain. In this context, “subdomain” refers to a sequence of amino acids which is less than the entire sequence of amino acids for a domain. Examples of subdomains include, but are not limited to, ligand binding domains. Other examples include one of the cytoplasmic loops or regions of the seven transmembrane domain. Therefore, in certain cases, a chimeric receptor has an extracellular domain, a seven transmembrane domain, and generally an intracellular cytoplasmic tail domain, which include subdomains. In one example of such chimeric receptors, at least one subdomain is homologous to a subdomain of a calcium receptor and the remaining subdomains and domains are homologous to subdomains and domains of a metabotropic glutamate receptor. In another example, at least one subdomain is homologous to a subdomain of a metabotropic glutamate receptor and the remaining subdomains and domains are homologous to subdomains and domains of a calcium receptor.

In another specific example, the seven transmembrane domain of a chimeric receptor includes three cytoplasmic loops; at least one cytoplasmic loop is homologous to a cytoplasmic loop of a metabotropic glutamate receptor; or at least one cytoplasmic loop is homologous to a cytoplasmic loop of a calcium receptor. In another specific example, the extracellular domain is homologous to the extracellular domain of a metabotropic glutamate receptor, the seven transmembrane domain is homologous to the seven transmembrane domain of a metabotropic glutamate receptor except that one or more of the cytoplasmic loops of the seven transmembrane domain is homologous to a cytoplasmic loop(s) of a calcium receptor, and the cytoplasmic tail is homologous to the cytoplasmic tail of a calcium receptor. Thus, any of cytoplasmic loops 1, 2, and 3 may be replaced, either singly or in any combination, with a cytoplasmic loop(s) of a calcium receptor.

In other cases, the chimeric receptor has a domain which has a sequence which is the same as or homologous to the sequence of a domain of an mGluR, or a CaR, or preferably, at least one domain from each of an mGluR and a CaR. More preferably, the chimeric receptor has two domains from one receptor type and one domain from the other receptor type. The compositions of certain embodiments of such chimeric receptors that include both mGluR and CaR sequences are described below. Such chimera can be used in the present invention with a signal peptide that is a non-native signal peptide for the mGluR corresponding to the extracellular domain sequence.

Thus, in certain embodiments, the invention provides a composition comprising a chimeric receptor having:
a. one domain homologous to an extracellular domain of a metabotropic glutamate receptor, one domain homologous to the seven transmembrane domain of a calcium receptor, and one domain homologous to the intracellular cytoplasmic tail domain of a calcium receptor; or
b. one domain homologous to an extracellular domain of a metabotropic glutamate receptor, one domain homologous to the seven transmembrane domain of a calcium receptor, and one domain homologous to the intracellular cytoplasmic tail domain of a metabotropic glutamate receptor; or
c. one domain homologous to the extracellular domain of a metabotropic glutamate receptor, one domain homologous to the seven transmembrane domain of a metabotropic glutamate receptor, and one domain homologous to the intracellular cytoplasmic tail domain of a calcium receptor; or
d. one domain homologous to the extracellular domain of a metabotropic glutamate receptor, one domain homologous to the seven transmembrane domain of a metabotropic glutamate receptor except that one or more cytoplasmic loops are replaced with a cytoplasmic loop(s) homologous to a cytoplasmic loop(s) of a calcium receptor, and one domain homologous to the intracellular cytoplasmic tail domain of a calcium receptor.

B. Nucleic Acids Encoding Chimeric Receptors

Compositions which include isolated nucleic acid molecules which code for chimeric receptors as described herein are also useful in this invention. Such nucleic acid molecules can be isolated, purified, or enriched. In some cases, the nucleic acid is provided as a substantially purified preparation representing at least 75%, 85%, or 95% of the total nucleic acids present in the preparation.

Such nucleic acid molecules may also be present in a replicable expression vector. The replicable expression vector can be transformed into a suitable host cell to provide a recombinant host cell. Using such transformed host cells, the invention also provides a process for the production of a chimeric receptor, which includes growing, under suitable nutrient conditions, procaryotic or eucaryotic host cells transformed or transfected with a replicable expression vector comprising the nucleic acid molecule in a manner allowing expression of the chimeric receptor.

Uses of nucleic acids encoding chimeric receptors or receptor fragments include one or more of the following: producing receptor proteins which can be used, for example, for structure determination, to assay a molecule's activity on a receptor, to screen for molecules useful as therapeutics and to obtain antibodies binding to the receptor. The chimeras of the present invention are useful for identifying compounds active at metabotropic glutamate receptors. Also, the fragments of the present invention are useful for identifying compounds which bind to or modulate metabotropic glutamate receptors.

Thus, the invention also provides, for example, an isolated nucleic acid encoding an extracellular domain of a metabotropic glutamate receptor linked with a non-native signal peptide. Similarly, nucleic acid molecules are provided that include the non-native signal peptide, the mGluR extracellular domain sequence and CaR sequences, e.g., that are substantially free of the seven transmembrane domain and intracellular cytoplasmic tail domain of that metabotropic glutamate receptor. Similarly, the isolated nucleic acid can encode a metabotropic glutamate receptor that is substantially free of at least one membrane spanning domain portion.

C. Metabotropic Glutamate Receptor Fragments and Calcium Receptor Fragments

Receptor fragments are portions of metabotropic glutamate receptors or of calcium receptors. Receptor fragments preferably bind to one or more binding agents which bind to a full-length receptor. Binding agents include ligands, such as glutamate, quisqualate, agonists and antagonists, and antibodies which bind to the receptor. Fragments have different uses such as to select other molecules able to bind to a receptor.

Fragments can be generated using standard techniques such as expression of cloned partial sequences of receptor DNA and proteolytic cleavage of a receptor protein. Proteins are specifically cleaved by proteolytic enzymes, such as trypsin, chymotrypsin or pepsin. Each of these enzymes is specific for the type of peptide bond it hydrolyzes. Trypsin catalyzes the hydrolysis of peptide bonds whose carbonyl group is from a basic amino acid, usually arginine or lysine. Pepsin and chymotrypsin catalyze the hydrolysis of peptide bonds from aromatic amino acids, particularly tryptophan, tyrosine and phenylalanine.

Alternate sets of cleaved protein fragments are generated by preventing cleavage at a site which is susceptible to a proteolytic enzyme. For example, reaction of the ε-amino group of lysine with ethyltrifluorothioacetate in mildly basic solution yields a blocked amino acid residue whose adjacent peptide bond is no longer susceptible to hydrolysis by trypsin. Goldberger et al., Biochemistry 1:401, 1962). Treatment of such a polypeptide with trypsin thus cleaves only at the arginyl residues.

Polypeptides also can be modified to create peptide linkages that are susceptible to proteolytic enzyme-catalyzed hydrolysis. For example, alkylation of cysteine residues with haloethylamines yields peptide linkages that are hydrolyzed by trypsin. (Lindley, Nature 178:647, 1956).

In addition, chemical reagents that cleave polypeptide chains at specific residues can be used. (Witcop, Adv. Protein Chem. 16:221, 1961). For example, cyanogen bromide cleaves polypeptides at methionine residues. (Gross & Witkip, J. Am. Chem. Soc. 83: 1510, 1961).

Thus, by treating a metabotropic glutamate receptor, or fragments thereof, with various combinations of modifiers, proteolytic enzymes and/or chemical reagents, numerous discrete overlapping peptides of varying sizes are generated. These peptide fragments can be isolated and purified from such digests by chromatographic methods. Alternatively, fragments can be synthesized using an appropriate solid-state synthetic procedure.

Fragments may be selected to have desirable biological activities. For example, a fragment may include just a ligand binding site. Such fragments are readily identified by those of ordinary skill in the art using routine methods to detect specific binding to the fragment. For example, in the case of a metabotropic glutamate receptor, nucleic acid encoding a receptor fragment can be expressed to produce the polypeptide fragment which is then contacted with a receptor ligand under appropriate association conditions to determine whether the ligand binds to the fragment. Such fragments are useful in screening assays for agonists and antagonists of glutamate, and for therapeutic effects where it is useful to remove glutamate from serum, or other bodily tissues.

Other useful fragments include those having only the external portion, membrane-spanning portion, or intracellular portion of the receptor. These portions are readily identified by comparison of the amino acid sequence of the receptor with those of known receptors, or by other standard methodology. These fragments are useful for forming chimeric receptors with fragments of other receptors to create a receptor with an intracellular portion which performs a desired function within that cell, and an extracellular portion which causes that cell to respond to the presence of glutamate, or those agonists or antagonists described herein. Chimeric receptor genes when appropriately formulated are useful in genetic therapies for a variety of diseases involving dysfunction of receptors or where modulation of receptor function provides a desirable effect in the patient.

Additionally, chimeric receptors can be constructed such that the intracellular domain is coupled to a desired enzymatic process which can be readily detected by colorimetric, radiometric, luminometric, spectrophotometric or fluorimetric assays and is activated by interaction of the extracellular portion with its native ligand (e.g., glutamate) or agonist and/or antagonists of the invention. Cells expressing such chimeric receptors can be used to facilitate screening of metabotropic glutamate receptor agonists and antagonists, and in some cases inorganic ion receptor agonists and antagonists.

Thus, this invention also provides fragments, or purified polypeptides of metabotropic glutamate receptors, or chimeric receptors including calcium receptor sequences and metabotropic glutamate receptor sequences, that include a non-native signal peptide. The fragments may be used to screen for compounds that are active at either metabotropic glutamate or calcium receptors. For example, a fragment including the extracellular domain of a calcium receptor or a metabotropic glutamate receptor may be used in a soluble receptor binding assay to identify which molecules in a combinatorial library can bind the receptor within the region assayed. Such “binding” molecules may be predicted to affect the function of the receptor. Preferred receptor fragments include those having functional receptor activity, a binding site, epitope for antibody recognition (typically at least six amino acids), and/or a site which binds a metabotropic glutamate receptor agonist, antagonist or modulator. Other preferred receptor fragments include those having only an extracellular portion, a transmembrane portion, an intracellular portion, and/or a multiple transmembrane portion (e.g., seven transmembrane portion). Such receptor fragments have various uses such as being used to obtain antibodies to a particular region and being used to form chimeric receptors and fragments of other receptors to create a new receptor having unique properties.

For chimeric receptors that include CaR sequence, the purified polypeptides or fragments preferably have at least six contiguous amino acids of a calcium receptor.

By “purified” in reference to a polypeptide is meant that the polypeptide is in a form (i.e., its association with other molecules) distinct from naturally occurring polypeptide. In some cases, the polypeptide is provided as a substantially purified preparation representing at least 75%, 85%, or 95%, of the total protein in the preparation.

In many applications, it is preferable that the purified polypeptide or fragment have more than 6 contiguous amino acids from the calcium receptor or chimeric receptor. For example, the purified polypeptide can have at least 12, 18, 14, 30, 36, 54, 72, 96, or more contiguous amino acids of the “parent” receptor.

Certain fragments of metabotropic glutamate receptors and calcium receptors retain the functions of activating one or more of the cellular responses normally activated by the “parent” receptor when contacted with a compound which interacts. Thus, for example, a cellular expressed fragment which includes the 7TMD and CT of an mGluR or a CaR, but do not include the ECD, may activate a cellular response(s) when contacted with a compound which interacts with the 7TMD. Thus, incorporation of such fragments in a cell-based method of screening for compounds which bind to or modulate a metabotropic glutamate receptor or calcium receptor, such as that described herein for chimeric receptors, is useful to identify active compounds which interact with the fragment rather than the deleted sequence. In such cases where the extracellular domain is absent, the signal peptide is linked to the N-terminus of the remaining sequence.

D. Screening Procedures to Identify Compounds Which Modulate Metabotropic Glutamate Receptor Activities Using Chimeric Receptors

The mGluR agonist and antagonist compounds described in the scientific literature are related to the endogenous agonist, glutamate (for reviews see: Cockcroft et al., Neurochem. Int. 23:583-594, 1993; Schoepp and Conn, TIPS 14:13-20, 1993; Hollmann and Heinemann, Annu. Rev. Neurosci. 17:31-108, 1994). Such agonist and antagonist compounds have an acidic moiety, usually a carboxylic acid, but sometimes a phosphatidic acid. Presumably then, such compounds bind mGluRs at the same site as the amino acid, glutamate. This has been confirmed for methylcarboxyphenylglycine, which was shown to be a competitive antagonist of glutamate (Eaton et al., Eur. J. Pharm.-Mol. Pharm. Sect. 244:195-197, 1993). Conersely, compounds active at mGluRs, lacking negative charges, and not resembling the amino acid glutamate, may not act at the glutamate binding site.

Compounds targeted to the metabotropic glutamate receptor have several uses including diagnostic uses and therapeutic use. The syntheses of many of the compounds is described by Nemeth et al., entitled “Calcium Receptor Active Molecule” International Publication Number WO 93/04373, hereby incorporated by reference herein. Those compounds binding to a metabotropic glutamate receptor and those compounds efficacious in modulating metabotropic receptor glutamate activity can be identified using the procedures described herein. Those compounds which can selectively bind to the metabotropic glutamate receptor can be used diagnostically to determine the presence of the metabotropic glutamate receptor versus other glutamate receptors.

The following is a description of procedures which can be used to obtain compounds modulating metabotropic glutamate receptor activity. Various screening procedures can be carried out to assess the ability of a compound to modulate activity of chimeric receptors of the invention by measuring its ability to have one or more activities of a metabotropic glutamate receptor modulating agent or a calcium receptor modulating agent. In cells expressing chimeric receptors of the invention, such activities include the effects on intracellular calcium, inositol phosphates and cyclic AMP.

Measuring [Ca²⁺]ᵢ with fura-2 provides a very rapid means of screening new organic molecules for activity. In a half day, 10-15 compounds (or molecule types) can be examined and their ability to mobilize or inhibit mobilization of intracellular Ca²⁺ can be assessed by a single experiment. The sensitivity of observed increases in [Ca²⁺]ᵢ to depression can also be assessed.

For example, recombinant cells expressing chimeric receptors loaded with fura-2 are initially suspended in buffer containing 0.5 mM CaCl₂. A test substance is added to the cuvette in a small volume (5-15 μl) and changes in the fluorescence signal are measured. Cumulative increases in the concentration of the test substance are made in the cuvette until some predetermined concentration is achieved or no further changes in fluorescence are noted. If no changes in fluorescence are noted, the molecule is considered inactive and no further testing is performed.

In the initial studies, molecules were tested at concentrations as high as 5 or 10 mM. As more potent molecules became known, the ceiling concentration was lowered. For example, newer molecules are tested at concentrations no greater than 500 μM. If no changes in fluorescence are noted at this concentration, the molecule can be considered inactive.

Molecules causing increases in [Ca²⁺]ᵢ are subjected to additional testing. Two characteristics of a molecule which can be considered in screening for a positive modulating agent of a chimeric receptor of the invention are the mobilization of intracellular Ca²⁺ and sensitivity to PKC activators.

A single preparation of cells can provide data on [Ca²⁺]ᵢ cyclic AMP levels, IP₃ and other intracellular messengers. A typical procedure is to load cells with fura-2 and then divide the cell suspension in two; most of the cells are used for measurement of [Ca²⁺]ᵢ and the remainder are incubated with molecules to assess their effects on cyclic AMP.

Measurements of inositol phosphates are a time-consuming aspect of the screening. However, ion-exchange columns eluted with chloride (rather than formate) provide a very rapid means of screening for IP₃ formation, since rotary evaporation (which takes around 30 hours) is not required. This method allows processing of nearly 100 samples in a single afternoon by a single experimenter. Those molecules that prove interesting, as assessed by measurements of [Ca²⁺]ᵢ, cyclic AMP, and IP₃ can be subjected to a more rigorous analysis by examining formation of various inositol phosphates and assessing their isomeric form by HPLC.

The following is illustrative of methods useful in these screening procedures.

i. Measurement of Cyclic AMP

This section describes measuring cyclic AMP levels. Cells are incubated as above and at the end of the incubation, a 0.15-ml sample is taken and transferred to 0.85 ml of hot (70° C.) water and heated at this temperature for 5-10 minutes. The tubes are subsequently frozen and thawed several times and the cellular debris sedimented by centrifugation. Portions of the supernatant are acetylated and cyclic AMP concentrations determined by radioimmunoassay.

ii. Measurement of Inositol Phosphate Formation

This section describes procedures measuring inositol phosphate formation. Membrane phospholipids are labeled by incubating parathyroid or other appropriate cells with 4 μCi/ml ³H-myo-inositol for 20-24 hours. Cells are then washed and resuspended in PCB containing 0.5 mM CaCl₂ and 0.1% BSA. Incubations are performed in microfuge tubes in the absence or presence of various concentrations of organic polycation for different times. Reactions are terminated by the addition of 1 ml chloroform-methanol-12 N HCl (200:100:1; v/v/v). Aqueous phytic acid hydrolysate (200 μl; 25 μg phosphate/tube). The tubes are centrifuged and 600 μl of the aqueous phase is diluted into 10 ml water.

Inositol phosphates are separated by ion-exchange chromatography using AG1-X8 in either the chloride- or formate-form. When only IP₃ levels are to be determined, the chloride-form was used, whereas the formate form is used to resolve the major inositol phosphates (IP₃, IP₂, and IP₁). For determination of just IP₃, the diluted sample is applied to the chloride-form column and the column is washed with 10 ml 30 mM HCl followed by 6 ml 90 mM HCl and the IP₃ is eluted with 3 ml 500 mM HCl. The last eluate is diluted and counted. For determination of all major inositol phosphates, the diluted sample is applied to the formate-form column and IP₁, IP₂, and IP₃ eluted sequentially by increasing concentrations of formate buffer. The eluted samples from the formate columns are rotary evaporated, the residues brought up in scintillation cocktail, and counted.

The isomeric forms of IP₃ are evaluated by HPLC. The reactions are terminated by the addition of 1 ml 0.45 M perchloric acid and stored on ice for 10 minutes. Following centrifugation, the supernatant is adjusted to pH 7-8 with NaHCO₃. The extract is then applied to a Partisil SAX anion-exchange column and eluted with a linear gradient of ammonium formate. The various fractions are then desalted with Dowex followed by rotary evaporation prior to liquid scintillation counting in a Packard Tri-carb 1500 LSC.

For all inositol phosphate separation methods, appropriate controls using authentic standards were used to determine if organic polycations interfered with the separation. If so, the samples were treated with cation-exchange resin to remove the offending molecule prior to separation of inositol phosphates.

iii. Use of Lead Molecules

By systematically measuring the ability of a lead molecule to mimic or antagonize the effect of a natural ligand, the importance of different functional groups for agonists and antagonists can be identified. Of the molecules tested, some are suitable as drug candidates while others are not necessarily suitable as drug candidates. The suitability of a molecule as a drug candidate depends on factors such as efficacy and toxicity. Such factors can be evaluated using standard techniques. Thus, lead molecules can be used to demonstrate that the hypothesis underlying receptor-based therapies is correct and to determine the structural features that enable the receptor-modulating agents to act on the receptor and, thereby, to obtain other molecules useful in this invention.

The examples described herein demonstrate the general design of molecules useful as modulators of the activity of mGluRs. The examples also describe screening procedures to obtain additional molecules, such as the screening of natural product libraries. Using these procedures, those of ordinary skill in the art can identify other useful modulators of mGluRs.

Cell lines expressing calcium receptors have been obtained and methods applicable to their use in high throughput screening to identify compounds which modulate the activity of calcium receptors are disclosed (See U.S. Pat. No. 6,011,068, hereby incorporated by reference herein). Cell lines expressing metabotropic glutamate receptors have been obtained and methods applicable to their potential use to identify compounds which modulate activity of metabotropic glutamate receptors are disclosed (European Patent Publication No. 0 568 384 A1; European Patent Publication No. 0 569 240 A1; PCT Publication No. WO 94/29449; and PCT Publication No. WO 92/10583).

Thus, recombinant cell-based assays which use biochemical, spectrophotometric or other physical measurements to detect the modulation of activity of an expressed receptor, especially by measuring changes in affected intracellular messengers, are known to those in the art and can be constructed such that they are suitable for high throughput functional screening of compounds and compound libraries. It will be appreciated by those in the art that each functional assay has advantages and disadvantages for high throughput screening which will vary depending on the receptor of interest, the cell lines employed, the nature of the biochemical and physical measurements used to detect modulation of receptor function, the nature of the compound library being screened and various other parameters. An exceptionally useful and practical method is the use of fluorescent indicators of intracellular Ca²⁺ to detect modulation of the activity of receptors coupled to phospholipase-C.

The use of [³H]glutamate, or any other compound found to modulate the mGluR discovered by the methods described herein, as a lead compound is expected to result in the discovery of other compounds having similar or more potent activity which in turn can be used as lead compounds. Lead compounds or other modulating compounds such as [³H]glutamate can be used for molecular modeling using standard procedures and to screen compound libraries. Radioligand binding techniques (a radiolabeled binding assay) can be used to identify compounds binding at the glutamate binding site. While such binding assays are useful for finding new compounds binding to the glutamate binding site on mGluRs, the current invention provides for the discovery of novel compounds with unique and useful activities at mGluRs which can be radiolabeled and used similarly in radioligand assays to find additional compounds binding to the mGluR. This screening test allows vast numbers of potentially useful compounds to be screened for their ability to bind to the glutamate binding site. Other rapid assays for detection of binding to the glutamate binding site on metabotropic glutamate receptors can be devised using standard detection techniques.

Other compounds can be identified which act at the glutamate binding using the procedures described in this section. A high-throughput assay is first used to screen product libraries (e.g., natural product libraries and compound files) to identify compounds with activity at the glutamate (or lead compound) binding site. These compounds are then utilized as chemical lead structures for a drug development program targeting the glutamate or lead compound binding site on metabotropic glutamate receptors. Routine experiments, including animal studies can be performed to identify those compounds having the desired activities.

The following assay can be utilized as a high-throughput assay. Rat brain membranes are prepared according to the method of Williams et al. (Molec. Pharmacol. 36:575, 1989), with the following alterations: Male Sprague-Dawley rats (Harlan Laboratories) weighing 100-200 g are sacrificed by decapitation. The cortex or cerebellum from 20 rats are cleaned and dissected. The resulting brain tissue is homogenized at 4° C. with a polytron homogenizer at the lowest setting in 300 ml 0.32 M sucrose containing 5 mM K-EDTA (pH 7.0). The homogenate is centrifuged for 10 min at 1,000×g and the supernatant removed and centrifuged at 30,000×g for 30 minutes. The resulting pellet is resuspended in 250 ml 5 mM K-EDTA (pH 7.0) stirred on ice for 15 minutes, and then centrifuged at 30,000×g for 30 minutes. The pellet is resuspended in 300 ml 5 mM K-EDTA (pH 7.0) and incubated at 32° C. for 30 minutes. The suspension is then centrifuged at 100,000×g for 30 minutes. Membranes are washed by resuspension in 500 ml 5 mM K-EDTA (pH 7.0), incubated at 32° C. for 30 minutes, and centrifuged at 100,000×g for 30 minutes. The wash procedure, including the 30-minute incubation, is repeated. The final pellet is resuspended in 60 ml 5 mM K-EDTA (pH 7.0) and stored in aliquots at −80° C.

To perform a binding assay with [³H]glutamate (as an example of a lead compound), aliquots of SPMs (synaptic plasma membranes) are thawed, resuspended in 30 ml of 30 mM EPPS/1 mM K-EDTA, pH 7.0, and centrifuged at 100,000×g for 30 minutes. SPMs are resuspended in buffer A (30 mM EPPS/1 mM K-EDTA, pH 7.0). The [³H]-glutamate is added to this reaction mixture. Binding assays are carried out in polypropylene test tubes. The final incubation volume is 500 μl. Nonspecific binding is determined in the presence of 100 μM nonradioactive glutamate. Duplicate samples are incubated at 0° C. for 1 hour. Assays are terminated by adding 3 ml of ice-cold buffer A, followed by filtration over glass-fiber filters (Schleicher & Schuell No.30) that are presoaked in 0.33% polyethyleneimine (PEI). The filters are washed with another 3×3 ml of buffer A, and radioactivity is determined by scintillation counting at an efficiency of 35-40% for ³H.

In order to validate the above assay, the following experiments can also be performed:
(a) The amount of nonspecific binding of the [³H]glutamate to the filters is determined by passing 500 μl of buffer A containing various concentrations of [³H]glutamate through the presoaked glass-fiber filters. The filters are washed with another 4×3 ml of buffer A, and radioactivity bound to the filters is determined by scintillation counting at an efficiency of 35-40% for ³H.
(b) A saturation curve is constructed by resuspending SPMs in buffer A. The assay buffer (500 μl) contains 60 μg of protein. Concentrations of [³H]glutamate are used, ranging from 1.0 nM to 400 μM in half-log units. A saturation curve is constructed from the data, and an apparent K_{D} value and Bₘₐₓ value determined by Scatchard analysis (Scatchard, Ann. N.Y. Acad. Sci. 51: 660, 1949). The cooperativity of binding of the [³H]glutamate is determined by the construction of a Hill plot (Hill, J. Physiol. 40:190, 1910).
(c) The dependence of binding on protein (receptor) concentration is determined by resuspending SPMs in buffer A. The assay buffer (500 μl) contains a concentration of [³H]glutamate equal to its K_{D} value and increasing concentrations of protein. The specific binding of [³H]glutamate should be linearly related to the amount of protein (receptor) present.
(d) The time-course of ligand-receptor binding is determined by resuspending SPMs in buffer A. The assay buffer (500 μl) contains a concentration of [³H]glutamate equal to its K_{D} value and 100 μg of protein. Duplicate samples are incubated at 0° C. for varying lengths of time; the time at which equilibrium is reached is determined, and this time point is routinely used in all subsequent assays.
(e) The pharmacology of the binding site can be analyzed by competition experiments. In such experiments, the concentration of [3H]glutamate and the amount of protein are kept constant, while the concentration of test (competing) drug is varied. This assay allows for the determination of an IC₅₀ and an apparent K_{D} for the competing drug (Cheng and Prusoff, J. Biochem. Pharmacol. 22:3099, 1973). The cooperativity of binding of the competing drug is determined by Hill plot analysis.

Specific binding of the [³H]glutamate represents binding to the glutamate binding site on metabotropic glutamate receptors. As such, analogs of glutamate should compete with the binding of [³H]glutamate in a competitive fashion, and their potencies in this assay should correlate with their potencies in a functional assay of metabotropic glutamate receptor activity (e.g., electrophysiological assessment of the activity of cloned metabotropic glutamate receptors expressed in Xenopus oocytes). Conversely, compounds which have activity at the sites other that the glutamate binding site should not displace [³H]glutamate binding in a competitive manner. Rather, complex allosteric modulation of [³H]glutamate binding, indicative of noncompetitive interactions, might occur.
(f) Studies estimating the dissociation kinetics are performed by measuring the binding of [³H]glutamate after it is allowed to come to equilibrium (see (d) above), and a large excess of nonradioactive competing drug is added to the reaction mixture. Binding of the [³ H]glutamate is then assayed at various time intervals. With this assay, the association and dissociation rates of binding of the [³H]glutamate are determined (Titeler, Multiple Dopamine Receptors: Receptor Binding Studies in Dopamine Pharmacology. Marcel Dekker, Inc., New York, 1983). Additional experiments involve varying the reaction temperature (0° C. to 37° C.) in order to understand the temperature dependence of this parameter.

The following is one example of a rapid screening assay to obtain compounds modulating metabotropic glutamate receptor activity. The screening assay first measures the ability of compounds to bind to recombinant receptors, or receptor fragments containing the glutamate binding site. Compounds binding to the metabotropic glutamate receptor are then tested for their ability to modulate one or more activities at a metabotropic glutamate receptor.

In one procedure, a cDNA or gene clone encoding the chimeric receptor or fragment of a metabotropic glutamate receptor from a suitable organism such as a human is obtained using standard procedures. Distinct fragments of the clone are expressed in an appropriate expression vector to produce the smallest receptor polypeptide(s) obtainable able to bind glutamate. In this way, the polypeptide(s) containing the glutamate binding site is identified. Such experiments can be facilitated by utilizing a stably transfected mammalian cell line (e.g., HEK 293 cells) expressing the receptor.

Alternatively, the metabotropic glutamate receptor can be chemically reacted with glutamate chemically modified so that amino acid residues of the metabotropic glutamate receptor which contact (or are adjacent to) the selected compound are modified and thereby identifiable. The fragment(s) of the metabotropic glutamate receptor containing those amino acids which are determined to interact with glutamate and are sufficient for binding to glutamate, can then be recombinantly expressed using standard techniques.

The recombinant polypeptide(s) having the desired binding properties can be bound to a solid-phase support using standard chemical procedures. This solid-phase, or affinity matrix, may then be contacted with glutamate to demonstrate that this compound can bind to the column, and to identify conditions by which the compound may be removed from the solid-phase. This procedure may then be repeated using a large library of compounds to determine those compounds which are able to bind to the affinity matrix. Bound compounds can then can be released in a manner similar to glutamate. Alternative binding and release conditions may be utilized to obtain compounds capable of binding under conditions distinct from those used for glutamate binding (e.g., conditions which better mimic physiological conditions encountered especially in pathological states). Compounds binding to the glutamate binding site can thus be selected from a very large collection of compounds present in a liquid medium or extract.

In an alternate method, chimeric receptors are bound to a column or other solid phase support. Those compounds which are not competed off by reagents binding to the glutamate binding site on the receptor can then be identified. Such compounds define alternative binding sites on the receptor. Such compounds may be structurally distinct from known compounds and may define chemical classes of agonists or antagonists which may be useful as therapeutics agents.

Modulating metabotropic glutamate receptor activity causes an increase or decrease in a cellular response which occurs upon metabotropic glutamate receptor activation. Cellular responses to metabotropic glutamate receptor activation vary depending upon the type of metabotropic glutamate receptor activated. Generally, metabotropic glutamate receptor activation causes one or more of the following activities: (1) increase in PI hydrolysis; (2) activation of phospholipase C; (3) increases and decreases in the formation of cyclic adenosine monophosphate (cAMP); (4) decrease in the formation of cAMP; (5) changes in ion channel function; (6) activation of phospholipase D; (7) activation or inhibition of adenylyl cyclase; (8) activation of guanylyl cyclase; (9) increases in the formation of cyclic guanosine monophosphate (cGMP); (10) activation of phospholipase A₂; (11) increases in arachidonic acid release; (12) increases or decreases in the activity of voltage- and ligand- gated ion channels; (13) and increase in intracellular calcium. Inhibition of metabotropic glutamate receptor activation prevents one or more of these activities from occurring.

Activation of a particular metabotropic glutamate receptor refers to an event which subsequently causes the production of one or more activities associated with the type of receptor activated. Activation of mGluR1 can result in one or more of the following activities: increase in PI hydrolysis, increase in cAMP formation, increase in intracellular calcium (Ca²⁺) and increase in arachidonic acid formation. Compounds can modulate one or more metabotropic glutamate receptor activities by acting as an agonist or antagonist of glutamate binding site activation.

The chimeric receptors of the present invention provide a method of screening for compounds active at mGluRs by the detection of signals produced by CaRs. The chimeric receptors may be used in the screening procedures described in PCT/US93/01642 (WO94/18959), which are hereby incorporated by reference herein, including methods of screening using fura-2, and measurement of cytosolic Ca2+ using cell lines expressing calcium receptors and methods of screening using oocyte expression.

Active compounds identified by the screening methods described herein, may be useful as therapeutic molecules to modulate metabotropic glutamate receptor activity or as a diagnostic agents to diagnose those patients suffering from a disease characterized by an abnormal metabotropic glutamate receptor activity. Preferably the screening methods are used to identify metabotropic glutamate receptor modulators by screening potentially useful molecules for an ability to mimic or block an activity of extracellular glutamate or other metabotropic glutamate receptor agonists on a cell having a metabotropic glutamate receptor and determining whether the molecule has an EC₅₀ IC₅₀ of less than or equal to 100 μM. More preferably, the molecules tested for its ability to mimic or block an increase in [Ca²⁺]; elicited by extracellular glutamate or other mGluR agonists.

Identification of metabotropic glutamate receptor-modulating agents is facilitated by using a high-throughput screening system. High-throughput screening allows a large number of molecules to be tested. For example, a large number of molecules can be tested individually using rapid automated techniques or in combination using a combinatorial library. Individual compounds able to modulate metabotropic glutamate receptor activity present in a combinatorial library can be obtained by purifying and retesting fractions of the combinatorial library. Thus, thousands to millions of molecules can be screened in a single day. Active molecules can be used as models to design additional molecules having equivalent or increased activity. Preferably the identification method uses a recombinant chimeric metabotropic glutamate receptor. Chimeric receptors can be introduced into different cells using a vector encoding a receptor. Preferably, the activity of molecules in different cells is tested to identify a metabotropic glutamate receptor agonist or metabotropic glutamate receptor antagonist molecule which mimics or blocks one or more activities of glutamate at a first type of metabotropic glutamate receptor but not at a second type of metabotropic glutamate receptor.

As indicated above, the present invention provides a method of screening for compounds which modulate metabotropic glutamate receptor activity, by using a chimeric receptor having at least a portion of a metabotropic glutamate receptor linked with a non-natural signal peptide, and can also include portions of a calcium receptor. In particular receptors of this type, the signaling process of the calcium receptor portion is used to detect modulation of mGluR activity, as various compounds are tested for binding to the mGluR portion. The method of screening can be conducted in a variety of ways, such as utilizing chimeric receptors having different portions from the metabotropic glutamate receptor and calcium receptor. Certain preferred examples are described below.

In one example, the method of screening for a compound that binds to or modulates the activity of a metabotropic glutamate receptor involves preparing a chimeric receptor having an extracellular domain, a seven transmembrane domain, and usually an intracellular cytoplasmic tail domain. The extracellular domain sequence is the same as or homologous to a sequence from a metabotropic glutamate receptor and is linked to a non-native signal peptide. The chimeric receptor and a test compound are introduced into a acceptable medium, and the binding of the test compound to the receptor or the modulation of the receptor by the test compound is monitored by physically detectable means in order to identify such binding or modulating compounds. Generally, acceptable media will include those in which a natural ligand of an mGluR will interact with the mGluR.

It can be beneficial to use chimeric receptors which have longer sequences from the CaR. For example, the chimeric receptor can have a sequence of at least 12, 18, 24, 30, 36, 54, 72, 96 or more amino acids the same as or homologous a sequence from the CaR.

In a second example, the method of screening for a compound which binds to or modulates the activity of a metabotropic glutamate receptor utilizes a nucleic acid sequence which encodes a chimeric receptor as described herein. The nucleic acid is expressed in a cell, and binding or modulation by a test compound is observed by monitoring the effects of the test compound on the cell. Thus, generally the method includes preparing a nucleic acid sequence encoding a chimeric receptor. The encoded chimeric receptor has an mGluR extracellular domain linked to a non-native signal peptide, a seven transmembrane domain, and usually an intracellular cytoplasmic tail domain. The chimeric receptor sequence other than the signal peptide sequence can be from a particular mGluR; the the chimeric receptor can have sequences of at least 6 contiguous amino acids which are the same as or homologous to sequences from a CaR. The nucleic acid sequence is inserted into a replicable expression vector capable of expressing the chimeric receptor in a host cell, and a host cell is transformed with the vector. The transformed host cell and a test compound are introduced into an acceptable medium and the effect of the compound on the host cell is monitored (such as be techniques or assays described above). Preferably, though not necessarily, the host cell is a eukaryotic cell.

Thus, the method involves contacting a host cell expressing the chimeric receptor in an acceptable medium and monitoring, determining, or measuring the effect, if any of the presence of the test compound on the cell.

The chimeric metabotropic glutamate/calcium receptors can also be used to screen for compounds active at both metabotropic glutamate receptors and calcium receptors. This is particularly useful for screening for compounds which interact at different domains or subdomains in an mGluR as compared to in a CaR. Thus, such chimeras are useful for screening for compounds which, for example, act within the extracellular domain of a metabotropic glutamate receptor and also act within the seven transmembrane domain or the cytoplasmic tail domain of a calcium receptor. Such a chimera would include the extracellular domain of a metabotropic glutamate receptor linked to the seven transmembrane domain and cytoplasmic tail of a calcium receptor.

To screen for such compounds, active at both metabotropic glutamate receptors and calcium receptors, compounds would be screened according to the various methods of the present invention, against the chimeric receptor, the calcium receptor, and the metabotropic glutamate receptor. Compounds active at the seven transmembrane domain of the calcium receptor portion of the chimeric receptor should also be active when tested against the calcium receptor itself. An exemplary method of screening for such compounds is to first screen them according to the methods of the present invention against a chimeric molecule having the extracellular domain of the metabotropic glutamate receptor, and the seven transmembrane and cytoplasmic tail domains of the calcium receptor and to then screen the positive compounds against both chimeric molecule having the extracellular and seven transmembrane domains of the metabotropic glutamate receptor and the cytoplasmic tail domain of the calcium receptor, and the calcium receptor itself. Compounds active at both molecules will be positive when tested against all three chimeric receptors.

Thus in one aspect the invention features a method of screening for compounds active at both a metabotropic glutamate receptor and a calcium receptor, by preparing a nucleic acid sequence encoding a chimeric receptor. The chimeric receptor has an extracellular domain, a seven transmembrane domain, and an intracellular cytoplasmic tail domain, and at least the extracellular domain is homologous to the extracellular domain of the metabotropic glutamate receptor and at least one domain is homologous to a domain of a calcium receptor. The nucleic acid sequence is inserted into a replicable expression vector capable of expressing said chimeric receptor in a host cell, and a host cell is transformed with the vector. The transformed host cell and a test compound are introduced into an acceptable medium, and the effect of the test compound on the cell are monitored.

Thus, the method involves contacting a cell expressing a chimeric receptor as described herein with a test compound in a suitable medium and monitoring, determining, or measuring the effect, if any, of the presence of the test compound on the cell. The method can also include one or more of the preparatory steps.

In general, for each of the above screening methods using chimeric receptors, the portion of the chimeric receptor homologous to an mGluR and the portion, if any, homologous to a CaR are selected to provide the binding, modulation, and/or signal coupling characteristics appropriate for a particular application.

E. Site of Action

The chimeric receptor molecules are also useful in methods for determining the site-of-action of compounds already identified as metabotropic glutamate receptor or calcium receptor active compounds. For example, chimeras including the extracellular domain of a metabotropic glutamate receptor linked to the seven transmembrane domain and cytoplasmic tail of a calcium receptor, as well as chimeras including the extracellular domain of a calcium receptor linked to the seven transmembrane domain and cytoplasmic tail of a metabotropic glutamate receptor would be useful in determining the site-of-action of either metabotropic glutamate receptor or calcium receptor active compounds. Those of ordinary skill in the art will recognize that these are two examples of large sequence exchanges and that much smaller sequence exchanges may also be employed to further refine the determination of the site-of-action.

Thus, the invention provides a method of determining the site-of-action of a metabotropic glutamate receptor active compound by: preparing a nucleic acid sequence encoding a chimeric receptor wherein the chimeric receptor comprises at least a 6 amino acid sequence which is homologous to a sequence of amino acids of a calcium receptor and the remainder of the amino acid sequence is homologous to a sequence of amino acids of a metabotropic glutamate receptor; inserting the sequence into a replicable expression vector capable of expressing the chimeric receptor in a host cell; transforming a host cell with the vector; introducing the transformed host cell and the compound into an acceptable medium; and monitoring the effect of the compound on the cell.

As indicated above for methods of screening, in particular applications it is advantageous to use sequence exchanges of different sizes. Thus, in other applications, the sequence homologous to a sequence from a calcium receptor, may for example, be at least 12, 18, 24, 30, 36, 54, 72, 96 or more amino acids in length.

Conversely, a method of determining the site-of-action of a calcium receptor active compound can be performed in the same manner as described above, but using a nucleic acid encoding a chimeric receptor which includes at least a 6 amino acid sequence which is homologous to a sequence of amino acids of a metabotropic glutamate receptor and the remainder of the amino acid sequence is homologous to a sequence of amino acids of a calcium receptor. Also similar to the method above, the sequence homologous to a sequence from a metabotropic glutamate receptor can be of different lengths in various applications, for example, at least 12, 18, 24, 30, 36, or more amino acids in length.

F. Modulation of Metabotropic Glutamate Receptor Activity

Modulation of metabotropic glutamate receptor activity can be used to produce different effects such as anticonvulsant effects, neuroprotectant effects, analgesic effects, cognition-enhancement effects, and muscle-relaxation effects. Each of these effects has therapeutic applications. Compounds used therapeutically should have minimal side effects at therapeutically effective doses.

The ability of a compound to modulate metabotropic glutamate activity can be determined using electrophysiological and biochemical assays measuring one or more metabotropic glutamate activities. In general, such assays can be carried out using cells expressing the metabotropic glutamate receptor(s) of interest, but the assays can also be carried out using cells expressing a chimeric receptors of this invention which modulates the cellular activity which is to be monitored. Examples of such assays include the electrophysiological assessment of metabotropic glutamate receptor function in Xenopus oocytes expressing cloned metabotropic glutamate receptors, the electrophysiological assessment of metabotropic glutamate receptor function in transfected cell lines (e.g., CHO cells, HEK 293 cells, etc.) expressing cloned metabotropic glutamate receptors, the biochemical assessment of PI hydrolysis and cAMP accumulation in transfected cell lines expressing cloned metabotropic glutamate receptors, the biochemical assessment of PI hydrolysis and cAMP accumulation in rat brain (e.g., hippocampal, cortical, striatal, etc.) slices, fluorimetric measurements of cytosolic Ca²⁺ in cultured rat cerebellar granule cells, and fluorimetric measurements of cytosolic Ca²⁺ in transfected cell lines expressing cloned metabotropic glutamate receptors.

Prior to therapeutic use in a human, the compounds are preferably tested in vivo using animal models. Animal studies to evaluate a compound's effectiveness to treat different diseases or disorders, or exert an effect such as an analgesic effect, a cognition-enhancement effect, or a muscle-relaxation effect, can be carried out using standard techniques.

G. Novel Agents and Pharmaceutical Compositions

The chimeric receptors and screening methods described herein provide metabotropic glutamate receptor-binding agents (e.g., compounds and pharmaceutical compositions) discovered due to their ability to bind to a chimeric metabotropic glutamate receptor. Such binding agents are preferably modulators of a metabotropic glutamate receptor. Certain of these agents will be novel compounds identified by the screening methods described herein. In addition, other such compounds are derived by standard methodology from such identified compounds when such identified compounds are used as lead compounds in screening assays based on analogs of identified active compounds, or in medicinal chemistry developments using identified compounds as lead compounds.

Further, by providing novel and efficient screening methods using chimeric receptors, this invention provides a method for preparing a pharmaceutical agent active on a metabotropic glutamate receptor. Without such this efficient method, such agents would not be identified. The method involves identifying an active agent by screening using a chimeric receptor of the type described herein in a screening method as described above. The identified agent or an analog of that agent is synthesized in an amount sufficient to administer to a patient in a therapeutically effective amount.

H. Treatment of Diseases and Disorders

A preferred use of the compounds and methods of the present invention is in the treatment of neurological diseases and disorders. Patients suffering from a neurological disease or disorder can be diagnosed by standard clinical methodology.

Neurological diseases or disorders include neuronal degenerative diseases, glutamate excitotoxicity, global and focal ischemic and hemorrhagic stroke, head trauma, spinal cord injury, hypoxia-induced nerve cell damage, and epilepsy. These different diseases or disorders can be further medically characterized. For example, neuronal degenerative diseases include Alzheimer's disease and Parkinson's disease.

Another preferred use of the present invention is in the production of other therapeutic effects, such as analgesic effects, cognition-enhancement effects, or muscle-relaxation effects. The present invention is preferably used to produce one or more of these effects in a patient in need of such treatment.

Patients in need of such treatment can be identified by standard medical techniques. For example, the production of analgesic activity can be used to treat patients suffering from clinical conditions of acute and chronic pain including the following: preemptive preoperative analgesia; peripheral neuropathies such as occur with diabetes mellitus and multiple sclerosis; phantom limb pain; causalgia; neuralgias such as occur with herpes zoster; central pain such as that seen with spinal cord lesions; hyperalgesia; and allodynia.

In a method of treating a patient, a therapeutically effective amount of a compound which in vitro modulates the activity of a chimeric receptor having at least the extracellular domain of a metabotropic glutamate receptor is administered to the patient. Typically, the compound modulates metabotropic glutamate receptor activity by acting as an agonist or antagonist of glutamate binding site activation. Active compounds may act outside the extracellular domain, e.g., at the TMD. Preferably, the patient has a neurological disease or a disorder, preferably the compound has an effect on a physiological activity. Such physiological activity can be convulsions, neuroprotection, neuronal death, neuronal development, central control of cardiac activity, waking, control of movements and control of vestibo ocular reflex.

Diseases or disorders which can be treated by modulating metabotropic glutamate receptor activity include one or more of the following types: (1) those characterized by abnormal glutamate homeostasis; (2) those characterized by an abnormal amount of an extracellular or intracellular messenger whose production can be affected by metabotropic glutamate receptor activity; (3) those characterized by an abnormal effect (e.g., a different effect in kind or magnitude) of an intracellular or extracellular messenger which can itself be ameliorated by metabotropic glutamate receptor activity; and (4) other diseases or disorders in which modulation of metabotropic glutamate receptor activity will exert a beneficial effect, for example, in diseases or disorders where the production of an intracellular or extracellular messenger stimulated by receptor activity compensates for an abnormal amount of a different messenger.

The compounds and methods can also be used to produce other effects such as an analgesic effect, cognition-enhancement effect, and a muscle-relaxant effect.

A “patient” refers to a mammal in which modulation of an metabotropic glutamate receptor will have a beneficial effect. Patients in need of treatment involving modulation of metabotropic glutamate receptors can be identified using standard techniques known to those in the medical profession. Preferably, a patient is a human having a disease or disorder characterized by one more of the following: (1) abnormal glutamate receptor activity (2) an abnormal level of a messenger whose production or secretion is affected by metabotropic glutamate receptor activity; and (3) an abnormal level or activity of a messenger whose function is affected by metabotropic glutamate receptor activity.

By “therapeutically effective amount” is meant an amount of an agent which relieves to some extent one or more symptoms of the disease or disorder in the patient; or returns to normal either partially or completely one or more physiological or biochemical parameters associated with or causative of the disease.

More generally, this invention provides a method for modulating metabotropic glutamate receptor activity by providing to a cell having a metabotropic glutamate receptor an amount of a metabotropic glutamate receptor-modulating molecule sufficient to either mimic one or more effects of glutamate at the metabotropic glutamate receptor, or block one or more effects of glutamate at the metabotropic glutamate receptor. The method can carried out in vitro or in vivo.

I. Formulation and Administration

Active compounds as identified by the methods of this invention can be utilized as pharmaceutical agents or compositions to treat different diseases and disorders as described above. In this context, a pharmacological agent or composition refers to an agent or composition in a form suitable for administration to a mammal, preferably a human.

The optimal formulation and mode of administration of compounds of the present invention to a patient depend on factors known in the art such as the particular disease or disorder, the desired effect, and the type of patient. While the compounds will typically be used to treat human patients, they may also be used to treat similar or identical diseases in other vertebrates such as other primates, farm animals such as swine, cattle and poultry, and sports animals and pets such as horses, dogs and cats.

Preferably, the therapeutically effective amount is provided as a pharmaceutical composition. A pharmacological agent or composition refers to an agent or composition in a form suitable for administration into a multicellular organism such as a human. Suitable forms, in part, depend upon the use or the route of entry, for example oral, transdermal, or by injection. Such forms should allow the agent or composition to reach a target cell whether the target cell is present in a multicellular host or in culture. For example, pharmacological agents or compositions injected into the blood stream should be soluble. Other factors are known in the art, and include considerations such as toxicity and forms which prevent the agent or composition from exerting its effect.

The claimed compositions can also be formulated as pharmaceutically acceptable salts (e.g., acid addition salts) and/or complexes thereof. Pharmaceutically acceptable salts are non-toxic salts at the concentration at which they are administered. The preparation of such salts can facilitate the pharmacological use by altering the physical-chemical characteristics of the composition without preventing the composition from exerting its physiological effect. Examples of useful alterations in physical properties include lowering the melting point to facilitate transmucosal administration and increasing the solubility to facilitate the administration of higher concentrations of the drug.

Pharmaceutically acceptable salts include acid addition salts such as those containing sulfate, hydrochloride, phosphate, sulfamate, acetate, citrate, lactate, tartrate, methanesulfonate, ethanesulfonate, benzenesulfonate, p-toluenesulfonate, cyclohexylsulfamate and quinate. (See e.g., supra. PCT/US92/03736.) Pharmaceutically acceptable salts can be obtained from acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid.

Pharmaceutically acceptable salts can be prepared by standard techniques. For example, the free base form of a compound is dissolved in a suitable solvent, such as an aqueous or aqueous-alcohol solution, containing the appropriate acid and then isolated by evaporating the solution. In another example, a salt is prepared by reacting the free base and acid in an organic solvent.

Carriers or excipients can also be used to facilitate administration of the compound. Examples of carriers and excipients include calcium carbonate, calcium phosphate, various sugars such as lactose, glucose, or sucrose, or types of starch, cellulose derivatives, gelatin, vegetable oils, polyethylene glycols and physiologically compatible solvents. The compositions or pharmaceutical composition can be administered by different routes including intravenously, intraperitoneal, subcutaneous, and intramuscular, orally, topically, or transmucosally.

The compounds of the invention can be formulated for a variety of modes of administration, including systemic and topical or localized administration. Techniques and formulations generally may be found in Remington 's Pharmaceutical Sciences, 18^{th} Edition, Mack Publishing Co., Easton, Pa., 1990.

For systemic administration, oral administration is preferred. For oral administration, the compounds are formulated into conventional oral dosage forms such as capsules, tablets and tonics.

Alternatively, injection may be used, e.g., intramuscular, intravenous, intraperitoneal, subcutaneous, intrathecal, or intracerebroventricular. For injection, the compounds of the invention are formulated in liquid solutions, preferably in physiologically compatible buffers such as Hank's solution or Ringer's solution. Alternatively, the compounds of the invention are formulated in one or more excipients (e.g., propylene glycol) that are generally accepted as safe as defined by USP standards. In addition, the compounds may be formulated in solid form and redissolved or suspended immediately prior to use. Lyophilized forms are also included.

Systemic administration can also be by transmucosal or transdermal means, or the molecules can be administered orally. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration may be, for example, through nasal sprays or using suppositories. For oral administration, the molecules are formulated into conventional oral administration dosage forms such as capsules, tablets, and liquid preparations.

For topical administration, the compounds of the invention are formulated into ointments, salves, gels, or creams, as is generally known in the art.

The amounts of various compounds to be administered can be determined by standard procedures. Generally, a therapeutically effective amount is between about 1 nmole and 3 μmole of the molecule, preferably 0.1 mmole and 1 μmole depending on its EC₅₀ or IC₅₀ and on the age and size of the patient, and the disease or disorder associated with the patient. Generally, it is an amount between about 0.1 and 50 mg/kg, preferably 0.01 and 20 mg/kg of the animal to be treated.

J. Transgenic Animals

The invention also provides transgenic, nonhuman mammals containing a transgene encoding a chimeric receptor, particularly a chimeric metabotropic glutamate receptor. Transgenic nonhuman mammals are particularly useful as an in vivo test system for studying the effects of introducing a chimeric receptor. Experimental model systems may be used to study the effects in cell or tissue cultures, in whole animals, or in particular cells or tissues within whole animals or tissue culture systems. The effects can be studied over specified time intervals (including during embryogenesis).

The present invention provides for experimental model systems for studying the physiological effects of the receptors. Model systems can be created having varying degrees of receptor expression. For example, the nucleic acid encoding a receptor may be inserted into cells which naturally express the parent receptors, such that the chimeric gene is expressed at much higher levels. Also, a recombinant gene may be used to inactivate the endogenous gene by homologous recombination, and thereby create a receptor deficient cell, tissue, or animal.

Inactivation of a gene can be caused, for example, by using a recombinant gene engineered to contain an insertional mutation (e.g., the neo gene). The recombinant gene is inserted into the genome of a recipient cell, tissue or animal, and inactivates transcription of the receptor. Such a construct may be introduced into a cell, such as an embryonic stem cell, by techniques such as transfection, transduction, and injection. Stem cells lacking an intact receptor sequence may generate transgenic animals deficient in the receptor.

Preferred test models are transgenic animals. A transgenic animal has cells containing DNA which has been artificially inserted into a cell and inserted into the genome of the animal which develops from that cell. Preferred transgenic animals are primates, mice, rats, cows, pigs, horses, goats, sheep, dogs and cats.

A variety of methods are available for producing transgenic animals. For example, DNA can be injected into the pronucleus of a fertilized egg before fusion of the male and female pronuclei, or injected into the nucleus of an embryonic cell (e.g., the nucleus of a two-cell embryo) following the initiation of cell division (Brinster et al., Proc. Nat. Acad. Sci. USA 82: 4438-4442, 1985)). By way of another example, embryos can be infected with viruses, especially retroviruses, modified to carry chimeric receptor nucleotide sequences of the present invention.

Pluripotent stem cells derived from the inner cell mass of the embryo and stabilized in culture can be manipulated in culture to incorporate nucleotide sequences of the invention. A transgenic animal can be produced from such stem cells through implantation into a blastocyst that is implanted into a foster mother and allowed to come to term. Animals suitable for transgenic experiments can be obtained from standard commercial sources such as Charles River (Wilmington, Mass.), Taconic (Germantown, N.Y.), and Harlan Sprague Dawley (Indianapolis, Ind.).

Methods for the culturing of embryonic stem (ES) cells and the subsequent production of transgenic animals by the introduction of DNA into ES cells using methods such as electroporation, calcium phosphate/DNA precipitation and direct injection also are well known to those of ordinary skill in the art. See, for example, Teratocarcinomas and Embryonic Stem Cells, A Practical Approach, E. J. Robertson, ed., IRL Press (1987).

Procedures for embryo manipulations are well known in the art. The procedures for manipulation of the rodent embryo and for microinjection of DNA into the pronucleus of the zygote are well known to those of ordinary skill in the art (Hogan et al., supra). Microinjection procedures for fish, amphibian eggs and birds are detailed in Houdebine and Chourrout (Experientia 47:897-905, 1991). Other procedures for introduction of DNA into tissues of animals are described in U.S. Pat. No. 4,945,050 (Sandford et al., Jul. 30, 1990).

Transfection and isolation of desired clones can be carried out using standard techniques (e.g., E. J. Robertson, supra). For example, random gene integration can be carried out by co-transfecting the nucleic acid with a gene encoding antibiotic resistance. Alternatively, for example, the gene encoding antibiotic resistance is physically linked to a nucleic acid sequence encoding a chimeric receptor of the present invention.

DNA molecules introduced into ES cells can also be integrated into the chromosome through the process of homologous recombination. (Capecchi, Science 244: 1288-1292, 1989). Methods for positive selection of the recombination event (e.g., neomycin resistance) and dual positive-negative selection (e.g., neomycin resistance and gancyclovir resistance) and the subsequent identification of the desired clones by PCR have been described by Capecchi, supra and Joyner et al., Nature 338:153-156, 1989), the teachings of which are incorporated herein.

The final phase of the procedure is to inject targeted ES cells into blastocysts and to transfer the blastocysts into pseudopregnant females. The resulting chimeric animals are bred and the offspring are analyzed by Southern blotting to identify individuals that carry the transgene.

An example describing the preparation of a transgenic mouse is as follows. Female mice are induced to superovulate and placed with males. The mated females are sacrificed by CO₂ asphyxiation or cervical dislocation and embryos are recovered from excised oviducts. Surrounding cumulus cells are removed. Pronuclear embryos are then washed and stored until the time of injection.

Randomly cycling adult female mice paired with vasectomized males serve as recipients for implanted embryos. Recipient females are mated at the same time as donor females and embryos are transferred surgically to recipient females.

The procedure for generating transgenic rats is similar to that of mice. See Hammer et al., Cell 63:1099-1112, 1990). Procedures for the production of transgenic non-rodent mammals and other animals are known in art. See, for example, Houdebine and Chourrout, supra; Pursel et al., Science 244:1281-1288, 1989); and Simms et al., Bio/Technology 6:179-183, 1988).

K. Transfected Cell Lines

Nucleic acid expressing a functional chimeric receptor can be used to create transfected cell lines which functionally express a specific chimeric receptor. Such cell lines have a variety of uses such as being used for high-throughput screening for molecules able to modulate metabotropic glutamate receptor activity; and being used to assay binding to a metabotropic glutamate receptor.

A variety of cell lines are capable of coupling exogenously expressed receptors to endogenous functional responses. A number of these cell lines (e.g., NIH-3T3, HeLa, NG115, CHO, HEK 293 and COS7) can be tested to confirm that they lack an endogenous metabotropic glutamate receptor. Those lines lacking a response to external glutamate can be used to establish stably transfected cell lines expressing the cloned chimeric receptors of the invention.

Production of these stable transfectants is accomplished by transfection of an appropriate cell line with a eukaryotic expression vector, such as pMSG, in which the coding sequence for the chimeric metabotropic glutamate receptor cDNA has been cloned into the multiple cloning site. These expression vectors contain a promoter region, such as the mouse mammary tumor virus promoter (MMTV), that drive high-level transcription of cDNAS in a variety of mammalian cells. In addition, these vectors contain genes for the selection of cells that stably express the cDNA of interest. The selectable marker in the pMSG vector encodes an enzyme, xanthine-guanine phosphoribosyl transferase (XGPRT), that confers resistance to a metabolic inhibitor that is added to the culture to kill the nontransfected cells. A variety of expression vectors and selection schemes are usually assessed to determine the optimal conditions for the production of metabotropic glutamate receptor-expressing cell lines for use in high-throughput screening assays.

The most effective method for transfection of eukaryotic cell lines with plasmid DNA varies with the given cell type. The chimeric receptor expression construct will be introduced into cultured cells by the appropriate technique, either Ca²⁺ phosphate precipitation, DEAE-dextran transfection, lipofection or electroporation. One transfection approach is to use virally-mediated transfection.

Cells that have stably incorporated or are episomally maintaining the transfected DNA can be identified by their resistance to selection media, as described above, and clonal cell lines can be produced by expansion of resistant colonies. The expression of the chimeric metabotropic glutamate receptor cDNA by these cell lines can be assessed by solution hybridization and Northern blot analysis, radioligand binding, or cell staining with appropriate epitope-recognizing antibodies. Functional expression of the receptor protein can be determined by measuring the mobilization of intracellular Ca2+ in response to externally applied calcium receptor agonists.

The following examples illustrate the invention, but do not limit its scope.

### EXAMPLES

Examples are provided below to illustrate different aspects and embodiments of the present invention. These examples are not intended in any way to limit the disclosed invention. Rather, they illustrate methodologies by which the novel chimeric receptors of the present invention may be constructed and assessed for function. They also illustrate methodologies by which compounds may be screened to determine which compounds bind to or modulate a desired mGluR.

I. Methods for Analyzing Expression and/or Function of Chimeric Receptors

### Example 1

### Functional Expression in Oocytes

Oocytes suitable for injection were obtained from adult female Xenopus laevis toads using procedures described in C. J. Marcus-Sekura and M. J. M. Hitchcock, Methods in Enzymology, Vol. 152 (1987). Pieces of ovarian lobe were incubated for 30 minutes in Ca²⁺-free Modified Barths Saline (MBS) containing 1.5 mg/ml collagenase type IA (Worthington). Subsequently, 5 ng of RNA transcript prepared as described below, were injected into each oocyte. Following injection, oocytes were incubated at 16° C. in MBS containing 0.5 mM CaCl₂ for 2-7 days prior to electrophysiological examination.

RNA transcripts encoding the chimeric receptors, or the GIRK subunits described below, were produced by enzymatic transcription from plasmid templates using T7 polymerase supplied with the mMessage mMachine ™(Ambion). Each plasmid was treated with a restriction enzyme to make a single cut distal to the 3′ end of the cDNA insert to linearize the template. This DNA was incubated with T7 RNA polymerase in the presence of GpppG cap nucleotide, rATP, rCTP, rUTP and rGTP. The synthetic RNA transcript is purified by DNase treatment of the reaction mix and subsequent alcohol precipitations. RNA was quantitated by absorbance spectroscopy (OD₂₆₀) and visualized on an ethidium stained 1.2% formaldehyde gel.

A. PLC-Coupled Receptors

The ability of each PLC-coupled chimeric receptor to function was determined by voltage-recording of current-passing electrodes across the oocyte membrane in response to glutamate and calcium receptor agonists. Oocytes were voltage clamped at a holding potential of −60 mV with an Axoclamp 2A amplifier (Axon Instruments, Foster City, Calif.) using standard two electrode voltage-clamp techniques. Currents were recorded on a chart recorder. The standard control saline was MBS containing 0.3 mM CaCl₂ and 0.8 MgCl₂. Test substances were applied by superfusion at a flow rate of about 5 ml/min. All experiments were done at room temperature. The holding current was stable in a given oocyte and varied between +10 to −200 nA for different oocytes. Activation of I_{Cl} in response to activation of receptors and subsequent increases in intracellular Ca2+ ([Ca]ᵢₙ) was quantified by measuring the peak inward current stimulated by agonist or drug, relative to the holding current at −60 mV.

B. Gαi-Coupled Receptors

The ability of each Gαi-coupled mGlu or mGluR-CaR chimeric receptors to function was determined in Xenopus oocytes, with co-injection of the G-protein inward rectifying potassium channel subunits, GIRK 1 and GIRK4 (Kubo et al 1993 Nature 364: 802). Individual oocytes were injected with a mixture containing 5 ng receptor, 1.5 ng GIRK1 and 1.5 ng GIRK4. Following a 4-day incubation, oocytes were voltage clamped at a holding potential of −90 mV using standard two electrode voltage clamp techniques. Further details can be found in Saugstad, et al., J. Neurosci. 1996 16:5979-5985. Application of glutamate evoked inward potassium currents in oocytes expressing both the chimeric receptors and the potassium channels, but not in oocytes expressing only the receptor or only the channel subunits.

### Example 2

### Transfection and Growth of HEK293 Cells to Express Chimeric Receptors

A. Lipofectamine™ 2000 Transfections

Human embryonic kidney cells (HEK293, ATCC, CRL 1573) were maintained and propagated in culture in a routine manner. 20×10⁶ cells were plated in T150 cm² cell culture flasks in Dulbecco's Modified Eagle's Medium (DMEM from Gibco Life Technologies) containing 10 % fetal bovine serum (FBS from Hyclone Laboratories) to attain a monolayer of 95% confluence in 48-hours. To prepare plasmid DNA for transfection, the cDNA was precipitated with ethanol, rinsed and resuspended in sterile water at a concentration of 1 μg/ul. Sixty-three μg of cDNA was incubated with 197.5 μl of the liposome formulation Lipofectamine™ 2000 transfection reagent (Invitrogen) for 20 minutes in 4 ml serum-free Opti-MEM™ (Gibco Life Technologies) at room temperature allowing for the formation of the DNA-Cationic lipid complex. Post incubation, the 4 ml of complex was added to 40 ml of Opti-MEM™ in a T150cm² flask and incubated at 37° C. and 5.0% CO² over night. At this time the Opti-MEM™, DNA-cationic lipid complex was removed and 25 ml of DMEM (2.0 mM L-glutamine)+10% dialyzed fetal bovine serum (Hyclone Laboratories) was added. After an additional 24-hour incubation, the cells were enzymatically dissociated with 0.25% trypsin and plated in DMEM (2.0 mM L-glutamine)+10% dialyzed fetal bovine serum medium containing 200 μg/ml Hygromycin (Invitrogen). Cells that successfully grew out as a stable pool, under Hygromycin selection pressure, expressed the Hygromycin resistance gene contained on the expression vector used to express the heterologous gene product of interest. Individual clones, arising from a single cell, were then recovered and propagated to produce clonal cell lines using standard tissue culture techniques.

B. Amaxa Nucleofection Transfections

Human embryonic kidney cells (ATCC, CRL 1573) were maintained and propagated in culture in a routine manner. For a minimum of 48-hours post enzymatic dissociation, the cells were grown in Dulbecco's Modified Eagle's Medium (D-MEM from Gibco Life Technologies) containing 10% fetal bovine serum (FBS from Hyclone Laboratories) as an adherent monolayer in T-flasks. Cells at 60 to 80% confluence were used for transfection utilizing the Amaxa Nucleofector™ gene delivery technology (Amaxa Biosystems, Germany). 0.25% trypsin was used to enzymatically dissociate the cells and prepare a suspension of 1×10⁶ cells/ml. 2ml, or 2×10⁶ cells, were centrifuged in a 15 ml conical tube at 1200 RPM for 3 minutes to form a pellet. The supernatant was completely removed and 100 μl of Nucleofector™ Solution V was used to resuspend the cell pellet. 4 μg of cDNA, sterilized via ethanol precipitation and at a concentration of 1 μg/μl, was added to the cells and the entire volume was transferred to an Amaxa certified cuvette for electroporation. An appropriate amount of voltage over time was applied to the cells to facilitate gene delivery of cDNA to the nucleus. Post electroporation, 0.4 ml of RPMI-1640 (Gibco, Life Technologies) was added to the cuvette and the entire volume was removed and placed in an additional 1.5 ml RPMI-1640 achieving a final concentration of 1×10⁶ cell/ml. Cells were then plated at 100 μ/well in Collagen-1 coated, 96-well plates (BD Biosciences) at 37 degrees C. and 5.0% CO₂ for transient, functional analysis at 24 hours post gene delivery. Alternatively, for stably expressing cell line development, the cells were placed in T75 flasks for 24-hour outgrowth in an additional 18 ml RPMI-1640 to achieve a seeding concentration of 1×10⁵ cells/ml for stable pool outgrowth. After 24 hours, the RPMI-1640 medium was removed and replacedby DMEM+10% FBS. After an additional 24-hours, cells were dissociated and plated in a T150 flask containing 25 ml of DMEM (2.0 mM L-glutamine)+10 dialyzed FBS+200 μg/ml Hygromycin (Invitrogen Corp.). Cells, which successfully grew out as a stable pool, contained the Hygromycin resistance gene contained on the expression vector used to express the heterologous gene product of interest. Individual clones, arising from a single cell were recovered and propagated using standard tissue culture techniques.

### Example 3

### Measuring Changes in Intracellular Calcium Caused by Activation of Chimeric Receptors by the Fura Assay

Measurements of intracellular calcium release in response to increases in extracellular calcium is quantitated using the Fura assay (Parks et al. 1989). Stably transfected cells containing chimeric receptors are loaded with 2 μM fura-2 acetoxymethylester by incubation for 20-30 minutes at 37° C. in SPF-PCB (126 mM NaCl, 5 mM KCl, 1 mM MgCl₂, 20 mM HEPES, pH 7.4), containing 1.25 mM CaCl₂, 1 mg/ml glucose, 0.5% BSA¹. The cells are then washed 1 to 2 times in SPF-PCB containing 0.5 mM CaCl₂, 0.5% BSA and resuspended to a density of 4 to 5 million cells/ml and kept at 22° C. in a plastic beaker. For recording fluorescent signals, the cells are diluted fivefold into a quartz cuvette with BSA-free 37° C. SPF-PCB to achieve a final BSA concentration of 0.1% (1.2 ml of 37° C. BSA-free SPF-PCB+0.3 ml cell suspension). Measurements of fluorescence are performed at 37° C. with constant stirring using a custom-built spectrofluorimeter (Biomedical Instrumentation Group, University of Pennsylvania). Excitation and emission wavelengths are 340 and 510 nm, respectively. To calibrate fluorescence signals, digitonin (Sigma, St. Louis, Mo.; catalog # D 5628; 50 μg/ml, final) is added to obtain Fₘₐₓ, and the apparent Fₘᵢₙ, is determined by adding EGTA (10 mM, final) and Tris base (pH˜10, final). Concentrations of released intracellular Ca²⁺ is calculated using a dissociation constant (Kd) of 224 nM and the equation:
[Ca²⁺]ᵢ=(F−Fₘᵢₙ/Fₘₐₓ−F)×Kd

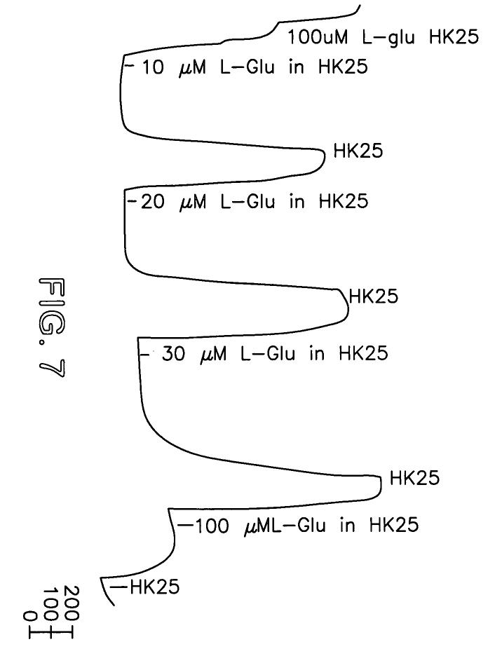

The results are graphically represented in FIG. 7.

### Example 4

### Recombinant Receptor Binding Assay

The following is one example of a rapid screening assay to obtain compounds modulating metabotropic glutamate receptor activity. The screening assay first measures the ability of compounds to bind to recombinant chimeric receptors, or receptor fragments or mGluR or chimeric receptors. Compounds binding to such receptors or fragments can then be tested for their ability to modulate one or more activities at a metabotropic glutamate receptor.

In one procedure, a cDNA or gene clone encoding a metabotropic glutamate receptor is obtained. Distinct fragments of the clone are expressed in an appropriate expression vector to produce the smallest receptor polypeptide(s) obtainable able to bind glutamate. Such experiments can be facilitated by utilizing a stably transfected mammalian cell line (e.g., HEK 293 cells) expressing the receptor.

The recombinant polypeptide(s) having the desired binding properties can be bound to a solid-phase support using standard chemical procedures. This solid-phase, or affinity matrix, may then be contacted with glutamate to demonstrate that glutamate can bind to the column, and to identify conditions by which glutamate may be removed from the solid-phase. This procedure may then be repeated using a large library of compounds to determine those compounds which are able to bind to the affinity matrix. Bound compounds can then can be released in a manner similar to glutamate. Alternative binding and release conditions may be utilized to obtain compounds capable of binding under conditions distinct from those used for glutamate binding (e.g., conditions which better mimic physiological conditions encountered especially in pathological states). Compounds binding to the mGluR can thus be selected from a very large collection of compounds present in a liquid medium or extract.

In an alternate method, chimeric metabotropic glutamate/calcium receptors are bound to a column or other solid phase support. Those compounds which are not competed off by reagents binding to the glutamate binding site on the receptor can then be identified. Such compounds define alternative binding sites on the receptor. Such compounds may be structurally distinct from known compounds and may define chemical classes of agonists or antagonists which may be useful as therapeutics agents.

II. Construction of Chimeric Receptors

### Example 5

### CaSPhmGluR7 and p8SPhmGluR7

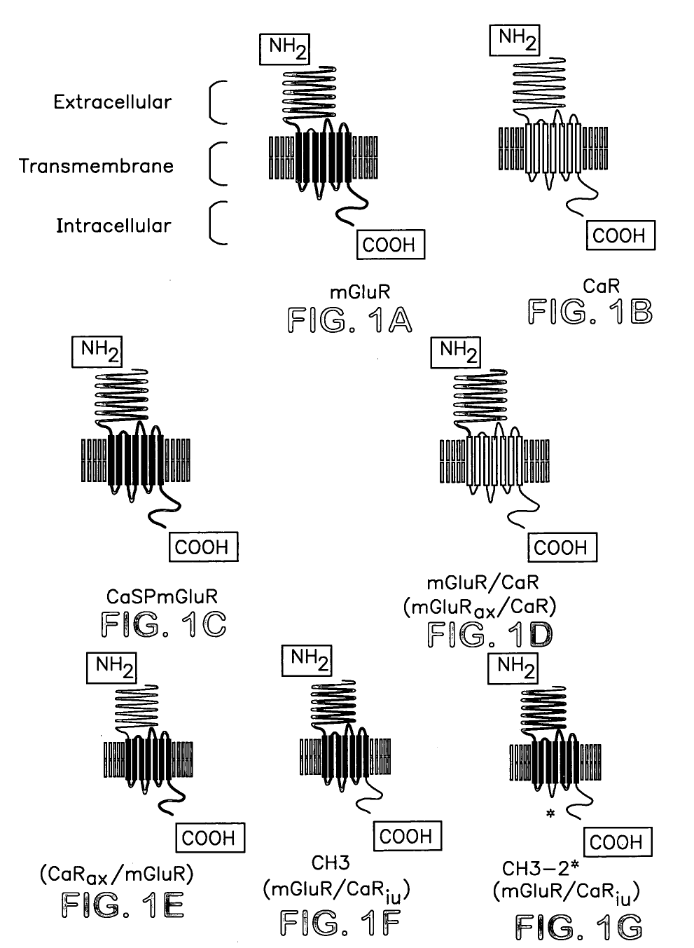

This example describes the preparation of chimeric receptors that includes a non-native signal peptide from a calcium receptor ( designated CaSP) or mGluR8 (designated 8SP) linked to a human mGluR7 receptor sequence (designated hmGluR7). Chimeric receptors including a non-native signal peptide, e.g., a CaR signal peptide, along with mGluR (e.g., mGluR7) receptor sequences are particularly advantageous because the inclusion of the CaR or other non-native signal peptide can provide higher levels of functional receptor in a cell across a variety of different mGluR receptors. While the constructs described below utilized full-length mGluR7, shortened sequences and/or other mGluR receptors, e.g., mGluR2, can also be used. A depiction of these CaSPmGluR constructs is shown in FIG. 1C.

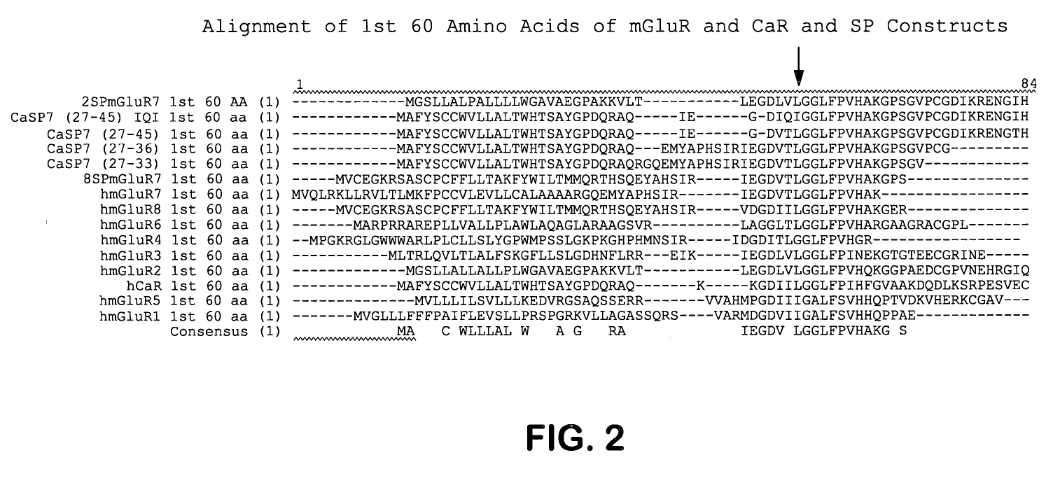

Alignments of the amino termini of CaR and mGluRs allow one to make predictions about which chimeric junctions in these constructs are likely to work. In some cases, the alignments are ambiguous and thus several chimeric constructs have been made in order to obtain a functional receptor. An example of one such alignments is shown in FIG. 2.

The generic scheme to create the recombinant junction utilizes “recombinant PCR” in which one or two hybrid primers that contain sequence from each of the two receptors to be chimerized are combined with an upstream or a downstream oligonucleotide, in separate reactions, whose specific location and sequence are important only in that a unique restriction site needs to be included between the junction and the upstream or downstream primer. In the second step, the two fragments from the primary reactions are annealed together in the presence of the upstream and downstream primers and extended in a typical PCR reaction with DNA polymerase to create the recombinant fragment which now contains the recombinant junction, variable lengths of each of the two receptors upstream and downstream of the junction, and two unique restriction sites to be utilized for cloning the recombinant receptor into a full-length receptor to create the chimeric receptor. An overview of recombinant PCR is presented by R. Higuchi in PCR Protocols: A Guide to Methods and Applications, (1990) Academic Press, Inc.

For clarity, the specific details are given for creation of the mGluR7 chimeras; for brevity only enough detail to enable one skilled in the art to reproduce these chimeras is given for other examples.

phmGluR7:

A full length human mGluR7 cDNA was amplified from human hippocampus MarathonReady cDNA (Clontech) using PCR primers based on the human mGluR7 cDNA sequence (Genbank Accession #X94552) and cloned into the pT7 Blue plasmid (Novagen). The primers, “7-1” (sense 18-mer, 5′-CTC ACC CTC TCT GGT CGC -3′ and “7-2” (antisense 21-mer, 5′-TCT TCC TCC TCC ATG GTA CCA-3—) amplified a 2944 bp fragment including UTRs. This construct is referred to as phmGluR7/pT7Blue. This was subsequently subcloned into the pPCR SCRIPT Amp vector (Stratagene).

phmGluR8b:

Construction of this human mGluR8b cDNA is described in U.S. Pat. No. 6,051,688, which is incorporated herein by reference in its entirety, including any drawings.

p8SPhmGluR7:

The predicted signal peptide of mGluR7 was replaced with the predicted signal peptide and 87 bp of 5′ UTR from hmGluR8 using a recombinant PCR strategy.

The first reaction used a phmGluR8b construct (Sequence in U.S. Pat. No. 6,051,688) with two primers, T7 (sense 20-mer, complementary to vector sequence upstream of the hmGluR8 insert; sequence 5′-TAA TAC GAC TCA CTA TAG GG-3′), and the hybrid primer 8SP (antisense 42-mer, containing 21 nucleotides complementary to human mGluR8 and 21 nucleotides complementary human mGluR7; sequence 5′-GAG GGT GAC GTC CCC CTC GAT CCG TAT GGA ATG GGC ATA CTC-3′). These primers were used to amplify an approximately 300 bp PCR fragment of human mGluR8. In a separate PCR reaction using phmGluR7 in pPCR SCRIPT Amp vector as template, an approximately 200 bp fragment of human mGluR7 was amplified using a hybrid primer 7SP (sense 42-mer, exactly complementary to primer 8SP) and oligo 7-352m, (antisense 19-mer, complementary to the human mGluR7 cDNA; sequence 5′-GTT CGA GCG CGT AAG TGT C-3′). The two PCR products generated from the above two reactions were annealed together in equimolar ratios in the presence of the external primers T7 and 7-352m, and Turbo Pfu DNA polymerase (Stratagene). The resulting chimeric PCR product was digested with XhoI and AatI (New England Biolabs) and subcloned into phmGluR7 digested with the same two restriction enzymes. The sequence of the resultant chimeric construct, p8SPhmGluR7, was verified by ABI automated DNA sequence analysis. The replacement of the predicted signal peptide of mGluR7 with that of mGluR8 greatly increased the activity of the chimeric receptor in in vitro assays (e.g., Xenopus oocyte assay).

pCaSPhmGluR7:

Several variations of CaSPhmGluR7 were made; CaSPhmGluR7(27-33), CaSPhmGluR7(27-36) and the preferred construct, CaSPhmGluR7(27-45). In these construct designations, the “CaSP” refers to a CaR signal peptide and “hmGluR7” refers to human mGluR7. The first number in parentheses refers to the number of amino acids residues of CaR sequence linked to the N-terminus of the mGluR, while the second number refers to the amino acid residue within the full length mGluR7 receptor to which the CaR signal peptide is linked.

CaSPhmGluR7(27-33)

The CaSPhmGluR7(27-33) has the first 27 amino acids of human CaR (Genbank Accession #U20759) joined to the 33^{rd} amino acid of full length hmGluR7. The junction was made by recombinant PCR. The first reaction used a human parathyroid CaR construct (Sequence in U.S. Pat. No. 6,051,688) with two primers, T7 (sense 20-mer, complementary to vector sequence upstream of the hCaR insert; sequence 5′-TAA TAC GAC TCA CTA TAG GG-3′), and the hybrid primer “CaSP7-33” (antisense 42-mer, containing 21 nucleotides complementary to human CaR and 21 nucleotides complementary human mGluR7; sequence 5′-GGC GTA CAT CTC CTG GCC GCG TTG GGC TCG CTG GTC TGG CCC-3′). These primers were used to amplify a 215 bp PCR fragment of human CaR. In a separate PCR reaction using phmGluR7 in pPCR SCRIPT Amp vector as template, a 274 bp fragment of human mGluR7 was amplified using a hybrid primer “7CaSP-33” (sense 42-mer, exactly complementary to primer CaSP7-33) and oligo 7-352m, (antisense 19-mer, complementary to the human mGluR7 cDNA; sequence 5′-GTT CGA GCG CGT AAG TGT C-3′). The two PCR products generated from the above two reactions were annealed together in equimolar ratios in the presence of the external primers T7 and 7-352m, and High Fidelity Taq polymerase (Roche). The resulting chimeric PCR product was digested with NheI and BssHII (New England Biolabs) and subdloned into p8SPhmGluR7 digested with the same two restriction enzymes. The sequence of the resultant chimeric construct, pCaSPhmGluR7(27-33), was verified by ABI automated DNA sequence analysis.

CaSPhmGluR7(27-36):

The CaSPhmGluR7(27-36) has the first 27 amino acids of human CaR joined to the 36^{th} amino acid of full length hmGluR7. It was made as above except for the use of slightly different hybrid primers, “CaSP7-36” (antisense 42-mer, containing 21 nucleotides complementary to human CaR and 21 nucleotides complementary human mGluR7; sequence 5′-TGA GTG CGG GGC GTA CAT CTC TTG GOC TCG CTG GTC TGG CCC-3′) and the complement, “7CaSP-36”.

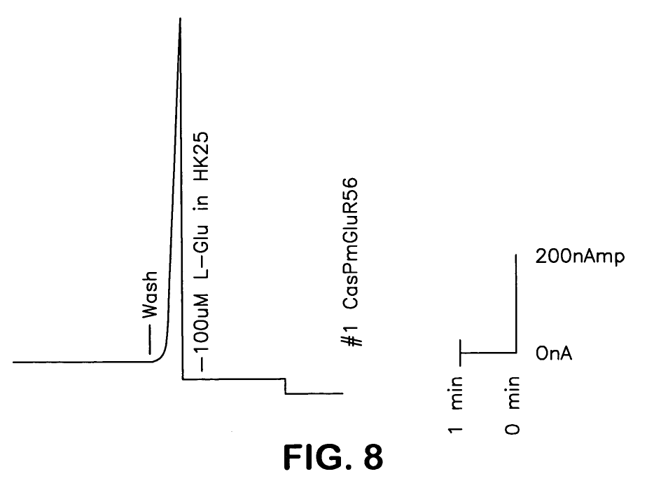

CaSPhmGluR7(27-33), and CaSPhmGluR7(27-36) were then each subcloned into a vector using the restriction sites NheI and NotI (New England Biolabs). A variety of vectors can be used for this purpose, including, for example, pIREShyg3 (Clontech). A diagram of this vector is shown in FIG. 8.

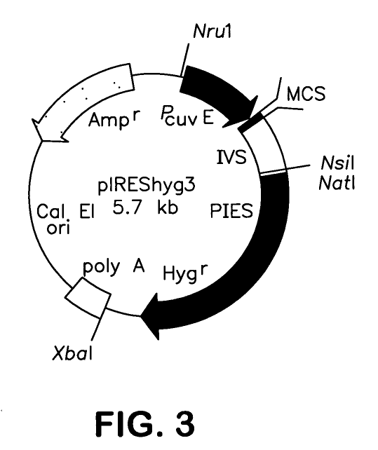

The mammalian expression vector pIREShyg3 was obtained from Clontech and is depicted in FIG. 3. This vector contains the human cytomegalovirus (CMV) major immediate early promoter/enhancer followed by a multiple cloning site (MCS) that precedes stop codons in all three reading frames, a synthetic intron known to enhance the stability of the mRNA, the ECMV IRES followed by the hygromycin B phosphotransferase gene, and the polyadenylation signal from SV40. Ribosomes can enter the bicistronic mRNA at the 5′ end to translate the gene of interest and at the ECMV IRES to translate the antibiotic resistance marker. After selection with hygromycin B, nearly all surviving colonies will stably express the gene of interest, thus decreasing the need to screen large numbers of colonies to find functional clones. To select for cells that express high levels of the gene of interest, the selective pressure for antibiotic resistance was increased by shifting the hygromycin B phosphotransferase gene downstream to a less optimal position for translation as directed by the IRES sequence. By decreasing the level of expression of the antibiotic resistance marker, the selective pressure on the entire expression cassette is increased, resulting in selection for cells that express the entire transcript, including the gene of interest, at high levels.

CaSPhmGluR7(27-45):

The CaSPhmGluR7(27-45) construct has the first 27 amino acids of human CaR joined to the 45^{th} amino acid of hmGluR7. This construct was made by site directed mutagenesis using the Quik Change Site Directed Mutagenesis XL kit (Stratagene) to delete 27 nucleotides (9 amino acids) from the CaSPhmGluR7(27-36) vector construct. The primers used in the reaction are “C7D27P” (36-mer 5′-CCA GAC CAG CGA GCC CAA ATC GAG GGG GAC GTC ACC-3′) and the complementary primer “C7D27M”.

The sequence of these resultant chimeric constructs, CaSPhmGluR7(27-33), CaSPhmGluR7(27-36) and CaSPhmGluR7(27-45), were verified by ABI automated DNA sequence analysis.

In preparing nucleic acid sequences encoding the present receptors, cloning of the N-terminus of the mGluR (e.g., human mGluR7) is not necessary. For example, in making constructs such as those described above, PCR primers to amplify only the portion of mGluR7 used could be employed. Thus, to make the construct, CaSPhmGluR7(27-45), only nucleotides encoding amino acids 45 through 915 of full length mGluR7 are needed. This can, for example, be done using primers complementary to nucleotides 133-153 to make a sense primer (5′-atcgagggggacgtcaccctc-3′) and then PCR amplifying as in the above example for the cloning of human mGluR7 using the same antisense primer, “7-2”. In this way, 132 nucleotides of the human mGluR7 would not have been used.

Expression of the exemplary chimeric receptors described above in host cells provided more consistent and substantially higher levels of functional receptor than expression of native mGluR7 in the same host cells. Thus, such chimeric receptors are highly advantageous for cell-based screening for mGluR modulators, such as modulators of mGluR7.

Sequences

Nucleic acid and amino acid sequences of the receptors utilized in making the above chimeric constructs and the resulting chimeric receptors are shown in SEQ ID NO. 1-12.

Functional Expression

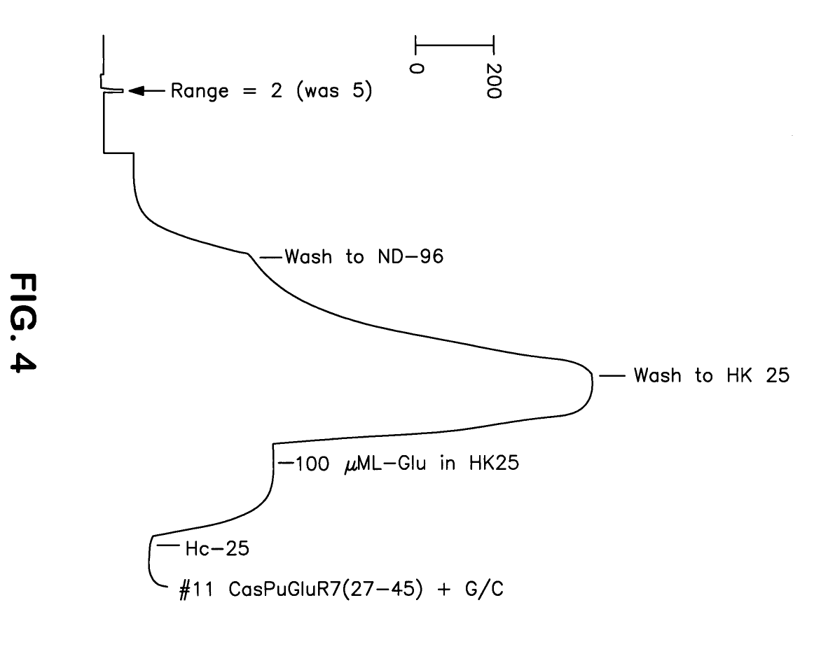

CaSPhmGluR7(27-45) was analyzed for function in the oocyte assay for Gαi-coupled receptors (method detailed in Example 1B). As shown in FIG. 4, robust activation of the chimeric receptor was seen with application of 100 uM L-glutamate, showing that this receptor is indeed functional.

### Example 6

### CaSPhmGluR3

Three similar CaSPhmGluR3 chimeras were made, CaSPhmGluR3(27-21), CaSPhmGluR3(27-23) and CaSPhmGluR3(27-33). They each contain the first 27 amino acids of CaR joined to either the 21^{st} or 23^{rd} amino acid of hmGluR3 (GenBank Accession number NM 000840). The hybrid primers used to make the recombinant junctions in these examples were “CaSP3-21” (antisense 42-mer, containing 21 nucleotides complementary to human CaR and 21 nucleotides complementary human mGluR3; sequence 5′-TAG AAA GTT ATG GTC CCC TAA TTG GGC TCG CTG GTC TGG CCC-3′) and the complement, “3CaSP-21” or “CaSP3-23” (antisense 42-mer, containing 21 nucleotides complementary to human CaR and 21 nucleotides complementary human mGluR3; sequence 5′-TCT CCT TAG AAA GTT ATG GTC TTG GGC TCG CTG GTC TGG CCC-3′) and the complement, “3CaSP-23”. CaSPhmGluR3(27-33) was made by site directed mutagenesis using the Quik Change Site Directed Mutagenesis XL kit (Stratagene) to delete 30 nucleotides (10 amino acids) from the CaSPhmGluR3(27-23) construct.

Sequences

Nucleic acid and amino acid sequences of the CaSPhmGluR3 chimeric receptors are shown in SEQ ID NO. 13-18.

Functional Expression

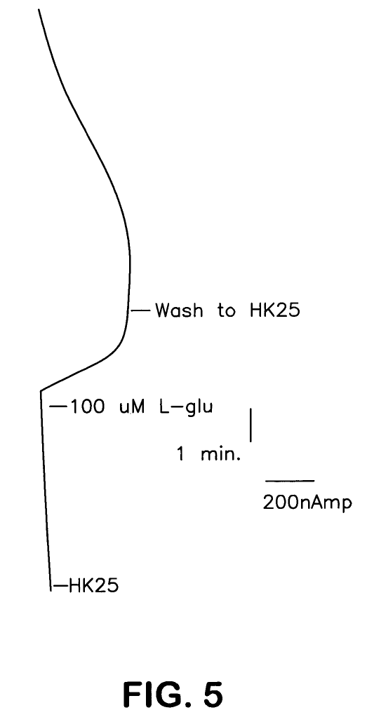

CaSPhmGluR3(27-33) was also analyzed in the oocyte assay for Gαi-coupled receptors (method detailed in Example IB). As shown in FIG. 5, activation of the chimeric receptor was seen with application of 100 uM L-glutamate, showing that this receptor is indeed functional.

### Example 7

### CaSPhmGluR2

The CaSPhmGluR2(27-19) chimera was made with the first 27 amino acids of CaR joined to the 19th amino acid of hmGluR2 (GenBank Accession number NM 000839). The hybrid primers used to create the recombinant junction in this example included a sense primer of 42 nucleotides, containing 21 nucleotides complementary to human CaR and 21 nucleotides complementary human mGluR2 (sequence 5′-GGG CCA GAC CAG CGC GCC CAA GAG GGC CCA GCC AAG AAG GTG-3′) and a downstream primer to amplify a fragment of mGluR2 from a plasmid template. The complementary hybrid primer was used in the other recombinant junction reaction with an upstream primer and the CaR plasmid as template.

Sequences

Nucleic acid and amino acid sequences of the CaSPhmGluR2(27-19) chimeric receptor are shown in SEQ ID NO.19-20.

Functional Expression

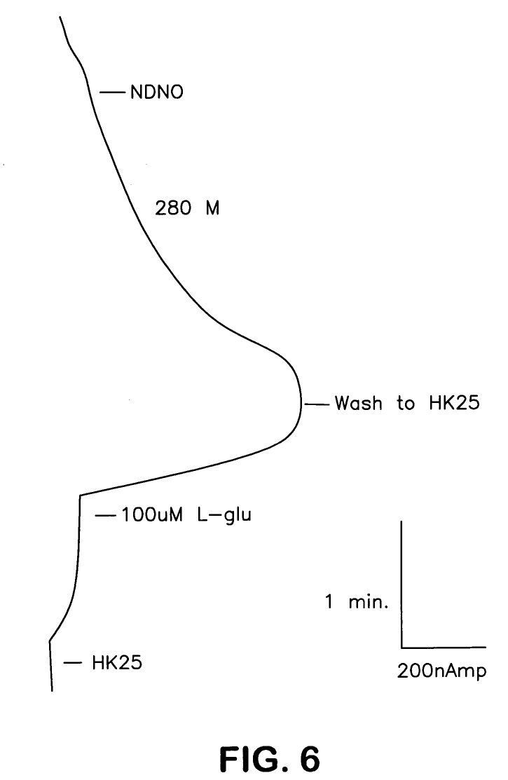

FIG. 6 shows functional expression of CaSPhmGluR2(27-19) in the oocyte assay for Gαi-coupled receptors (method detailed in Example 1B). Activation of the receptor by 100 uM L-glutamate confirms that this receptor is functional.

### Example 8

### CaSPhmGluR6

CaSPhmGluR6(27-35) chimera contains the first 27 amino acids of CaR joined to the 35th amino acid of hmGluR3 (GenBank Accession number NM 000843).

Sequences

Nucleic acid and amino acid sequences of the CaSPhmGluR6(27-35) chimera are shown in SEQ ID NO. 21-22.

Functional Expression

FIG. 7 shows functional expression of CaSPhmGluR6(27-35) in the oocyte assay for Gαi-coupled receptors (method detailed in Example 1B). Activation of the chimeric receptor by 100 uM L-glutamate confirms that this receptor is functional.

### Example 9

### CaSPhmGluR5

CaSPhmGluR5(27-22) chimera contains the first 27 amino acids of CaR joined to the 22nd amino acid of hmGluR5 (GenBank Accession #NM 000842).

Sequences

Nucleic acid and amino acid sequences of the CaSPhmGluR5(27-22) chimera are shown in SEQ ID NO. 23-24.

Functional Expression

FIG. 8 shows functional expression of CaSPhmGluR5(27-22) in the oocyte assay for PLC-coupled receptors (method detailed in Example 1A). Activation of the chimeric receptor by 100 uM L-glutamate confirms that this receptor is functional.

Chimeric receptors that include an mGluR extracellular domain and CaR sequences are described below. Such chimeric receptors can be designed to also include a non-native signal peptide as for the exemplary receptors described in Examples 1-9.

### Example 10

### pmGluR1/CaR

phPCaR4.0

Plasmid phPCaR4.0 (Garrett et al., J. Biol. Chem., 270:12919, 1995, hereby incorporated by reference herein) was isolated from E. coli bacterial cells containing the plasmid grown up in nutrient broth containing 100 ug/ml ampicillin (Boerhringer Mannheim). This plasmid DNA was used as the source for the DNA encoding the human calcium receptor which was cloned into the EcoRI site of vector pBluescript SK (Stratagene) in the T7 orientation. All restriction enzymes and modification enzymes were purchased from New England Biolabs unless otherwise noted.

pmGluR1 (Rat and Human)

Plasmid p7-3/6A was assembled in pBluescript SK from two overlapping subclones of rat mGluR1 obtained from an oligonucleotide screen of a commercially available rat olfactory bulb cDNA library (Stratagene). This plasmid DNA was used as the source of the metabotropic glutamate receptor, mGluR1. It was also used to screen a commercially available human cerebellar cDNA library for the human analogue. The human cerebellar library was screened with a radioactively labeled rat mGluR1 by a method described in Sambrook et al., Molecular Cloning: A Laboratory Manual, Chapter 1, 1989. Positive plaques were rescued using the manufacturer's protocol and restriction mapped to compare them against the published human mGluR1 sequence (Eur. Patent publications 0 569 240 A1 and 0 568 384 A1). Two subclones were assembled to create a complete human mGluR1.

Alternatively, the sequence of human mGluR1 may be obtained from European Publication Nos. 0 569 240 A1 and 0 568 384 A1. Probes prepared using this sequence may be used to probe human cDNA libraries to obtain the full length human clone. In addition, the relevant sequences may be synthesized using the sequence described therein.

pmGluR1/CaR (“pR1/CaR”)

Chimeric receptors were constructed using recombinant PCR and a multi-step cloning strategy. An overview of recombinant PCR is presented by R. Higuchi in PCR Protocols: A Guide to Methods and Applications, (1990) Academic Press, Inc. In the first construct recombinant PCR was used to combine the sequences of mGluR1 and the CaR across the junction of the extracellular and transmembrane domains. The first chimera, pR1/CaR, contained the extracellular domain of mGluR1 and the transmembrane and intracellular region of the calcium receptor as depicted in Figure. The chimeric junction was created using three separate PCR reactions. The first reaction used two primers specific for rat mGluR1, A4, a 22 mer encoding nucleotides 1146 to 1167, and an antisense primer, oligoB, a 43 mer containing 22 bases of mGluR1 (nucleotides −1755 to −1776) and 21 bases from the CaR (nucleotides −1837 to −1857). These primers were used to amplify a 650 bp fragment of rat mGluR1. In a separate PCR reaction, a 500 bp fragment of the CaR was amplified using hybrid primer C, a 43 mer which was the complement of oligo B, and D4, an antisense primer corresponding to nucleotides −2256 to −2279 of the CaR. These two PCR products were purified from an agarose gel and annealed together in equal molar ratio in the presence of the external primers A4 and D4 and the proof-reading DNA polymerase, Pfu (Stratagene). The 1,100 bp chimeric PCR product was digested with Nsi I and subdloned into phCar4.0 digested with EcoRV and Nsi I. The resultant subclone was subsequently digested with Xho I and Sfi I to remove the extracellular domain of the CaR which was then replaced with the Xho I- Sfi I fragment of rat mGluR1. The resultant chimera, pR1/CaR was validated by restriction mapping and double-stranded DNA sequencing with Sequenase Version 2.0 (US Biochemical).

Sequences

The DNA sequence for pR1/CaR is shown in SEQ ID NO: 25; the corresponding amino acid sequence is shown in SEQ ID NO: 26.

Functional expression

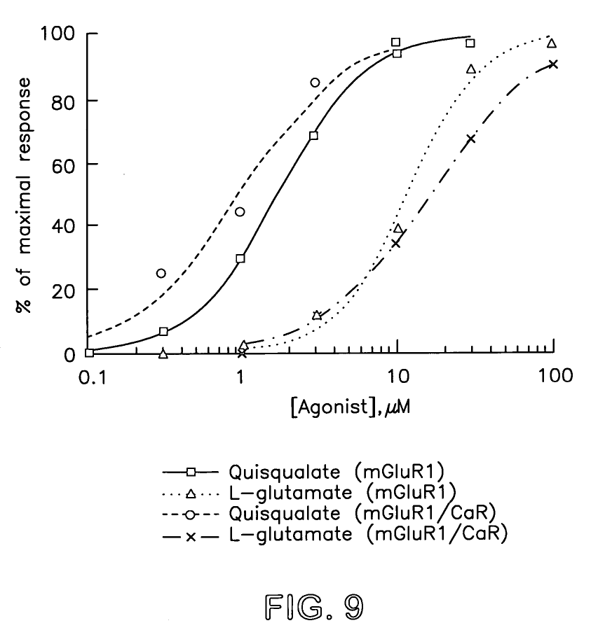

FIG. 9 compares the pmGluR1/CaR chimera to rat mGluR1 using the PLC-coupled oocyte assay. Both receptors respond comparably to L-glutamate and quisqualate demonstrating that the chimera is functional.

### Example 11

### pratCH3 and phCH4

These chimeras are a result of swapping the CaR cytoplasmic tail onto the extracellular and transmembrane domains of either rat or human mGluR 1. Recombinant PCR was used to attach the C-terminal tail of the CaR onto human mGluR1 (which encodes the rat mGluR1 signal sequence) after nucleotide 2535. The first PCR reaction used two primers specific for human mGluR1, M-1rev a 24 mer corresponding to nucleotides 2242 to 2265, and an antisense primer, CH3R1, a 36 mer composed of 18 bases of hmGluR1 (nucleotides −2518 to −2535) and 18 bases of CaR (nucleotides −2602 to −2619). These primers were used to amplify a 300 bp fragment of hmGluR1. In a separate PCR reaction, a 750 bp fragment of the CaR was amplified using hybrid primer CH3CaR, a 36 mer which is the complement of oligo CH3R1, and a commercially available T3 primer (Stratagene) which primes in the Bluescript vector in a region downstream from the 3′ end of the CaR. The two PCR products were purified from an agarose gel and annealed together in equal molar ratio in the presence of the external primers M-1 rev and T3 and the proof-reading DNA polymerase, Pfu (Stratagene). The 1 kb chimeric PCR product was digested with Nhe I and Not I and subcloned into phmGluR1 digested with the same enzymes. The resultant chimera, phCH4 was validated by restriction mapping and double-stranded DNA sequencing. To detect functional activity in the oocyte assay with this clone it was necessary to exchange the 5′ untranslated region and the signal sequence from rat mGluR1 with the same region of this human clone. This was done utilizing a Bsu36I restriction site. Additionally, an Acc I fragment of rat mGluR1 was subcloned into phCH4 to create a rat version of this same chimera. This chimera is referred to as ratCH3.

Sequences

The DNA sequence for pratCH3 is shown in SEQ ID NO: 27; the corresponding amino acid sequence is SEQ ID NO: 28. The DNA sequence for phCH4 is SEQ ID NO: 29 and the corresponding amino acid sequence is SEQ ID NO: 30.

Functional expression

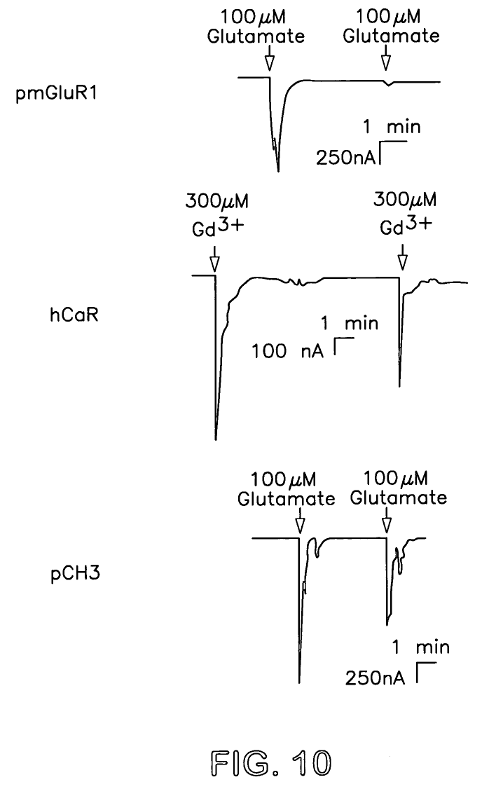

FIG. 10 compares the pratCH3 chimera to pmGluR1 and hCaR using the PLC-coupled oocyte assay. The chimera responds to L-glutamate demonstrating that the chimera is functional.

Using the techniques described in the above-mentioned examples, we therefore envision the construction, evaluation and screening utility of other mGluR/CaR chimeras. In this example we have taken a Group I metabotropic glutamate receptor which, similar to the calcium receptor, is coupled to the activation of phospholipase C and mobilization of intracellular calcium, and by swapping the C-terminal tail, maintained the integrity of the second messenger system. Additionally, when the CaR tail was added to mGluR1, the desensitization properties were lost. This demonstrates the feasibility of changing specific G-protein coupling of metabotropic glutamate receptors to those of the CaR by swapping intracellular domains. By example, Group II mGluRs, such as mGluR2 or mGluR3 which are Gᵢ coupled, could be changed to Gq coupled receptors. This can be done by exchanging onto these receptors the C-terminal cytosolic tail of the CaR using the protocol described in examples 2, 3 and 4. Effective Gq coupling could be evaluated in the oocyte as described in examples 5 and 6. Activation of a Group II by L-CCG-I (their most potent agonist), should induce mobilization of intracellular Ca2+ which will cause the detectable inward rectifying Cl— current measured in the voltage-clamped oocyte.

To increase the effectiveness of G-protein binding it may be useful to swap one or more additional intracellular (cytoplasmic) loops of the CaR onto the mGluR1. By example, such substitution can involve any of: intracellular loop 1, intracellular loop 2 and intracellular loop 3 from a calcium receptor, substituted alone or in any combination of loops. Such subdomain swapping may be necessary for the most effective transference of G-protein binding specificity. See FIG. 1 for graphical depictions of such chimeras.

### Example 12

### Construction of pCEPCaR/R1 from uCaR/R1

The DNA from plasmid pCaR/R1 was digested and cloned into the commercially available episomal mammalian expression vector, pCEP4 (Invitrogen), using the restriction enzymes Kpn I and Not I. The ligation products were transfected into DH5a cells which had been made competent for DNA transformation. These cells were plated on Luria-Bertani Media (LB) plates (described in Sambrook et al., Molecular Cloning: A Laboratory Manual, 1989)) containing 100 ug/ml ampicillin. A clone was selected from the colonies which grew. This clone, pCEPCaR/R1 was characterized by restriction enzyme digestion.

Functional expression

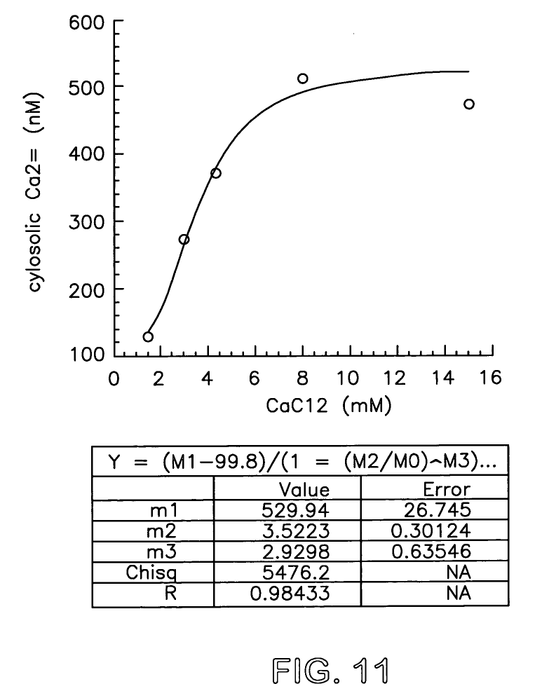

FIG. 11 shows that pCEPCaR/R1 is functional using the Fura assay described in Example 3.

TABLE 1

Human mGluR7 nucleic acid sequence (including 5′ and
3′ UTRs; from primers 7-1 to 7-2) (SEQ ID NO 1)

ctcaccctctctggtcgcccctccccggattcccccaccctccgtgcctgcaggagcccc
tgggctttcccggaggagctcgccctgaagggcccggacctcggcgagcccaccaccgtt
ccctccagcgccgccgccgccaccgcagcagccggagcagcatggtccagctgaggaagc
tgctccgcgtcctgactttgatgaagttcccctgctgcgtgctggaggtgctcctgtgcg
cgctggcggcggcggcgcgcggccaggagatgtacgccccgcactcaatccggatcgagg
gggacgtcaccctcggggggctgttccccgtgcacgccaagggtcccagcggagtgccct
gcggcgacatcaagagggaaaacgggatccacaggctggaagcgatgctctacgccctgg
accagatcaacagtgatcccaacctactgcccaacgtgacgctgggcgcgcggatcctgg
acacttgttccagggacacttacgcgctcgaacagtcgcttactttcgtccaggcgctca
tccagaaggacacctccgacgtgcgctgcaccaacggcgaaccgccggttttcgtcaagc
cggagaaagtagttggagtgattggggcttcggggagttcggtctccatcatggtagcca
acatcctgaggctcttccagatcccccagattagttatgcatcaacggcacccgagctaa
gtgatgaccggcgctatgacttcttctctcgcgtggtgccacccgattccttccaagccc
aggccatggtagacattgtaaaggccctaggctggaattatgtgtctaccctcgcatcgg
aaggaagttatggagagaaaggtgtggagtccttcacgcagatttccaaagaggcaggtg
gactctgcattgcccagtccgtgagaatcccccaggaacgcaaagacaggaccattgact
ttgatagaattatcaaacagctcctggacacccccaactccagggccgtcgtgatttttg
ccaacgatgaggatataaagcagatccttgcagcagccaaaagagctgaccaagttggcc
attttctttgggtgggatcagacagctggggatccaaaataaacccactgcaccagcatg
aagatatcgcagaaggggccatcaccattcagcccaagcgagccacggtggaagggtttg
atgcctactttacgtcccgtacacttgaaaacaacagaagaaatgtatggtttgccgaat
actgggaggaaaacttcaactgcaagttgacgattagtgggtcaaaaaaagaagacacag
atcgcaaatgcacaggacaggagagaattggaaaagattccaactatgagcaggagggta
aagtccagttcgtgattgacgcagtctatgctatggctcacgcccttcaccacatgaaca
aggatctctgtgctgactaccggggtgtctgcccagagatggagcaagctggaggcaaga
agttgctgaagtatatacgcaatgttaatttcaatggtagtgctggcactccagtgatgt
ttaacaagaacggggatgcacctgggcgttatgacatctttcagtaccagaccacaaaca
ccagcaacccgggttaccgtctgatcgggcagtggacagacgaacttcagctcaatatag
aagacatgcagtggggtaaaggagtccgagagatacccgcctcagtgtgcacactaccat
gtaagccaggacagagaaagaagacacagaaaggaactccttgctgttggacctgtgagc
cttgcgatggttaccagtaccagtttgatgagatgacatgccagcattgcccctatgacc
agaggcccaatgaaaatcgaaccggatgccaggatattcccatcatcaaactggagtggc
actccccctgggctgtgattcctgtcttcctggcaatgttggggatcattgccaccatct
ttgtcatggccactttcatccgctacaatgacacgcccattgtccgggcatctgggcggg
aactcagctatgttcttttgacgggcatctttctttgctacatcatcactttcctgatga
ttgccaaaccagatgtggcagtgtgttctttccggcgagttttcttgggcttgggtatgt
gcatcagttatgcagccctcttgacgaaaacaaatcggatttatcgcatatttgagcagg
gcaagaaatcagtaacagctcccagactcataagcccaacatcacaactggcaatcactt
ccagtttaatatcagttcagcttctaggggtgttcatttggtttggtgttgatccaccca
acatcatcatagactacgatgaacacaagacaatgaaccctgagcaagccagaggggttc
tcaagtgtgacattacagatctccaaatcatttgctccttgggatatagcattcttctca
tggtcacatgtactgtgtatgccatcaagactcggggtgtacccgagaattttaacgaag
ccaagcccattggattcactatgtacacgacatgtatagtatggcttgccttcattccaa
ttttttttggcaccgctcaatcagcggaaaagctctacatacaaactaccacgcttacaa
tctccatgaacctaagtgcatcagtggcgctggggatgctatacatgccgaaagtgtaca
tcatcattttccaccctgaactcaatgtccagaaacggaagcgaagcttcaaggcggtag
tcacagcagccaccatgtcatcgaggctgtcacacaaacccagtgacagacccaacggtg
aggcaaagaccgagctctgtgaaaacgtagacccaaacagccctgctgcaaaaaagaagt
atgtcagttataataacctggttatctaacctgttccattccatggaaccatggaggagg
aaga

TABLE 2
Human mGluR7 amino acid sequence (SEQ ID NO 2)

mvqlrkllrvltlmkfpccvlevllcalaaaargqemyaphsiriegdvtlgglfpvhak
gpsgvpcgdikrengihrleamlyaldqinsdpnllpnvtlgarildtcsrdtyaleqsl
tfvqaliqkdtsdvrctngeppvfvkpekvvgvigasgssvsimvanilrlfqipqisya
stapelsddrrydffsrvvppdsfqaqamvdivkalgwnyvstlasegsygekgvesftq
iskeagglciaqsvripqerkdrtidfdriikqlldtpnsravvifandedikqilaaak
radqvghflwvgsdswgskinplhqhediaegaitiqpkratvegfdayftsrtlennrr
nvwfaeyweenfnckltisgskkedtdrkctgqerigkdsnyeqegkvqfvidavyamah
alhhmnkdlcadyrgvcpemeqaggkkllkyirnvnfngsagtpvmfnkngdapgrydif
qyqttntsnpgyrligqwtdelqlniedmqwgkgvreipasvctlpckpgqrkktqkgtp
ccwtcepcdgyqyqfdemtcqhcpydqrpnenrtgcqdipiiklewhspwavipvflaml
giiatifvmatfiryndtpivrasgrelsyvlltgiflcyiitflmiakpdvavcsfrrv
flglgmcisyaalltktnriyrifeqgkksvtaprlisptsqlaitsslisvqllgvfiw
fgvdppniiidydehktmnpeqargvlkcditdlqiicslgysillmvtctvyaiktrgv
penfneakpigftmyttcivwlafipiffgtaqsaeklyiqtttltismnlsasvalgml
ympkvyiiifhpelnvqkrkrsfkavvtaatmssrlshkpsdrpngeaktelcenvdpns
paakkkyvsynnlvi

TABLE 3

Human mGluR8b nucleic acid sequence (with 87 nt 5′UTR)
(SEQ ID NO 3)

ccagaaggtgcagcctcaggtggtgccctttcttctgtggcaagaataaactttgggtct
tggattgcaataccacctgtggagaaa
atggtatgcgagggaaagcgatcagcctcttgcccttgtttcttcctcttgaccgccaag
ttctactggatcctcacaatgatgcaaagaactcacagccaggagtatgcccattccata
cgggtggatggggacattattttggggggtctcttccctgtccacgcaaagggagagaga
ggggtgccttgtggggagctgaagaaggaaaaggggattcacagactggaggccatgctt
tatgcaattgaccagattaacaaggaccctgatctcctttccaacatcactctgggtgtc
cgcatcctcgacacgtgctctagggacacctatgctttggagcagtctctaacattcgtg
caggcattaatagagaaagatgcttcggatgtgaagtgtgctaatggagatccacccatt
ttcaccaagcccgacaagatttctggcgtcataggtgctgcagcaagctccgtgtccatc
atggttgctaacattttaagactttttaagatacctcaaatcagctatgcatccacagcc
ccagagctaagtgataacaccaggtatgactttttctctcgagtggttccgcctgactcc
taccaagcccaagccatggtggacatcgtgacagcactgggatggaattatgtttcgaca
ctggcttctgaggggaactatggtgagagcggtgtggaggccttcacccagatctcgagg
gagattggtggtgtttgcattgctcagtcacagaaaatcccacgtgaaccaagacctgga
gaatttgaaaaaattatcaaacgcctgctagaaacacctaatgctcgagcagtgattatg
tttgccaatgaggatgacatcaggaggatattggaagcagcaaaaaaactaaaccaaagt
gggcattttctctggattggctcagatagttggggatccaaaatagcacctgtctatcag
caagaggagattgcagaaggggctgtgacaattttgcccaaacgagcatcaattgatgga
tttgatcgatactttagaagccgaactcttgccaataatcgaagaaatgtgtggtttgca
gaattctgggaggagaattttggctgcaagttaggatcacatgggaaaaggaacagtcat
ataaagaaatgcacagggctggagcgaattgctcgggattcatcttatgaacaggaagga
aaggtccaatttgtaattgatgctgtatattccatggcttacgccctgcacaatatgcac
aaagatctctgccctggatacattggcctttgtccacgaatgagtaccattgatgggaaa
gagctacttggttatattcgggctgtaaattttaatggcagtgctggcactcctgtcact
tttaatgaaaacggagatgctcctggacgttatgatatcttccagtatcaaataaccaac
aaaagcacagagtacaaagtcatcggccactggaccaatcagcttcatctaaaagtggaa
gacatgcagtgggctcatagagaacatactcacccggcgtctgtctgcagcctgccgtgt
aagccaggggagaggaagaaaacggtgaaaggggtcccttgctgctggcactgtgaacgc
tgtgaaggttacaactaccaggtggatgagctgtcctgtgaactttgccctctggatcag
agacccaacatgaaccgcacaggctgccagcttatccccatcatcaaattggagtggcat
tctccctgggctgtggtgcctgtgtttgttgcaatattgggaatcatcgccaccaccttt
gtgatcgtgacctttgtccgctataatgacacacctatcgtgagggcttcaggacgcgaa
cttagttacgtgctcctaacggggatttttctctgttattcaatcacgtttttaatgatt
gcagcaccagatacaatcatatgctccttccgacgggtcttcctaggacttggcatgtgt
ttcagctatgcagcccttctgaccaaaacaaaccgtatccaccgaatatttgagcagggg
aagaaatctgtcacagcgcccaagttcattagtccagcatctcagctggtgatcaccttc
agcctcatctccgtccagctccttggagtgtttgtctggtttgttgtggatcccccccac
atcatcattgactatggagagcagcggacactagatccagagaaggccaggggagtgctc
aagtgtgacatttctgatctctcactcatttgttcacttggatacagtatcctcttgatg
gtcacttgtactgtttatgccattaaaacgagaggtgtcccagagactttcaatgaagcc
aaacctattggatttaccatgtataccacctgcatcatttggttagctttcatccccatc
ttttttggtacagcccagtcagcagaaaagatgtacatccagacaacaacacttactgtc
tccatgagtttaagtgcttcagtatctctgggcatgctctatatgcccaaggtttatatt
ataatttttcatccagaacagaatgttcaaaaacgcaagaggagcttcaaggctgtggtg
acagctgccaccatgcaaagcaaactgatccaaaaaggaaatgacagaccaaatggcgag
gtgaaaagtgaactctgtgagagtcttgaaaccaacagtaagtcatctgtagagtttccg
atggtcaagagcgggagcacttcc

TABLE 4
Human mGluR8b amino acid sequence (SEQ ID NO 4)

mvcegkrsascpcfflltakfywiltmmqrthsqeyahsirvdgdiilgglfpvhakger
gvpcgelkkekgihrleamlyaidqinkdpdllsnitlgvrildtcsrdtyaleqsltfv
qaliekdasdvkcangdppiftkpdkisgvigaaassvsimvanilrlfkipqisyasta
pelsdntrydffsrvvppdsyqaqamvdivtalgwnyvstlasegnygesgveaftqisr
eiggvciaqsqkipreprpgefekiikrlletpnaravimfaneddirrileaakklnqs
ghflwigsdswgskiapvyqqeeiaegavtilpkrasidgfdryfrsrtlannrrnvwfa
efweenfgcklgshgkrnshikkctgleriardssyeqegkvqfvidavysmayalhnmh
kdlcpgyiglcprmstidgkellgyiravnfngsagtpvtfnengdapgrydifqyqitn
ksteykvighwtnqlhlkvedmqwahrehthpasvcslpckpgerkktvkgvpccwhcer
cegynyqvdelscelcpldqrpnmnrtgcqlipiiklewhspwavvpvfvailgiiattf
vivtfvryndtpivrasgrelsyvlltgiflcysitflmiaapdtiicsfrrvflglgmc
fsyaalltktnrihrifeqgkksvtapkfispasqlvitfslisvqllgvfvwfvvdpph
iiidygeqrtldpekargvlkcdisdlslicslgysillmvtctvyaiktrgvpetfnea
kpigftmyttciiwlafipiffgtaqsaekmyiqtttltvsmslsasvslgmlympkvyi
iifhpeqnvqkrkrsfkavvtaatmqskliqkgndrpngevkselcesletnskssvefp
mvksgsts

TABLE 5

8SPhmGluR7 nucleic acid sequence (with 87 nt 5′UTR of
hmGluR8) (SEQ ID NO 5)

ccagaaggtgcagcctcaggtggtgccctttcttctgtggcaagaataaactttgggtct
tggattgcaataccacctgtggagaaaatggtatgcgagggaaagcgatcagcctcttgc
ccttgtttcttcctcttgaccgccaagttctactggatcctcacaatgatgcaaagaact
cacagccaggagtatgcccattccatacggatcgagggggacgtcaccctcggggggctg
ttccccgtgcacgccaagggtcccagcggagtgccctgcggcgacatcaagagggaaaat
gggatccacaggctggaagcgatgctctacgccctggaccagatcaacagtgatcccaac
ctactgcccaacgtgacgctgggcgcgcggatcctggacacttgttccagggacacttac
gcgctcgaacagtcgcttactttcgtccaggcgctcatccagaaggacacctccgacgtg
cgctgcaccaacggcgaaccgccggttttcgtcaagccggagaaagtagttggagtgatt
ggggcttcggggagttcggtctccatcatggtagccaacatcctgaggctcttccagatc
ccccagattagttatgcatcaacggcacccgagctaagtgatgaccggcgctatgacttc
ttctctcgcgtggtgccacccgattccttccaagcccaggccatggtagacattgtaaag
gccctaggctggaattatgtgtctaccctcgcatcggaaggaagttatggagagaaaggt
gtggagtccttcacgcagatttccaaagaggcaggtggactctgcattgcccagtccgtg
agaatcccccaggaacgcaaagacaggaccattgactttgatagaattatcaaacagctc
ctggacacccccaactccagggccgtcgtgatttttgccaacgatgaggatataaagcag
atccttgcagcagccaaaagagctgaccaagttggccattttctttgggtgggatcagac
agctggggatccaaaataaacccactgcaccagcatgaagatatcgcagaaggggccatc
accattcagcccaagcgagccacggtggaagggtttgatgcctactttacgtcccgtaca
cttgaaaacaacagaagaaatgtatggtttgccgaatactgggaggaaaacttcaactgc
aagttgacgattagtgggtcaaaaaaagaagacacagatcgcaaatgcacaggacaggag
agaattggaaaagattccaactatgagcaggagggtaaagtccagttcgtgattgacgca
gtctatgctatggctcacgcccttcaccacatgaacaaggatctctgtgctgactaccgg
ggtgtctgcccagagatggagcaagctggaggcaagaagttgctgaagtatatacgcaat
gttaatttcaatggtagtgctggcactccagtgatgtttaacaagaacggggatgcacct
gggcgttatgacatctttcagtaccagaccacaaacaccagcaacccgggttaccgtctg
atcgggcagtggacagacgaacttcagctcaatatagaagacatgcagtggggtaaagga
gtccgagagatacccgcctcagtgtgcacactaccatgtaagccaggacagagaaagaag
acacagaaaggaactccttgctgttggacctgtgaaccttgcgatggttaccagtaccag
tttgatgagatgacatgccagcattgcccctatgaccagaggcccaatgaaaatcgaacc
ggatgccaggatattcccatcatcaaactggagtggcactccccctgggctgtgattcct
gtcttcctggcaatgttggggatcattgccaccatctttgtcatggccactttcatccgc
tacaatgacacgcccattgtccgggcatctgggcgggaactcagctatgttcttttgacg
ggcatctttctttgctacatcatcactttcctgatgattgccaaaccagatgtggcagtg
tgttctttccggcgagttttcttgggcttgggtatgtgcatcagttatgcagccctcttg
acgaaaacaaatcggatttatcgcatatttgagcagggcaagaaatcagtaacagctccc
agactcataagcccaacatcacaactggcaatcacttccagtttaatatcagttcagctt
ctaggggtgttcatttggtttggtgttgatccacccaacatcatcatagactacgatgaa
cacaagacaatgaaccctgagcaagccagaggggttctcaagtgtgacattacagatctc
caaatcatttgctccttgggatatagcattcttctcatggtcacatgtactgtgtatgcc
atcaagactcggggtgtacccgagaattttaacgaagccaagcccattggattcactatg
tacacgacatgtatagtatggcttgccttcattccaattttttttggcaccgctcaatca
gcggaaaagctctacatacaaactaccacgcttacaatctccatgaacctaagtgcatca
gtggcgctggggatgctatacatgccgaaagtgtacatcatcattttccaccctgaactc
aatgtccagaaacggaagcgaagcttcaaggcggtagtcacagcagccaccatgtcatcg
aggctgtcacacaaacccagtgacagacccaacggtgaggcaaagaccgagctctgtgaa
aacgtagacccaaacagccctgctgcaaaaaagaagtatgtcagttataataacctggtt
atc

TABLE 6
8SPhmGluR7 amino acid sequence (SEQ ID NO 6)

mvcegkrsascpcfflltakfywiltmmqrthsqeyahsiriegdvtlgglfpvhakgps
gvpcgdikrengihrleamlyaldqinsdpnllpnvtlgarildtcsrdtyaleqsltfv
qaliqkdtsdvrctngeppvfvkpekvvgvigasgssvsimvanilrlfqipqisyasta
pelsddrrydffsrvvppdsfqaqamvdivkalgwnyvstlasegsygekgvesftqisk
eagglciaqsvripqerkdrtidfdriikqlldtpnsravvifandedikqilaaakrad
qvghflwvgsdswgskinplhqhediaegaitiqpkratvegfdayftsrtlennrrnvw
faeyweenfnckltisgskkedtdrkctgqerigkdsnyeqegkvqfvidavyamahalh
hmnkdlcadyrgvcpemeqaggkkllkyirnvnfngsagtpvmfnkngdapgrydifqyq
ttntsnpgyrligqwtdelqlniedmqwgkgvreipasvctlpckpgqrkktqkgtpccw
tcepcdgyqyqfdemtcqhcpydqrpnenrtgcqdipiiklewhspwavipvflamlgii
atifvmatfiryndtpivrasgrelsyvlltgiflcyiitflmiakpdvavcsfrrvflg
lgmcisyaalltktnriyrifeqgkksvtaprlisptsqlaitsslisvqllgvfiwfgv
dppniiidydehktmnpeqargvlkcditdlqiicslgysillmvtctvyaiktrgvpen
fneakpigftmyttcivwlafipiffgtaqsaeklyiqtttltismnlsasvalgmlymp
kvyiiifhpelnvqkrkrsfkavvtaatmssrlshkpsdrpngeaktelcenvdpnspaa
kkkyvsynnlvi

TABLE 7

CaSPhmGluR7(27-33) nucleic acid sequence with 40 bp
5′UTR of hCaR (SEQ ID NO 7)

ctagctgtctcatcccttgccctggagagacggcagaaccatggcattttatagctgctg
ctgggtcctcttggcactcacctggcacacctctgcctacgggccagaccagcgagccca
acgcggccaggagatgtacgccccgcactcaatccggatcgagggggacgtcaccctcgg
ggggctgttccccgtgcacgccaagggtcccagcggagtgccctgcggcgacatcaagag
ggaaaatgggatccacaggctggaagcgatgctctacgccctggaccagatcaacagtga
tcccaacctactgcccaacgtgacgctgggcgcgcggatcctggacacttgttccaggga
cacttacgcgctcgaacagtcgcttactttcgtccaggcgctcatccagaaggacacctc
cgacgtgcgctgcaccaacggcgaaccgccggttttcgtcaagccggagaaagtagttgg
agtgattggggcttcggggagttcggtctccatcatggtagccaacatcctgaggctctt
ccagatcccccagattagttatgcatcaacggcacccgagctaagtgatgaccggcgcta
tgacttcttctctcgcgtggtgccacccgattccttccaagcccaggccatggtagacat
tgtaaaggccctaggctggaattatgtgtctaccctcgcatcggaaggaagttatggaga
gaaaggtgtggagtccttcacgcagatttccaaagaggcaggtggactctgcattgccca
gtccgtgagaatcccccaggaacgcaaagacaggaccattgactttgatagaattatcaa
acagctcctggacacccccaactccagggccgtcgtgatttttgccaacgatgaggatat
aaagcagatccttgcagcagccaaaagagctgaccaagttggccattttctttgggtggg
atcagacagctggggatccaaaataaacccactgcaccagcatgaagatatcgcagaagg
ggccatcaccattcagcccaagcgagccacggtggaagggtttgatgcctactttacgtc
ccgtacacttgaaaacaacagaagaaatgtatggtttgccgaatactgggaggaaaactt
caactgcaagttgacgattagtgggtcaaaaaaagaagacacagatcgcaaatgcacagg
acaggagagaattggaaaagattccaactatgagcaggagggtaaagtccagttcgtgat
tgacgcagtctatgctatggctcacgcccttcaccacatgaacaaggatctctgtgctga
ctaccggggtgtctgcccagagatggagcaagctggaggcaagaagttgctgaagtatat
acgcaatgttaatttcaatggtagtgctggcactccagtgatgtttaacaagaacgggga
tgcacctgggcgttatgacatctttcagtaccagaccacaaacaccagcaacccgggtta
ccgtctgatcgggcagtggacagacgaacttcagctcaatatagaagacatgcagtgggg
taaaggagtccgagagatacccgcctcagtgtgcacactaccatgtaagccaggacagag
aaagaagacacagaaaggaactccttgctgttggacctgtgaaccttgcgatggttacca
gtaccagtttgatgagatgacatgccagcattgcccctatgaccagaggcccaatgaaaa
tcgaaccggatgccaggatattcccatcatcaaactggagtggcactccccctgggctgt
gattcctgtcttcctggcaatgttggggatcattgccaccatctttgtcatggccacttt
catccgctacaatgacacgcccattgtccgggcatctgggcgggaactcagctatgttct
tttgacgggcatctttctttgctacatcatcactttcctgatgattgccaaaccagatgt
ggcagtgtgttctttccggcgagttttcttgggcttgggtatgtgcatcagttatgcagc
cctcttgacgaaaacaaatcggatttatcgcatatttgagcagggcaagaaatcagtaac
agctcccagactcataagcccaacatcacaactggcaatcacttccagtttaatatcagt
tcagcttctaggggtgttcatttggtttggtgttgatccacccaacatcatcatagacta
cgatgaacacaagacaatgaaccctgagcaagccagaggggttctcaagtgtgacattac
agatctccaaatcatttgctccttgggatatagcattcttctcatggtcacatgtactgt
gtatgccatcaagactcggggtgtacccgagaattttaacgaagccaagcccattggatt
cactatgtacacgacatgtatagtatggcttgccttcattccaattttttttggcaccgc
tcaatcagcggaaaagctctacatacaaactaccacgcttacaatctccatgaacctaag
tgcatcagtggcgctggggatgctatacatgccgaaagtgtacatcatcattttccaccc
tgaactcaatgtccagaaacggaagcgaagcttcaaggcggtagtcacagcagccaccat
gtcatcgaggctgtcacacaaacccagtgacagacccaacggtgaggcaaagaccgagct
ctgtgaaaacgtagacccaaacagccctgctgcaaaaaagaagtatgtcagttataataa
cctggttatc

TABLE 8
CaSPhmGluR7(27-33) amino acid sequence (SEQ ID NO 8)

mafysccwvllaltwhtsaygpdqraqrgqemyaphsiriegdvtlgglfpvhakgpsgv
pcgdikrengihrleamlyaldqinsdpnllpnvtlgarildtcsrdtyaleqsltfvqa
liqkdtsdvrctngeppvfvkpekvvgvigasgssvsimvanilrlfqipqisyastape
lsddrrydffsrvvppdsfqaqamvdivkalgwnyvstlasegsygekgvesftqiskea
gglciaqsvripqerkdrtidfdriikqlldtpnsravvifandedikqilaaakradqv
ghflwvgsdswgskinplhqhediaegaitiqpkratvegfdayftsrtlennrrnvwfa
eyweenfnckltisgskkedtdrkctgqerigkdsnyeqegkvqfvidavyamahalhhm
nkdlcadyrgvcpemeqaggkkllkyirnvnfngsagtpvmfnkngdapgrydifqyqtt
ntsnpgyrligqwtdelqlniedmqwgkgvreipasvctlpckpgqrkktqkgtpccwtc
epcdgyqyqfdemtcqhcpydqrpnenrtgcqdipiiklewhspwavipvflamlgiiat
ifvmatfiryndtpivrasgrelsyvlltgiflcyiitflmiakpdvavcsfrrvflglg
mcisyaalltktnriyrifeqgkksvtaprlisptsqlaitsslisvqllgvfiwfgvdp
pniiidydehktmnpeqargvlkcditdlqiicslgysillmvtctvyaiktrgvpenfn
eakpigftmyttcivwlafipiffgtaqsaeklyiqtttltismnlsasvalgmlympkv
yiiifhpelnvqkrkrsfkavvtaatmssrlshkpsdrpngeaktelcenvdpnspaakk
kyvsynnlvi

TABLE 9

CaSPhmGluR7(27-36) nucleic acid sequence with 40 bp
5′UTR of hCaR (SEQ ID NO 9)

ctagctgtctcatcccttgccctggagagacggcagaaccatggcattttatagctgctg
ctgggtcctcttggcactcacctggcacacctctgcctacgggccagaccagcgagccca
agagatgtacgccccgcactcaatccggatcgagggggacgtcaccctcggggggctgtt
ccccgtgcacgccaagggtcccagcggagtgccctgcggcgacatcaagagggaaaatgg
gatccacaggctggaagcgatgctctacgccctggaccagatcaacagtgatcccaacct
actgcccaacgtgacgctgggcgcgcggatcctggacacttgttccagggacacttacgc
gctcgaacagtcgcttactttcgtccaggcgctcatccagaaggacacctccgacgtgcg
ctgcaccaacggcgaaccgccggttttcgtcaagccggagaaagtagttggagtgattgg
ggcttcggggagttcggtctccatcatggtagccaacatcctgaggctcttccagatccc
ccagattagttatgcatcaacggcacccgagctaagtgatgaccggcgctatgacttctt
ctctcgcgtggtgccacccgattccttccaagcccaggccatggtagacattgtaaaggc
cctaggctggaattatgtgtctaccctcgcatcggaaggaagttatggagagaaaggtgt
ggagtccttcacgcagatttccaaagaggcaggtggactctgcattgcccagtccgtgag
aatcccccaggaacgcaaagacaggaccattgactttgatagaattatcaaacagctcct
ggacacccccaactccagggccgtcgtgatttttgccaacgatgaggatataaagcagat
ccttgcagcagccaaaagagctgaccaagttggccattttctttgggtgggatcagacag
ctggggatccaaaataaacccactgcaccagcatgaagatatcgcagaaggggccatcac
cattcagcccaagcgagccacggtggaagggtttgatgcctactttacgtcccgtacact
tgaaaacaacagaagaaatgtatggtttgccgaatactgggaggaaaacttcaactgcaa
gttgacgattagtgggtcaaaaaaagaagacacagatcgcaaatgcacaggacaggagag
aattggaaaagattccaactatgagcaggagggtaaagtccagttcgtgattgacgcagt
ctatgctatggctcacgcccttcaccacatgaacaaggatctctgtgctgactaccgggg
tgtctgcccagagatggagcaagctggaggcaagaagttgctgaagtatatacgcaatgt
taatttcaatggtagtgctggcactccagtgatgtttaacaagaacggggatgcacctgg
gcgttatgacatctttcagtaccagaccacaaacaccagcaacccgggttaccgtctgat
cgggcagtggacagacgaacttcagctcaatatagaagacatgcagtggggtaaaggagt
ccgagagatacccgcctcagtgtgcacactaccatgtaagccaggacagagaaagaagac
acagaaaggaactccttgctgttggacctgtgaaccttgcgatggttaccagtaccagtt
tgatgagatgacatgccagcattgcccctatgaccagaggcccaatgaaaatcgaaccgg
atgccaggatattcccatcatcaaactggagtggcactccccctgggctgtgattcctgt
cttcctggcaatgttggggatcattgccaccatctttgtcatggccactttcatccgcta
caatgacacgcccattgtccgggcatctgggcgggaactcagctatgttcttttgacggg
catctttctttgctacatcatcactttcctgatgattgccaaaccagatgtggcagtgtg
ttctttccggcgagttttcttgggcttgggtatgtgcatcagttatgcagccctcttgac
gaaaacaaatcggatttatcgcatatttgagcagggcaagaaatcagtaacagctcccag
actcataagcccaacatcacaactggcaatcacttccagtttaatatcagttcagcttct
aggggtgttcatttggtttggtgttgatccacccaacatcatcatagactacgatgaaca
caagacaatgaaccctgagcaagccagaggggttctcaagtgtgacattacagatctcca
aatcatttgctccttgggatatagcattcttctcatggtcacatgtactgtgtatgccat
caagactcggggtgtacccgagaattttaacgaagccaagcccattggattcactatgta
cacgacatgtatagtatggcttgccttcattccaattttttttggcaccgctcaatcagc
ggaaaagctctacatacaaactaccacgcttacaatctccatgaacctaagtgcatcagt
ggcgctggggatgctatacatgccgaaagtgtacatcatcattttccaccctgaactcaa
tgtccagaaacggaagcgaagcttcaaggcggtagtcacagcagccaccatgtcatcgag
gctgtcacacaaacccagtgacagacccaacggtgaggcaaagaccgagctctgtgaaaa
cgtagacccaaacagccctgctgcaaaaaagaagtatgtcagttataataacctggttat
c

TABLE 10
CaSPhmGluR7(27-36) amino acid sequence (SEQ ID NO 10)

mafysccwvllaltwhtsaygpdqraqemyaphsiriegdvtlgglfpvhakgpsgvpcg
dikrengihrleamlyaldqinsdpnllpnvtlgarildtcsrdtyaleqsltfvqaliq
kdtsdvrctngeppvfvkpekvvgvigasgssvsimvanilrlfqipqisyastapelsd
drrydffsrvvppdsfqaqamvdivkalgwnyvstlasegsygekgvesftqiskeaggl
ciaqsvripqerkdrtidfdriikqlldtpnsravvifandedikqilaaakradqvghf
lwvgsdswgskinplhqhediaegaitiqpkratvegfdayftsrtlennrrnvwfaeyw
eenfnckltisgskkedtdrkctgqerigkdsnyeqegkvqfvidavyamahalhhmnkd
lcadyrgvcpemeqaggkkllkyirnvnfngsagtpvmfnkngdapgrydifqyqttnts
npgyrligqwtdelqlniedmqwgkgvreipasvctlpckpgqrkktqkgtpccwtcepc
dgyqyqfdemtcqhcpydqrpnenrtgcqdipiiklewhspwavipvflamlgiiatifv
matfiryndtpivrasgrelsyvlltgiflcyiitflmiakpdvavcsfrrvflglgmci
syaalltktnriyrifeqgkksvtaprlisptsqlaitsslisvqllgvfiwfgvdppni
iidydehktmnpeqargvlkcditdlqiicslgysillmvtctvyaiktrgvpenfneak
pigftmyttcivwlafipiffgtaqsaeklyiqtttltismnlsasvalgmlympkvyii
ifhpelnvqkrkrsfkavvtaatmssrlshkpsdrpngeaktelcenvdpnspaakkkyv
synnivi

TABLE 11

CaSPhmGluR7(27-45) nucleic acid sequence with 40 bp
5′UTR of hCaR (SEQ ID NO 11)

ctagctgtctcatcccttgccctggagagacggcagaaccatggcattttatagctgctg
ctgggtcctcttggcactcacctggcacacctctgcctacgggccagaccagcgagccca
aatcgagggggacgtcaccctcggggggctgttccccgtgcacgccaagggtcccagcgg
agtgccctgcggcgacatcaagagggaaaatgggatccacaggctggaagcgatgctcta
cgccctggaccagatcaacagtgatcccaacctactgcccaacgtgacgctgggcgcgcg
gatcctggacacttgttccagggacacttacgcgctcgaacagtcgcttactttcgtcca
ggcgctcatccagaaggacacctccgacgtgcgctgcaccaacggcgaaccgccggtttt
cgtcaagccggagaaagtagttggagtgattggggcttcggggagttcggtctccatcat
ggtagccaacatcctgaggctcttccagatcccccagattagttatgcatcaacggcacc
cgagctaagtgatgaccggcgctatgacttcttctctcgcgtggtgccacccgattcctt
ccaagcccaggccatggtagacattgtaaaggccctaggctggaattatgtgtctaccct
cgcatcggaaggaagttatggagagaaaggtgtggagtccttcacgcagatttccaaaga
ggcaggtggactctgcattgcccagtccgtgagaatcccccaggaacgcaaagacaggac
cattgactttgatagaattatcaaacagctcctggacacccccaactccagggccgtcgt
gatttttgccaacgatgaggatataaagcagatccttgcagcagccaaaagagctgacca
agttggccattttctttgggtgggatcagacagctggggatccaaaataaacccactgca
ccagcatgaagatatcgcagaaggggccatcaccattcagcccaagcgagccacggtgga
agggtttgatgcctactttacgtcccgtacacttgaaaacaacagaagaaatgtatggtt
tgccgaatactgggaggaaaacttcaactgcaagttgacgattagtgggtcaaaaaaaga
agacacagatcgcaaatgcacaggacaggagagaattggaaaagattccaactatgagca
ggagggtaaagtccagttcgtgattgacgcagtctatgctatggctcacgcccttcacca
catgaacaaggatctctgtgctgactaccggggtgtctgcccagagatggagcaagctgg
aggcaagaagttgctgaagtatatacgcaatgttaatttcaatggtagtgctggcactcc
agtgatgtttaacaagaacggggatgcacctgggcgttatgacatctttcagtaccagac
cacaaacaccagcaacccgggttaccgtctgatcgggcagtggacagacgaacttcagct
caatatagaagacatgcagtggggtaaaggagtccgagagatacccgcctcagtgtgcac
actaccatgtaagccaggacagagaaagaagacacagaaaggaactccttgctgttggac
ctgtgaaccttgcgatggttaccagtaccagtttgatgagatgacatgccagcattgccc
ctatgaccagaggcccaatgaaaatcgaaccggatgccaggatattcccatcatcaaact
ggagtggcactccccctgggctgtgattcctgtcttcctggcaatgttggggatcattgc
caccatctttgtcatggccactttcatccgctacaatgacacgcccattgtccgggcatc
tgggcgggaactcagctatgttcttttgacgggcatctttctttgctacatcatcacttt
cctgatgattgccaaaccagatgtggcagtgtgttctttccggcgagttttcttgggctt
gggtatgtgcatcagttatgcagccctcttgacgaaaacaaatcggatttatcgcatatt
tgagcagggcaagaaatcagtaacagctcccagactcataagcccaacatcacaactggc
aatcacttccagtttaatatcagttcagcttctaggggtgttcatttggtttggtgttga
tccacccaacatcatcatagactacgatgaacacaagacaatgaaccctgagcaagccag
aggggttctcaagtgtgacattacagatctccaaatcatttgctccttgggatatagcat
tcttctcatggtcacatgtactgtgtatgccatcaagactcggggtgtacccgagaattt
taacgaagccaagcccattggattcactatgtacacgacatgtatagtatggcttgcctt
cattccaattttttttggcaccgctcaatcagcggaaaagctctacatacaaactaccac
gcttacaatctccatgaacctaagtgcatcagtggcgctggggatgctatacatgccgaa
agtgtacatcatcattttccaccctgaactcaatgtccagaaacggaagcgaagcttcaa
ggcggtagtcacagcagccaccatgtcatcgaggctgtcacacaaacccagtgacagacc
caacggtgaggcaaagaccgagctctgtgaaaacgtagacccaaacagccctgctgcaaa
aaagaagtatgtcagttataataacctggttatc

TABLE 12
CaSPhmGluR7(27-45) amino acid sequence (SEQ ID NO 12)

mafysccwvllaltwhtsaygpdqraqiegdvtlgglfpvhakgpsgvpcgdikrengih
rleamlyaldqinsdpnnllpnvtlgarildtcsrdtyaleqsltfvqaliqkdtsdvrct
ngeppvfvkpekvvgvigasgssvsimvanilrlfqipqisyastapelsddrrydffsr
vvppdsfqaqamvdivkalgwnyvstlasegsygekgvesftqiskeagglciaqsvrip
qerkdrtidfdriikqlldtpnsravvifandedikqilaaakradqvghflwvgsdswg
skinplhqhediaegaitiqpkratvegfdayftsrtlennrrnvwfaeyweenfncklt
isgskkedtdrkctgqerigkdsnyeqegkvqfvidavyamahalhhmnkdlcadyrgvc
pemeqaggkkllkyirnvnfngsagtpvmfnkngdapgrydifqyqttntsnpgyrligq
wtdelqlniedmqwgkgvreipasvctlpckpgqrkktqkgtpccwtcepcdgyqyqfde
mtcqhcpydqrpnenrtgcqdipiiklewhspwavipvflamlgiiatifvmatfirynd
tpivrasgrelsyvlltgiflcyiitflmiakpdvavcsfrrvflglgmcisyaalltkt
nriyrifeqgkksvtaprlisptsqlaitsslisvqllgvfiwfgvdppniiidydehkt
mnpeqargvlkcditdlqiicslgysillmvtctvyaiktrgvpenfneakpigftmytt
civwlafipiffgtaqsaeklyiqtttltismnlsasvalgmlympkvyiiifhpelnvq
krkrsfkavvtaatmssrlshkpsdrpngeaktelcenvdpnspaakkkyvsynnlvi

TABLE 13
CaSPhmGluR3(27-21) nucleic acid sequence (SEQ ID NO 13)

atggcattttatagctgctgctgggtcctcttggcactcacctggcacacctctgcctacgggccagacc
agcgagcccaattaggggaccataactttctaaggagagagattaaaatagaaggtgaccttgttttagg
gggcctgtttcctattaacgaaaaaggcactggaactgaagaatgtgggcgaatcaatgaagaccgaggg
attcaacgcctggaagccatgttgtttgctattgatgaaatcaacaaagatgattacttgctaccaggag
tgaagttgggtgttcacattttggatacatgttcaagggatacctatgcattggagcaatcactggagtt
tgtcagggcatctttgacaaaagtggatgaagctgagtatatgtgtcctgatggatcctatgccattcaa
gaaaacatcccacttctcattgcaggggtcattggtggctcttatagcagtgtttccatacaggtggcaa
acctgctgcggctcttccagatccctcagatcagctacgcatccaccagcgccaaactcagtgataagtc
gcgctatgattactttgccaggaccgtgccccccgacttctaccaggccaaagccatggctgagatcttg
cgcttcttcaactggacctacgtgtccacagtagcctccgagggtgattacggggagacagggatcgagg
ccttcgagcaggaagcccgcctgcgcaacatctgcatcgctacggcggagaaggtgggccgctccaacat
ccgcaagtcctacgacagcgtgatccgagaactgttgcagaagcccaacgcgcgcgtcgtggtcctcttc
atgcgcagcgacgactcgcgggagctcattgcagccgccagccgcgccaatgcctccttcacctgggtgg
ccagcgacggctggggcgcgcaggagagcatcatcaagggcagcgagcatgtggcctacggcgccatcac
cctggagctggcctcccagcctgtccgccagttcgaccgctacttccagagcctcaacccctacaacaac
caccgcaacccctggttccgggacttctgggagcaaaagtttcagtgcagcctccagaacaaacgcaacc
acaggcgcgtctgcgacaagcacctggccatcgacagcagcaactacgagcaagagtccaagatcatgtt
tgtggtgaacgcggtgtatgccatggcccacgctttgcacaaaatgcagcgcaccctctgtcccaacact
accaagctttgtgatgctatgaagatcctggatgggaagaagttgtacaaggattacttgctgaaaatca
acttcacggctccattcaacccaaataaagatgcagatagcatagtcaagtttgacacttttggagatgg
aatggggcgatacaacgtgttcaatttccaaaatgtaggtggaaagtattcctacttgaaagttggtcac
tgggcagaaaccttatcgctagatgtcaactctatccactggtcccggaactcagtccccacttcccagt
gcagcgacccctgtgcccccaatgaaatgaagaatatgcaaccaggggatgtctgctgctggatttgcat
cccctgtgaaccctacgaatacctggctgatgagtttacctgtatggattgtgggtctggacagtggccc
actgcagacctaactggatgctatgaccttcctgaggactacatcaggtgggaagacgcctgggccattg
gcccagtcaccattgcctgtctgggttttatgtgtacatgcatggttgtaactgtttttatcaagcacaa
caacacacccttggtcaaagcatcgggccgagaactctgctacatcttattgtttggggttggcctgtca
tactgcatgacattcttcttcattgccaagccatcaccagtcatctgtgcattgcgccgactcgggctgg
ggagttccttcgctatctgttactcagccctgctgaccaagacaaactgcattgcccgcatcttcgatgg
ggtcaagaatggcgctcagaggccaaaattcatcagccccagttctcaggttttcatctgcctgggtctg
atcctggtgcaaattgtgatggtgtctgtgtggctcatcctggaggccccaggcaccaggaggtataccc
ttgcagagaagcgggaaacagtcatcctaaaatgcaatgtcaaagattccagcatgttgatctctcttac
ctacgatgtgatcctggtgatcttatgcactgtgtacgccttcaaaacgcggaagtgcccagaaaatttc
aacgaagctaagttcataggttttaccatgtacaccacgtgcatcatctggttggccttcctccctatat
tttatgtgacatcaagtgactacagagtgcagacgacaaccatgtgcatctctgtcagcctgagtggctt
tgtggtcttgggctgtttgtttgcacccaaggttcacatcatcctgtttcaaccccagaagaatgttgtc
acacacagactgcacctcaacaggttcagtgtcagtggaactgggaccacatactctcagtcctctgcaa
gcacgtatgtgccaacggtgtgcaatgggcgggaagtcctcgactccaccacctcatctctg

TABLE 14
CaSPhmGluR3(27-21) amino acid sequence (SEQ ID NO 14)

MAFYSCCWVLLALTWHTSAYGPDQRAQLGDHNFLRREIKIEGDLVLGGLFPINEKGTGTEECGRINEDRG
IQRLEAMLFAIDEINKDDYLLPGVKLGVHILDTCSRDTYALEQSLEFVRASLTKVDEAEYMCPDGSYAIQ
ENIPLLIAGVIGGSYSSVSIQVANLLRLFQIPQISYASTSAKLSDKSRYDYFARTVPPDFYQAKAMAEIL
RFFNWTYVSTVASEGDYGETGIEAFEQEARLRNICIATAEKVGRSNIRKSYDSVIRELLQKPNARVVVLF
MRSDDSRELIAAASRANASFTWVASDGWGAQESIIKGSEHVAYGAITLELASQPVRQFDRYFQSLNPYNN
HRNPWFRDFWEQKFQCSLQNKRNHRRVCDKHLAIDSSNYEQESKIMFVVNAVYAMAHALHKMQRTLCPNT
TKLCDAMKILDGKKLYKDYLLKINFTAPFNPNKDADSIVKFDTFGDGMGRYNVFNFQNVGGKYSYLKVGH
WAETLSLDVNSIHWSRNSVPTSQCSDPCAPNEMKNMQPGDVCCWICIPCEPYEYLADEFTCMDCGSGQWP
TADLTGCYDLPEDYIRWEDAWAIGPVTIACLGFMCTCMVVTVFIKHNNTPLVKASGRELCYILLFGVGLS
YCMTFFFIAKPSPVICALRRLGLGSSFAICYSALLTKTNCIARIFDGVKNGAQRPKFISPSSQVFICLGL
ILVQIVMVSVWLILEAPGTRRYTLAEKRETVILKCNVKDSSMLISLTYDVILVILCTVYAFKTRKCPENF
NEAKFIGFTMYTTCIIWLAFLPIFYVTSSDYRVQTTTMCISVSLSGFVVLGCLFAPKVHIILFQPQKNVV
THRLHLNRFSVSGTGTTYSQSSASTYVPTVCNGREVLDSTTSSL

TABLE 15
CaSPhmGluR3(27-23) nucleic acid sequence (SEQ ID NO 15)

atggcattttatagctgctgctgggtcctcttggcactcacctggcacacctctgcctacgggccagacc
agcgagcccaagaccataactttctaaggagagagattaaaatagaaggtgaccttgttttagggggcct
gtttcctattaacgaaaaaggcactggaactgaagaatgtgggcgaatcaatgaagaccgagggattcaa
cgcctggaagccatgttgtttgctattgatgaaatcaacaaagatgattacttgctaccaggagtgaagt
tgggtgttcacattttggatacatgttcaagggatacctatgcattggagcaatcactggagtttgtcag
ggcatctttgacaaaagtggatgaagctgagtatatgtgtcctgatggatcctatgccattcaagaaaac
atcccacttctcattgcaggggtcattggtggctcttatagcagtgtttccatacaggtggcaaacctgc
tgcggctcttccagatccctcagatcagctacgcatccaccagcgccaaactcagtgataagtcgcgcta
tgattactttgccaggaccgtgccccccgacttctaccaggccaaagccatggctgagatcttgcgcttc
ttcaactggacctacgtgtccacagtagcctccgagggtgattacggggagacagggatcgaggccttcg
agcaggaagcccgcctgcgcaacatctgcatcgctacggcggagaaggtgggccgctccaacatccgcaa
gtcctacgacagcgtgatccgagaactgttgcagaagcccaacgcgcgcgtcgtggtcctcttcatgcgc
agcgacgactcgcgggagctcattgcagccgccagccgcgccaatgcctccttcacctgggtggccagcg
acggctggggcgcgcaggagagcatcatcaagggcagcgagcatgtggcctacggcgccatcaccctgga
gctggcctcccagcctgtccgccagttcgaccgctacttccagagcctcaacccctacaacaaccaccgc
aacccctggttccgggacttctgggagcaaaagtttcagtgcagcctccagaacaaacgcaaccacaggc
gcgtctgcgacaagcacctggccatcgacagcagcaactacgagcaagagtccaagatcatgtttgtggt
gaacgcggtgtatgccatggcccacgctttgcacaaaatgcagcgcaccctctgtcccaacactaccaag
ctttgtgatgctatgaagatcctggatgggaagaagttgtacaaggattacttgctgaaaatcaacttca
cggctccattcaacccaaataaagatgcagatagcatagtcaagtttgacacttttggagatggaatggg
gcgatacaacgtgttcaatttccaaaatgtaggtggaaagtattcctacttgaaagttggtcactgggca
gaaaccttatcgctagatgtcaactctatccactggtcccggaactcagtccccacttcccagtgcagcg
acccctgtgcccccaatgaaatgaagaatatgcaaccaggggatgtctgctgctggatttgcatcccctg
tgaaccctacgaatacctggctgatgagtttacctgtatggattgtgggtctggacagtggcccactgca
gacctaactggatgctatgaccttcctgaggactacatcaggtgggaagacgcctgggccattggcccag
tcaccattgcctgtctgggttttatgtgtacatgcatggttgtaactgtttttatcaagcacaacaacac
acccttggtcaaagcatcgggccgagaactctgctacatcttattgtttggggttggcctgtcatactgc
atgacattcttcttcattgccaagccatcaccagtcatctgtgcattgcgccgactcgggctggggagtt
ccttcgctatctgttactcagccctgctgaccaagacaaactgcattgcccgcatcttcgatggggtcaa
gaatggcgctcagaggccaaaattcatcagccccagttctcaggttttcatctgcctgggtctgatcctg
gtgcaaattgtgatggtgtctgtgtggctcatcctggaggccccaggcaccaggaggtatacccttgcag
agaagcgggaaacagtcatcctaaaatgcaatgtcaaagattccagcatgttgatctctcttacctacga
tgtgatcctggtgatcttatgcactgtgtacgccttcaaaacgcggaagtgcccagaaaatttcaacgaa
gctaagttcataggttttaccatgtacaccacgtgcatcatctggttggccttcctccctatattttatg
tgacatcaagtgactacagagtgcagacgacaaccatgtgcatctctgtcagcctgagtggctttgtggt
cttgggctgtttgtttgcacccaaggttcacatcatcctgtttcaaccccagaagaatgttgtcacacac
agactgcacctcaacaggttcagtgtcagtggaactgggaccacatactctcagtcctctgcaagcacgt
atgtgccaacggtgtgcaatgggcgggaagtcctcgactccaccacctcatctctg

TABLE 16
CaSPhmGluR3(27-23) amino acid sequence (SEQ ID NO 16)

MAFYSCCWVLLALTWHTSAYGPDQRAQDHNFLRREIKIEGDLVLGGLFPINEKGTGTEECGRINEDRGIQ
RLEAMLFAIDEINKDDYLLPGVKLGVHILDTCSRDTYALEQSLEFVRASLTKVDEAEYMCPDGSYAIQEN
IPLLIAGVIGGSYSSVSIQVANLLRLFQIPQISYASTSAKLSDKSRYDYFARTVPPDFYQAKAMAEILRF
FNWTYVSTVASEGDYGETGIEAFEQEARLRNICIATAEKVGRSNIRKSYDSVIRELLQKPNARVVVLFMR
SDDSRELIAAASRANASFTWVASDGWGAQESIIKGSEHVAYGAITLELASQPVRQFDRYFQSLNPYNNHR
NPWFRDFWEQKFQCSLQNKRNHRRVCDKHLAIDSSNYEQESKIMFVVNAVYAMAHALHKMQRTLCPNTTK
LGDAMKILDGKKLYKDYLLKINFTAPFNPNKDADSIVKFDTFGDGMGRYNVFNFQNVGGKYSYLKVGHWA
ETLSLDVNSIHWSRNSVPTSQCSDPCAPNEMKNMQPGDVCCWICIPCEPYEYLADEFTCMDCGSGQWPTA
DLTGCYDLPEDYIRWEDAWAIGPVTIACLGFMCTCMVVTVFIKHNNTPLVKASGRELCYILLFGVGLSYC
MTFFFIAKPSPVICALRRLGLGSSFAICYSALLTKTNCIARIFDGVKNGAQRPKFISPSSQVFICLGLIL
VQIVMVSVWLILEAPGTRRYTLAEKRETVILKCNVKDSSMLISLTYDVILVILCTVYAFKTRKCPENFNE
AKFIGFTMYTTCIIWLAFLPIFYVTSSDYRVQTTTMCISVSLSGFVVLGCLFAPKVHIILFQPQKNVVTH
RLHLNRFSVSGTGTTYSQSSASTYVPTVCNGREVLDSTTSSL

TABLE 17
CaSPhmGluR3(27-33) nucleic acid sequence (SEQ ID NO 17)

atggcattttatagctgctgctgggtcctcttggcactcacctggcacacctctgcctacgggccagacc
agcgagcccaaatagaaggtgaccttgttttagggggcctgtttcctattaacgaaaaaggcactggaac
tgaagaatgtgggcgaatcaatgaagaccgagggattcaacgcctggaagccatgttgtttgctattgat
gaaatcaacaaagatgattacttgctaccaggagtgaagttgggtgttcacattttggatacatgttcaa
gggatacctatgcattggagcaatcactggagtttgtcagggcatctttgacaaaagtggatgaagctga
gtatatgtgtcctgatggatcctatgccattcaagaaaacatcccacttctcattgcaggggtcattggt
ggctcttatagcagtgtttccatacaggtggcaaacctgctgcggctcttccagatccctcagatcagct
acgcatccaccagcgccaaactcagtgataagtcgcgctatgattactttgccaggaccgtgccccccga
cttctaccaggccaaagccatggctgagatcttgcgcttcttcaactggacctacgtgtccacagtagcc
tccgagggtgattacggggagacagggatcgaggccttcgagcaggaagcccgcctgcgcaacatctgca
tcgctacggcggagaaggtgggccgctccaacatccgcaagtcctacgacagcgtgatccgagaactgtt
gcagaagcccaacgcgcgcgtcgtggtcctcttcatgcgcagcgacgactcgcgggagctcattgcagcc
gccagccgcgccaatgcctccttcacctgggtggccagcgacggctggggcgcgcaggagagcatcatca
agggcagcgagcatgtggcctacggcgccatcaccctggagctggcctcccagcctgtccgccagttcga
ccgctacttccagagcctcaacccctacaacaaccaccgcaacccctggttccgggacttctgggagcaa
aagtttcagtgcagcctccagaacaaacgcaaccacaggcgcgtctgcgacaagcacctggccatcgaca
gcagcaactacgagcaagagtccaagatcatgtttgtggtgaacgcggtgtatgccatggcccacgcttt
gcacaaaatgcagcgcaccctctgtcccaacactaccaagctttgtgatgctatgaagatcctggatggg
aagaagttgtacaaggattacttgctgaaaatcaacttcacggctccattcaacccaaataaagatgcag
atagcatagtcaagtttgacacttttggagatggaatggggcgatacaacgtgttcaatttccaaaatgt
aggtggaaagtattcctacttgaaagttggtcactgggcagaaaccttatcgctagatgtcaactctatc
cactggtcccggaactcagtccccacttcccagtgcagcgacccctgtgcccccaatgaaatgaagaata
tgcaaccaggggatgtctgctgctggatttgcatcccctgtgaaccctacgaatacctggctgatgagtt
tacctgtatggattgtgggtctggacagtggcccactgcagacctaactggatgctatgaccttcctgag
gactacatcaggtgggaagacgcctgggccattggcccagtcaccattgcctgtctgggttttatgtgta
catgcatggttgtaactgtttttatcaagcacaacaacacacccttggtcaaagcatcgggccgagaact
ctgctacatcttattgtttggggttggcctgtcatactgcatgacattcttcttcattgccaagccatca
ccagtcatctgtgcattgcgccgactcgggctggggagttccttcgctatctgttactcagccctgctga
ccaagacaaactgcattgcccgcatcttcgatggggtcaagaatggcgctcagaggccaaaattcatcag
ccccagttctcaggttttcatctgcctgggtctgatcctggtgcaaattgtgatggtgtctgtgtggctc
atcctggaggccccaggcaccaggaggtatacccttgcagagaagcgggaaacagtcatcctaaaatgca
atgtcaaagattccagcatgttgatctctcttacctacgatgtgatcctggtgatcttatgcactgtgta
cgccttcaaaacgcggaagtgcccagaaaatttcaacgaagctaagttcataggttttaccatgtacacc
acgtgcatcatctggttggccttcctccctatattttatgtgacatcaagtgactacagagtgcagacga
caaccatgtgcatctctgtcagcctgagtggctttgtggtcttgggctgtttgtttgcacccaaggttca
catcatcctgtttcaaccccagaagaatgttgtcacacacagactgcacctcaacaggttcagtgtcagt
ggaactgggaccacatactctcagtcctctgcaagcacgtatgtgccaacggtgtgcaatgggcgggaag
tcctcgactccaccacctcatctctg

TABLE 18
CaSPhmGluR3(27-33) amino acid sequence (SEQ ID NO 18)

MAFYSCCWVLLALTWHTSAYGPDQRAQIEGDLVLGGLFPINEKGTGTEECGRINEDRGIQRLEAMLFAID
EINKDDYLLPGVKLGVHILDTCSRDTYALEQSLEFVRASLTKVDEAEYMCPDGSYAIQENIPLLIAGVIG
GSYSSVSIQVANLLRLFQIPQISYASTSAKLSDKSRYDYFARTVPPDFYQAKAMAEILRFFNWTYVSTVA
SEGDYGETGIEAFEQEARLRNICIATAEKVGRSNIRKSYDSVIRELLQKPNARVVVLFMRSDDSRELIAA
ASRANASFTWVASDGWGAQESIIKGSEHVAYGAITLELASQPVRQFDRYFQSLNPYNNHRNPWFRDFWEQ
KFQCSLQNKRNHRRVCDKHLAIDSSNYEQESKIMFVVNAVYAMAHALHKMQRTLCPNTTKLCDAMKILDG
KKLYKDYLLKINFTAPFNPNKDADSIVKFDTFGDGMGRYNVFNFQNVGGKYSYLKVGHWAETLSLDVNSI
HWSRNSVPTSQCSDPCAPNEMKNMQPGDVCCWICIPCEPYEYLADEFTCMDCGSGQWPTADLTGCYDLPE
DYIRWEDAWAIGPVTIACLGFMCTCMVVTVFIKHNNTPLVKASGRELCYILLFGVGLSYCMTFFFIAKPS
PVICALRRLGLGSSFAICYSALLTKTNCIARIFDGVKNGAQRPKFISPSSQVFICLGLILVQIVMVSVWL
ILEAPGTRRYTLAEKRETVILKCNVKDSSMLISLTYDVILVILCTVYAFKTRKCPENFNEAKFIGFTMYT
TCIIWLAFLPIFYVTSSDYRVQTTTMCISVSLSGFVVLGCLFAPKVHIILFQPQKNVVTHRLHLNRFSVS
GTGTTYSQSSASTYVPTVCNGREVLDSTTSSL

TABLE 19
CaSPhmGluR2(27-19) nucleic acid sequence (SEQ ID NO 19)

atggcattttatagctgctgctgggtcctcttggcactcacctggcacacctctgcctacgggccagacc
agcgcgcccaagagggcccagccaagaaggtgctgaccctggagggagacttggtgctgggtgggctgtt
cccagtgcaccagaagggcggcccagcagaggactgtggtcctgtcaatgagcaccgtggcatccagcgc
ctggaggccatgctttttgcactggaccgcatcaaccgtgacccgcacctgctgcctggcgtgcgcctgg
gtgcacacatcctcgacagttgctccaaggacacacatgcgctggagcaggcactggactttgtgcgtgc
ctcactcagccgtggtgctgatggctcacgccacatctgccccgacggctcttatgcgacccatggtgat
gctcccactgccatcactggtgttattggcggttcctacagtgatgtctccatccaggtggccaacctct
tgaggctatttcagatcccacagattagctacgcctctaccagtgccaagctgagtgacaagtcccgcta
tgactactttgcccgcacagtgcctcctgacttcttccaagccaaggccatggctgagattctccgcttc
ttcaactggacctatgtgtccactgtggcgtctgagggcgactatggcgagacaggcattgaggcctttg
agctagaggctcgtgcccgcaacatctgtgtggccacctcggagaaagtgggccgtgccatgagccgcgc
ggcctttgagggtgtggtgcgagccctgctgcagaagcccagtgcccgcgtggctgtcctgttcacccgt
tctgaggatgcccgggagctgcttgctgccagccagcgcctcaatgccagcttcacctgggtggccagtg
atggttggggggccctggagagtgtggtggcaggcagtgagggggctgctgagggtgctatcaccatcga
gctggcctcctaccccatcagtgactttgcctcctacttccagagcctggacccttggaacaacagccgg
aacccctggttccgtgaattctgggagcagaggttccgctgcagcttccggcagcgagactgcgcagccc
actctctccgggctgtgccctttgagcaggagtccaagatcatgtttgtggtcaatgcagtgtacgccat
ggcccatgcgctccacaacatgcaccgtgccctctgccccaacaccacccggctctgtgacgcgatgcgg
ccagttaacgggcgccgcctctacaaggactttgtgctcaacgtcaagtttgatgccccctttcgcccag
ctgacacccacaatgaggtccgctttgaccgctttggtgatggtattggccgctacaacatcttcaccta
tctgcgtgcaggcagtgggcgctatcgctaccagaaggtgggctactgggcagaaggcttgacztctggac
accagcctcatcccatgggcctcaccctcagccggccccctgcccgcctctcgctgcagtgagccctgcc
tccagaatgaggtgaagagtgtgcagccgggcgaagtctgctgctggctctgcattccgtgccagcccta
tgagtaccgattggacgaattcacttgcgctgattgtggcctgggctactggcccaatgccagcctgact
ggctgcttcgaactgccccaggagtacatccgctggggcgatgcctgggctgtgggacctgtcaccatcg
cctgcctcggtgccctggccaccctctttgtgctgggtgtctttgtgcggcacaatgccacaccagtggt
caaggcctcaggtcgggagctctgctacatcctgctgggtggtgtcttcctctgctactgcatgaccttc
atcttcattgccaagccatccacggcagtgtgtaccttacggcgtcttggtttgggcactgccttctctg
tctgctactcagccctgctcaccaagaccaaccgcattgcacgcatcttcggtggggcccgggagggtgc
ccagcggccacgcttcatcagtcctgcctcacaggtggccatctgcctggcacttatctcgggccagctg
ctcatcgtggtcgcctggctggtggtggaggcaccgggcacaggcaaggagacagcccccgaacggcggg
aggtggtgacactgcgctgcaaccaccgcgatgcaagtatgttgggctcgctggcctacaatgtgctcct
catcgcgctctgcacgctttatgccttcaagactcgcaagtgccccgaaaacttcaacgaggccaagttc
attggcttcaccatgtacaccacctgcatcatctggctggcattcctgcccatcttctatgtcacctcca
gtgactaccgggtacagaccaccaccatgtgcgtgtcagtcagcctcagcggctccgtggtgcttggctg
cctctttgcgcccaagctgcacatcatcctcttccagccgcagaagaacgtggttagccaccgggcaccc
accagccgctttggcagtgctgctgccagggccagctccagccttggccaagggtctggctcccagtttg
tccccactgtttgcaatggccgtgaggtggtggactcgacaacgtcatcgctt

TABLE 20
CaSPhmGluR2(27-19) amino acid sequence (SEQ ID NO 20)

MAFYSCCWVLLALTWHTSAYGPDQRAQEGPAKKVLTLEGDLVLGGLFPVHQKGGPAEDCGPVNEHRGIQR
LEAMLFALDRINRDPHLLPGVRLGAHILDSCSKDTHALEQALDFVRASLSRGADGSRHICPDGSYATHGD
APTAITGVIGGSYSDVSIQVANLLRLFQIPQISYASTSAKLSDKSRYDYFARTVPPDFFQAKAMAEILRF
FNWTYVSTVASEGDYGETGIEAFELEARARNICVATSEKVGRAMSRAAFEGVVRALLQKPSARVAVLFTR
SEDARELLAASQRLNASFTWVASDGWGALESVVAGSEGAAEGAITIELASYPISDFASYFQSLDPWNNSR
NPWFREFWEQRFRCSFRQRDCAAHSLRAVPFEQESKIMFVVNAVYAMAHALHNMHRALCPNTTRLCDAMR
PVNGRRLYKDFVLNVKFDAPFRPADTHNEVRFDRFGDGIGRYNIFTYLRAGSGRYRYQKVGYWAEGLTLD
TSLIPWASPSAGPLPASRCSEPCLQNEVKSVQPGEVCCWLCIPCQPYEYRLDEFTCADCGLGYWPNASLT
GCFELPQEYIRWGDAWAVGPVTIACLGALATLFVLGVFVRHNATPVVKASGRELCYILLGGVFLCYCMTF
IFIAKPSTAVCTLRRLGLGTAFSVCYSALLTKTNRIARIFGGAREGAQRPRFISPASQVATCLALISGQL
LIVVAWLVVEAPGTGKETAPERREVVTLRCNHRDASMLGSLAYNVLLIALCTLYAFKTRKCPENFNEAKF
IGFTMYTTCIIWLAFLPIFYVTSSDYRVQTTTMCVSVSLSGSVVLGCLFAPKLHIILFQPQKNVVSHRAP
TSRFGSAAARASSSLGQGSGSQFVPTVCNGREVVDSTTSSL

TABLE 21
CaSPhmGluR6(27-35) nucleic acid sequence (SEQ ID NO 21)

atggcattttatagctgctgctgggtcctcttggcactcacctggcacacctctgcctacgggccagacc
agcgagcccaactggcgggcggcctgacgctgggcggcctgttcccggtgcacgcgcggggcgcggcggg
ccgggcgtgcgggccgctgaagaaggagcagggcgtgcaccggctggaggccatgctgtacgcgctggac
cgcgtcaacgccgaccccgagctgctgcccggcgtgcgcctgggcgcgcggctgctggacacctgctcgc
gggacacctacgcgctggagcaggcgctgagcttcgtgcaggcgctgatccgtggccgcggcgacggcga
cgaggtgggcgtgcgctgcccgggaggcgtccctccgctgcgccccgcgccccccgagcgcgtcgtggcc
gtcgtgggcgcctcggccagctccgtctccatcatggtcgccaacgtgctgcgcctgtttgcgatacccc
agatcagctatgcctccacagccccggagctcagcgactccacacgctatgacttcttctcccgggtggt
gccacccgactcctaccaggcgcaggccatggtggacatcgtgagggcactgggatggaactatgtgtcc
acgctggcctccgagggcaactatggcgaaagtggggttgaggccttcgttcagatctcccgagaggctg
ggggggtctgtattgcccagtctatcaagattcccagggaaccaaagccaggagagttcagcaaggtgat
caggagactcatggagacgcccaacgcccggggcatcatcatctttgccaatgaggatgacatcaggcgg
gtcctggaggcagctcgccaggccaacctgaccggccacttcctgtgggtcggctcagacagctggggag
ccaagacctcacccatcttgagcctggaggacgtggccgttggggccatcaccatcctgcccaaaagggc
ctccatcgacggatttgaccagtacttcatgactcgatccctggagaacaaccgcaggaacatctggttc
gccgagttctgggaagagaattttaactgcaaactgaccagctcaggtacccagtcagatgattccaccc
gcaaatgcacaggcgaggaacgcatcggccgggactccacctacgagcaggagggcaaggtgcagtttgt
gattgatgcggtgtatgccattgcccacgccctccacagcatgcaccaggcgctctgccctgggcacaca
ggcctgtgcccggcgatggaacccaccgatgggcggatgcttctgcagtacattcgagctgtccgcttca
acggcagcgcaggaacccctgtgatgttcaacgagaacggggatgcgcccgggcggtacgacatcttcca
gtaccaggcgaccaatggcagtgccagcagtggcgggtaccaggcagtgggccagtgggcagagaccctc
agactggatgtggaggccctgcagtggtctggcgacccccacgaggtgccctcgtctctgtgcagcctgc
cctgcgggccgggggagcggaagaagatggtgaagggcgtcccctgctgttggcactgcgaggcctgtga
cgggtaccgcttccaggtggacgagttcacatgcgaggcctgtcctggggacatgaggcccacgcccaac
cacacgggctgccgccccacacctgtggtgcgcctgagctggtcctccccctgggcagccccgccgctcc
tcctggccgtgctgggcatcgtggccactaccacggtggtggccaccttcgtgcggtacaacaacacgcc
catcgtccgggcctcgggccgagagctcagctacgtcctcctcaccggcatcttcctcatctacgccatc
accttcctcatggtggctgagcctggggccgcggtctgtgccgcccgcaggctcttcctgggcctgggca
cgaccctcagctactctgccctgctcaccaagaccaaccgtatctaccgcatctttgagcagggcaagcg
ctcggtcacaccccctcccttcatcagccccacctcacagctggtcatcaccttcagcctcacctccctg
caggtggtggggatgatagcatggctgggggcccggcccccacacagcgtgattgactatgaggaacagc
ggacggtggaccccgagcaggccagaggggtgctcaagtgcgacatgtcggatctgtctctcatcggctg
cctgggctacagcctcctgctcatggtcacgtgcacagtgtacgccatcaaggcccgtggcgtgcccgag
accttcaacgaggccaagcccatcggcttcaccatgtacaccacctgcatcatctggctggcattcgtgc
ccatcttctttggcactgcccagtcagctgaaaagatctacatccagacaaccacgctaaccgtgtcctt
gagcctgagtgcctcggtgtccctcggcatgctctacgtacccaaaacctacgtcatcctcttccatcca
gagcagaatgtgcagaagcgaaagcggagcctcaaggccacctccacggtggcagccccacccaagggcg
aggatgcagaggcccacaag

TABLE 22
CaSPhmGluR6(27-35) amino acid sequence (SEQ ID NO 22)

MAFYSCCWVLLALTWHTSAYGPDQRAQLAGGLTLGGLFPVHARGAAGRACGPLKKEQGVHRLEAMLYALD
RVNADPELLPGVRLGARLLDTCSRDTYALEQALSFVQALIRGRGDGDEVGVRCPGGVPPLRPAPPERVVA
VVGASASSVSIMVANVLRLFAIPQISYASTAPELSDSTRYDFFSRVVPPDSYQAQAVDIVRALGWNYVS
TLASEGNYGESGVEAFVQISREAGGVCIAQSIKIPREPKPGEFSKVIRRLMETPNARGIIIFANEDDIRR
VLEAARQANLTGHFLWVGSDSWGAKTSPILSLEDVAVGAITILPKRASIDGFDQYFMTRSLENNRRNIWF
AEFWEENFNCKLTSSGTQSDDSTRKCTGEERIGRDSTYEQEGKVQFVIDAVYAIAHALHSMHQALCPGHT
GLCPAMEPTDGRMLLQYIRAVRFNGSAGTPVMFNENGDAPGRYDIFQYQATNGSASSGGYQAVGQEAETL
RLDVEALQWSGDPHEVPSSLCSLPCGPGERKKMVKGVPCCWHCEACDGYRFQVDEFTCEACPGDMRPTPN
HTGCRPTPVVRLSWSSPWAAPPLLLAVLGIVATTTVVATFVRYNNTPIVRASGRELSYVLLTGIFLIYAI
TFLMVAEPGAAVCAARRLFLGLGTTLSYSALLTKTNRIYRIFEQGKRSVTPPPFISPTSQLVITFSLTSL
QVVGMIAWLGARPPHSVIDYEEQRTVDPEQARGVLKCDMSDLSLIGCLGYSLLLMVTCTVYAIKARGVPE
TFNEAKPIGFTMYTTCIIWLAFVPIFFGTAQSAEKIYIQTTTLTVSLSLSASVSLGMLYVPKTYVILFHP
EQNVQKRKRSLKATSTVAAPPKGEDAEAHK

TABLE 23
CaSPhmGluR5(27-22) nucleic acid sequence (SEQ ID NO 23)

atggcattttatagctgctgctgggtcctcttggcactcacctggcacacctctgcctacgggccagacc
agcgagcccaatccagtgagaggagggtggtggctcacatgccgggtgacatcattattggagctctctt
ttctgttcatcaccagcctactgtggacaaagttcatgagaggaagtgtggggcggtccgtgaacagtat
ggcattcagagagtggaggccatgctgcataccctggaaaggatcaattcagaccccacactcttgccca
acatcacactgggctgtgagataagggactcctgctggcattcggctgtggccctagagcagagcattga
gttcataagagattccctcatttcttcagaagaggaagaaggcttggtacgctgtgtggatggctcctcc
tcttccttccgctccaagaagcccatagtaggggtcattgggcctggctccagttctgtagccattcagg
tccagaatttgctccagcttttcaacatacctcagattgcttactcagcaaccagcatggatctgagtga
caagactctgttcaaatatttcatgagggttgtgccttcagatgctcagcaggcaagggccatggtggac
atagtgaagaggtacaactggacctatgtatcagccgtgcacacagaaggcaactatggagaaagtggga
tggaagccttcaaagatatgtcagcgaaggaagggatttgcatcgcccactcttacaaaatctacagtaa
tgcaggggagcagagctttgataagctgctgaagaagctcacaagtcacttgcccaaggcccgggtggtg
gcctgcttctgtgagggcatgacggtgagaggtctgctgatggccatgaggcgcctgggtctagcgggag
aatttctgcttctgggcagtgatggctgggctgacaggtatgatgtgacagatggatatcagcgagaagc
tgttggtggcatcacaatcaagctccaatctcccgatgtcaagtggtttgatgattattatctgaagctc
cggccagaaacaaaccaccgaaacccttggtttcaagaattttggcagcatcgttttcagtgccgactgg
aagggtttccacaggagaacagcaaatacaacaagacttgcaatagttctctgactctgaaaacacatca
tgttcaggattccaaaatgggatttgtgatcaacgccatctattcgatggcctatgggctccacaacatg
cagatgtccctctgcccaggctatgcaggactctgtgatgccatgaagccaattgatggacggaaacttt
tggagtccctgatgaaaaccaattttactggggtttctggagatacgatcctattcgatgagaatggaga
ctctccaggaaggtatgaaataatgaatttcaaggaaatgggaaaagattactttgattatatcaacgtt
ggaagttgggacaatggagaattaaaaatggatgatgatgaagtatggtccaagaaaagcaacatcatca
gatctgtgtgcagtgaaccatgtgagaaaggccagatcaaggtgatccgaaagggagaagtcagctgttg
ttggacctgtacaccttgtaaggagaatgagtatgtctttgatgagtacacatgcaaggcatgccaactg
gggtcttggcccactgatgatctcacaggttgtgacttgatcccagtacagtatcttcgatggggtgacc
ctgaacccattgcagctgtggtgtttgcctgccttggcctcctggccaccctgtttgttactgtagtctt
catcatttaccgtgatacaccagtagtcaagtcctcaagcagggaactctgctacattatccttgctggc
atctgcctgggctacttatgtaccttctgcctcattgcgaagcccaaacagatttactgctaccttcaga
gaattggcattggtctctccccagccatgagctactcagcccttgtaacaaagaccaaccgtattgcaag
gatcctggctggcagcaagaagaagatctgtaccaaaaagcccagattcatgagtgcctgtgcccagcta
gtgattgctttcattctcatatgcatccagttgggcatcatcgttgccctctttataatggagcctcctg
acataatgcatgactacccaagcattcgagaagtctacctgatctgtaacaccaccaacctaggagttgt
cactccacttggatacaatggattgttgattttgagctgcaccttctatgcgttcaagaccagaaatgtt
ccagctaacttcaacgaggccaagtatatcgccttcacaatgtacacgacctgcattatatggctagctt
ttgtgccaatctactttggcagcaactacaaaatcatcaccatgtgtttctcggtcagcctcagtgccac
agtggccctaggctgcatgtttgtgccgaaggtgtacatcatcctggccaaaccagagagaaacgtgcgc
agcgccttcaccacatctaccgtggtgcgcatgcatgtaggggatggcaagtcatcctccgcagccagca
gatccagcagcctagtcaacctgtggaagagaaggggctcctctggggaaaccttaaggtacaaagacag
gagactggcccagcacaagtcggaaatagagtgtttcacccccaaagggagtatggggaatggtgggaga
gcaacaatgagcagttccaatggaaaatccgtcacgtgggcccagaatgagaagagcagccgggggcagc
acctgtggcagcgcctgtccatccacatcaacaagaaagaaaaccccaaccaaacggccgtcatcaagcc
cttccccaagagcacggagagccgtggcctgggcgctggcgctggcgcaggcgggagcgctgggggcgtg
ggggccacgggcggtgcgggctgcgcaggcgccggcccaggcgggcccgagtccccagacgccggcccca
aggcgctgtatgatgtggccgaggctgaggagcacttcccggcgcccgcgcggccgcgctcaccgtcgcc
catcagcacgctgagccaccgcgcgggctcggccagccgcacggacgacgatgtgccgtcgctgcactcg
gagcctgtggcgcgcagcagctcctcgcagggctccctcatggagcagatcagcagtgtggtcacccgct
tcacggccaacatcagcgagctcaactccatgatgctgtccaccgcggcccccagccccggcgtcggcgc
cccgctctgctcgtcctacctgatccccaaagagatccagttgcccacgaccatgacgacctttgccgaa
atccagcctctgccggccatcgaagtcacgggcggcgcgcagcccgcggcaggggcgcaggcggctgggg
acgcggcccgggagagccccgcggccggtcccgaggctgcggccgccaagccagacctggaggagctggt
ggctctcaccccgccgtcccccttcagagactcggtggactcggggagcacaacccccaactcaccagtg
tccgagtcggccctctgtatcccgtcgtctcccaaatatgacactcttatcataagagattacactcaga
gctcctcgtcgttg

TABLE 24
CaSPhmGluR5(27-22) amino acid sequence (SEQ ID NO 24)

MAFYSCCWVLLALTWHTSAYGPDQRAQSSERRVVAHMPGDIIIGALFSVHHQPTVDKVHERKCGAVREQY
GIQRVEAMLHTLERINSDPTLLPNITLGCEIRDSCWHSAVALEQSIEFIRDSLISSEEEEGLVRCVDGSS
SSFRSKKPIVGVIGPGSSSVAIQVQNLLQLFNIPQIAYSATSMDLSDKTLFKYFMRVVPSDAQQARAMVD
IVKRYNWTYVSAVHTEGNYGESGMEAFKDMSAKEGICIAHSYKIYSNAGEQSFDKLLKKLTSHLPKARVV
ACFCEGMTVRGLLMAMRRLGLAGEFLLLGSDGWADRYDVTDGYQREAVGGITIKLQSPDVKWFDDYYLKL
RPETNHRNPWFQEFWQHRFQCRLEGFPQENSKYNKTCNSSLTLKTHHVQDSKMGFVINAIYSMAYGLHNM
QMSLCPGYAGLCDAMKPIDGRKLLESLMKTNFTGVSGDTILFDENGDSPGRYEIMNFKEMGKDYFDYINV
GSWDNGELKMDDDEVWSKKSNIIRSVCSEPCEKGQIKVIRKGEVSCCWTCTPCKENEYVFDEYTCKACQL
GSWPTDDLTGCDLIPVQYLRWGDPEPIAAVVFACLGLLATLFVTVVFIIYRDTPVVKSSSRELCYIILAG
ICLGYLCTFCLIAKPKQIYCYLQRIGIGLSPAMSYSALVTKTNRIARILAGSKKKICTKKPRFMSACAQL
VIAFILICIQLGIIVALFIMEPPDIMHDYPSIREVYLICNTTNLGVVTPLGYNGLLILSCTFYAFKTRNV
PANFNEAKYIAFTMYTTCIIWLAFVPIYFGSNYKIITMCFSVSLSATVALGCMFVPKVYIILAKPERNVR
SAFTTSTVVRMHVGDGKSSSAASRSSSLVNLWKRRGSSGETLRYKDRRLAQHKSEIECFTPKGSMGNGGR
ATMSSSNGKSVTWAQNEKSSRGQHLWQRLSIHINKKENPNQTAVIKPFPKSTESRGLGAGAGAGGSAGGV
GATGGAGCAGAGPGGPESPDAGPKALYDVAEAEEHFPAPARPRSPSPISTLSHRAGSASRTDDDVPSLHS
EPVARSSSSQGSLMEQISSVVTRFTANISELNSMMLSTAAPSPGVGAPLCSSYLTPKEIQLPTTMTTFAE
IQPLPAIEVTGGAQPAAGAQAAGDAARESPAAGPEAAAAKPDLEELVALTPPSPFRDSVDSGSTTPNSPV
SESALCIPSSPKYDTLIIRDYTQSSSSL

TABLE 25

Nucleotide sequence (SEQ ID NO:25) and corresponding amino
acid sequence (SEQ ID NO:26) of pmGluR1/CaR

```
Sequence Range: −7 to 3379
                    3 13 23 33
        * * * * * * * * *
CGCCACA ATG GTC CGG CTC CTC TTG ATT TTC TTC CCA ATG ATC TTT TTG
                Met Val Arg Leu Leu Leu Ile Phe Phe Pro Met Ile Phe Leu>
                    b CODING SEQUENCE CHIMERA:JUNCTION NUC.1776 b >
              a a a −8 TO 1775 OF MCRATMGL-1 30 a a a40 >
43 53 63 73 83
  * * * * * * * * * *
  GAG ATG TCC ATT TTG CCC AGG ATG CCT GAC AGA AAA GTA TTG CTG GCA
  Glu Met Ser Ile Leu Pro Arg Met Pro Asp Arg Lys Val Leu Leu Ala>
        b b CODING SEQUENCE CHIMERA:JUNCTION NUC.1776 b b >
        a a 50a a −8 TO 1775 OF MCRATMGL-1 a 80a a a >
  93 103 113 123 133
      * * * * * * * * * *
  GGT GCC TCG TCC CAG CGC TCC GTG GCG AGA ATG GAC GGA GAT GTC ATC
  Gly Ala Ser Ser Gln Arg Ser Val Ala Arg Met Asp Gly Asp Val Ile>
      b b CODING SEQUENCE CHIMERA:JUNCTION NUC.1776 b b >
90 a a 100 a −8 TO 1175 OF MCRATMGL-1 a a 130 a a >
        143 153 163 173 183
            * * * * * * * * *
  ATC GGA GCC CTC TTC TCA GTC CAT CAC CAG CCT CCA GGC GAG AAG GTA
  Ile Gly Ala Leu Phe Ser Val His His Gln Pro Pro Ala Glu Lys Val>
        b b CODING SEQUENCE CHIMERA:JUNCTION NUC.1776 b b >
  140a a a 150 −8 TO 1775 OF MCRATMGL-1 a a 180 a >
            193 203 213 223 233
    * * * * * * * * * *
  CCC GAA AGG AAG TGT GGG GAG ATC AGG GAA CAG TAT GGT ATC CAG AGG
  Pro Glu Arg Lys Cys Gly Glu Ile Arg Glu Gln Tyr Gly Ile Gln Arg>
        b b CODING SEQUENCE CHIMERA:JUNCTION NUC.1776 b b >
        190 a a a20 −8 TO 1775 OF MCRATMGL-1 20 a a a230a >
                  243 253 263 273
          * * * * * * * * *
  GTG GAG CCC ATG TTC CAC ACG TTG GAT AAG ATT AAC GCG GAC CCG GTG
  Val Glu Ala Met Phe His Thr Leu Asp Lys Ile Asn Ala Asp Pro Val>
    b b CODING SEQUENCE CHIMERA:JUNCTION NUC.1776 b b >
    a 240 a a −8 TO 1775 OF MCRATMGL-1 270 a a 280 >
283 293 303 313 323
    * * * * * * * * * *
    CTC CTG CCC AAC ATC ACT CTG GGC AGT GAG ATC CGG GAC TCC TGC TGG
    Leu Leu Pro Asn Ile Thr Leu Gly Ser Glu Ile Arg Asp Ser Cys Trp>
          b b CODING SEQUENCE CHIMERA:JUNCTION NUC.1776 b b >
          a a290a a −8 TO 1775 OF MCRATMGL-1 a320a a a >
    333 343 353 363 373
        * * * * * * * * * *
    CAC TCT TCA GTG GCT CTC GAA CAG AGC ATC GAA TTC ATC AGA GAC TCC
    His Ser Ser Val Ala Leu Glu Gln Ser Ile Glu Phe Ile Arg Asp Ser>
        b b CODING SEQUENCE CHIMERA:JUNCTION NUC.1776 b b >
330 a a 340 a −8 TO 1775 OF MCRATMGL-1 a a 370 a a >
          383 393 403 413 423
              * * * * * * * * *
    CTG ATT TCC ATC CGA GAT GAG AAG GAT GGG CTG AAC CGA TGC CTG CCT
    Leu Ile Ser Ile Arg Asp Glu Lys Asp Gly Leu Asn Arg Cys Leu Pro>
          b b CODING SEQUENCE CHIMERA:JUNCTION NUC.1776 b b >
    380a a a 390 −8 TO 1775 OF MCRATMGL-1 a a 420 a >
                433 443 453 463 473
      * * * * * * * * * *
    GAT GGC CAG ACC CTG CCC CCT GGC AGG ACT AAG AAG CCT ATT GCT GGA
    Asp Gly Gln Thr Leu Pro Pro Gly Arg Thr Lys Lys Pro Ile Ala Gly>
      b b CODING SEQUENCE CHIMERA:JUNCTION NUC.1776 b b >
            430 a a a44 −8 TO 1775 OF MCRATMGL-1 60 a a a470a >
                    483 493 503 513
            * * * * * * * * *
    GTG ATC GGC CCT GGC TCC AGC TCT GTG GCC ATT CAA GTC CAG AAT CTT
    Val Ile Gly Pro Gly Ser Ser Ser Val Ala Ile Gln Val Gln Asn Leu>
          b b CODING SEQUENCE CHIMERA:JUNCTION NUC.1776 b b >
          a 480 a a −8 TO 1775 OF MCRATMGL-1 510 a a 520 >
523 533 543 553 563
    CTC CAG CTG TTC GAC ATC CCA CAG ATC GCC TAT TCT GCC ACA AGC ATA
    Leu Gln Leu Phe Asp Ile Pro Gln Ile Ala Tyr Ser Ala Thr Ser Ile>
          b b CODING SEQUENCE CHIMERA:JUNCTION NUC.1776 b b >
          a a530a a −8 TO 1775 OF MCRATMGL-1 a560a a a >
    573 583 593 603 613
        * * * * * * * * * *
    GAC CTG AGT GAC AAA ACT TTG TAC AAA TAC TTC CTG AGG GTG GTC CCT
    Asp Leu Ser Asp Lys Thr Leu Tyr Lys Tyr Phe Leu Arg Val Val Pro>
          b b CODING SEQUENCE CHIMERA:JUNCTION NUC.1776 b b >
570 a a 580 a −8 TO 1775 OF MCRATMGL-1 a a 610 a a >
          623 633 643 653 663
              * * * * * * * * *
    TCT GAC ACT TTG CAG GCA AGG GCG ATG CTC GAC ATA GTC AAG CGT TAC
    Ser Asp Thr Leu Gln Ala Arg Ala Met Leu Asp Ile Val Lys Arg Tyr>
        b b CODING SEQUENCE CHIMERA:JUNCTION NUC.1776 b b >
    620a a a 630 −8 TO 1775 OF MCRATMGL-1 a a 660 a >
                673 683 693 703 713
      * * * * * * * * * *
    AAC TGG ACC TAT GTC TCA GCA GTC CAC ACA GAA GGG AAT TAC GGC GAG
    Asn Trp Thr Tyr Val Ser Ala Val His Thr Glu Gly Asn Tyr Cly Glu>
          b b CODING SEQUENCE CHIMERA:JUNCTION NUC.1776 b b >
          670 a a a68 −8 TO 1775 OF MCRATMGL-1 00 a a a710a >
                    723 733 743 753
            * * * * * * * * *
    AGT GGA ATG GAT GCT TTC AAA GAA CTG GCT GCC CAG GAA GGC CTC TGC
    Ser Gly Met Asp Ala Phe Lys Glu Leu Ala Ala Gln Glu Gly Leu Cys>
          b b CODING SEQUENCE CHIMERA:JUNCTION NUC.1776 b b >
          a 720 a a −8 TO 1775 OF MCRATMGL-1 750 a a 760 >
763 773 783 793 803
    * * * * * * * * * *
    ATC GCA CAC TCG GAC AAA ATC TAC AGC AAT GCT GGC GAG AAG AGC TTT
    Ile Ala His Ser Asp Lys Ile Tyr Ser Asn Ala Gly Glu Lys Ser Phe>
          b b CODING SEQUENCE CHIMERA:JUNCTION NUC.1776 b b >
          a a770a a −8 TO 1775 OF MCRATMGL-1 a800a a a >
    813 823 833 843 853
        * * * * * * * * * *
    GAC CGG CTC CTG CGT AAA CTC CGG GAG CGG CTT CCC AAG GCC AGG GTT
    Asp Arg Leu Leu Arg Lys Leu Arg Glu Arg Leu Pro Lys Ala Arg Val>
          b b CODING SEQUENCE CHIMERA:JUNCTION NUC.1776 b b >
810 a a 820 a −8 TO 1775 OF MCRATMGL-1 a a 850 a a >
          863 873 883 893 903
              * * * * * * * * *
    GTG GTC TGC TTC TGC GAG GGC ATG ACA GTG CGG GGC TTA CTG AGT GCC
    Val Val Cys Phe Cys Glu Gly Met Thr Val Arg Gly Leu Leu Ser Ala>
          b b CODING SEQUENCE CHIMERA:JUNCTION NUC.1776 b b >
    860a a a 870 −8 TO 1775 OF MCRATMGL-1 a a 900 a >
                913 923 933 943 953
      * * * * * * * * * *
    ATG CGC CGC CTG GGC GTC GTG GGC GAG TTC TCA CTC ATT GGA AGT GAT
    Met Arg Arg Leu Gly Val Val Gly Glu Phe Ser Leu Ile Gly Ser Asp>
          b b CODING SEQUENCE CHIMERA:JUNCTION NUC.1776 b b >
          910 a a a92 −8 TO 1775 OF MCRATMGL-1 40 a a a950a >
                    963 973 983 993
            * * * * * * * * *
    GGA TGG GCA GAC AGA GAT GAA GTC ATC GAA GGC TAT GAG GTG GAA GCC
    Gly Trp Ala Asp Arg Asp Glu Val Ile Glu Gly Tyr Glu Val Glu Ala>
          b b CODING SEQUENCE CHIMERA:JUNCTION NUC.1776 b b >
          a 960 a a −8 TO 1775 OF MCRATMGL-1 990 a a 1000 >
1003 1013 1023 1033 1043
      * * * * * * * * * *
      AAC GGA GGG ATC ACA ATA AAG CTT CAG TCT CCA GAG GTC AGG TCA TTT
    Asn Gly Gly Ile Thr Ile Lys Leu Gln Ser Pro Glu Val Arg Ser Phe>
            b b CODING SEQUENCE CHIMERA:JUNCTION NUC.1776 b b >
            a 1010a a −8 TO 1775 OF MCRATMGL-1 1040a a a >
    1053 1063 1073 1083 1093
          * * * * * * * * * *
      GAT GAC TAC TTC CTG AAG CTG AGG CTG GAC ACC AAC ACA AGG AAT CCT
      Asp Asp Tyr Phe Leu Lys Leu Arg Leu Asp Thr Asn Thr Arg Asn Pro>
          b b CODING SEQUENCE CHIMERA:JUNCTION NUC.1776 b b >
1050 a a 1060 a −8 TO 1775 OF MCRATMGL-1 a a 1090 a a >
          1103 1113 1123 1133 1143
                * * * * * * * * *
      TGG TTC CCT GAG TTC TGG CAA CAT CGC TTC CAG TGT CGC CTA CCT GGA
      Trp Phe Pro Glu Phe Trp Gln His Arg Phe Gln Cys Arg Leu Pro Gly>
          b b CODING SEQUENCE CHIMERA:JUNCTION NUC.1776 b b >
    1100a a a 1110 −8 TO 1775 OF MCRATMGL-1 a a a 1140 a >
                1153 1163 1173 1183 1193
        * * * * * * * * * *
      CAC CTC TTG GAA AAC CCC AAC TTT AAG AAA GTG TGC ACA GGA AAT GAA
His Leu Leu Glu Asn Pro Asn Phe Lys Lys Val Cys Thr Gly Asn Glu
            b b CODING SEQUENCE CHIMERA:JUNCTION NUC.1776 b b >
          1150 a a 116 −8 TO 1775 OF MCRATMGL-1 80 a a 1190a >
                    1203 1213 1223 1233
              * * * * * * * * *
      AGC TTG GAA GAA AAC TAT GTC CAG GAC AGC AAA ATG GGA TTT GTC ATC
      Ser Leu Glu Glu Asn Tyr Val Gln Asp Ser Lys Met Gly Phe Val Ile>
            b b CODING SEQUENCE CHIMERA:JUNCTION NUC.1776 b b >
            a 1200 a a −8 TO 1775 OF MCRATMGL-1 1230 a a 1240 >
1243 1253 1263 1273 1283
      * * * * * * * * * *
      AAT GCC ATC TAT GCC ATG GCA CAT GGG CTG CAG AAC ATG CAC CAT GCT
      Asn Ala Ile Tyr Ala Met Ala His Gly Leu Gln Asn Met His His Ala>
          b b CODING SEQUENCE CHIMERA:JUNCTION NUC.1776 b b >
          a 1250a a −8 TO 1775 OF MCRATMGL-1 1280a a a >
    1293 1303 1313 1323 1333
          * * * * * * * * * *
      CTG TGT CCC GGC CAT GTG GGC CTG TGT GAT GCT ATG AAA CCC ATT GAT
      Leu Cys Pro Gly His Val Gly Leu Cys Asp Ala Met Lys Pro Ile Asp>
          b b CODING SEQUENCE CHIMERA:JUNCTION NUC.1776 b b >
1290 a a 1300 a −8 TO 1775 OF MCRATMGL-1 a a 1330 a a >
            1343 1353 1363 1373 1383
                  * * * * * * * * *
      GGC AGG AAG CTC CTG GAT TTC CTC ATC AAA TCC TCT TTT GTC GGA GTG
      Gly Arg Lys Leu Leu Asp Phe Leu Ile Lys Ser Ser Phe Val Gly Val>
            b b CODING SEQUENCE CHIMERA:JUNCTION NUC.1776 b b >
    1340a a a 1.350 −8 TO 1775 OF MCRATMGL-1 a a a 1380 a >
                1393 1403 1413 1423 1433
        * * * * * * * * * *
      TCT GGA GAG GAG GTG TGG TTC GAT GAG AAG GGG GAT GCT CCC GGA AGG
      Ser Gly Glu Glu Val Trp Phe Asp Glu Lys Gly Asp Ala Pro Gly Arg>
            b b CODING SEQUENCE CHIMERA:JUNCTION NUC.1776 b b >
          1390 a a 140 −8 TO 1775 OF MCRATMGL-1 20 a a 1430a >
                    1443 1453 1463 1473
              * * * * * * * * *
      TAT GAC ATT ATG AAT CTG CAG TAC ACA GAA GCT AAT CGC TAT GAC TAT
      Tyr Asp Ile Met Asn Leu Gln Tyr Thr Glu Ala Asn Arg Tyr Asp Tyr>
            b b CODING SEQUENCE CHIMERA:JUNCTION NUC.1776 b b >
            a 1440 a a −8 TO 1775 OF MCRATMGL-1 1470 a a 1480 >
1483 1493 1503 1513 1523
      * * * * * * * * * *
      GTC CAC GTG GGG ACC TGG CAT GAA GGA GTG CTG AAT ATT GAT GAT TAC
      Val His Val Gly Thr Trp His Glu Gly Val Leu Asn Ile Asp Asp Tyr>
            b b CODING SEQUENCE CHIMERA:JUNCTION NUC.1776 b b >
            a 1490a a −8 TO 1775 OF MCRATMGL-1 1520a a a >
    1533 1543 1553 1563 1573
          * * * * * * * * * *
      AAA ATC CAG ATG AAC AAA AGC GGA ATG GTA CGA TCT GTG TGC AGT GAG
      Lys Ile Gln Met Asn Lys Ser Gly Met Val Arg Ser Val Cys Ser Glu>
            b b CODING SEQUENCE CHIMERA:JUNCTION NUC.1776 b b >
1530 a a 1540 a −8 TO 1775 OF MCRATMGL-1 a a 1570 a a >
          1583 1593 1603 1613 1623
                * * * * * * * * *
      CCT TGC TTA AAG GGT CAG ATT AAG GTC ATA CGG AAA GGA GAA GTG AGC
      Pro Cys Leu Lys Gly Gln Ile Lys Val Ile Arg Lys Gly Glu Val Ser>
            b b CODING SEQUENCE CHIMERA:JUNCTION NUC.1776 b b >
    1580a a a 1590 −8 TO 1775 OF MCRATMGL-1 a a a 1620 a >
                1633 1643 1653 1663 1673
        * * * * * * * * * *
      TGC TGC TGG ATC TGC ACG GCC TGC AAA GAG AAT GAG TTT GTG CAG GAC
      Cys Cys Trp Ile Cys Thr Ala Cys Lys Glu Asn Glu Phe Val Gln Asp>
            b b CODING SEQUENCE CHIMERA:JUNCTION NUC.1776 b b >
          1630 a a 164 −8 TO 1775 OF MCRATMGL-1 60 a a 1670a >
                  1683 1693 1703 1713
              * * * * * * * * *
      GAG TTC ACC TGC AGA GCC TGT GAC CTG GGG TGG TGG CCC AAC GCA GAG
      Glu Phe Thr Cys Arg Ala Cys Asp Leu Gly Trp Trp Pro Asn Ala Glu>
            b b CODING SEQUENCE CHIMERA:JUNCTION NUC.1776 b b >
            a 1680 a a −8 TO 1775 OF MCRATMGL-1 1710 a a 1720 >
1723 1733 1743 1753 1763
      * * * * * * * * * *
      CTC ACA GGC TGT GAG CCC ATT CCT GTC CGT TAT CTT GAG TGG AGT GAC
      Leu Thr Gly Cys Glu Pro Ile Pro Val Arg Tyr Leu Glu Trp Ser Asp>
            b b CODING SEQUENCE CHIMERA:JUNCTION NUC.1776 b b >
            a 1730a a −8 TO 1775 OF MCRATMGL-1 1760a a a >
    1773 1783 1793 1803 1813
          * * * * * * * * * *
      ATA GAA GGG ATC GCA CTC ACC CTC TTT GCC GTG CTG GGC ATT TTC CTG
      Ile Glu Gly Ile Ala Leu Thr Leu Phe Ala Val Leu Gly Ile Phe Leu>
            b b CODING SEQUENCE CHIMERA:JUNCTION NUC.1776 b b >
1770 a >
                        1840 c 1837 TO 3437 OF MCPHUPCAR4.0 FINAL c c >
          1823 1833 1843 1853 1863
                * * * * * * * * *
    ACA GCC TTT GTG CTG GGT GTG TTT ATC AAG TTC CGC AAC ACA CCC ATT
    Thr Ala Phe Val Leu Gly Val Phe Ile Lys Phe Arg Asn Thr Pro Ile>
          b b CODING SEQUENCE CHIMERA:JUNCTION NUC.1776 b b >
1880 c c 1 1837 TO 3437 OF MCPHUPCAR4.0 FINAL 192Cc c >
              1873  1883 1893 1903 1913
      * * * * * * * * * *
    GTC AAG GCC ACC AAC CGA GAG CTC TCC TAC CTC CTC CTC TTC TCC CTG
    Val Lys Ala Thr Asn Arg Glu Leu Ser Tyr Leu Leu Leu Phe Ser Leu>
          b b CODING SEQUENCE CHIMERA:JUNCTION NUC.1776 b b >
      1930 c c 1837 TO 3437 OF MCPHUPCAR4.0 FINAL c 1970 c >
                    1923 1933 1943 1953
              * * * * * * * * *
      CTC TGC TGC TTC TCC AGC TCC CTG TTC TTC ATC GGG GAG CCC CAG GAC
      Leu Cys Cys Phe Ser Ser Ser Leu Phe Phe Ile Gly Glu Pro Gln Asp>
            b b CODING SEQUENCE CHIMERA:JUNCTION NUC.1776 b b >
            1980c c 1837 TO 3437 OF MCPHUPCAR4.0 FINAL c c 2020 >
1963 1973 1983 1993 2003
      * * * * * * * * * *
      TGG ACG TGC CGC CTG CGC CAG CCG GCC TTT GGC ATC AGC TTC GTG CTC
      Trp Thr Cys Arg Leu Arg Gln Pro Ala Phe Gly Ile Ser Phe Val Leu>
            b b CODING SEQUENCE CHIMERA:JUNCTION NUC.1776 b b >
            c 2030 c 1837 TO 3437 OF MCPHUPCAR4.0 FINAL 0 c c 2070>
    2013 2023 2033 2043 2053
          * * * * * * * * * *
      TGC ATC TCA TGC ATC CTG GTG AAA ACC AAC CGT GTC CTC CTG GTG TTT
      Cys Ile Ser Cys Ile Leu Val Lys Thr Asn Arg Val Leu Leu Val Phe>
            b b CODING SEQUENCE CHIMERA:JUNCTION NUC.1776 b b >
            c c 2080 1837 TO 3437 OF MCPHUPCAR4.0 FINAL 2110  c c >
          2063 2073 2083 2093 2103
                * * * * * * * * *
      GAG GCC AAG ATC CCC ACC AGC TTC CAC CGC AAG TGG TGG GGG CTC AAC
      Glu Ala Lys Ile Pro Thr Ser Phe His Arg Lys Trp Trp Gly Leu Asn>
            b b CODING SEQUENCE CHIMERA:JUNCTION NUC.1776 b b >
2120 c c 2 1837 TO 3437 OF MCPHUPCAR4.0 FINAL 2160c c >
                2113 2123 2133 2143 2153
        * * * * * * * * * *
      CTG CAG TTC CTG CTG GTT TTC CTC TGC ACC TTC ATG CAG ATT GTC ATC
      Leu Gln Phe Leu Leu Val Phe Leu Cys Thr Phe Met Gln Ile Val Ile>
          b b CODING SEQUENCE CHIMERA:JUNCTION NUC.1776 b b >
        2170 c c 1837 TO 3437 OF MCPHUPCAR4.0 FINAL c 2210 c >
                    2163 2173 2183 2193
              * * * * * * * * *
      TGT GTG ATC TGG CTC TAC ACC GCG CCC CCC TCA AGC TAC CGC AAC CAG
      Cys Val Ile Trp Leu Tyr Thr Ala Pro Pro Ser Ser Tyr Arg Asn Gln>
            b b CODING SEQUENCE CHIMERA:JUNCTION NUC.1776 b b >
            2220c c 1837 TO 3437 OF MCPHUPCAR4.0 FINAL c c 2260 >
2203 2213 2223 2233 2243
      * * * * * * * * * *
      GAG CTG GAG GAT GAG ATC ATC TTC ATC ACG TGC CAC GAG GGC TCC CTC
      Glu Leu Glu Asp Glu Ile IIe Phe Ile Thr Cys His Glu Gly Ser Leu>
          b b CODING SEQUENCE CHIMERA:JUNCTION NUC.1776 b b >
            c 2270 c 1837 TO 3437 OF MCPHUPCAR4.0 FINAL 0 c c 2310>
2253 2263 2273 2283 2293
        * * * * * * * * * *
      ATG GCC CTG GGC TTC CTG ATC GGC TAC ACC TGC CTG CTG GCT GCC ATC
      Met Ala Leu Gly Phe Leu Ile Gly Tyr Thr Cys Leu Leu Ala Ala Ile>
            b b CODING SEQUENCE CHIMERA:JUNCTION NUC.1776 b b >
            c c 2320 1837 TO 3437 OF MCPHUPCAR4.0 FINAL 2350 c c >
        2303 2313 2323 2333 2243
              * * * * * * * * *
    TGC TTC TTC TTT GCC TTC AAG TCC CGG AAG CTG CCG GAG AAC TTC AAT
    Cys Phe Phe Phe Ala Phe Lys Ser Arg Lys Leu Pro Glu Asn Phe Asn>
          b b CODING SEQUENCE CHIMERA:JUNCTION NUC.1776 b b >
2360 c c 2 1837 TO 3427 OF MCPHUPCAR4.0 FINAL 2400c c >
                2353 2363 2373 2383 2393
        * * * * * * * * * *
      GAA GCC AAG TTC ATC ACC TTC AGC ATG CTC ATC TTC TTC ATC GTC TGG
      Glu Ala Lys Phe Ile Thr Phel Ser Met Leu Ile Phe Phe Ile Val Trp>
            b b CODING SEQUENCE CHIMERA:JUNCTION NUC.1776 b b >
        2410 c c 1837 TO 3427 OF MCPHUPCAR4.0 FINAL c 2450 c >
                    2403 2413 2423 2433
              * * * * * * * * *
      ATC TCC TTC ATT CCA GCC TAT GCC AGC ACC TAT GGC AAG TTT GTC TCT
      Ile Ser Phe Ile Pro Ala Tyr Ala Ser Thr Tyr Gly Lys Phe Val Ser>
            b b CODING SEQUENCE CHIMERA:JUNCTION NUC.1776 b b >
            2460c c 1837 TO 3437 OF MCPHUPCAR4.0 FINAL c c 2500 >
2443 2453 2463 2473 2483
      * * * * * * * * * *
      GCC GTA GAG GTG ATT GCC ATC CTG GCA GCC AGC TTT GGC TTG CTG GCG
      Ala Val Glu Val Ile Ala Ile Leu Ala Ala Ser Phe Gly Leu Leu Ala>
            b b CODING SEQUENCE CHIMERA:JUNCTION NUC.1776 b b >
            c 2510 c 1837 TO 3427 OF MCPHUPCAR4.0 FINAL 0 c c 2550>
    2493 2503 2513 2523 2533
          * * * * * * * * * *
      TGC ATC TTC TTC AAC AAG ATC TAC ATC ATT CTC TTC AAG CCA TCC CGC
      Cys Ile Phe Phe Asn Lys Ile Tyr Ile Ile Leu Phe Lys Pro Ser Arg>
            b b CODING SEQUENCE CHIMERA:JUNCTION NUC.1776 b b >
            c c 2560 1837 TO 3437 OF MCPHUPCAR4.0 FINAL 2590 c c >
          2543 2553 2563 2573 2583
                * * * * * * * * *
      AAC ACC ATC GAG GAG GTG CGT TGC AGC ACC GCA GCT CAC GCT TTC AAG
      Asn Thr Ile Glu Glu Val Arg Cys Ser Thr Ala Ala His Ala Phe Lys>
            b b CODING SEQUENCE CHIMERA:JUNCTION NUC.1776 b b >
2600 c c 2 1837 70 3437 OF MCPHUPCAR4.0 FINAL 2640c c >
                2593 2603 2613 2623 2633
        * * * * * * * * * *
      GTG GCT GCC CGG GCC ACG CTG CGC CGC AGC AAC GTC TCC CGC AAG CGG
      Val Ala Ala Arg Ala Thr Leu Arg Arg Ser Asn Val Ser Arg Lys Arg>
            b b CODING SEQUENCE CHIMERA:JUNCTION NUC.1776 b b >
        2650 c c 1837 TO 3437 OF MCPHUPCAR4.0 FINAL c 2690 c >
                    2643 2653 2663 2673
              * * * * * * * * *
      TCC AGC AGC CTT GGA GGC TCC ACG GGA TCC ACC CCC TCC TCC TCC ATC
      Ser Ser Ser Leu Gly Gly Ser Thr Gly Ser Thr Pro Ser Ser Ser Ile>
            b b CODING SEQUENCE CHIMERA:JUNCTION NUC.1776 b b >
            270Cc c 1837 TO 3437 OF MCPHUPCAR4.0 FINAL c c 2740 >
2683 2693 2703 2713 2723
      * * * * * * * * * *
      AGC AGC AAG AGC AAC AGC GAA GAC CCA TTC CCA CAG CCC GAG AGG CAG
      Ser Ser Lys Ser Asn Ser Glu Asp Pro Phe Pro Gln Pro Glu Arg Gln>
            b b CODING SEQUENCE CHIMERA:JUNCTION NUC.1776 b b >
            c 2750 c 1837 70 3437 OF MCPHUPCAR4.0 FINAL 0 c c 2790>
    2733 2743 2753 2763 2773
          * * * * * * * * * *
      AAG CAG CAG CAG CCG CTG GCC CTA ACC CAG CAA GAG CAG CAG CAG CAG
      Lys Gln Gln Gln Pro Leu Ala Leu Thr Gln Gln Glu Gln Gln Gln Gln>
            b b CODING SEQUENCE CHIMERA:JUNCTION NUC.1776 b b >
            c c 2800 1837 TO 3437 MCPHUPCAR4.0 FINAL 2830 c c >
          2783 2793 2803 2813 2823
                * * * * * * * * *
      CCC CTG ACC CTC CCA CAG CAG CAA CGA TCT CAG CAG CAG CCC AGA TGC
      Pro Leu Thr Leu Pro Gln Gln Gln Arg Ser Gln Gln Gln Pro Arg Cys>
            b b CODING SEQUENCE CHIMERA:JUNCTION NUC.1776 b b >
  2840 c c 2 1837 TO 3437 OF MCPHUPCAR4.0 FINAL 2880c c >
                2833 2843 2853 2863 2873
        * * * * * * * * * *
      AAG CAG AAG GTC ATC TTT GGC AGC GGC ACG GTC ACC TTC TCA CTG AGC
      Lys Gln Lys Val Ile Phe Gly Ser Gly Thr Val Thr Phe Ser Leu Ser>
            b b CODING SEQUENCE CHIMERA:JUNCTION NUC.1776 b b >
        2890 c c 1837 70 3437 OF MCPHUPCAR4.0 FINAL c 2930 c >
                    2883 2893 2903 2913
              * * * * * * * * *
      TTT GAT GAG CCT CAG AAG AAC GCC ATG GCC CAC GGG AAT TCT ACG CAC
      Phe Asp Glu Pro Gln Lys Asn Ala Met Ala His Gly Asn Ser Thr His>
            b b CODING SEQUENCE CHIMERA:JUNCTION NUC.1776 b b >
            2940c c 1837 TO 3437 OF MCPHUPCAR4.0 FINAL c c 2980 >
2923 2933 2943 2953 2963
      * * * * * * * * * *
      CAG AAC TCC CTG GAG GCC CAG AAA AGC AGC GAT ACG CTG ACC CGA CAC
      Gln Asn Ser Leu Glu Ala Gln Lys Ser Ser Asp Thr Leu Thr Arg His>
        b b CODING SEQUENCE CHIMERA:JUNCTION NUC.1776 b b
        c 2990 c 1837 TO 3437 OF MCPHUPCAR4.0 FINAL 0 c c 303(
    2973 2983 2993 3003 3013
          * * * * * * * * *
      CAG CCA TTA CTC CCG CTG CAG TGC GGG GAA ACG GAC TTA GAT CTG AC(
      Gln Pro Leu Leu Pro Leu Gln Cys Gly Glu Thr Asp Leu Asp Leu Thr
          b b CODING SEQUENCE CHIMERA:JUNCTION NUC.1776 b b
          c c 3040 1837 TO 3437 OF MCPHUPCAR4.0 FINAL 3070 c c
          3023 3033 3043 3053 3063
              * * * * * * * * *
      GTC CAG GAA ACA GGT CTG CAA GGA CCT GTG GGT GGA GAC CAG CGG CC
      Val Gln Glu Thr Gly Leu Gln Gly Pro Val Gly Gly Asp Gln Arg Pro
            b b CODING SEQUENCE CHIMERA:JUNCTION NUC.1776 b b
  3080 c c 3 1837 TO 3437 0 OF MCPHUPCAR4.0 FINAL 3120c c
                3073 3083 3093 3103 3113
        * * * * * * * * * *
      GAG GTG GAG GAC CCT GAA GAG TTG TCC CCA GCA CTT GTA GTG TCC AG
      Glu Val Glu Asp Pro Glu Glu Leu Ser Pro Ala Leu Val Val Ser Se
            b b CODING SEQUENCE CHIMERA:JUNCTION NUC.1776 b b
        3130 c c 1837 TO 3437 OF MCPHUPCAR4.0 FINAL c 3170 c
                    3123 3133 3143 3153
              * * * * * * * * *
      TCA CAG AGC TTT GTC ATC AGT GGT GGA GGC AGC ACT GTT ACA GAA AA
      Ser Gln Ser Phe Val Ile Ser Gly Gly Gly Ser Thr Val Thr Glu Asp
            b b CODING SEQUENCE CHIMERA:JUNCTION NUC.1776 b b
            3180c c 1837 TO 3437 OF MCPHUPCAR4.0 FINAL c c 3220
3163 3173 3183 3193 3203 3213
      * * * * * * * * * * *
      GTA GTG AAT TCA T AAAATGGA AGGAGAAGAC TGGGCTAGGG AGAATGCAGA
      Val Val Asn Ser Xxx>
            CODING SEQ b >
            c 3230 1837 TO 3437 OF MCPHUPCAR4.0 FINAL c 3270 >
                  3223 3233 3243 3253 3263 3
              * * * * * * * * * * *
      GAGGTTTCTT GGGGTCCCAG GGATGAGGAA TCGCCCCAGA CTCCTTTCCT CTGAGGA
            3280 c 1837 TO 3437 OF MCPHUPCAR4.0 FINAL 0 c 3330
                    3283 3293 3303 3313 3323 3
              * * * * * * * * * * *
      AGGGATAATA GACACATCAA ATGCCCCGAA TTTAGTCACA CCATCTTAAA TGACAGT
            3340 c 1837 TO 3437 OF MCPHUPCAR4.0 FINAL 0 c 3390
                  3343 3353 3363 3373
              * * * * * * * * *
      TTGACCCATG TTCCCTTTAA AAAAAAAAAA AAAAAAGCGG CCGC--
            34 1837 TO 3437 OF MCPHUPCAR4.0 FINAL c >
```

TABLE 26

Nucleotide sequence (SEQ ID NO:27) and corresponding amino
acid sequence (SEQ ID NO:28) of pratCH3

| | | | | |
|---|---|---|---|---|
| Sequence Range: −24 to 3195 | | | | |
| −15 −5 6 16 26 | | | | |
| * * * * * * * * * * | | | | |
| GCGGTGGACC GCGTCTTCGC CACA ATG GTC CGG CTC CTC TTG ATT TTC TTC C | | | | |
| Met Val Arg Leu Leu Leu Ile Phe Phe P | | | | |
| a TRANSLATION OF PRATCH3 [A] a | | | | |
| 36 46 56 66 76 | | | | |
| * * * * * * * * * * | | | | |
| ATG ATC TTT TTG GAG ATG TCC ATT TTG CCC AGG ATG CCT GAC AGA AAA | | | | |
| Met Ile Phe Leu Glu Met Ser Ile Leu Pro Arg Met Pro Asp Arg Lys> | | | | |
| a a a a TRANSLATION OF PRATCH3 [A] a a a a > | | | | |
| 86 96 106 116 126 | | | | |
| * * * * * * * * * * | | | | |
| GTA TTG CTG GCA GGT GCC TCG TCC CAG CGC TCC GTG GCG AGA ATG GAC | | | | |
| Val Leu Leu Ala Gly Ala Ser Ser Gln Arg Ser Val Ala Arg Met Asp> | | | | |
| a a a a TRANSLATION OF PRATCH3 [A] a a a a > | | | | |
| 136 146 156 166 | | | | |
| * * * * * * * * * | | | | |
| GGA GAT GTC ATC ATC GGA GCC CTC TTC TCA GTC CAT CAC CAG CCT CCA | | | | |
| Gly Asp Val Ile Ile Gly Ala Leu Phe Ser Val His His Gln Pro Pro> | | | | |
| a a a a TRANSLATION OF PRATCH3 [A] a a a a > | | | | |
| 176 186 196 206 216 | | | | |
| * * * * * * * * * * | | | | |
| GCC GAG AAG GTA CCC GAA AGG AAG TGT GGG GAG ATC AGG GAA CAG TAT | | | | |
| Ala Glu Lys Val Pro Glu Arg Lys Cys Gly Glu Ile Arg Glu Gln Tyr> | | | | |
| a a a a TRANSLATION OF PRATCH3 [A] a a a a > | | | | |
| 226 236 246 256 266 | | | | |
| * * * * * * * * * | | | | |
| GGT ATC CAG AGG GTG GAG GCC ATG TTC CAC ACG TTG GAT AAG ATT AAC | | | | |
| Gly Ile Gln Arg Val Glu Ala Met Phe His Thr Leu Asp Lys Ile Asn> | | | | |
| a a a a TRANSLATION OF PRATCH3 [A] a a a a > | | | | |
| 276 286 296 306 316 | | | | |
| * * * * * * * * * * | | | | |
| GCG GAC CCG GTG CTC CTG CCC AAC ATC ACT CTG GGC AGT GAG ATC CGG | | | | |
| Ala Asp Pro Val Leu Leu Pro Asn Ile Thr Leu Gly Ser Glu Ile Arg> | | | | |
| a a a a TRANSLATION OF PRATCH3 [A] a a a a > | | | | |
| 326 336 346 356 366 | | | | |
| * * * * * * * * * * | | | | |
| GAC TCC TGC TGG CAC TCT TCA GTG GCT CTC GAA CAG AGC ATC GAA TTC | | | | |
| Asp Ser Cys Trp His Ser Ser Val Ala Leu Glu Gln Ser Ile Glu Phe> | | | | |
| a a a a TRANSLATION OF PRATCH3 [A] a a a a > | | | | |
| 376 386 396 406 | | | | |
| * * * * * * * * * | | | | |
| ATC AGA GAC TCC CTG ATT TCC ATC CGA GAT GAG AAG GAT GGG CTG AAC | | | | |
| Ile Arg Asp Ser Leu Ile Ser Ile Arg Asp Glu Lys Asp Gly Leu Asn> | | | | |
| a a a a TRANSLATION OF PRATCH3 [A] a a a a > | | | | |
| 416 426 436 446 456 | | | | |
| * * * * * * * * * * | | | | |
| CGA TGC CTG CCT GAT GGC CAG ACC CTG CCC CCT GGC AGG ACT AAG AAG | | | | |
| Arg Cys Leu Pro Asp Gly Gln Thr Leu Pro Pro Gly Arg Thr Lys Lys> | | | | |
| a a a a TRANSLATION OF PRATCH3 [A] a a a a > | | | | |
| 466 476 486 496 506 | | | | |
| * * * * * * * * * | | | | |
| CCT ATT GCT GGA GTG ATC GGC CCT GGC TCC AGC TCT GTG GCC ATT CAA | | | | |
| Pro Ile Ala Gly Val IIe Gly Pro Gly Ser Ser Ser Val Ala Ile Gln> | | | | |
| a a a a TRANSLATION OF PRATCH3 [A] a a a a > | | | | |
| 516 526 536 546 556 | | | | |
| * * * * * * * * * * | | | | |
| GTC CAG AAT CTT CTC CAG CTG TTC GAC ATC CCA CAG ATC GCC TAT TCT | | | | |
| Val Gln Asn Leu Leu Gln Leu Phe Asp Ile Pro Gln Ile Ala Tyr Ser> | | | | |
| a a a a TRANSLATION OF PRATCH3 [A] a a a a > | | | | |
| 566 576 586 596 606 | | | | |
| * * * * * * * * * * | | | | |
| GCC ACA AGC ATA GAC CTG AGT GAC AAA ACT TTG TAC AAA TAC TTC CTG | | | | |
| Ala Thr Ser Ile Asp Leu Ser Asp Lys Thr Leu Tyr Lys Tyr Phe Leu> | | | | |
| a a a a TRANSLATION OF PRATCH3 [A] a a a a > | | | | |
| 616 626 636 646 | | | | |
| * * * * * * * * * | | | | |
| AGG GTG GTC CCT TCT GAC ACT TTG CAG GCA AGG GCG ATG CTC GAC ATA | | | | |
| Arg Val Val Pro Ser Asp Thr Leu Gln Ala Arg Ala Met Leu Asp Ile> | | | | |
| a a a a TRANSLATION OF PRATCH3 [A] a a a a > | | | | |
| 656 666 676 686 696 | | | | |
| * * * * * * * * * * | | | | |
| GTC AAG CGT TAC AAC TGG ACC TAT GTC TCA GCA GTC CAC ACA GAA GGG | | | | |
| Val Lys Arg Tyr Asn Trp Thr Tyr Val Ser Ala Val His Thr Glu Gly> | | | | |
| a a a a TRANSLATION OF PRATCH3 [A] a a a a > | | | | |
| 706 716 726 736 746 | | | | |
| * * * * * * * * * | | | | |
| AAT TAC GGC GAG AGT GGA ATG GAT GCT TTC AAA GAA CTG GCT GCC CAG | | | | |
| Asn Tyr Gly Glu Ser Gly Met Asp Ala Phe Lys Glu Leu Ala Ala Gln> | | | | |
| a a a a TRANSLATION OF PRATCH3 [A] a a a a > | | | | |
| 756 766 776 786 796 | | | | |
| * * * * * * * * * * | | | | |
| GAA GGC CTC TGC ATC GCA CAC TCG GAC AAA ATC TAC AGC AAT GCT GGC | | | | |
| Glu Gly Leu Cys Ile Ala His Ser Asp Lys Ile Tyr Ser Asn Ala Gly> | | | | |
| a a a a TRANSLATION OF PRATCH3 [A] a a a a > | | | | |
| 806 816 826 836 846 | | | | |
| * * * * * * * * * * | | | | |
| GAG AAG AGC TTT GAC CGG CTC CTG CGT AAA CTC CGG GAG CGG CTT CCC | | | | |
| Glu Lys Ser Phe Asp Arg Leu Leu Arg Lys Leu Arg Glu Arg Leu Pro> | | | | |
| a a a a TRANSLATION OF PRATCH3 [A] a a a a > | | | | |
| 856 866 876 886 | | | | |
| * * * * * * * * * | | | | |
| AAG GCC AGG GTT GTG GTC TGC TTC TGC GAG GGC ATG ACA GTG CGG GGC | | | | |
| Lys Ala Arg Val Val Val Cys Phe Cys Glu Gly Met Thr Val Arg Gly> | | | | |
| a a a a TRANSLATION OF PRATCH3 [A] a a a a > | | | | |
| 896 906 916 926 936 | | | | |
| * * * * * * * * * * | | | | |
| TTA CTG AGT GCC ATG CGC CGC CTG GGC GTC GTG GGC GAG TTC TCA CTC | | | | |
| Leu Leu Ser Ala Me Arg Arg Leu Gly Val Val Gly Glu Phe Ser Leu> | | | | |
| a a a a TRANSLATION OF PRATCH3 [A] a a a a > | | | | |
| 946 956 966 976 986 | | | | |
| * * * * * * * * * | | | | |
| ATT GGA AGT GAT GGA TGG GCA GAC AGA GAT GAA GTC ATC GAA GGC TAT | | | | |
| Ile Gly Ser Asp Gly Trp Ala Asp Arg Asp Glu Val Ile Glu Gly Tyr> | | | | |
| a a a a TRANSLATION OF PRATCH3 [A] a a a a > | | | | |
| 996 1006 1016 1026 1036 | | | | |
| * * * * * * * * * * | | | | |
| GAG GTG GAA GCC AAC GGA GGG ATC ACA ATA AAG CTT CAG TCT CCA GAG | | | | |
| Glu Val Glu Ala Asn Gly Gly Ile Thr Ile Lys Leu Gln Ser Pro Glu> | | | | |
| a a a a TRANSLATION OF PRATCH3 [A] a a a a > | | | | |
| 1046 1056 1066 1076 1086 | | | | |
| * * * * * * * * * * | | | | |
| GTC AGG TCA TTT GAT GAC TAC TTC CTG AAG CTG AGG CTG GAC ACC AAC | | | | |
| Val Arg Ser Phe Asp Asp Tyr Phe Leu Lys Leu Arg Leu Asp Thr Asn> | | | | |
| a a a a TRANSLATION OF PRATCH3 [A] a a a a > | | | | |
| 1096 1106 1116 1126 | | | | |
| * * * * * * * * * | | | | |
| ACA AGG AAT CCT TGG TTC CCT GAG TTC TGG CAA CAT CGC TTC CAG TGT | | | | |
| Thr Arg Asn Pro Trp Phe Pro Glu Phe Trp Gln His Arg Phe Gln Cys> | | | | |
| a a a a TRANSLATION OF PRATCH3 [A] a a a a > | | | | |
| 1136 1146 1156 1166 1176 | | | | |
| * * * * * * * * * * | | | | |
| CGC CTA CCT GGA CAC CTC TTG GAA AAC CCC AAC TTT AAG AAA GTG TGC | | | | |
| Arg Leu Phe Gly His Leu Leu Leu Asp Pro Asn Phe Lys Lys Val Cys> | | | | |
| a a a a TRANSLATION OF PRATCH3 [A] a a a a > | | | | |
| 1186 1196 1206 1216 1226 | | | | |
| * * * * * * * * * | | | | |
| ACA GGA AAT GAA AGC TTG GAA GAA AAC TAT GTC CAG GAC AGC AAA ATG | | | | |
| Thr Gly Asn Glu Ser Leu Glu Glu Asn Tyr Val Gln Asp Ser Lys Met> | | | | |
| a a a a TRANSLATION OF PRATCH3 [A] a a a a > | | | | |
| 1236 1246 1256 1266 1276 | | | | |
| * * * * * * * * * * | | | | |
| GGA TTT GTC ATC AAT GCC ATC TAT GCC ATG GCA CAT GGG CTG CAG AAC | | | | |
| Gly Phe Val Ile Asn Ala Ile Tyr Ala Met Ala His Gly Leu Gln Asn> | | | | |
| a a a a TRANSLATION OF PRATCH3 [A] a a a a > | | | | |
| a a a a TRANSLATION OF PRATCH3 [A] a a a a > | | | | |
| 1286 1296 1306 1316 1326 | | | | |
| * * * * * * * * * * | | | | |
| ATG CAC CAT GCT CTG TGT CCC GGC CAT GTG GGC CTG TGT GAT GCT ATG | | | | |
| Met His His Ala Leu Cys Pro Gly His Val Gly Leu Cys Asp Ala Met> | | | | |
| a a a a TRANSLATION OF PRATCH3 [A] a a a a > | | | | |
| 1336 1346 1356 1366 | | | | |
| * * * * * * * * * | | | | |
| AAA CCC ATT GAT GGC AGG AAG CTC CTG GAT TTC CTC ATC AAA TCC TCT | | | | |
| Lys Pro Ile Asp Gly Arg Lys Leu Leu Asp Phe Leu Ile Lys Ser Ser> | | | | |
| a a a a TRANSLATION OF PRATCH3 [A] a a a a > | | | | |
| 1376 1386 1396 1406 1416 | | | | |
| * * * * * * * * * * | | | | |
| TTT GTC GGA GTG TCT GGA GAG GAG GTG TGG TTC GAT GAG AAG GGG GAT | | | | |
| Phe Val Gly Val Ser Gly Glu Glu Val Trp Phe Asp Glu Lys Gly Asp> | | | | |
| a a a a TRANSLATION OF PRATCH3 [A] a a a a > | | | | |
| 1426 1436 1446 1456 1466 | | | | |
| * * * * * * * * * | | | | |
| GCT CCC GGA AGG TAT GAC ATT ATG AAT CTG CAG TAC ACA GAA GCT AAT | | | | |
| Ala Pro Gly Arg Tyr Asp Ile Met Asn Leu Gln Tyr Thr Glu Ala Asn> | | | | |
| a a a a TRANSLATION OF PRATCH3 [A] a a a a > | | | | |
| 1476 1486 1496 1506 1516 | | | | |
| * * * * * * * * * * | | | | |
| CGC TAT GAC TAT GTC CAC GTG GGG ACC TGG CAT GAA GGA GTG CTG AAT | | | | |
| Arg Tyr Asp Tyr Val His Val Gly Thr Trp His Glu Gly Val Leu Asn> | | | | |
| a a a a TRANSLATION OF PRATCH3 [A] a a a a > | | | | |
| 1526 1536 1546 1556 1566 | | | | |
| * * * * * * * * * * | | | | |
| ATT GAT GAT TAC AAA ATC CAG ATG AAG AAA AGC GGA ATG GTA CGA TCT | | | | |
| Ile Asp Asp Tyr Lys Ile Gln Met Asn Lys Ser Gly Met Val Arg Ser> | | | | |
| a a a a TRANSLATION OF PRATCH3 [A] a a a a > | | | | |
| 1576 1586 1596 1606 | | | | |
| * * * * * * * * * | | | | |
| GTG TGC AGT GAG CCT TGC TTA AAG GGT CAG ATT AAG GTC ATA CGG AAA | | | | |
| Val Cys Ser Glu Pro Cys Leu Lys Gly Gln Ile Lys Val Ile Arg Lys> | | | | |
| a a a a TRANSLATION OF PRATCH3 [A] a a a a > | | | | |
| 1616 1626 1636 1646 1656 | | | | |
| * * * * * * * * * * | | | | |
| GGA GAA GTG AGC TGC TGC TGG ATC TGC ACG GCC TGC AAA GAG AAT GAG | | | | |
| Gly Glu Val Ser Cys Cys Trp Ile Cys Thr Ala Cys Lys Glu Asn Glu> | | | | |
| a a a a TRANSLATION OF PRATCH3 [A] a a a a > | | | | |
| 1666 1676 1686 1696 1706 | | | | |
| * * * * * * * * * | | | | |
| TTT GTG CAG GAC GAG TTC ACC TGC AGA GCC TGT GAC CTG GGG TGG TGG | | | | |
| Phe Val Gln Asp Glu Phe Cys Arg Ala Cys Asp Leu Gly Trp Trp> | | | | |
| a a a a TRANSLATION OF PRATCH3 [A] a a a a > | | | | |
| 1716 1726 1736 1746 1756 | | | | |
| * * * * * * * * * * | | | | |
| CCC AAC GCA GAG CTC ACA GGC TGT GAG CCC ATT CCT GTC CGT TAT CTT | | | | |
| Pro Asn Ala Glu Leu Thr Gly Cys Glu Pro Ile Pro Val Arg Tyr Leu> | | | | |
| a a a a TRANSLATION OF PRATCH3 [A] a a a a > | | | | |
| 1766 1776 1786 1796 1806 | | | | |
| * * * * * * * * * * | | | | |
| GAG TGG AGT GAC ATA GAA TCT ATC ATA GCC ATC GCC TTT TCT TGC CTG | | | | |
| Glu Trp Ser Asp Ile Glu Ser Ile Ile Ala Ile Ala Phe Ser Cys Leu> | | | | |
| a a a a TRANSLATION OF PRATCH3 [A] a a a a > | | | | |
| 1816 1826 1836 1846 | | | | |
| * * * * * * * * * | | | | |
| GGC ATC CTC GTG ACG CTG TTT GTC ACC CTC ATC TTC GTT CTG TAC CGG | | | | |
| Gly Ile Leu Val Thr Leu Phe Val Thr Leu Ile Phe Val Leu Tyr Arg> | | | | |
| a a a a TRANSLATION OF PRATCH3 [A] a a a a > | | | | |
| 1856 1866 1876 1886 1896 | | | | |
| * * * * * * * * * * | | | | |
| GAC ACA CCC GTG GTC AAA TCC TCC AGT AGG GAG CTC TGC TAT ATC ATT | | | | |
| Asp Thr Pro Val Val Lys Ser Ser Ser Arg Glu Leu Cys Tyr Ile Ile> | | | | |
| a a a a TRANSLATION OF PRATCH3 [A] a a a a > | | | | |
| 1906 1916 1926 1936 1946 | | | | |
| * * * * * * * * * | | | | |
| CTG GCT GGT ATT TTC CTC GGC TAT GTG TGC CCT TTC ACC CTC ATC GCC | | | | |
| Leu Ala Gly Ile Phe Leu Gly Tyr Val Cys Pro Phe Thr Leu Ile Ala> | | | | |
| a a a a TRANSLATION OF PRATCH3 [A] a a a a > | | | | |
| 1956 1966 1976 1986 1996 | | | | |
| * * * * * * * * * * | | | | |
| AAA CCT ACT ACC ACA TCC TGC TAC CTC CAG CGC CTC CTA GTT GGC CTC | | | | |
| Lys Pro Thr Thr Thr Ser Cys Tyr Leu Gln Arg Leu Leu Val Gly Leu> | | | | |
| a a a a TRANSLATION OF PRATCH3 [A] a a a a > | | | | |
| 2006 2016 2026 2036 2046 | | | | |
| * * * * * * * * * * | | | | |
| TCT TCT GCC ATG TGC TAC TCT GCT TTA GTG ACC AAA ACC AAT CGT ATT | | | | |
| Ser Ser Ala Met Cya Tyr Ser Ala Leu Val Thr Lys Thr Asn Arg Ile> | | | | |
| a a a a TRANSLATION OF PRATCH3 [A] a a a a > | | | | |
| 2056 2066 2076 2086 | | | | |
| * * * * * * * * * | | | | |
| GCA CGC ATC CTG GCT GGC AGC AAG AAG AAG ATC TGC ACC CGG AAG CCC | | | | |
| Ala Arg Ile Leu Ala Gly Ser Lys Lys Lys Ile Cys Thr Arg Lys Pro> | | | | |
| a a a a TRANSLATION OF PRATCH3 [A] a a a a > | | | | |
| 2096 2106 2116 2126 2136 | | | | |
| * * * * * * * * * * | | | | |
| AGA TTC ATG AGC GCT TGG GCC CAA GTG ATC ATA GCC TCC ATT CTG ATT | | | | |
| Arg Phe Met Ser Ala Trp Ala Gln Val Ile Ile Ala Ser Ile Leu Ile> | | | | |
| a a a a TRANSLATION OF PRATCH3 [A] a a a a > | | | | |
| 2146 2156 2166 2176 2186 | | | | |
| * * * * * * * * * | | | | |
| AGT GTA CAG CTA ACA CTA GTG GTG ACC TTG ATC ATC ATG GAG CCT CCC | | | | |
| Ser Val Gln Leu Thr Leu Val Val Thr Leu Ile Ile Met Glu Pro Pro> | | | | |
| a a a a TRANSLATION OF PRATCH3 [A] a a a a > | | | | |
| 2196 2206 2216 2226 2236 | | | | |
| * * * * * * * * * * | | | | |
| ATG CCC ATT TTG TCC TAC CCG AGT ATC AAG GAA GTC TAC CTT ATC TGC | | | | |
| Met Pro Ile Leu Ser Tyr Pro Ser Ile Lys Glu Val Tyr Leu Ile Cys> | | | | |
| a a a a TRANSLATION OF PRATCH3 [A] a a a a > | | | | |
| 2246 2256 2266 2276 2286 | | | | |
| * * * * * * * * * * | | | | |
| AAT ACC AGC AAC CTG GGT GTG GTG GCC CCT TTG GGC TAC AAT GGA CTC | | | | |
| Asn Thr Ser Asn Leu Gly Val Val Ala Pro Leu Gly Tyr Asn Gly Leu> | | | | |
| a a a a TRANSLATION OF PRATCH3 [A] a a a a > | | | | |
| 2296 2306 2316 2326 | | | | |
| * * * * * * * * * | | | | |
| CTC ATC ATG AGC TGT ACC TAC TAT GCC TTC AAG ACC CGC AAC GTG CCC | | | | |
| Leu Ile Met Ser Cys Thr Tyr Tyr Ala Phe Lys Thr Arg Asn Val Pro> | | | | |
| a a a a TRANSLATION OF PRATCH3 [A] a a a a > | | | | |
| 2336 2346 2356 2366 2376 | | | | |
| * * * * * * * * * * | | | | |
| GCC AAC TTC AAC GAG GCC AAA TAT ATC GCG TTC ACC ATG TAC ACC ACC | | | | |
| Ala Asn Phe Asn Glu Ala Lys Tyr Ile Ala Phe Thr Met Tyr Thr Thr> | | | | |
| a a a a TRANSLATION OF PRATCH3 [A] a a a a > | | | | |
| 2386 2396 2406 2416 2426 | | | | |
| * * * * * * * * * | | | | |
| TGT ATC ATC TGG CTA GCT TTT GTG CCC ATT TAC TTT GGG AGC AAC TAC | | | | |
| Cys Ile Ile Trp Leu Ala Phe Val Pro Ile Tyr Phe Gly Ser Asn Tyr> | | | | |
| a a a a TRANSLATION OF PRATCH3 [A] a a a a > | | | | |
| 2436 2446 2456 2466 2476 | | | | |
| * * * * * * * * * * | | | | |
| AAG ATC ATC ACA ACT TCC TTT GCA GTG AGT CTC AGT GTA ACA GTG GCT | | | | |
| Lys Ile Ile Thr Thr Cys Phe Ala Val Ser Leu Ser Val Thr Val Ala> | | | | |
| a a a a TRANSLATION OF PRATCH3 [A] a a a a > | | | | |
| 2486 2496 2506 2516 2526 | | | | |
| * * * * * * * * * * | | | | |
| CTG GGG TGC ATC TTC ACT CCC AAG ATG TAC ATC ATT ATT GCC AAG CCT | | | | |
| Leu Gly Cys Met Phe Thr Pro Lys Met Tyr Ile Ile Ile Ala Lys Pro> | | | | |
| a a a a TRANSLATION OF PRATCH3 [A] a a a a > | | | | |
| 2536 2546 2556 2566 | | | | |
| * * * * * * * * * | | | | |
| GAG AGG AAT ACC ATC GAG GAG GTG CGT TGC AGC ACC GCA GCT CAC GCT | | | | |
| Glu Arg Asn Thr Ile Glu Glu Val Arg Cys Ser Thr Ala Ala His Ala> | | | | |
| a a a a TRANSLATION OF PRATCH3 [A] a a a a > | | | | |
| 2576 2586 2596 2606 2616 | | | | |
| * * * * * * * * * * | | | | |
| TTC AAG GTG GCT GCC CGG GCC ACG CTG CGC CGC AGC AAC GTC TCC CGC | | | | |
| Phe Lys Val Ala Ala Arg Ala Thr Leu Arg Arg Ser Asn Val Ser Arg> | | | | |
| a a a a TRANSLATION OF PRATCH3 [A] a a a a > | | | | |
| 2626 2636 2646 2656 2666 | | | | |
| * * * * * * * * * | | | | |
| AAG CGG TCC AGC AGC CTT GGA GGC TCC ACG GGA TCC ACC CCC TCC TCC | | | | |
| Lys Arg Ser Ser Ser Leu Gly Gly Ser Thr Gly Ser Thr Pro Ser Ser> | | | | |
| a a a a TRANSLATION OF PRATCH3 [A] a a a a > | | | | |
| 2676 2686 2696 2706 2716 | | | | |
| * * * * * * * * * * | | | | |
| TCC ATC AGC AGC AAG AGC AAC AGC GAA GAC CCA TTC CCA CAG CCC GAG | | | | |
| Ser Ile Ser Ser Lys Ser Asn Ser Glu Asp Pro Phe Pro Gln Pro Glu> | | | | |
| a a a a TRANSLATION OF PRATCH3 [A] a a a a > | | | | |
| 2726 2736 2746 2756 2766 | | | | |
| * * * * * * * * * * | | | | |
| AGG CAG AAG CAG CAG CAG CCG CTG GCC CTA ACC CAG CAA GAG CAG CAG | | | | |
| Arg Gln Lys Gln Gln Gln Pro Leu Ala Leu Thr Gln Gln Glu Gln Gln> | | | | |
| a a a a TRANSLATION OF PRATCH3 [A] a a a a > | | | | |
| 2776 2786 2796 2806 | | | | |
| * * * * * * * * * | | | | |
| CAG CAG CCC CTG ACC CTC CCA CAG CAG CAA CGA TCT CAG CAG CAG CCC | | | | |
| Gln Gln Pro Leu Thr Leu Pro Gln Gln Gln Arg Ser Gln Gln Gln Pro> | | | | |
| a a a a TRANSLATION OF PRATCH3 [A] a a a a > | | | | |
| 2816 2826 2836 2846 2856 | | | | |
| * * * * * * * * * * | | | | |
| AGA TGC AAG CAG AAG GTC ATC TTT GGC AGC GGC ACG GTC ACC TTC TCA | | | | |
| Arg Cys Lys Gln Lys Val Ile Phe Gly Ser Gly Thr Val Thr Phe Ser> | | | | |
| a a a a TRANSLATION OF PRATCH3 [A] a a a a > | | | | |
| 2866 2876 2886 2896 2906 | | | | |
| * * * * * * * * * | | | | |
| CTG AGC TTT GAT GAG CCT CAG AAG AAC GCC ATG GCC CAC GGG AAT TCT | | | | |
| Leu Ser Phe Asp Glu Pro Gln Lys Asn Ala Met Ala His Gly Asn Ser> | | | | |
| a a a a TRANSLATION OF PRATCH3 [A] a a a a > | | | | |
| 2916 2926 2936 2946 2956 | | | | |
| * * * * * * * * * * | | | | |
| ACG CAC CAG AAC TCC CTG GAG GCC CAG AAA AGC AGC GAT ACG CTG ACC | | | | |
| Thr His Gln Asn Ser Leu Glu Ala Gln Lys Ser Ser Asp Thr Leu Thr> | | | | |
| a a a a TRANSLATION OF PRATCH3 [A] a a a a > | | | | |
| 2966 2976 2986 2996 3006 | | | | |
| 2966 2976 2986 2996 3006 | | | | |
| CGA CAC CAG CCA TTA CTC CCG CTG CAG TGC GGG GAA ACG GAC TTA GA | | | | |
| Arg His Gln Pro Leu Leu Pro Leu Gln Cys Gly Glu Thr Asp Leu Asp | | | | |
| a a a a TRANSLATION OF PRATCH3 [A] a a a a > | | | | |
| 3016 3026 3036 3046 | | | | |
| * * * * * * * * * | | | | |
| CTG ACC GTC CAG GAA ACA GGT CTG CAA GGA CCT GTG GGT GGA GAC CA | | | | |
| Leu Thr Val Gln Glu Thr Gly Leu Gln Gly Pro Val Gly Gly Asp Gl | | | | |
| a a a a TRANSLATION OF PRATCH3 [A] a a a a > | | | | |
| 3056 3066 3076 3086 3096 | | | | |
| * * * * * * * * * * | | | | |
| CGG CCA GAG GTG GAG GAC CCT GAA GAG TTG TCC CCA GCA CTT GTA GT | | | | |
| Arg Pro Glu Val Glu Asp Pro Glu Glu Leu Ser Pro Ala Leu Val Val | | | | |
| a a a a TRANSLATIC OF PRATCX3 (A | a | a | a | a |
| 3106 3116 3126 3136 3146 | | | | |
| * * * * * * * * * | | | | |
| TCC AGT TCA CAG AGC TTT GTC ATC AGT GGT GGA GGC AGC ACT GTT AC | | | | |
| Ser Ser Ser Gln Ser Phe Val Ile Ser Gly Gly Gly Ser Thr Val Thr | | | | |
| a a a a TRANSLATION OF PRATCH3 [A] a a a a > | | | | |
| 3156 3166 3176 3186 | | | | |
| * * * * * * * * * | | | | |
| GAA AAC GTA GTG AAT TCA TAAAATGG AAGGAGAAGA CTGGGCTAG | | | | |
| Glu Asn Val Val Asn Ser> | | | | |
| TRANSLATION OF P | | | | |

TABLE 27

Nucleotide sequence (SEQ ID NO:29) and corresponding amino
acid sequence (SEQ ID NO:30) of phCH4

```
Sequence Range: −24 to 3155
                  −15 −5 6 16 26
            * * * * * * * * * *
    GCGGTGGACC GCGTCTTCGC CACA ATG GTC CGG CTC CTC TTG ATT TTC TTC C
                                                          Met Val Arg Leu Leu Leu Ile Phe Phe P
                                                              a TRANSLATION OF PHCH4 [A] a
              36 46 56 66 76
    * * * * * * * * * *
    ATG ATC TTT TTG GAG ATG TCC ATT TTG CCC AGG ATG CCT GAC AGA AAA
    Met Ile Phe Leu Glu Met Ser Ile Leu Pro Arg Met Pro Asp Arg Lys>
          a a a a TRANSLATION OF PHCH4 [A] a a a a >
                    86 96 106 116 126
        * * * * * * * * * *
    GTA TTG CTG GCA GGT GCC TCG TCC CAG CGC TCC GTG GCG AGA ATG GAC
    Val Leu Leu Ala Gly Ala Ser Ser Gln Arg Ser Val Ala Arg Met Asp>
          a a a a TRANSLATION OF PHCH4 [A] a a a a >
                        136 146 156 166
              * * * * * * * * *
    GGA GAT GTC ATC ATC GGA GCC CTC TTC TCA GTC CAT CAC CAG CCT CCA
    Gly Asp Val Ile Ile Gly Ala Leu Phe Ser Val His His Gln Pro Pro>
          a a a a TRANSLATION OF PHCH4 [A] a a a a >
  176 186 196 206 216
      * * * * * * * * * *
    GCC GAG AAG GTA CCC GAA AGG AAG TGT GGG GAG ATC AGG GAA CAG TAT
    Ala Glu Lys Val Pro Glu Arg Lys Cys Gly Glu Ile Arg Glu Gln Tyr>
          a a a a TRANSLATION OF PHCH4 [A] a a a a >
        226 236 246 256 266
            * * * * * * * * *
    GGT ATC CAG AGG GTG GAG GCC ATG TTC CAC ACG TTG GAT AAG ATT AAC
    Gly Ile Gln Arg Val Glu Ala Met Phe His Thr Leu Asp Lys Ile Asn>
          a a a a TRANSLATION OF PHCH4 [A] a a a a >
            276 286 296 306 316
    * * * * * * * * * *
    GCG GAC CCG GTG CTC CTG CCC AAC ATC ACT CTG GGC AGT GAG ATC CGG
    Ala Asp Pro Val Leu Leu Pro Asn Ile Thr Leu Gly Ser Glu Ile Arg>
          a a a a TRANSLATION OF PHCH4 [A] a a a a >
                  326 336 346 356 366
        * * * * * * * * * *
    GAC TCC TGC TGG CAC TCT TCA GTG GCT CTC GAA CAG AGC ATC GAA TTC
    Asp Ser Cys Trp His Ser Ser Val Ala Leu Glu Gln Ser Ile Glu Phe>
          a a a a TRANSLATION OF PHCH4 [A] a a a a >
                        376 386 396 406
              * * * * * * * * *
    ATC AGA GAC TCC CTG ATT TCC ATC CGA GAT GAG AAG GAT GGG CTG AAC
    Ile Arg Asp Ser Leu Ile Ser Ile Arg Asp Glu Lys Asp Gly Leu Asn>
          a a a a TRANSLATION OF PHCH4 [A] a a a a >
  416 426 436 446 456
      * * * * * * * * * *
    CGA TGC CTG CCT GAT GGC CAG ACC CTG CCC CCT GGC AGG ACT AAG AAG
    Arg Cys Leu Pro Asp Gly Gln Thr Leu Pro Pro Gly Arg Thr Lys Lys>
          a a a a TRANSLATION OF PHCH4 [A] a a a a >
        466 476 486 496 506
            * * * * * * * * *
    CCT ATT GCT GGA GTG ATC GGC CCT GGC TCC AGC TCT GTG GCC ATT CAA
    Pro Ile Ala Gly Val Ile Gly Pro Gly Ser Ser Ser Val Ala Ile Gln>
          a a a a TRANSLATION OF PHCH4 [A] a a a a >
            516 526 536 546 556
    * * * * * * * * * *
    GTC CAG AAT CTT CTC CAG CTG TTC GAC ATC CCA CAG ATC GCC TAT TCT
    Val Gln Asn Leu Leu Gln Leu Phe Asp Ile Pro Gln Ile Ala Tyr Ser>
          a a a a TRANSLATION OF PHCH4 [A] a a a a >
                  566 576 586 596 606
        * * * * * * * * * *
    GCC ACA AGC ATA GAC CTG AGT GAC AAA ACT TTG TAC AAA TAC TTC CTG
    Ala Thr Ser Ile Asp Leu Ser Asp Lys Thr Leu Tyr Lys Tyr Phe Leu>
          a a a a TRANSLATION OF PHCH4 [A] a a a a >
                        616 626 636 646
              * * * * * * * * *
    AGG GTT GTC CCT TCT GAC ACT TTG CAG GCA AGG GCC ATG CTT GAC ATA
    Arg Val Val Pro Ser Asp Thr Leu Gln Ala Arg Ala Met Leu Asp Ile>
          a a a a TRANSLATION OF PHCH4 [A] a a a a >
  656 666 676 686 696
      * * * * * * * * * *
    GTC AAA CGT TAC AAT TGG ACC TAT GTC TCT GCA GTC CAC ACG GAA GGG
    Val Lys Arg Tyr Asn Trp Thr Tyr Val Ser Ala Val His Thr Glu Gly>
          a a a a TRANSLATION OF PHCH4 [A] a a a a >
        706 716 726 736 746
            * * * * * * * * *
    AAT TAT GGG GAG AGC GGA ATG GAC GCT TTC AAA GAG CTG GCT GCC CAG
    Asn Tyr Gly Glu Ser Gly Met Asp Ala Phe Lys Glu Leu Ala Ala Gln>
          a a a a TRANSLATION OF PHCH4 [A] a a a a >
            756 766 776 786 796
    * * * * * * * * * *
    GAA GGC CTC TGT ATC GCC CAT TCT GAC AAA ATC TAC AGC AAC GCT GGG
    Glu Gly Leu Cys Ile Ala His Ser Asp Lys Ile Tyr Ser Asn Ala Gly>
          a a a a TRANSLATION OF PHCH4 [A] a a a a >
                  806 816 826 836 846
        * * * * * * * * * *
    GAG AAG AGC TTT GAC CGA CTC TTG CGC AAA CTC CGA GAG AGG CTT CCC
    Glu Lys Ser Phe Asp Arg Leu Leu Arg Lys Leu Arg Glu Arg Leu Pro>
          a a a a TRANSLATION OF PHCH4 [A] a a a a >
                        856 866 876 886
              * * * * * * * * *
    AAG GCT AGA GTG GTG GTC TGC TTC TGT GAA GGC ATG ACA GTG CGA GGA
    Lys Ala Arg Val Val Val Cys Phe Cys Glu Gly Met Thr Val Arg Gly>
          a a a a TRANSLATION OF PHCH4 [A] a a a a >
  896 906 916 926 936
      * * * * * * * * * *
    CTC CTG AGC GCC ATG CGG CGC CTT GGC GTC GTG GGC GAG TTC TCA CTC
    Leu Leu Ser Ala Met Arg Arg Leu Gly Val Val Gly Glu Phe Ser Leu>
          a a a a TRANSLATION OF PHCH4 [A] a a a a >
        946 956 956 976 986
            * * * * * * * * *
    ATT GGA AGT GAT GGA TGG GCA GAC AGA GAT GAA GTC ATT GAA GGT TAT
    Ile Gly Ser Asp Gly Trp Ala Asp Arg Asp Glu Val Ile Glu Gly Tyr>
          a a a a TRANSLATION OF PHCH4 [A] a a a a >
            996 1006 1016 1026 1036
    * * * * * * * * * *
    GAG GTG GAA GCC AAC GGG GGA ATC ACG ATA AAG CTG CAG TCT CCA GAG
    Glu Val Glu Ala Asn Gly Gly Ile Thr Ile Lys Leu Gln Ser Pro Glu>
          a a a a TRANSLATION OF PHCH4 [A] a a a a >
                1046 1056 1066 1076 1086
        * * * * * * * * * *
    GTC AGG TCA TTT GAT GAT TAT TTC CTG AAA CTG AGG CTG GAC ACT AAC
    Val Arg Ser Phe Asp Asp Tyr Phe Leu Lys Leu Arg Leu Asp Thr Asn>
          a a a a TRANSLATION OF PHCH4 [A] a a a a >
                      1096 1106 1116 1126
              * * * * * * * * *
    ACG AGG AAT CCC TGG TTC CCT GAG TTC TGG CAA CAT CGG TTC CAG TGC
    Thr Arg Asn Pro Trp Phe Pro Glu Phe Trp Gln His Arg Phe Gln Cys>
          a a a a TRANSLATION OF PHCH4 [A] a a a a >
1136 1146 1156 1166 1176
      * * * * * * * * * *
    CGC CTT CCA GGA CAC CTT CTG GAA AAT CCC AAC TTT AAA CGA ATC TGC
    Arg Leu Pro Gly His Leu Leu Glu Asn Pro Asn Phe Lys Arg Ile Cys>
          a a a a TRANSLATION OF PHCH4 [A] a a a a >
      1186 1196 1206 1216 1226
            * * * * * * * * *
    ACA GGC AAT GAA AGC TTA GAA GAA AAC TAT GTC CAG GAC AGT AAG ATG
    Thr Gly Asn Glu Ser Leu Glu Glu Asn Tyr Val Gln Asp Ser Lys Met>
          a a a a TRANSLATION OF PHCH4 [A] a a a a >
          1236 1246 1256 1266 1276
    * * * * * * * * * *
    GGG TTT GTC ATC AAT GCC ATC TAT GCC ATG GCA CAT GGG CTG CAG AAC
    Gly Phe Val Ile Asn Ala Ile Tyr Ala Met Ala His Gly Leu Gln Asn>
          a a a a TRANSLATION OF PHCH4 [A] a a a a >
                1286 1296 1306 1316 1326
        * * * * * * * * * *
    ATG CAC CAT GCC CTC TGC CCT GGC CAC GTG GGC CTC TGC GAT GCC ATG
    Met His His Ala Leu Cys Pro Gly His Val Gly Leu Cys Asp Ala Met>
          a a a a TRANSLATION OF PHCH4 [A] a a a a >
                      1336 1346 1356 1366
              * * * * * * * * *
    AAG CCC ATC GAC GGC AGC AAG CTG CTG GAC TTC CTC ATC AAG TCC TCA
    Lys Pro Ile Asp Gly Ser Lys Leu Leu Asp Phe Leu Ile Lys Ser Ser>
          a a a a TRANSLATION OF PHCH4 [A] a a a a >
1376 1386 1396 1406 1416
      * * * * * * * * * *
    TTC ATT GGA GTA TCT GGA GAG GAG GTG TGG TTT GAT GAG AAA GGA GAC
    Phe Ile Gly Val Ser Gly Glu Glu Val Trp Phe Asp Glu Lys Gly Asp>
          a a a a TRANSLATION OF PHCH4 [A] a a a a >
      1426 1436 1446 1456 1466
            * * * * * * * * *
    GCT CCT GGA AGG TAT GAT ATC ATG AAT CTG CAG TAC ACT GAA GCT AAT
    Ala Pro Gly Arg Tyr Asp Ile Met Asn Leu Gln Tyr Thr Glu Ala Asn>
          a a a a TRANSLATION OF PHCH4 [A] a a a a >
          1476 1486 1496 1506 1516
    * * * * * * * * * *
    CGC TAT GAC TAT GTG CAC GTT GGA ACC TGG CAT GAA GGA GTG CTG AAC
    Arg Tyr Asp Tyr Val His Val Gly Thr Trp His Glu Gly Val Leu Asn>
          a a a a TRANSLATION OF PHCH4 [A] a a a a >
                1526 1536 1546 1556 1566
        * * * * * * * * * *
    ATT GAT GAT TAC AAA ATC CAG ATG AAC AAG AGT GGA GTG GTG CGG TCT
    Ile Asp Asp Tyr Lys Ile Gln Met Asn Lys Ser Gly Val Val Arg Ser>
          a a a a TRANSLATION OF PHCH4 [A] a a a a >
                      1576 1586 1596 1606
              * * * * * * * * *
    GTG TGC AGT GAG CCT TGC TTA AAG GGC CAG ATT AAG GTT ATA CGG AAA
    Val Cys Ser Glu Pro Cys Leu Lys Gly Gln Ile Lys Val Ile Arg Lys>
          a a a a TRANSLATION OF PHCH4 [A] a a a a >
1616 1626 1636 1646 1656
      * * * * * * * * * *
    GGA GAA GTG AGC TGC TGC TGG ATT TGC ACG GCC TGC AAA GAG AAT GAA
    Gly Glu Val Ser Cys Cys Trp Ile Cys Thr Ala Cys Lys Glu Asn Glu>
          a a a a TRANSLATION OF PHCH4 [A] a a a a >
      1666 1676 1686 1696 1706
            * * * * * * * * *
    TAT GTG CAA GAT GAG TTC ACC TGC AAA GCT TGT GAC TTG GGA TGG TGG
    Tyr Val Gln Asp Glu Phe Thr Cys Lys Ala Cys Asp Leu Gly Trp Trp>
          a a a a TRANSLATION OF PHCH4 [A] a a a a >
          1716 1726 1736 1746 1756
    * * * * * * * * * *
    CCC AAT GCA GAT CTA ACA GGC TGT GAG CCC ATT CCT GTG CGC TAT CTT
    Pro Asn Ala Asp Leu Thr Gly Cys Glu Pro Ile Pro Val Arg Tyr Leu>
          a a a a TRANSLATION OF PHCH4 [A] a a a a >
                1766 1776 1786 1796 1806
        * * * * * * * * * *
    GAG TGG AGC AAC ATC GAA CCC ATT ATA GCC ATC GCC TTT TCA TGC CTG
    Glu Trp Ser Asn Ile Glu Pro Ile Ile Ala Ile Ala Phe Ser Cys Leu>
          a a a a TRANSLATION OF PHCH4 [A] a a a a >
                      1816 1826 1836 1846
              * * * * * * * * *
    GGA ATC CTT GTT ACC TTG TTT GTC ACC CTA ATC TTT GTA CTG TAC CGG
    Gly Ile Leu Val Thr Leu Phe Val Thr Leu Ile Phe Val Leu Tyr Arg>
          a a a a TRANSLATION OF PHCH4 [A] a a a a >
1856 1866 1876 1886 1896
      * * * * * * * * * *
    GAC ACA CCA GTG GTC AAA TCC TCC AGT CGG GAG CTC TGC TAC ATC ATC
    Asp Thr Pro Val Val Lys Ser Ser Ser Arg Glu Leu Cys Tyr Ile Ile>
          a a a a TRANSLATION OF PHCH4 [A] a a a a >
      1906 1916 1926 1936 1946
            * * * * * * * * *
    CTA GCT GGC ATC TTC CTT GGT TAT GTG TGC CCA TTC ACT CTC ATT GCC
    Leu Ala Gly Ile Phe Leu Gly Tyr Val Cys Pro Phe Thr Leu Ile Ala>
          a a a a TRANSLATION OF PHCH4 [A] a a a a >
          1956 1966 1976 1986 1996
    * * * * * * * * * *
    AAA CCT ACT ACC ACC TCC TGC TAC CTC CAG CGC CTC TTG GTT GGC CTC
    Lys Pro Thr Thr Thr Ser Cys Tyr Leu Gln Arg Leu Leu Val Gly Leu>
          a a a a TRANSLATION OF PHCH4 [A] a a a a >
                2006 2016 2026 2036 2046
        * * * * * * * * * *
    TCC TCT GCG ATG TGC TAC TCT GCT TTA GTG ACT AAA ACC AAT CGT ATT
    Ser Ser Ala Met Cya Tyr Ser Ala Leu Val Thr Lys Thr Asn Arg Ile>
          a a a a TRANSLATION OF PHCH4 [A] a a a a >
                      2056 2066 2076 2086
              * * * * * * * * *
    GCA CGC ATC CTG GCT GGC AGC AAG AAG AAG ATC TGC ACC CGG AAG CCC
    Ala Arg Ile Leu Ala Gly Ser Lys Lys Lys Ile Cys Thr Arg Lys Pro>
          a a a a TRANSLATION OF PHCH4 [A] a a a a >
2096 2106 2116 2126 2136
      * * * * * * * * * *
    AGG TTC ATG AGT GCC TGG GCT CAG GTG ATC ATT GCC TCA ATT CTG ATT
    Arg Phe Met Ser Ala Trp Ala Gln Val Ile Ile Ala Ser Ile Leu Ile>
          a a a a TRANSLATION OF PHCH4 [A] a a a a >
      2146 2156 2166 2176 2186
            * * * * * * * * *
    AGT GTG CAA CTA ACC CTG GTG GTA ACC CTG ATC ATC ATG GAA CCC CCT
    Ser Val Gln Leu Thr Leu Val Val Thr Leu Ile Ile Met Glu Pro Pro>
          a a a a TRANSLATION OF PHCH4 [A] a a a a >
          2196 2206 2216 2226 2236
    * * * * * * * * * *
    ATG CCC ATT CTG TCC TAC CCA AGT ATC AAG GAA GTC TAC CTT ATC TGC
    Met Pro Ile Leu Ser Tyr Pro Ser Ile Lys Glu Val Tyr Leu Ile Cys>
          a a a a TRANSLATION OF PHCH4 [A] a a a a >
                2246 2256 2266 2276 2286
        * * * * * * * * * *
    AAT ACC AGC AAC CTG GGT GTG GTG GCC CCT TTG GCC TAC AAT GGA CTC
    Asn Thr Ser Asn Leu Gly Val Val Ala Pro Leu Gly Tyr Asn Gly Leu>
          a a a a TRANSLATION OF PHCH4 [A] a a a a >
                      2296 2306 2316 2326
              * * * * * * * * *
    CTC ATC ATG AGC TGT ACC TAC TAT GCC TTC AAG ACC CGC AAC GTG CCC
    Leu Ile Met Ser Cys Thr Tyr Tyr Ala Phe Lys Thr Arg Asn Val Pro>
          a a a a TRANSLATION OF PHCH4 [A] a a a a >
2336 2346 2356 2366 2376
      * * * * * * * * * *
    GCC AAC TTC AAC GAG GCC AAA TAT ATC GCG TTC ACC ATG TAC ACC ACC
    Ala Asn Phe Asn Glu Ala Lys Tyr Ile Ala Phe Thr Met Tyr Thr Thr>
          a a a a TRANSLATION OF PHCH4 [A] a a a a >
      2386 2396 2406 2416 2426
            * * * * * * * * *
    TGT ATC ATC TGG CTA GCT TTT GTG CCC ATT TAC TTT GGG AGC AAC TAC
    Cys Ile Ile Trp Leu Ala Phe Val Pro Ile Tyr Phe Gly Ser Asn Tyr>
          a a a a TRANSLATION OF PHCH4 [A] a a a a >
          2436 2446 2456 2466 2476
    * * * * * * * * * *
    AAG ATC ATC ACA ACT TGC TTT GCA GTG AGT CTC AGT GTA ACA GTG GCT
    Lys Ile Ile Thr Thr Cys Phe Ala Val Ser Leu Ser Val Thr Val Ala>
          a a a a TRANSLATION OF PHCH4 [A] a a a a >
                2486 2496 2506 2516 2526
        * * * * * * * * * *
    CTG GGG TGC ATG TTC ACT CCC AAG ATG TAC ATC ATT ATT GCC AAG CCT
    Leu Gly Cys Met Phe Thr Pro Lys Met Tyr Ile Ile Ile Ala Lys Pro>
          a a a a TRANSLATION OF PHCH4 [A] a a a a >
                      2536 2546 2556 2566
              * * * * * * * * *
    GAG AGG AAT ACC ATC GAG GAG GTG CGT TGC AGC ACC GCA GCT CAC GCT
    Glu Arg Asn Thr Ile Glu Glu Val Arg Cys Ser Thr Ala Ala His Ala>
          a a a a TRANSLATION OF PHCH4 [A] a a a a >
2576 2586 2596 2606 2616
      * * * * * * * * * *
    TTC AAG GTG GCT GCC CGG GCC ACG CTC CGC CGC AGC AAC GTC TCC CGC
    Phe Lys Val Ala Ala Arg Ala Thr Leu Arg Arg Ser Asn Val Ser Arg>
          a a a a TRANSLATION OF PHCH4 [A] a a a a >
      2626 2636 2646 2656 2666
            * * * * * * * * *
    AAG CGG TCC AGC AGC CTT GGA GGC TCC ACG GGA TCC ACC CCC TCC TCC
    Lys Arg Ser Ser Ser Leu Gly Gly Ser Thr Gly Ser Thr Pro Ser Ser>
          a a a a TRANSLATION OF PHCH4 [A] a a a a >
          2676 2686 2696 2706 2716
    * * * * * * * * * *
    TCC ATC AGC AGC AAG AGC AAC AGC GAA GAC CCA TTC CCA CAG CCC GAG
    Ser Ile Ser Ser Lys Ser Asn Ser Glu Asp Pro Phe Pro Gln Pro CIu>
          a a a a TRANSLATION OF PHCH4 [A] a a a a >
                2726 2736 2746 2756 2766
        * * * * * * * * * *
    AGG CAG AAG CAG CAG CAG CCG CTG GCC CTA ACC CAG CAA GAG CAG CAG
    Arg Gln Lys Gln Gln Gln Pro Leu Ala Leu Thr Gln Gln Glu Gln Gln>
          a a a a TRANSLATION OF PHCH4 [A] a a a a >
                      2776 2786 2796 2806
              * * * * * * * * *
    CAG CAG CCC CTG ACC CTC CCA CAG CAG CAA CGA TCT CAG CAC CAG CCC
    Gln Gln Pro Leu Thr Leu Pro Gln Gln Gln Arg Ser Gln Gln Gln Pro>
          a a a a TRANSLATION OF PHCH4 [A] a a a a >
2816 2826 2836 2846 2856
      * * * * * * * * * *
    AGA TGC AAG CAG AAG GTC ATC TTT GGC AGC GGC ACG GTC ACC TTC TCA
    Arg Cys Lys Gln Lys Val Ile Phe Gly Ser Gly Thr Val Thr Phe Ser>
          a a a a TRANSLATION OF PHCH4 [A] a a a a >
      2866 2876 2886 2896 2906
            * * * * * * * * *
    CTG AGC TTT GAT GAG CCT CAG AAG AAC GCC ATG GCC CAC GGG AAT TCT
    Leu Ser Phe Asp Glu Pro Gln Lys Asn Ala Met Ala His Gly Asn Ser>
          a a a a TRANSLATION OF PHCH4 [A] a a a a >
          2916 2926 2936 2946 2956
    * * * * * * * * * *
    ACG CAC CAG AAC TCC CTG GAG GCC CAG AAA AGC AGC GAT ACG CTG ACC
    Thr His Gln Asn Ser Leu Glu Ala Gln Lys Ser Ser Asp Thr Leu Thr>
          a a a a TRANSLATION OF PHCH4 [A] a a a a >
                2966 2976 2986 2996 3006
        * * * * * * * * *
    CGA CAC CAG CCA TTA CTC CCG CTG CAG TGC GGG GAA ACG GAC TTA GA
    Arg His Gln Pro Leu Leu Pro Leu Gln Cys Gly Glu Thr Asp Leu As
          a a a a TRANSLATION OF PHCH4 [A] a a a a >
                      3016 3026 3036 3046
              * * * * * * * * *
    CTG ACC GTC CAG GAA ACA GGT CTG CAA GGA CCT GTG GGT GGA GAC CA
    Leu Thr Val Gln Glu Thr Gly Leu Gln Gly Pro Val Gly Gly Asp Gl
          a a a a TRANSLATION OF PHCH4 [A] a a a a >
3056 3066 3076 3086 3096
      * * * * * * * * * *
    CGG CCA GAG GTG GAG GAC CCT GAA GAG TTG TCC CCA GCA CTT GTA GT
    Arg Pro Glu Val Glu Asp Pro Glu Glu Leu Ser Pro Ala Leu Val Val
          a a a a TRANSLATION OF PHCH4 [A] a a a a >
      3106 3116 3126 3136 3146
            * * * * * * * * *
    TCC AGT TCA CAG AGC TTT GTC ATC AGT GGT GGA GGC AGC ACT GTT AC
    Ser Ser Ser Gln Ser Phe Val Ile Ser Gly Gly Gly Ser Thr Val Th
          a a a a TRANSLATION OF PHCH4 [A] a a a a >
          3156 3166 3176 3186
    * * * * * * * * *
    GAA AAC GTA GTG AAT TCA T AAAATGG AAGGAGAAGA CTGGGCTAG
    Glu Asn Val Val Asn Ser Xxx>
          TRANSLATION OF PHC a >
```

The invention illustratively described herein may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entireties to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

Furthermore, where a set of values is used in connection with an embodiment or limitation, the set of values also describes a set of ranges where each such range is specified by taking two of the values from the set of values as the inclusive endpoints of the range. Likewise, where a set of ranges is described, the description includes additional ranges where each such additional range is specified by taking two different endpoints of the initially described ranges as the endpoints of such an additional range. Likewise, specification of a range of integer values is deemed to include description of each integer value within that range, including the endpoints.

The invention illustratively described herein may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entireties to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

Furthermore, where a set of values is used in connection with an embodiment or limitation, the set of values also describes a set of ranges where each such range is specified by taking two of the values from the set of values as the inclusive endpoints of the range. Likewise, where a set of ranges is described, the description includes additional ranges where each such additional range is specified by taking two different endpoints of the initially described ranges as the endpoints of such an additional range. Likewise, specification of a range of integer values is deemed to include description of each integer value within that range, including the endpoints.

Other embodiments are within the following claims.

1. A composition comprising a chimeric receptor, wherein said chimeric receptor comprises a signal peptide, an extracellular domain, a transmembrane domain, and an intracellular domain, wherein
said extracellular domain comprises a sequence at least 20 amino acid residues in length that is at least 70% identical to a metabotropic glutamate receptor (mGluR) extracellular domain sequence, wherein said extracellular domain is linked to a non-native signal peptide;
said transmembrane domain comprises a sequence at least 70% identical to a mGluR transmembrane domain or a CaR transmembrane domain; and
said intracellular domain, when present, comprises a sequence at least 10 residues in length of a mGluR intracellular domain or a CaR intracellular domain.

2. The composition of claim 1, wherein said intracellular domain is present and is fused to a G-protein that links to phospholipase-C.

3. The composition of claim 1, wherein said intracellular domain is absent and said transmembrane domain is fused to a G-protein that links to phospholipase-C.

4. The composition of claim 1, wherein said extracellular domain sequence is from an mGluR7.

5. The composition of claim 1, wherein said extracellular domain sequence is from human mGluR7.

6. The composition of claim 1, wherein said extracellular domain sequence is from an mGluR2.

7. The composition of claim 1, where said extracellular domain sequence is from human mGluR2.

8. The composition of claim 1, wherein said extracellular domain sequence and said transmembrane domain sequence are from an mGluR7.

9. The composition of claim 1, wherein said extracellular domain sequence and said transmembrane domain sequence are from a human mGluR7.

10. The composition of claim 1, wherein said extracellular domain sequence and said transmembrane domain sequence are from an mGluR2.

11. The composition of claim 1, wherein said extracellular domain sequence and said transmembrane domain sequence are from a human mGluR2.

12. The composition of claim 1, wherein said signal peptide comprises a sequence at least 70% identical to a CaR signal peptide sequence.

13. The composition of claim 1, wherein said signal peptide is from a CaR, and said extracellular domain, transmembrane domain, and cytoplasmic tail domain are from a mGluR.

14. The composition of claim 1, wherein said signal peptide is from a CaR, and said extracellular domain, transmembrane domain, and cytoplasmic tail domain are from a mGluR7.

15. The composition of claim 1, wherein said signal peptide is from a CaR, and said extracellular domain, transmembrane domain, and cytoplasmic tail domain are from a human mGluR7.

16. The composition of claim 1, wherein said signal peptide is from an mGluR.

17. The composition of claim 1, wherein said signal peptide is from mGluR8.

18. The composition of claim 1, wherein said chimeric receptor comprises a sequence at least CaR signal peptide linked to an mGluR7 amino acid residue in the range of residues 36-50.

19. The composition of claim 1, wherein said chimeric receptor comprises a sequence at least CaR signal peptide linked to an mGluR7 amino acid residue in the range of residues 40-50.

20. A chimeric receptor comprising a calcium receptor (CaR) signal peptide, an extracellular domain, a transmembrane domain, and an intracellular domain, wherein
said extracellular domain comprises a sequence at least 20 amino acid residues in length that is at least 70% identical to a metabotropic glutamate receptor (mGluR) extracellular domain sequence, wherein said extracellular domain is linked to a non-native signal peptide;
said transmembrane domain comprises a sequence at least 70% identical to a mGluR transmembrane domain or a CaR transmembrane domain; and
said intracellular domain, when present, comprises a sequence at least 10 residues in length of a mGluR intracellular domain or a CaR intracellular domain.

21. A chimeric receptor as specified in any one of claims 1-19.

22. A composition comprising a nucleic acid molecule coding for the chimeric receptor as specified in any one of claims 1-19.

23. A replicable expression vector comprising a nucleic acid molecule coding for the chimeric receptor as specified in any one of claims 1-19.

24. A method of manufacturing a chimeric receptor, comprising
growing under suitable nutrient conditions prokaryotic or eukaryotic host cells transformed or transfected with an expression vector comprising a nucleic acid sequence encoding a chimeric receptor as specified in any one of claims 1-19.

25. A method of screening for a compound that binds to or modulates the activity of a metabotropic glutamate receptor, comprising
contacting a chimeric receptor as specified in any one of claims 1-19 with a test compound in an acceptable medium and determining whether said test compound binds to or modulates said chimeric receptor, wherein said binding or modulation is indicative that said test compound binds to or modulates said metabotropic glutamate receptor.

26. A method of screening for a compound that binds to or modulates the activity of a metabotropic glutamate receptor, comprising
introducing a host cell expressing a chimeric receptor as specified in any one of claims 1-19 into an acceptable medium with a test compound; and
monitoring an effect in said host cell indicative of binding or modulation of said test compound with said chimeric receptor, wherein said binding or modulation is indicative that said test compound binds to or modulates said metabotropic glutamate receptor.

27. A kit comprising a host cell transformed or transfected with an expression vector comprising a nucleic acid sequence encoding a chimeric receptor as specified in any one of claims 1-19 in a container.

28. The kit of claim 27, further comprising a growth medium.
